(19) United States
(12) Patent Application Publication
(10) Pub. No.: US 2020/0157086 A1
(43) Pub. Date: May 21, 2020

(54) BENZAMIDE COMPOUNDS AND THEIR USE AS HERBICIDES

(71) Applicant: BASF SE, Ludwigshafen am Rhein (DE)

(72) Inventors: Markus KORDES, Ludwigshafen (DE); Thomas ZIERKE, Ludwigshafen (DE); Thomas SEITZ, Ludwigshafen (DE)

(21) Appl. No.: 16/617,437

(22) PCT Filed: May 29, 2018
(86) PCT No.: PCT/EP2018/064045

§ 371 Date: Nov. 26, 2019

(30) Foreign Application Priority Data
May 30, 2017 (EP) 17173417.1

(51) Int. Cl. C07D 413/12; A01N 43/84; A01N 43/82; A01N 43/713; A01N 43/653; A01N 47/30; C07D 271/113; C07D 257/06; C07D 249/14

(52) U.S. Cl. CPC C07D 413/12; A01N 43/84; A01N 43/82; A01N 43/713; C07D 249/14; A01N 47/30; C07D 271/113; C07D 257/06; A01N 43/653

(57) ABSTRACT

The invention relates to benzamides of formula I,

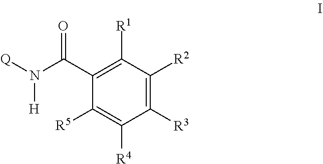

wherein
Q is Q¹ or Q² or Q³ or Q⁴,

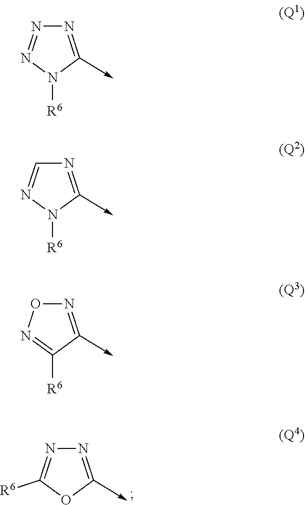

and their use as herbicides. In said formula I, R² is R^{2c}R^{2d}NC(O)NR²ⁿ—Z², both R⁴ and R⁵ are hydrogen, R¹, R³ and R⁶ represent groups such as hydrogen, halogen or organic groups such as alkyl or phenyl. The invention further refers to a composition comprising such compound and to the use thereof for controlling unwanted vegetation.

The present invention relates to benzamide compounds and salts thereof and to compositions comprising the same. The invention also relates to the use of the benzamide compounds or of the compositions comprising such compounds for controlling unwanted vegetation. Furthermore, the invention relates to methods of applying such compounds.

For the purposes of controlling unwanted vegetation, especially in crops, there is an ongoing need for new herbicides which have high activities and selectivities together with a substantial lack of toxicity for humans and animals.

WO 2011/035874 describes N-(1,2,5-oxadiazol-3-yl)benzamides carrying 3 substituents in the 2-, 3- and 4-positions of the phenyl ring and their use as herbicides.

WO 2012/028579 describes N-(tetrazol-4-yl)- and N-(triazol-3-yl)arylcarboxylic acid amides carrying 3 substituents in the 2-, 3- and 4-positions of the aryl ring and their use as herbicides.

WO2013/017559 describes N-(tetrazol-5-yl)- and N-(triazol-5-yl)arylcarboxylic acid amides carrying 3 substituents in the 2-, 3- and 4-positions of the aryl ring and their use as herbicides.

WO2013/124245 describes N-(tetrazol-5-yl)-, N-(triazol-5-yl)- and N-(1,3,4-oxadiazol-2-yl)arylcarboxylic acid amides carrying 1, 2, or 3 substituents in the 2-, 3- and 5-positions of the aryl ring and a nitro group in the 4 position of the aryl ring and their use as herbicides.

The compounds of the prior art often suffer from insufficient herbicidal activity in particular at low application rates and/or unsatisfactory selectivity resulting in a low compatibility with crop plants.

Accordingly, it is an object of the present invention to provide further benzamide compounds having a strong herbicidal activity, in particular even at low application rates, a sufficiently low toxicity for humans and animals and/or a high compatibility with crop plants. The benzamide compounds should also show a broad activity spectrum against a large number of different unwanted plants.

These and further objectives are achieved by the compounds of formula I defined below and their agriculturally suitable salts.

Therefore, in a first aspect the present invention relates to compounds of formula I,

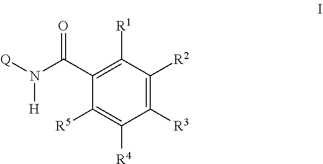

wherein
Q is Q¹ or Q² or Q³ or Q⁴,

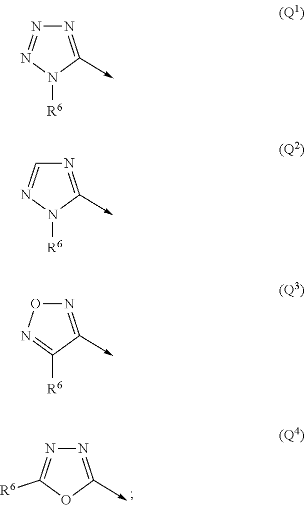

R¹ is selected from the group consisting of cyano, halogen, nitro, C₁-C₈-alkyl, C₁-C₈-haloalkyl, C₁-C₄-alkoxy-C₁-C₄-alkyl, C₁-C₄-haloalkoxy-C₁-C₄-alkyl, C₁-C₈-alkoxy, C₁-C₄-alkoxy-C₁-C₄-alkoxy-Z¹, C₁-C₆-haloalkoxy, R^{1b}—S(O)ₖ—Z¹;
R² is R^{2c}R^{2d}NC(O)NR²ⁿ—Z²;
R³ is selected from the group consisting of hydrogen, cyano, thiocyanato, halogen, hydroxy-Z², nitro, C₁-C₆-alkyl, C₂-C₈-alkenyl, C₂-C₈-alkynyl, C₃-C₁₀-cycloalkyl-Z², C₃-C₆-cycloalkenyl-Z², C₃-C₁₀-cycloalkoxy-Z², C₃-C₁₀-cycloalkyl-C₁-C₂-alkoxy, where the cyclic groups of the four aforementioned radicals are unsubstituted or partially or completely halogenated, C₁-C₄-cyanoalkyl, C₁-C₈-haloalkyl, C₂-C₈-haloalkenyl, C₃-C₈-haloalkynyl, C₁-C₈-alkoxy-Z², C₁-C₈-haloalkoxy-Z², C₁-C₄-alkoxy-C₁-C₄-alkoxy-Z², C₁-C₄-haloalkoxy-C₁-C₄-alkoxy-Z², C₂-C₈-alkenyloxy-Z², C₂-C₈-alkynyloxy-Z², C₂-C₈-haloalkenyloxy-Z², C₃-C₈-haloalkynyloxy-Z², R^{2b}—S(O)ₖ—Z², R^{2c}—C(O)—Z², R^{2d}O—C(O)Z², R^{2d}O—N═CH—Z², R^{2e}R^{2f}N—C(O)—Z², R^{2g}R^{2h}N—Z², R²²C(O)O—Z², R²⁵OC(O)O—Z², (R²²)₂NC(O)O—Z², R²⁵S(O)₂O—Z², R²²OS(O)₂—Z², (R²²)₂NS(O)₂—Z², R²⁵OC(O)N(R²²)—Z², (R²²)₂NC(O)N(R²²)—Z², (R²²)₂NS(O)₂N(R²²)—Z², (OH)₂P(O)—Z², (C₁-C₄-alkoxy)₂P(O)—Z², phenyl-Z^{2a}, heterocyclyl-Z^{2a}, where heterocyclyl is a 3-, 4-, 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where cyclic groups in phenyl-Z^{2a} and heterocyclyl-Z^{2a} are unsubstituted or substituted by 1, 2, 3 or 4 groups R²¹, which are identical or different;
R⁴ is hydrogen;
R⁵ is hydrogen;
R⁶ is selected from the group consisting of cyano, C₁-C₆-alkyl, C₃-C₇-cycloalkyl, C₃-C₇-cycloalkyl-C₁-C₄-alkyl, where the C₃-C₇-cycloalkyl groups of the two aforementioned radicals are unsubstituted or partially or completely halogenated, C₁-C₆-haloalkyl, C₂-C₆-alkenyl, C₂-C₆-alkynyl, C₂-C₆-haloalkenyl, C₃-C₆-haloalkynyl, C₁-C₄-alkoxy-C₁-C₄-alkyl, C₁-C₄-haloalkoxy-C₁-C₄-alkyl, R^{b}—S(O)ₙ—C₁-C₃-alkyl, phenyl and benzyl, where phenyl and benzyl are unsubstituted or substituted by 1, 2, 3 or 4 groups, which are identical or different and selected from the group consisting of halogen, C₁—C₄-alkyl, C₁-C₄-haloalkyl, C₁-C₄-alkoxy and C₁-C₄-haloalkoxy;
R²¹ is selected from the group consisting of cyano, halogen, nitro, C₁-C₆-alkyl, C₃-C₇-cycloalkyl, C₃-C₇-halocycloalkyl, C₁-C₆-haloalkyl, C₂-C₆-alkenyl, C₂-C₆-haloalkenyl, C₂-C₆-alkynyl, C₃-C₆-haloalkynyl, C₁-C₆-alkoxy, C₁-C₄-alkoxy-C₁-C₄-alkyl, C₁-C₄-alkylthio-C₁-C₄-alkyl, C₁-C₄-haloalkoxy-C₁-C₄-alkyl, C₁-C₄-alkoxy-C₁-C₄-alkoxy, C₃-C₇-cycloalkoxy and C₁-C₆-haloalkoxy, or two radicals R²¹ bound to the same carbon atom together may form a group ═O;
Z¹, Z² independently of each other are selected from the group consisting of a covalent bond and C₁-C₄-alkanediyl;
Z^{2a} is selected from the group consisting of a covalent bond, C₁-C₄-alkanediyl, O—C₁-C₄-alkanediyl, C₁-C₄-alkanediyl-O and C₁-C₄-alkanediyl-O—C₁-C₄-alkanediyl;
R^{b}, R^{1b}, R^{2b} independently of each other are selected from the group consisting of C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₃-C₆-haloalkynyl, phenyl and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2c} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₁-C₆-alkoxy, C₂-C₆-alkenyl, C₁-C₄-alkyl-C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylS(O)ₙ—C₁-C₄-alkyl, C₁-C₄-alkylamino-C₁-C₄-alkyl, C₁-C₄-dialkylamino-C₁-C₄-alkyl, C₁-C₆-cyanoalkyl, phenyl, benzyl and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, benzyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2d} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁—C₆-haloalkyl, C₁-C₆-alkoxy, C₂-C₆-alkenyl, C₁-C₄-alkyl-C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylS(O)ₙ—C₁-C₄-alkyl, C₁-C₄-alkylamino-C₁-C₄-alkyl, C₁-C₄-dialkylamino-C₁-C₄-alkyl, C₁-C₆-cyanoalkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2c}, R^{2d} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R²ⁿ is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-cyanoalkyl, C₁-C₆-haloalkyl, C₂-C₆-alkenyl, C₂-C₆-haloalkenyl, C₂-C₆-alkynyl, C₁-C₄-alkoxy-C₁-C₄-alkyl;
R^{2e}, R^{2f} independently of each other are selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2e}, R^{2f} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R^{2g} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylsulfonyl, C₁-C₄-alkylcarbonyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2h} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylsulfonyl, C₁-C₄-alkylcarbonyl, a radical C(O)R^{k}, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2g}, R^{2h} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R²² is selected from the group consisting of hydrogen, C₁-C₆-alkyl, C₁-C₆-haloalkyl, C₂-C₆-alkenyl, C₂-C₆-haloalkenyl, C₂-C₆-alkynyl, C₃-C₆-haloalkynyl, C₃-C₆-cycloalkyl, C₃-C₆-cycloalkenyl, C₃-C₆-halocycloalkyl, C₃-C₆-cycloalkyl-C₁-C₆-alkyl, C₁-C₆-alkoxy-C₁-C₆-alkyl, C₃-C₆-cycloalkyl-C₁-C₆-alkoxy-C₁-C₆-alkyl, phenyl-Z¹, phenyl-O—C₁-C₆-alkyl, phenyl-N(R²³)—C₁-C₆-alkyl, phenyl-S(O)ₙ—C₁-C₆-alkyl, heterocyclyl-Z¹, heterocyclyl-N(R²³)—C₁-C₆-alkyl, heterocyclyl-O—C₁-C₆-alkyl, heterocyclyl-S(O)ₙ—C₁-C₆-alkyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, heterocyclyl are unsubstituted or substituted by 1, 2, 3 or 4 groups, which are identical or different and selected from the group consisting of cyano, halogen, nitro, thiocyanato, C₁-C₆-alkyl, C₁-C₆-haloalkyl, C₃-C₆-cycloalkyl, C(O)OR²³, C(O)N(R²³)₂, OR²³, N(R²³)₂, S(O)ₙR²⁴, S(O)₂OR²³, S(O)₂N(R²³)₂, and R²³O—C₁-C₆-alkyl, and where heterocyclyl bears 0, 1 or 2 oxo groups;
R²³ is selected from the group consisting of hydrogen, C₁-C₆-alkyl, C₁-C₆-haloalkyl, C₂-C₆-alkenyl, C₂-C₆-alkynyl, C₃-C₆-cycloalkyl, C₃-C₆-cycloalkyl-C₁-C₆-alkyl, and phenyl;
R²⁴ is selected from the group consisting of C₁-C₆-alkyl, C₁-C₆-haloalkyl, C₂-C₆-alkenyl, C₂-C₆-alkynyl, C₃-C₆-cycloalkyl, C₃-C₆-cycloalkyl-C₁-C₆-alkyl, and phenyl;
R²⁵ is selected from the group consisting of C₁-C₆-alkyl, C₁-C₆-haloalkyl, C₂-C₆-alkenyl, C₂-C₆-haloalkenyl, C₂-C₆-alkynyl, C₃-C₆-haloalkynyl, C₃-C₆-cycloalkyl, C₃-C₆-cycloalkenyl, C₃-C₆-halocycloalkyl, C₃-C₆-cycloalkyl-C₁-C₆-alkyl, C₁-C₆-alkoxy-C₁-C₆-alkyl, C₃-C₆-cycloalkyl-C₁-C₆-alkoxy-C₁-C₆-alkyl, phenyl-Z¹, phenyl-O—C₁-C₆-alkyl, phenyl-N(R²³)—C₁-C₆-alkyl, phenyl-S(O)ₙ—C₁-C₆-alkyl, heterocyclyl-Z¹, heterocyclyl-N(R²³)—C₁-C₆-alkyl, heterocyclyl-O—C₁-C₆-alkyl, heterocyclyl-S(O)ₙ—C₁-C₆-alkyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, heterocyclyl are unsubstituted or substituted by 1, 2, 3 or 4 groups, which are identical or different and selected from the group consisting of cyano, halogen, nitro, thiocyanato, C₁-C₆-alkyl, C₁-C₆-haloalkyl, C₃-C₆-cycloalkyl, C(O)OR²³, C(O)N(R²³)₂, OR²³, N(R²³)₂, S(O)ₙR²⁴, S(O)₂OR²³, S(O)₂N(R²³)₂, and R²³O—C₁-C₆-alkyl, and where heterocyclyl bears 0, 1 or 2 oxo groups;
k is 0, 1 or 2;
n is 0, 1 or 2;
R^{k} has the meanings of R^{2c};
   with the exception of
Benzamide, 4-bromo-2-chloro-N-[1-(2-methoxyethyl)-1H-tetrazol-5-yl]-3-[[(methoxymethylamino)carbonyl]amino]-, CAS Registry Number: 1421777-21-0;
Benzamide, 2,4-dichloro-N-[1-(2-methoxyethyl)-1H-tetrazol-5-yl]-3-[[(methoxymethylamino)carbonyl]amino]-, CAS Registry Number: 1421776-96-6;
Benzamide, 2,4-dichloro-3-[[(methoxymethylamino)carbonyl]amino]-N-(1-phenyl-1H-tetrazol-5-yl)-, CAS Registry Number: 1361245-23-9;
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6;
Benzamide, 2,4-dichloro-3-[[(dimethylamino)carbonyl]amino]-N-(1-methyl-1H-tetrazol-5-yl)-, CAS Registry Number: 1361139-93-6;
Benzamide, 2,4-dichloro-3-[[(methoxymethylamino)carbonyl]amino]-N-(1-phenyl-1H-1,2,4-triazol-5-yl)-, CAS Registry Number: 1361103-06-1;
Benzamide, 2,4-dichloro-N-(1-ethyl-1H-1,2,4-triazol-5-yl)-3-[[(methoxymethylamino)carbonyl]amino]-, CAS Registry Number: 1361040-89-2;
Benzamide, N-(4-cyano-1,2,5-oxadiazol-3-yl)-2-methyl-3-[[[methyl(2-methylpropyl)amino]carbonyl]amino]-4-(methylsulfonyl)-, CAS Registry Number: 1279687-63-6;
Benzamide, 2-methyl-3-[[[methyl(2-methylpropyl)amino]carbonyl]amino]-4-(methylsulfonyl)-N-[4-(trifluoromethyl)-1,2,5-oxadiazol-3-yl]-, CAS Registry Number: 1279679-31-0;
Benzamide, N-[4-(1,1-dimethylethyl)-1,2,5-oxadiazol-3-yl]-2-methyl-3-[[[methyl(2-methylpropyl)amino]carbonyl]amino]-4-(methylsulfonyl)-, CAS Registry Number: 1279665-87-0;
Benzamide, N-[4-(chloromethyl)-1,2,5-oxadiazol-3-yl]-2-methyl-3-[[[methyl(2-methylpropyl)amino]carbonyl]amino]-4-(methylsulfonyl)-, CAS Registry Number: 1279659-60-7;
Benzamide, 2-methyl-3-[[[methyl(2-methylpropyl)amino]carbonyl]amino]-4-(methylsulfonyl)-N-(4-propyl-1,2,5-oxadiazol-3-yl)-, CAS Registry Number: 1279644-16-4;
Benzamide, 2-methyl-N-[4-(1-methylethyl)-1,2,5-oxadiazol-3-yl]-3-[[[methyl(2-methylpropyl)amino]carbonyl]amino]-4-(methylsulfonyl)-, CAS Registry Number: 1279635-24-3;
Benzamide, N-(4-ethyl-1,2,5-oxadiazol-3-yl)-2-methyl-3-[[[methyl(2-methylpropyl)amino]carbonyl]amino]-4-(methylsulfonyl)-, CAS Registry Number: 1279127-99-9;
Benzamide, 2-methyl-3-[[[methyl(2-methylpropyl)amino]carbonyl]amino]-N-(4-methyl-1,2,5-oxadiazol-3-yl)-4-(methylsulfonyl)-, CAS Registry Number: 1279114-20-3.

The compounds of the present invention, i.e. the compounds of formula I and their agriculturally suitable salts, are particularly useful for controlling unwanted vegetation. Therefore, the invention also relates to the use of a compound of formula I or an agriculturally suitable salt thereof or of a composition comprising at least one compound of formula I or an agriculturally suitable salt thereof for combating or controlling unwanted vegetation.

The invention also relates to a composition comprising at least one compound of formula I, or a salt thereof, and at least one auxiliary. In particular, the invention relates to an agricultural composition comprising at least one compound of formula I or an agriculturally suitable salt thereof, and at least one auxiliary customary for crop protection formulations.

The present invention also relates to a method for combating or controlling unwanted vegetation, which method comprises allowing a herbicidally effective amount of at least one compound of formula I or a salt thereof, to act on unwanted plants, their seed and/or their habitat.

Depending on the substitution pattern, the compounds of formula I may have one or more centers of chirality, in which case they are present as mixtures of enantiomers or diastereomers. The invention provides both the pure enantiomers or pure diastereomers of the compounds of formula I, and their mixtures and the use according to the invention of the pure enantiomers or pure diastereomers of the compound of formula I or its mixtures. Suitable compounds of formula I also include all possible geometrical stereoisomers (cis/trans isomers) and mixtures thereof. Cis/trans isomers may be present with respect to an alkene, carbon-nitrogen double-bond, nitrogen-sulfur double bond or amide group. The term “stereoisomer(s)” encompasses both optical isomers, such as enantiomers or diastereomers, the latter existing due to more than one center of chirality in the molecule, as well as geometrical isomers (cis/trans isomers).

Depending on the substitution pattern, the compounds of formula I may be present in the form of their tautomers. Hence the invention also relates to the tautomers of compounds of formula I and the stereoisomers and salts of said tautomers.

The present invention moreover relates to compounds as defined herein, wherein one or more of the atoms depicted in formula I have been replaced by its stable, preferably nonradioactive isotope (e.g., hydrogen by deuterium, ¹²C by ¹³C, ¹⁴N by ¹⁵N, ¹⁶O by ¹⁸O) and in particular wherein at least one hydrogen atom has been replaced by a deuterium atom. Of course, the compounds according to the invention contain more of the respective isotope than this naturally occurs and thus is anyway present in the compounds of formula I.

The compounds of the present invention may be amorphous or may exist in one ore more different crystalline states (polymorphs) which may have different macroscopic properties such as stability or show different biological properties such as activities. The present invention includes both amorphous and crystalline compounds of formula I, their enantiomers or diastereomers, mixtures of different crystalline states of the respective compound of formula I, its enantiomers or diastereomers, as well as amorphous or crystalline salts thereof.

Salts of the compounds of the present invention are preferably agriculturally suitable salts. They can be formed in a customary method, e.g. by reacting the compound with an acid if the compound of the present invention has a basic functionality or by reacting the compound with a suitable base if the compound of the present invention has an acidic functionality.

Useful agriculturally suitable salts are especially the salts of those cations or the acid addition salts of those acids whose cations and anions, respectively, do not have any adverse effect on the herbicidal action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of the present invention with an acid of the corresponding anion, preferably with hydrochloric acid, hydrobromic acid, sulfuric acid, phosphoric acid or nitric acid.

The term “undesired vegetation” (“weeds”) is understood to include any vegetation growing in non-crop-areas or at a crop plant site or locus of seeded and otherwise desired crop, where the vegetation is any plant species, including their germinant seeds, emerging seedlings and established vegetation, other than the seeded or desired crop (if any). Weeds, in the broadest sense, are plants considered undesirable in a particular location.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “partially or completely halogenated” will be taken to mean that 1 or more, e.g. 1, 2, 3, 4 or 5 or all of the hydrogen atoms of a given radical have been replaced by a halogen atom, in particular by fluorine or chlorine. A partially or completely halogenated radical is termed below also “halo-radical”. For example, partially or completely halogenated alkyl is also termed haloalkyl.

The term “alkyl” as used herein (and in the alkyl moieties of other groups comprising an alkyl group, e.g. alkoxy, alkylamino, alkylcarbonyl, alkoxycarbonyl, alkylthio, alkylsulfonyl and alkoxyalkyl) denotes in each case a straight-chain or branched alkyl group having usually from 1 to 10 carbon atoms, frequently from 1 to 6 carbon atoms, preferably 1 to 4 carbon atoms and in particular from 1 to 3 carbon atoms. Examples of C₁-C₄-alkyl are methyl, ethyl, n-propyl, isopropyl, n-butyl, 2-butyl (sec-butyl), isobutyl and tert-butyl. Examples for C₁-C₆-alkyl are, apart those mentioned for C₁-C₄-alkyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Examples for C₁-C₁₀-alkyl are, apart those mentioned for C₁-C₆-alkyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, n-octyl, 1-methyloctyl, 2-methylheptyl, 1-ethylhexyl, 2-ethylhexyl, 1,2-dimethylhexyl, 1-propylpentyl, 2-propylpentyl, nonyl, decyl, 2-propylheptyl and 3-propylheptyl.

The term “alkylene” (or alkanediyl) as used herein in each case denotes an alkyl radical as defined above, wherein one hydrogen atom at any position of the carbon backbone is replaced by one further binding site, thus forming a bivalent moiety.

The term “haloalkyl” as used herein (and in the haloalkyl moieties of other groups comprising a haloalkyl group, e.g. haloalkoxy, haloalkylthio, haloalkylcarbonyl, haloalkylsulfonyl and haloalkylsulfinyl) denotes in each case a straight-chain or branched alkyl group having usually from 1 to 8 carbon atoms (“C₁-C₈-haloalkyl”), frequently from 1 to 6 carbon atoms (“C₁-C₆-haloalkyl”), more frequently 1 to 4 carbon atoms (“C₁-C₄-haloalkyl”), wherein the hydrogen atoms of this group are partially or totally replaced with halogen atoms. Preferred haloalkyl moieties are selected from C₁-C₄-haloalkyl, more preferably from C₁-C₂-haloalkyl, more preferably from halomethyl, in particular from C₁-C₂-fluoroalkyl. Halomethyl is methyl in which 1, 2 or 3 of the hydrogen atoms are replaced by halogen atoms. Examples are bromomethyl, chloromethyl, dichloromethyl, trichloromethyl, fluoromethyl, difluoromethyl, trifluoromethyl, chlorofluoromethyl, dichlorofluoromethyl, chlorodifluoromethyl and the like. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl, pentafluoroethyl, and the like. Examples for C₁-C₂-haloalkyl are, apart those mentioned for C₁-C₂-fluoroalkyl, chloromethyl, dichloromethyl, trichloromethyl, bromomethyl, chlorofluoromethyl, dichlorofluoromethyl, chlorodifluoromethyl, 1-chloroethyl, 2-chloroethyl, 2,2,dichloroethyl, 2,2,2-trichloroethyl, 2-chloro-2-fluoroethyl, 2-chloro-2,2-difluoroethyl, 2,2-dichloro-2-fluoroethyl, 1-bromoethyl, and the like. Examples for C₁-C₄-haloalkyl are, apart those mentioned for C₁-C₂-haloalkyl, 1-fluoropropyl, 2-fluoropropyl, 3-fluoropropyl, 3,3-difluoropropyl, 3,3,3-trifluoropropyl, heptafluoropropyl, 1,1,1-trifluoroprop-2-yl, 3-chloropropyl, 4-chlorobutyl and the like.

The term “cycloalkyl” as used herein (and in the cycloalkyl moieties of other groups comprising a cycloalkyl group, e.g. cycloalkoxy and cycloalkylalkyl) denotes in each case a mono- or bicyclic cycloaliphatic radical having usually from 3 to 10 carbon atoms (“C₃-C₁₀-cycloalkyl”), preferably 3 to 7 carbon atoms (“C₃-C₇-cycloalkyl”) or in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 7 carbon atoms comprise cyclopropyl, cyclobutyl, cyclopentyl, cyclohexyl and cycloheptyl. Examples of bicyclic radicals having 7 or 8 carbon atoms comprise bicyclo[2.1.1]hexyl, bicyclo[2.2.1]heptyl, bicyclo[3.1.1]heptyl, bicyclo[2.2.1]heptyl, bicyclo[2.2.2]octyl and bicyclo[3.2.1]octyl.

The term “halocycloalkyl” as used herein (and in the halocycloalkyl moieties of other groups comprising an halocycloalkyl group, e.g. halocycloalkylmethyl) denotes in each case a mono- or bicyclic cycloaliphatic radical having usually from 3 to 10 carbon atoms, preferably 3 to 7 carbon atoms or in particular 3 to 6 carbon atoms, wherein at least one, e.g. 1, 2, 3, 4 or 5 of the hydrogen atoms are replaced by halogen, in particular by fluorine or chlorine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “cycloalkyl-alkyl” used herein denotes a cycloalkyl group, as defined above, which is bound to the remainder of the molecule via an alkylene group. The term “C₃-C₇-cycloalkyl-C₁-C₄-alkyl” refers to a C₃-C₇-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “alkenyl” as used herein denotes in each case a monounsaturated straight-chain or branched hydrocarbon radical having usually 2 to 8 (“C₂-C₈-alkenyl”), preferably 2 to 6 carbon atoms (“C₂-C₆-alkenyl”), in particular 2 to 4 carbon atoms (“C₂-C₄-alkenyl”), and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₈-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl and the positional isomers thereof.

The term “haloalkenyl” as used herein, which may also be expressed as “alkenyl which is substituted by halogen”, and the haloalkenyl moieties in haloalkenyloxy and the like refers to unsaturated straight-chain or branched hydrocarbon radicals having 2 to 8 (“C₂-C₈-haloalkenyl”) or 2 to 6 (“C₂-C₆-haloalkenyl”) or 2 to 4 (“C₂-C₄-haloalkenyl”) carbon atoms and a double bond in any position, where some or all of the hydrogen atoms in these groups are replaced by halogen atoms as mentioned above, in particular fluorine, chlorine and bromine, for example chlorovinyl, chloroallyl and the like.

The term “alkynyl” as used herein denotes unsaturated straight-chain or branched hydrocarbon radicals having usually 2 to 8 (“C₂-C₈-alkynyl”), frequently 2 to 6 (“C₂-C₆-alkynyl”), preferably 2 to 4 carbon atoms (“C₂-C₄-alkynyl”) and a triple bond in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “haloalkynyl” as used herein, which is also expressed as “alkynyl which is substituted by halogen”, refers to unsaturated straight-chain or branched hydrocarbon radicals having usually 3 to 8 carbon atoms (“C₃-C₈-haloalkynyl”), frequently 3 to 6 (“C₃-C₆-haloalkynyl”), preferably 3 to 4 carbon atoms (“C₃-C₄-haloalkynyl”), and a triple bond in any position (as mentioned above), where some or all of the hydrogen atoms in these groups are replaced by halogen atoms as mentioned above, in particular fluorine, chlorine and bromine.

The term “alkoxy” as used herein denotes in each case a straight-chain or branched alkyl group usually having from 1 to 8 carbon atoms (“C₁-C₈-alkoxy”), frequently from 1 to 6 carbon atoms (“C₁-C₆-alkoxy”), preferably 1 to 4 carbon atoms (“C₁-C₄-alkoxy”), which is bound to the remainder of the molecule via an oxygen atom. C₁-C₂-Alkoxy is methoxy or ethoxy. C₁-C₄-Alkoxy is additionally, for example, n-propoxy, 1-methylethoxy (isopropoxy), butoxy, 1-methylpropoxy (sec-butoxy), 2-methylpropoxy (isobutoxy) or 1,1-dimethylethoxy (tert-butoxy).

C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof.

The term “haloalkoxy” as used herein denotes in each case a straight-chain or branched alkoxy group, as defined above, having from 1 to 8 carbon atoms (“C₁-C₈-haloalkoxy”), frequently from 1 to 6 carbon atoms (“C₁-C₆-haloalkoxy”), preferably 1 to 4 carbon atoms (“C₁-C₄-haloalkoxy”), more preferably 1 to 3 carbon atoms (“C₁-C₃-haloalkoxy”), wherein the hydrogen atoms of this group are partially or totally replaced with halogen atoms, in particular fluorine atoms. C₁-C₂-Haloalkoxy is, for example, OCH₂F, OCHF₂, OCF₃, OCH₂Cl, OCHC₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “alkoxyalkyl” as used herein denotes in each case alkyl usually comprising 1 to 6 carbon atoms, preferably 1 to 4 carbon atoms, wherein 1 carbon atom carries an alkoxy radical usually comprising 1 to 8, frequently 1 to 6, in particular 1 to 4, carbon atoms as defined above. “C₁-C₆-alkoxy-C₁-C₆-alkyl” is a C₁-C₆-alkyl group, as defined above, in which one hydrogen atom is replaced by a C₁-C₆-alkoxy group, as defined above. Examples are CH₂OCH₃, CH₂—OC₂H₅, n-propoxymethyl, CH₂—OCH(CH₃)₂, n-butoxymethyl, (1-methylpropoxy)-methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)-ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)propyl, 2-(1,1-dimethylethoxy)-propyl, 3-(methoxy)-propyl, 3-(ethoxy)-propyl, 3-(n-propoxy)propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(npropoxy)-butyl, 2-(1-methylethoxy)-butyl, 2-(n-butoxy)-butyl, 2-(1-methyl propoxy)-butyl, 2-(2-methyl-propoxy)-butyl, 2-(1,1-dimethylethoxy)-butyl, 3-(methoxy)-butyl, 3-(ethoxy)-butyl, 3-(npropoxy)-butyl, 3-(1-methylethoxy)-butyl, 3-(n-butoxy)-butyl, 3-(1-methylpropoxy)-butyl, 3-(2-methylpropoxy)-butyl, 3-(1,1-dimethylethoxy)-butyl, 4-(methoxy)-butyl, 4-(ethoxy)-butyl, 4-(npropoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “haloalkoxy-alkyl” as used herein denotes in each case alkyl as defined above, usually comprising 1 to 6 carbon atoms, preferably 1 to 4 carbon atoms, wherein 1 carbon atom carries an haloalkoxy radical as defined above, usually comprising 1 to 8, frequently 1 to 6, in particular 1 to 4, carbon atoms as defined above. Examples are fluoromethoxymethyl, difluoromethoxymethyl, trifluoromethoxymethyl, 1-fluoroethoxymethyl, 2-fluoroethoxymethyl, 1,1-difluoroethoxymethyl, 1,2-difluoroethoxymethyl, 2,2-difluoroethoxymethyl, 1,1,2-trifluoroethoxymethyl, 1,2,2-trifluoroethoxymethyl, 2,2,2-trifluoroethoxymethyl, pentafluoroethoxymethyl, 1-fluoroethoxy-1-ethyl, 2-fluoroethoxy-1-ethyl, 1,1-difluoroethoxy-1-ethyl, 1,2-difluoroethoxy-1-ethyl, 2,2-difluoroethoxy-1-ethyl, 1,1,2-trifluoroethoxy-1-ethyl, 1,2,2-trifluoroethoxy-1-ethyl, 2,2,2-trifluoroethoxy-1-ethyl, pentafluoroethoxy-1-ethyl, 1-fluoroethoxy-2-ethyl, 2-fluoroethoxy-2-ethyl, 1,1-difluoroethoxy-2-ethyl, 1,2-difluoroethoxy-2-ethyl, 2,2-difluoroethoxy-2-ethyl, 1,1,2-trifluoroethoxy-2-ethyl, 1,2,2-trifluoroethoxy-2-ethyl, 2,2,2-trifluoroethoxy-2-ethyl, pentafluoroethoxy-2-ethyl, and the like.

The term “alkylthio” (also alkylsulfanyl, “alkyl-S” or “alkyl-S(O)ₖ” (wherein k is 0)) as used herein denotes in each case a straight-chain or branched saturated alkyl group as defined above, usually comprising 1 to 8 carbon atoms (“C₁-C₈-alkylthio”), frequently comprising 1 to 6 carbon atoms (“C₁-C₆-alkylthio”), preferably 1 to 4 carbon atoms (“C₁-C₄-alkylthio”), which is attached via a sulfur atom at any position in the alkyl group. C₁-C₂-Alkylthio is methylthio or ethylthio. C₁-C₄-Alkylthio is additionally, for example, n-propylthio, 1-methylethylthio (isopropylthio), butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof.

The term “haloalkylthio” as used herein refers to an alkylthio group as defined above wherein the hydrogen atoms are partially or completely substituted by fluorine, chlorine, bromine and/or iodine. C₁-C₂-Haloalkylthio is, for example, SCH₂F, SCHF₂, SCF₃, SCH₂Cl, SCHC₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The terms “alkylsulfinyl” and “alkyl-S(O)ₖ” (wherein k is 1) are equivalent and, as used herein, denote an alkyl group, as defined above, attached via a sulfinyl [S(O)] group. For example, the term “C₁-C₂-alkylsulfinyl” refers to a C₁-C₂-alkyl group, as defined above, attached via a sulfinyl [S(O)] group. The term “C₁-C₄-alkylsulfinyl” refers to a C₁-C₄-alkyl group, as defined above, attached via a sulfinyl [S(O)] group. The term “C₁-C₆-alkylsulfinyl” refers to a C₁-C₆-alkyl group, as defined above, attached via a sulfinyl [S(O)] group. C₁-C₂-alkylsulfinyl is methylsulfinyl or ethylsulfinyl. C₁-C₄-alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl.

The terms “alkylsulfonyl” and “alkyl-S(O)ₖ” (wherein k is 2) are equivalent and, as used herein, denote an alkyl group, as defined above, attached via a sulfonyl [S(O)₂] group. The term “C₁-C₂-alkylsulfonyl” refers to a C₁-C₂-alkyl group, as defined above, attached via a sulfonyl [S(O)₂] group. The term “C₁-C₄-alkylsulfonyl” refers to a C₁-C₄-alkyl group, as defined above, attached via a sulfonyl [S(O)₂] group. The term “C₁-C₆-alkylsulfonyl” refers to a C₁-C₆-alkyl group, as defined above, attached via a sulfonyl [S(O)₂] group. C₁-C₂-alkylsulfonyl is methylsulfonyl or ethylsulfonyl. C₁-C₄-alkylsulfonyl is additionally, for example, n-propylsulfonyl, 1-methylethylsulfonyl (isopropylsulfonyl), butylsulfonyl, 1-methylpropylsulfonyl (secbutylsulfonyl), 2-methylpropylsulfonyl (isobutylsulfonyl) or 1,1-dimethylethylsulfonyl (tertbutylsulfonyl). C₁-C₆-alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl.

The term “alkylamino” as used herein denotes in each case a group R*HN—, wherein R* is a straight-chain or branched alkyl group usually having from 1 to 6 carbon atoms (“C₁-C₆-alkylamino”), preferably 1 to 4 carbon atoms(“C₁-C₄-alkylamino”). Examples of C₁-C₆-alkylamino are methylamino, ethylamino, n-propylamino, isopropylamino, n-butylamino, 2-butylamino, isobutylamino, tert-butylamino, and the like.

The term “dialkylamino” as used herein denotes in each case a group R*R^{∘}N—, wherein R* and R^{∘}, independently of each other, are a straight-chain or branched alkyl group each usually having from 1 to 6 carbon atoms (“di-(C₁-C₆-alkyl)-amino”), preferably 1 to 4 carbon atoms (“di(C₁-C₄-alkyl)-amino”). Examples of a di-(C₁-C₆-alkyl)-amino group are dimethylamino, diethylamino, dipropylamino, dibutylamino, methyl-ethyl-amino, methyl-propyl-amino, methylisopropylamino, methyl-butyl-amino, methyl-isobutyl-amino, ethyl-propyl-amino, ethylisopropylamino, ethyl-butyl-amino, ethyl-isobutyl-amino, and the like.

The suffix “-carbonyl” in a group denotes in each case that the group is bound to the remainder of the molecule via a carbonyl C═O group. This is the case e.g. in alkylcarbonyl, haloalkylcarbonyl, aminocarbonyl, alkylaminocarbonyl, dialkylaminocarbonyl, alkoxycarbonyl, haloalkoxycarbonyl.

The term “aryl” as used herein refers to a mono-, bi- or tricyclic aromatic hydrocarbon radical such as phenyl or naphthyl, in particular phenyl.

The term “het(ero)aryl” as used herein refers to a mono-, bi- or tricyclic heteroaromatic hydrocarbon radical, preferably to a monocyclic heteroaromatic radical, such as pyridyl, pyrimidyl and the like.

The term “3-, 4-, 5- or 6-membered monocyclic or 8-, 9- or 10-membered bicyclic saturated, unsaturated or aromatic heterocycle containing 1, 2, 3 or 4 heteroatoms as ring members selected from the groups consisting of N, O and S” as used herein denotes monocyclic or bicyclic radicals, the monocyclic or bicyclic radicals being saturated, unsaturated or aromatic where N can optionally be oxidized, i.e. in the form of an N-oxide, and S can also optionally be oxidized to various oxidation states, i.e. as SO or SO₂. An unsaturated heterocycle contains at least one C—C and/or C—N and/or N—N double bond(s). A fully unsaturated heterocycle contains as many conjugated C—C and/or C—N and/or N—N double bonds as allowed by the size(s) of the ring(s). An aromatic monocyclic heterocycle is a fully unsaturated 5- or 6-membered monocyclic heterocycle. An aromatic bicyclic heterocycle is an 8-, 9- or 10-membered bicyclic heterocycle consisting of a 5- or 6-membered heteroaromatic ring which is fused to a phenyl ring or to another 5- or 6-membered heteroaromatic ring. The heterocycle may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5- or 6-membered monocyclic saturated heterocycle include: oxirane-2-yl, aziridine-1-yl, aziridine-2-yl, oxetan-2-yl, azetidine-1-yl, azetidine-2-yl, azetidine-3-yl, thietane-1-yl, thietan-2-yl, thietane-3-yl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl and the like.

Examples of a 5- or 6-membered monocyclic partially unsaturated heterocycle include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl and 1,2,4-di- or tetrahydrotriazin-3-yl.

A 5- or 6-membered monocyclic fully unsaturated (including aromatic) heterocyclic ring is e.g. a 5- or 6-membered monocyclic fully unsaturated (including aromatic) heterocyclic ring. Examples are: 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl.

Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like.

If two radicals bound on the same nitrogen atom (for example R^{2c} and R^{2d} or R^{2e} and R^{2f} or R^{2g} and R^{2h}) together with the nitrogen atom, to which they are bound, form a 5-, 6 or 7-membered, saturated or unsaturated N-bound heterocyclic radical, which may carry as a ring member a further heteroatom selected from O, S and N, this is for example pyrrolidine-1-yl, pyrazolidin-1-yl, imidazolidin-1-yl, oxazolidin-3-yl, thiazolidin-3-yl, isoxazolidin-2-yl, isothiazolin-2-yl, [1,2,3]-triazolidin-1-yl, [1,2,3]-triazolidin-2-yl, [1,2,4]-triazolidin-1-yl, [1,2,4]-triazolidin-4-yl, [1,2,3]-oxadiazolidin-2-yl, [1,2,3]-oxadiazolidin-3-yl, [1,2,5]-oxadiazolidin-2-yl, [1,2,4]-oxadiazolidin-2-yl, [1,2,4]-oxadiazolidin-4-yl, [1,3,4]-oxadiazolidin-3-yl, [1,2,3]-thiadiazolidin-2-yl, [1,2,3]-thiadiazolidin-3-yl, [1,2,5]-thiadiazolidin-2-yl, [1,2,4]-thiadiazolidin-2-yl, [1,2,4]-thiadiazolidin-4-yl, [1,3,4]-thiadiazolidin-3-yl, piperdin-1-yl, piperazine-1-yl, morpholin-1-yl, thiomorpholin-1-yl, 1-oxothiomorpholin-1-yl, 1,1-dioxothiomorpholin-1-yl, azepan-1-yl, 1,4-diazepan-1-yl, pyrrolin-1-yl, pyrazolin-1-yl, imidazolin-1-yl, oxazolin-3-yl, isoxazolin-2-yl, thiazolin-3-yl, isothiazolin-1-yl, 1,2-dihydropyridin-1-yl, 1,2,3,4-tetrahydropyridin-1-yl, 1,2,5,6-tetrahydropyridin-1-yl, 1,2-dihydropyridazin, 1,6-dihydropyridazin, 1,2,3,4-tetrahydropyridazin-1-yl, 1,2,5,6-tetrahydropyridazin-1-yl, 1,2-dihydropyrimidin, 1,6-dihydropyrimidin, 1,2,3,4-tetrahydropyrimidin-1-yl, 1,2,5,6-tetrahydropyrimidin-1-yl, 1,2-dihydropyrazin-1-yl, 1,2,3,4-tetrahydropyrazin-1-yl, 1,2,5,6-tetrahydropyrazin-1-yl, pyrrol-1-yl, pyrazol-1-yl, imidazol-1-yl, [1,2,3]-1H-triazol-1-yl, [1,2,3]-2H-triazol-2-yl, [1,2,4]-1H-triazol-1-yl and [1,2,4]-4H-triazol-4-yl.

The remarks made below as to preferred embodiments of the variables (substituents) of the compounds of formula I are valid on their own as well as preferably in combination with each other, as well as in combination with the stereoisomers, salts, or tautomers thereof.

The remarks made below concerning preferred embodiments of the variables further are valid on their own as well as preferably in combination with each other concerning the compounds of formulae I, where applicable, as well as concerning the uses and methods according to the invention and the composition according to the invention.

Preferred compounds according to the invention are compounds of formula I or a stereoisomer, or salt thereof, wherein the salt is an agriculturally suitable salt. Particularly preferred compounds according to the invention are compounds of formula I or a salt thereof, especially an agriculturally suitable salt thereof.

According to a first group 1 of embodiments of the invention the variable Q in the compounds of formula I is Q¹:

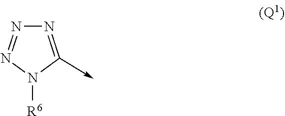

Herein, the arrow represents the binding site of the variable Q¹ conjugated to the remaining part of the compound of formula I. Compounds of formula I wherein Q is Q¹ have the following formula I.A, where the variables R¹, R², R³ and R⁶ are as defined herein:

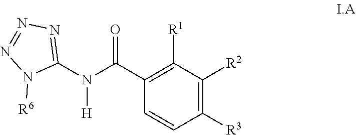

According to a second group 2 of embodiments of the invention the variable Q in the compounds of formula I is Q²:

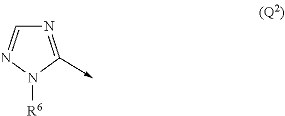

Herein, the arrow represents the binding site of the variable Q² conjugated to the remaining part of the compound of formula I. Compounds of formula I wherein Q is Q² have the following formula I.B, where the variables R¹, R², R³ and R⁶ are as defined herein:

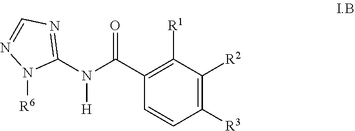

According to a thirds group 3 of embodiments of the invention the variable Q in the compounds of formula I is Q³:

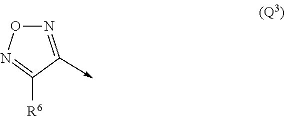

Herein, the arrow represents the binding site of the variable Q³ conjugated to the remaining part of the compound of formula I. Compounds of formula I wherein Q is Q³ have the following formula I.C, where the variables R¹, R², R³ and R⁶ are as defined herein:

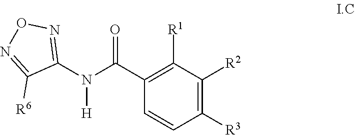

According to a fourth group 4 of embodiments of the invention the variable Q in the compounds of formula I is Q⁴:

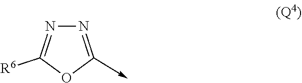

Herein, the arrow represents the binding site of the variable Q⁴ conjugated to the remaining part of the compound of formula I. Compounds of formula I wherein Q is Q⁴ have the following formula I.D, where the variables R¹, R², R³ and R⁶ are as defined herein:

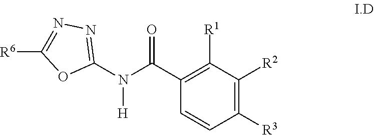

Particular preference is given to the compounds of first group of embodiments and also to the compounds of the fourth group of embodiments, i.e. to the compounds of formula I.A and to the compounds of formula I.D and to the agriculturally suitable salts thereof.

Preferred compounds according to the invention are compounds of formulae I, I.A, I.B, I.C and I.D and the agriculturally suitable salts thereof, wherein R¹ is selected from the group consisting of cyano, halogen, nitro, C₁-C₆-alkyl, C₁-C₆-haloalkyl, C₁-C₄-alkoxy-C₁-C₄-alkyl, C₁-C₄-haloalkoxy-C₁-C₄-alkyl, C₁-C₆-alkoxy, C₁-C₄-alkoxy-C₁-C₄-alkoxy-Z¹, R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond.

More preferably, R¹ is selected from halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond.

In particular, R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl, specifically R¹ is F, Cl, Br, I, nitro, CH₃, SCH₃, SCF₃, SO₂CH₃ or CH₂OCH₂CH₂OCH₃, and more specifically R¹ is F, Cl, Br, I, nitro, CH₃, SCH₃ or SO₂CH₃.

Especially, R¹ in formulae I, I.A, I.B, I.C and I.Di s CH₃ or Cl.

According to the present invention, R² is R^{2c}R^{2d}NC(O)NR²ⁿ—Z².

A particular group 5 of embodiments according to the invention are compounds of formula I and the agriculturally suitable salts thereof, and likeweise the compounds of formulae I.A, I.B, I.C and I.D and the agriculturally suitable salts thereof, wherein in R², Z² is C₁-C₄-alkanediyl; preferably, Z² is —CH₂— or —CH₂CH₂—, i.e. R² is R^{2c}R^{2d}NC(O)NR²ⁿCH₂— or R²CR^{2d}NC(O)NR²ⁿCH₂CH₂—;

According to a preferred group 6 of embodiments, Z² in formulae I, I.A, I.B, I.C and I.D is a covalent bond, i.e. R² is R^{2c}R^{2d}NC(O)NR²ⁿ—.

According to a more preferred embodiment, Z² is a covalent bond and R²ⁿ is selected from the group consisting of hydrogen and C₁-C₆-alkyl.

According to an even more preferred group 7 of embodiments, Z² in formulae I, I.A, I.B, I.C and I.D is a covalent bond and R²ⁿ is hydrogen, i.e. R² is R^{2c}R^{2d}NC(O)NH—.

In the context of R², and likewise in the context of groups 1, 2, 3, 4, 5, 6, and 7 of embodiments, R^{2c} is preferably selected from the group consisting of C₁-C₆-alkyl, C₃-C₇-cycloalkyl, C₃-C₇-cycloalkyl-C₁-C₄-alkyl, C₁-C₆-haloalkyl, C₁-C₆-alkoxy, phenyl, and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;

More preferably, R^{2c} is selected from the group consisting of C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy;

Even more preferably, R^{2c} is selected from the group consisting of C₁-C₄-alkyl, C₃-C₇-cycloalkyl, and C₁-C₄-haloalkyl.

Herein, particularly preferably, R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl, or R^{2c} is selected from the group consisting of methyl, ethyl, isopropyl (iPr), cyclopropyl (cPr), CHF₂CH₂— and CF₃CH₂—.

In the context of R², and likewise in the context of groups 1, 2, 3, 4, 5, 6, and 7 of embodiments, R^{2d} is preferably selected from the group consisting of C₁-C₆-alkyl, C₃-C₇-cycloalkyl, C₃-C₇-cycloalkyl-C₁-C₄-alkyl, C₁-C₆-haloalkyl and phenyl where phenyl is unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; more preferably, R^{2d} is selected from the group consisting of C₁-C₄-alkyl, C₃-C₇-cycloalkyl, and C₁-C₄-haloalkyl or R^{2d} is selected from the group consisting of C₂-C₆-alkyl, C₃-C₇-cycloalkyl, C₃-C₇-cycloalkyl-C₁-C₂-alkyl and C₁-C₆-haloalkyl, in particular from the group consisting of C₂-C₄-alkyl, C₃-C₇-cycloalkyl and C₁-C₄-haloalkyl; Herein, very particularly preferably, R^{2d} is ethyl, isopropyl (iPr), cyclopropyl (cPr), CHF₂CH₂—, or CF₃CH₂—.

In the context of R², and likewise in the context of groups 1, 2, 3, 4, 5, 6, and 7 of embodiments, the combination of R^{2c} and R^{2d} is preferably as follows:

R^{2c} is selected from the group consisting of C₁-C₄-alkyl, C₃-C₇-cycloalkyl and C₁-C₄-haloalkyl;

R^{2d} is selected from the group consisting of C₂-C₆-alkyl, C₃-C₇-cycloalkyl, C₃-C₇-cycloalkyl-C₁-C₂-alkyl and C₁-C₆-haloalkyl.

In the context of R², and likewise in the context of groups 1, 2, 3, 4, 5, 6, and 7 of embodiments, the combination of R^{2c} and R^{2d} is even more preferably as follows:

R^{2c} is selected from the group consisting of C₁-C₄-alkyl, C₃-C₇-cycloalkyl, and C₁-C₄-haloalkyl, with particular preference given to R^{2c} being methyl, ethyl, isopropyl (iPr), cyclopropyl (cPr), CHF₂CH₂—, or CF₃CH₂—; and

R^{2d} is selected from the group consisting of C₂-C₄-alkyl, C₃-C₇-cycloalkyl and C₁-C₄-haloalkyl, with particular preference given to R^{2d} being ethyl, isopropyl (iPr), cyclopropyl (cPr), CHF₂CH₂—, or CF₃CH₂—.

In the context of R², and likewise in the context of groups 1, 2, 3, 4, 5, 6, and 7 of embodiments, the combination of R^{2c} and R^{2d} is also preferably as follows:

R^{2c} is selected from the group consisting of C₁-C₄-alkyl, C₃-C₇-cycloalkyl, and C₁-C₄-haloalkyl, with particular preference given to R^{2c} being methyl, ethyl, isopropyl (iPr), cyclopropyl (cPr), CHF₂CH₂—, or CF₃CH₂—; and

R^{2d} is selected from the group consisting of C₃-C₇-cycloalkyl and C₁-C₄-haloalkyl, with particular preference given to R^{2d} being cyclopropyl (cPr), CHF₂CH₂—, or CF₃CH₂—.

In the context of R², and likewise in the context of groups 1, 2, 3, 4, 5, 6, and 7 of embodiments, R^{2c} and R^{2d} together with the nitrogen atom, to which they are bound may also form a 5- or 6-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy; preferably, R^{2c} and R^{2d} together with the nitrogen atom, to which they are bound form Nmorpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl.

Preferred compounds according to the invention are compounds of formulae I, I.A, I.B, I.C and I.D and the agriculturally suitable salts thereof, wherein R³ is selected from the group consisting of hydrogen, cyano, halogen, C₁-C₄-alkyl, C₁-C₄-haloalkyl, C₁-C₄-alkoxy, C₁-C₄-haloalkoxy, C₂-C₄-alkenyl, C₂-C₄-alkynyl, C₂-C₄-alkenyloxy, C₂-C₄-alkynyloxy or R^{2b}—S(O)ₖ, where the variables k and R^{2b} have one of the herein defined meanings; in particular, R³ is selected from the group consisting of halogen, nitro, C₁-C₄-alkyl, C₁-C₄-haloalkyl, C₁-C₄-haloalkoxy, or R^{2b}—S(O)ₖ, where the variables k and R^{2b} have one of the herein defined meanings.

More preferably, R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂.

In particular, R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, specifically from Cl, F, Br, I, NO₂, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, and more specifically from Cl, Br, I, CF₃, SCH₃, S(O)CH₃ and S(O)₂CH₃.

In particular, R³ is different from nitro (═NO₂).

Especially, R³ is selected from the group consisting of halogen and C₁-C₂-haloalkyl, with particular preference given to those compounds, where R³ is selected from the group consisting of Cl, Br and CF₃, with even more preference given to compounds of the formulae I.A and I.D and the agriculturally acceptable salts thereof, where R³ is selected from the group consisting of Cl, Br and CF₃.

Preferred compounds according to the invention are compounds of formulae I, I.A, I.B, I.C and I.D and the agriculturally suitable salts thereof, wherein R⁶ is selected from the group consisting of C₁-C₆-alkyl, C₁-C₄-alkoxy-C₁-C₄-alkyl, and phenyl. In particular, R⁶ is selected from the group consisting of C₁-C₂-alkyl, C₁-C₂-alkoxy-C₁-C₂-alkyl, specifically from CH₃, CH₃CH₂, CH₃OCH₂CH₂ and CH₃OCH₂. Especially preferred compounds according to the invention are compounds of formulae I, I.A, I.B, I.C and I.D and the agriculturally suitable salts thereof, where R⁶ is CH₃.

In this context, the variables R²¹, Z¹, Z², Z^{2a}, R^{b}, R^{1b}, R^{2b}, R^{2c}, R^{2d}, R^{2e}, R^{2f}, R^{2g}, R^{2h}, R^{k}, n and k, independently of each other, preferably have one of the following meanings:

R²¹ is selected from halogen, C₁-C₄-alkyl, C₃-C₆-cycloalkyl, C₃-C₆-halocycloalkyl, C₁-C₄-haloalkyl, C₁-C₄-alkoxy, C₁-C₄-alkoxy-C₁-C₄-alkyl, C₁-C₄-alkylthio-C₁-C₄-alkyl, C₁-C₄-alkoxy-C₁-C₄-alkoxy and C₁-C₆-haloalkyloxy, more preferably from halogen, C₁-C₄-alkyl, C₃-C₆-cycloalkyl, C₁-C₄-haloalkyl and C₁-C₄-alkoxy.

More preferably R²¹ is selected from the group consisting of halogen, C₁-C₄-alkyl, C₃-C₆-cycloalkyl, C₁-C₄-haloalkyl, C₁-C₄-alkoxy, C₁-C₄-alkoxy-C₁-C₄-alkyl, C₁-C₄-alkylthio-C₁-C₄-alkyl and C₁-C₄-alkoxy-C₁-C₄-alkoxy; in particular from halogen, C₁-C₄-alkyl, C₁-C₄-alkoxy, C₁-C₄-haloalkyl, C₁-C₄-alkoxy-C₁-C₄-alkyl and C₁-C₄-alkoxy-C₁-C₄-alkoxy; and specifically from Cl, F, Br, methyl, ethyl, methoxy and trifluoromethyl.

Z¹, Z² independently of each other are selected from a covalent bond, methanediyl and ethanediyl, and in particular are a covalent bond.

Z^{2a} is selected from a covalent bond, C₁-C₂-alkanediyl, O—C₁-C₂-alkanediyl, C₁-C₂-alkanediyl-O and C₁-C₂-alkanediyl-O—C₁-C₂-alkanediyl; more preferably from a covalent bond, methanediyl, ethanediyl, O-methanediyl, O-ethanediyl, methanediyl-O, and ethanediyl-O; and in particular from a covalent bond, methanediyl and ethanediyl.

R^{b}, R^{1b}, R^{2b} independently of each other are selected from C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₂-C₆-haloalkynyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S, where phenyl and heterocyclyl are unsubstituted or substituted by 1, 2 or 3 groups, which are identical or different and selected from the group consisting of halogen, C₁-C₄-alkyl, C₁-C₂-haloalkyl and C₁-C₂-alkoxy.

More preferably R^{b}, R^{1b}, R^{2b} independently of each other are selected from the group consisting of C₁-C₄-alkyl, C₂-C₄-alkenyl, C₂-C₄-alkynyl, C₁-C₄-haloalkyl, C₂-C₄-haloalkenyl, C₂-C₄-haloalkynyl, C₃-C₆-cycloalkyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S.

In particular, R^{b}, R^{1b}, R^{2b} independently of each other are selected from C₁-C₄-alkyl, C₁-C₄-haloalkyl, C₂-C₄-alkenyl, C₂-C₄-haloalkenyl, C₂-C₄-alkynyl, C₃-C₆-cycloalkyl, phenyl and heterocyclyl, where heterocyclyl is a 5- or 6-membered aromatic heterocyclic radical having 1 or 2 nitrogen atoms as ring members.

R^{2c}, R^{k} independently of each other are selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl, benzyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S, where phenyl, benzyl and heterocyclyl are unsubstituted or substituted by 1, 2 or 3 groups, which are identical or different and selected from the group consisting of halogen, C₁-C₄-alkyl, C₁-C₄-haloalkyl and C₁-C₄-alkoxy.

More preferably R^{2c}, R^{k} independently of each other are selected from hydrogen, C₁-C₄-alkyl, C₁-C₄-haloalkyl, C₂-C₄-alkenyl, C₂-C₄-haloalkenyl, C₂-C₄-alkynyl, C₃-C₆-cycloalkyl, phenyl and heterocyclyl, where heterocyclyl is a 5- or 6-membered monocyclic saturated, partially unsaturated or aromatic heterocycle, which contains 1, 2 or 3 heteroatoms as ring members, which are selected from the group consisting of O, N and S.

In particular, R^{2c}, R^{k} independently of each other are selected from hydrogen, C₁-C₄-alkyl, C₁-C₄-haloalkyl, C₂-C₄-alkenyl, C₂-C₄-haloalkenyl, C₃-C₆-cycloalkyl, phenyl and heterocyclyl, where heterocyclyl is a 5- or 6-membered aromatic heterocyclic radical having 1 or 2 nitrogen atoms as ring members.

R^{2d} is selected from C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl and benzyl.

More preferably R^{2d} is selected from C₁-C₆-alkyl, C₁-C₆-haloalkyl, C₂-C₆-alkenyl, C₂-C₆-haloalkenyl, C₂-C₆-alkynyl, C₁-C₄-alkoxy-C₁-C₄-alkyl and C₃-C₇-cycloalkyl, which is unsubstituted or partly or completely halogenated, and in particular selected from C₁-C₄-alkyl, C₁-C₄-haloalkyl, C₂-C₄-alkenyl, C₂-C₄-haloalkenyl, C₂-C₄-alkynyl and C₃-C₆-cycloalkyl.

R^{2e}, R^{2f} independently of each other are selected from the group consisting of hydrogen, C₁-C₆-alkyl, C₃-C₇-cycloalkyl, which is unsubstituted or partially or completely halogenated, C₁-C₆-haloalkyl, C₂-C₆-alkenyl, C₂-C₆-haloalkenyl, C₁-C₄-alkoxy-C₁-C₄-alkyl, phenyl and benzyl, where phenyl and benzyl are unsubstituted or substituted by 1, 2 or 3 groups, which are identical or different and selected from the group consisting of halogen, C₁-C₄-alkyl, C₁-C₄-haloalkyl and C₁-C₄-alkoxy, or R^{2e} and R^{2f} together with the nitrogen atom, to which they are bound may form a 5-, 6 or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of halogen, C₁-C₄-alkyl, C₁-C₄-haloalkyl and C₁-C₄-alkoxy.

More preferably R^{2e}, R^{2f} independently of each other are selected from hydrogen, C₁-C₆-alkyl, C₁-C₆-haloalkyl and benzyl, or R^{2e} and R^{2f} together with the nitrogen atom, to which they are bound may form a 5- or 6-membered, saturated or unsaturated heterocyclic radical, which may carry as a ring member a further heteroatom selected from O, S and N and which is unsubstituted or may carry 1, 2 or 3 groups, which are identical or different and selected from the group consisting of halogen, C₁-C₄-alkyl and C₁-C₄-haloalkyl.

In particular, R^{2e}, R^{2f} independently of each other are selected from hydrogen and C₁-C₄-alkyl, or R^{2e} and R^{2f} together with the nitrogen atom, to which they are bound may form a 5- or 6-membered, saturated heterocyclic radical, which may carry as a ring member a further heteroatom selected from O, S and N and which is unsubstituted or may carry 1, 2 or 3 methyl groups.

R^{2g} is selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl and benzyl.

More preferably R^{2g} is selected from hydrogen, C₁-C₆-alkyl, C₁-C₆-haloalkyl, C₂-C₆-alkenyl, C₂-C₆-haloalkenyl, benzyl, C₁-C₄-alkoxy-C₁-C₄-alkyl and C₃-C₇-cycloalkyl, which is unsubstituted or partly or completely halogenated, and in particular selected from hydrogen, C₁-C₄-alkyl, C₁-C₄-haloalkyl, C₂-C₄-alkenyl, C₂-C₄-haloalkenyl, benzyl and C₃-C₆-cycloalkyl.

R^{2h} is selected from hydrogen, C₁-C₆-alkyl, C₃-C₇-cycloalkyl, which is unsubstituted or partly or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl, benzyl and a radical C(═O)—R^{k}, where R^{k} is H, C₁-C₄-alkyl, C₁-C₄-haloalkyl or phenyl.

More preferably R^{2h} is selected from hydrogen, C₁-C₆-alkyl, C₁-C₆-haloalkyl, C₂-C₆-alkenyl, C₂-C₆-haloalkenyl, benzyl, C₁-C₄-alkoxy-C₁-C₄-alkyl and C₃-C₇-cycloalkyl, which is unsubstituted or partly or completely halogenated, and in particular selected from hydrogen, C₁-C₄-alkyl, C₁-C₄-haloalkyl, C₂-C₄-alkenyl, C₂-C₄-haloalkenyl, benzyl and C₃-C₆-cycloalkyl; or

R^{2g} and R^{2h} together with the nitrogen atom, to which they are bound may form a 5-, 6 or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl and C₁-C₄-haloalkyl and C₁-C₄-alkoxy;

more preferably R^{2g} and R^{2h} together with the nitrogen atom, to which they are bound may form a 5- or 6-membered, saturated or unsaturated heterocyclic radical, which may carry as a ring member a further heteroatom selected from O, S and N and which is unsubstituted or may carry 1, 2 or 3 groups, which are identical or different and selected from the group consisting of halogen, C₁-C₄-alkyl and C₁-C₄-haloalkyl;

and in particular, R^{2g} and R^{2h} together with the nitrogen atom, to which they are bound may form a 5- or 6-membered, saturated heterocyclic radical, which may carry as a ring member a further heteroatom selected from O, S and N and which is unsubstituted or may carry 1, 2 or 3 methyl groups.

n and k independently of each other are 0 or 2, and in particular 2.

Particularly preferred are compounds of formulae I, I.A, I.B, I.C and I.D and the agriculturally suitable salts thereof, wherein the variables R¹ and R³ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond. In particular, R¹ is selected from F, Cl, Br, I, nitro, CH₃, SCH₃, SCF₃, SO₂CH₃, and CH₂OCH₂CH₂OCH₃ with most preference given to CH₃ and Cl; and

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂.

In particular, R³ is selected from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃.

Also particularly preferred are compounds of formulae I, I.A, I.B, I.C and I.D and the agriculturally suitable salts thereof, wherein the variables R¹ and R³ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl, in particular from F, Cl, Br, I, nitro, CH₃, SCH₃ and SO₂CH₃ with most preference given to CH₃ and Cl; and

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, most particular from Cl, Br, I, CF₃, SCH₃, S(O)CH₃ and S(O)₂CH₃ with most preference given to Cl, Br and CF₃.

Particular preference is given to the compounds of formulae I, I.A, I.B, I.C and I.D and the agriculturally suitable salts thereof, where the combination of R¹ and R³ is as follows:

R¹ is methyl and R³ is selected from the group consisting of halogen and C₁-C₂-haloalkyl, with particular preference given to R³ being selected from the group consisting of Cl, Br and CF₃.

Particular preference is also given to the compounds of formulae I, I.A, I.B, I.C and I.D and the agriculturally suitable salts thereof, where the combination of R¹ and R³ is as follows:

R¹ is Cl and R³ is selected from the group consisting of Br and C₁-C₂-haloalkyl, with particular preference given to R³ being selected from the group consisting of Br and CF₃.

Particular preference is also given to the compounds of formulae I, I.A, I.B, I.C and I.D and the agriculturally suitable salts thereof, where the combination of R¹ and R³ is as follows:

R¹ is Cl and R³ is Cl.

Especially preferred are compounds of formula I and the agriculturally suitable salts thereof, wherein Q is Q¹, Q², Q³ or Q⁴ and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₂-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, NO₂, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂, with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₂-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from from the group consisting of ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NR²ⁿ—;

R²ⁿ is C₁-C₆-alkyl, preferably methyl or ethyl;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NR²ⁿ—;

R²ⁿ is C₁-C₆-alkyl, preferably methyl or ethyl;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NR²ⁿ—;

R²ⁿ is C₁-C₆-alkyl, preferably methyl or ethyl;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from from the group consisting of methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:
R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;
R² is R^{2c}R^{2d}NC(O)NR²ⁿ—Z²;
Z² is C₁-C₄-alkanediyl, preferably —CH₂— or —CH₂CH₂—;
R²ⁿ is selected from hydrogen and C₁-C₆-alkyl, preferably from hydrogen, methyl and ethyl;
R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;
R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or
R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;
R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;
R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:
R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;
R² is R^{2c}R^{2d}NC(O)NR²ⁿ—Z²;
Z² is C₁-C₄-alkanediyl, preferably —CH₂— or —CH₂CH₂—;
R²ⁿ is selected from hydrogen and C₁-C₆-alkyl, preferably from hydrogen, methyl and ethyl;
R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;
R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or
R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;
R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;
R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹, Q², Q³ or Q⁴ and the variables R¹, R², R³ and R⁶ have the following meanings:
R¹ is F, Cl, Br, I, nitro, CH₃, SCH₃, SCF₃, SO₂CH₃, and CH₂OCH₂CH₂OCH₃ with most preference given to CH₃ and Cl;
R² is R^{2c}R^{2d}NC(O)NR²ⁿ—Z²;
Z² is C₁-C₄-alkanediyl, preferably —CH₂— or —CH₂CH₂—;
R²ⁿ is selected from hydrogen and C₁-C₆-alkyl, preferably from hydrogen, methyl and ethyl;
R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;
R^{2d} is selected from the group consisting of methyl, ethyl, isopropyl (iPr), cyclopropyl (cPr), CHF₂CH₂—, and CF₃CH₂—; or
R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;
R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;
R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹ (formula I.A) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;
with the exception of
Benzamide, 4-bromo-2-chloro-N-[1-(2-methoxyethyl)-1H-tetrazol-5-yl]-3-[[(methoxymethylamino)carbonyl]amino]-, CAS Registry Number: 1421777-21-0;
Benzamide, 2,4-dichloro-N-[1-(2-methoxyethyl)-1H-tetrazol-5-yl]-3-[[(methoxymethylamino)carbonyl]amino]-, CAS Registry Number: 1421776-96-6;
Benzamide, 2,4-dichloro-3-[[(methoxymethylamino)carbonyl]amino]-N-(1-phenyl-1H-tetrazol-5-yl)-, CAS Registry Number: 1361245-23-9;
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6;
Benzamide, 2,4-dichloro-3-[[(dimethylamino)carbonyl]amino]-N-(1-methyl-1H-tetrazol-5-yl)-, CAS Registry Number: 1361139-93-6.

Also especially preferred are compounds of formula I and the agriculturally suitable salts thereof, wherein Q is Q¹ (formula I.A) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6.

Also especially preferred are compounds of formula I and the agriculturally suitable salts thereof, wherein Q is Q¹ (formula I.A) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, 4-bromo-2-chloro-N-[1-(2-methoxyethyl)-1H-tetrazol-5-yl]-3-[[(methoxymethylamino)carbonyl]amino]-, CAS Registry Number: 1421777-21-0;
Benzamide, 2,4-dichloro-N-[1-(2-methoxyethyl)-1H-tetrazol-5-yl]-3-[[(methoxymethylamino)carbonyl]amino]-, CAS Registry Number: 1421776-96-6;
Benzamide, 2,4-dichloro-3-[[(methoxymethylamino)carbonyl]amino]-N-(1-phenyl-1H-tetrazol-5-yl)-, CAS Registry Number: 1361245-23-9;
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6;
Benzamide, 2,4-dichloro-3-[[(dimethylamino)carbonyl]amino]-N-(1-methyl-1H-tetrazol-5-yl)-, CAS Registry Number: 1361139-93-6.

Also especially preferred are compounds of formula I and the agriculturally suitable salts thereof, wherein Q is Q¹ (formula I.A) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6.

Also especially preferred are compounds of formula I and the agriculturally suitable salts thereof, wherein Q is Q¹ (formula I.A) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₂-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q¹ (formula I.A) and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from the group consisting of ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q² (formula I.B) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, 2,4-dichloro-3-[[(methoxymethylamino)carbonyl]amino]-N-(1-phenyl-1H-1,2,4-triazol-5-yl)-, CAS Registry Number: 1361103-06-1;
Benzamide, 2,4-dichloro-N-(1-ethyl-1H-1,2,4-triazol-5-yl)-3-[[(methoxymethylamino)carbonyl]amino]-, CAS Registry Number: 1361040-89-2.

Also especially preferred are compounds of formula I and the agriculturally suitable salts thereof, wherein Q is Q² (formula I.B) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Also especially preferred are compounds of formula I and the agriculturally suitable salts thereof, wherein Q is Q² (formula I.B) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, 2,4-dichloro-3-[[(methoxymethylamino)carbonyl]amino]-N-(1-phenyl-1H-1,2,4-triazol-5-yl)-, CAS Registry Number: 1361103-06-1;
Benzamide, 2,4-dichloro-N-(1-ethyl-1H-1,2,4-triazol-5-yl)-3-[[(methoxymethylamino)carbonyl]amino]-, CAS Registry Number: 1361040-89-2.

Also especially preferred are compounds of formula I and the agriculturally suitable salts thereof, wherein Q is Q² (formula I.B) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₂-C₄-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q² (formula I.B) and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from the group consisting of ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q² (formula I.B) and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, 4-Cl-phenyl, 4-CH₃O-phenyl, and phenyl;

R^{2d} is selected from the group consisting of methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q³ (formula I.C) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, N-[4-(1,1-dimethylethyl)-1,2,5-oxadiazol-3-yl]-2-methyl-3-[[[methyl(2-methylpropyl)amino]carbonyl]amino]-4-(methylsulfonyl)-, CAS Registry Number: 1279665-87-0;
Benzamide, 2-methyl-3-[[[methyl(2-methylpropyl)amino]carbonyl]amino]-4-(methylsulfonyl)-N-(4-propyl-1,2,5-oxadiazol-3-yl)-, CAS Registry Number: 1279644-16-4;
Benzamide, 2-methyl-N-[4-(1-methylethyl)-1,2,5-oxadiazol-3-yl]-3-[[[methyl(2-methylpropyl)amino]carbonyl]amino]-4-(methylsulfonyl)-, CAS Registry Number: 1279635-24-3;
Benzamide, N-(4-ethyl-1,2,5-oxadiazol-3-yl)-2-methyl-3-[[[methyl(2-methylpropyl)amino]carbonyl]amino]-4-(methylsulfonyl)-, CAS Registry Number: 1279127-99-9;
Benzamide, 2-methyl-3-[[[methyl(2-methylpropyl)amino]carbonyl]amino]-N-(4-methyl-1,2,5-oxadiazol-3-yl)-4-(methylsulfonyl)-, CAS Registry Number: 1279114-20-3.

Also especially preferred are compounds of formula I and the agriculturally suitable salts thereof, wherein Q is Q³ (formula I.C) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃;

with the exception of
Benzamide, N-[4-(1,1-dimethylethyl)-1,2,5-oxadiazol-3-yl]-2-methyl-3-[[[methyl(2-methylpropyl)amino]carbonyl]amino]-4-(methylsulfonyl)-, CAS Registry Number: 1279665-87-0;
Benzamide, 2-methyl-3-[[[methyl(2-methylpropyl)amino]carbonyl]amino]-4-(methylsulfonyl)-N-(4-propyl-1,2,5-oxadiazol-3-yl)-, CAS Registry Number: 1279644-16-4;
Benzamide, 2-methyl-N-[4-(1-methylethyl)-1,2,5-oxadiazol-3-yl]-3-[[[methyl(2-methylpropyl)amino]carbonyl]amino]-4-(methylsulfonyl)-, CAS Registry Number: 1279635-24-3;
Benzamide, N-(4-ethyl-1,2,5-oxadiazol-3-yl)-2-methyl-3-[[[methyl(2-methylpropyl)amino]carbonyl]amino]-4-(methylsulfonyl)-, CAS Registry Number: 1279127-99-9;
Benzamide, 2-methyl-3-[[[methyl(2-methylpropyl)amino]carbonyl]amino]-N-(4-methyl-1,2,5-oxadiazol-3-yl)-4-(methylsulfonyl)-, CAS Registry Number: 1279114-20-3.

Also especially preferred are compounds of formula I and the agriculturally suitable salts thereof, wherein Q is Q³ (formula I.C) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₃-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₃-alkyl, C₃-C₇-cycloalkyl, and C₁-C₄-haloalkyl, preferably from ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q³ (formula I.C) and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from the group consisting of methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃.

Especially preferred are compounds of formula I and the agriculturally suitable salts thereof, wherein Q is Q⁴ (formula I.D) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-Z¹ and R^{1b}—S(O)ₖ—Z¹, where k and Z¹ are as defined herein and where R^{1b} is as defined above and in particular selected from the group consisting of C₁-C₄-alkyl and C₁-C₄-haloalkyl. In this context Z¹ is in particular a covalent bond; specifically R¹ is selected from F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₄-alkyl, C₁-C₄-haloalkyl, C₁-C₄-haloalkoxy, C₁-C₄-alkylthio, C₁-C₄-haloalkylthio, C₁-C₄-alkyl-S(O), C₁-C₄-haloalkyl-S(O), C₁-C₄-alkyl-S(O)₂, and C₁-C₄-haloalkyl-S(O)₂, in particular from Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Also especially preferred are compounds of formula I and the agriculturally suitable salts thereof, wherein Q is Q⁴ (formula I.D) and the variables R¹, R², R³ and R⁶ have the following meanings:

R¹ is selected from the group consisting of halogen, nitro, C₁-C₄-alkyl, C₁-C₄-alkoxy-C₁-C₄-alkoxy-C₁-C₄-alkyl, C₁-C₄-alkylthio, C₁-C₄-haloalkylthio and C₁-C₄-alkylsulfonyl; specifically R¹ is F, Cl, Br, I, nitro, CH₃, SCH₃ or SO₂CH₃ with most preference given to CH₃ and Cl;

R² is R^{2c}R^{2d}NC(O)NH—;

R^{2c} is selected from C₁-C₄-alkyl, C₃-C₇-cycloalkyl, C₁-C₄-haloalkyl, C₁-C₄-alkoxy, and phenyl, where phenyl is unsubstituted or substituted by 1 or 2 groups, which are identical or different and selected from the group consisting of halogen and C₁-C₄-alkoxy; specifically, R^{2c} is selected from methyl, ethyl, isopropyl (iPr), cyclopropyl (cPr), CHF₂CH₂—, CF₃CH₂—, CH₃O—, 4-Cl-phenyl, 4-CH₃O-phenyl, or phenyl;

R^{2d} is selected from C₁-C₄-alkyl, C₃-C₇-cycloalkyl, and C₁-C₄-haloalkyl, preferably from methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of halogen, C₁-C₂-alkyl, C₁-C₂-haloalkyl, C₁-C₂-haloalkoxy, C₁-C₂-alkylthio, C₁-C₂-haloalkylthio, C₁-C₂-alkyl-S(O), C₁-C₂-haloalkyl-S(O), C₁-C₂-alkyl-S(O)₂ and C₁-C₂-haloalkyl-S(O)₂, and more specifically from Cl, Br, I, CF₃, SCH₃, S(O)CH₃ and S(O)₂CH₃, with most preference given to Cl, Br and CF₃;

R⁶ is selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Especially preferred are compounds of formula I and the agriculturally suitable salts thereof, wherein Q is Q⁴ (formula I.D) and the variables R¹, R², R³ and R⁶ have the following meanings:

R¹ is F, Cl, Br, I, nitro, CH₃, SCH₃, SCF₃, SO₂CH₃, and CH₂OCH₂CH₂OCH₃ with most preference given to CH₃ and Cl;

R² is R^{2c}R^{2d}NC(O)NH—;

R^{2c} is selected from the group consisting of methyl, ethyl, isopropyl (iPr), cyclopropyl (cPr), CHF₂CH₂—, CF₃CH₂—, CH₃O—, 4-Cl-phenyl, 4-CH₃O-phenyl, and phenyl;

R^{2d} is selected from the group consisting of methyl, ethyl, isopropyl (iPr), cyclopropyl (cPr), CHF₂CH₂—, and CF₃CH₂—; or

R^{2c} and R^{2d} together with the nitrogen atom, to which they are bound form N-morpholinyl, N-pyrrolidinyl or N-piperidinyl; N-morpholinyl, N-pyrrolidinyl and N-piperidinyl may carry 1, 2, 3 or 4 groups, which are identical or different and selected from the group consisting of C₁-C₄-alkyl and C₁-C₄-haloalkyl, preferably from methyl, ethyl and trifluoromethyl;

R³ is selected from the group consisting of Cl, F, Br, I, CH₃, CF₃, CHF₂, OCF₃, OCHF₂, SCH₃, SCF₃, SCHF₂, S(O)CH₃, S(O)CH₂CH₃, S(O)₂CH₃ and S(O)₂CH₂CH₃, with most preference given to Cl, Br and CF₃;

R⁶ is selected from the group consisting of CH₃, CH₃CH₂, CH₃OCH₂CH₂ and CH₃OCH₂ with most preference given to CH₃;

A particular group 8 of embodiments relates to the compounds of formulae I, I.A, I.B, I.C and I.D and to the agriculturally suitable salts thereof, where the combination of R¹, R², R^{2c}, R^{2d} and R³ is as follows:
R¹ is C₁-C₂-alkyl, especially CH₃;
R² is R^{2c}R^{2d}NC(O)NH—, i.e. Z² is a covalent bond R²ⁿ is hydrogen;
R^{2c} is selected from the group consisting C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂— and CF₃CH₂—;
R^{2d} is selected from the group consisting of C₁-C₆-alkyl, C₃-C₇-cycloalkyl, C₃-C₇-cycloalkyl-C₁-C₂-alkyl and C₁-C₆-haloalkyl, in particular from the group consisting of C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂—, and CF₃CH₂—;
R³ is selected from the group consisting of halogen and C₁-C₂-haloalkyl, especially from the group consisting of Cl, Br and CF₃.

In this particular group 8 of embodiments R⁶ is preferably selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

A particular group 9 of embodiments relates to the compounds of formulae I, I.A, I.B, I.C and I.D and to the agriculturally suitable salts thereof, where the combination of R¹, R², R^{2c}, R^{2d} and R³ is as follows:
R¹ is halogen, especially Cl;
R² is R^{2c}R^{2d}NC(O)NH—, i.e. Z² is a covalent bond R²ⁿ is hydrogen;
R^{2c} is selected from the group consisting C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂— and CF₃CH₂—;
R^{2d} is selected from the group consisting of C₂-C₆-alkyl, C₃-C₇-cycloalkyl, C₃-C₇-cycloalkyl-C₁-C₂-alkyl and C₁-C₆-haloalkyl, in particular from the group consisting of C₂-C₄-alkyl, C₃-C₇-cycloalkyl and C₁-C₄-haloalkyl and especially from the group consisting of ethyl, isopropyl (iPr), cyclopropyl (cPr), CHF₂CH₂—, and CF₃CH₂—;
R³ is selected from the group consisting of halogen and C₁-C₂-haloalkyl, especially from the group consisting of Cl, Br and CF₃.

In this particular group 9 of embodiments R⁶ is preferably selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

A particular group 9a of embodiments relates to the compounds of formulae I, I.A, I.B, I.C and I.D and to the agriculturally suitable salts thereof, where the combination of R¹, R², R^{2c}, R^{2d} and R³ is as follows:
R¹ is Cl;
R² is R^{2c}R^{2d}NC(O)NH—, i.e. Z² is a covalent bond R²ⁿ is hydrogen;
R^{2c} is selected from the group consisting C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂— and CF₃CH₂—;
R^{2d} is selected from the group consisting of C₂-C₆-alkyl, C₃-C₇-cycloalkyl, C₃-C₇-cycloalkyl-C₁-C₂-alkyl and C₁-C₆-haloalkyl, in particular from the group consisting of C₂-C₄-alkyl, C₃-C₇-cycloalkyl and C₁-C₄-haloalkyl and especially from the group consisting of ethyl, isopropyl (iPr), cyclopropyl (cPr), CHF₂CH₂—, and CF₃CH₂—;
R³ is Cl.

In this particular group 9a of embodiments R⁶ is preferably selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

A particular group 10 of embodiments relates to the compounds of formulae I, I.A, I.B, I.C and I.D and to the agriculturally suitable salts thereof, where the combination of R¹, R², R^{2c}, R^{2d} and R³ is as follows:
R¹ is Cl;
R² is R^{2c}R^{2d}NC(O)NH—, i.e. Z² is a covalent bond R²ⁿ is hydrogen;
R^{2c} is selected from the group consisting C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂— and CF₃CH₂—;
R^{2d} is selected from the group consisting of C₁-C₆-alkyl, C₃-C₇-cycloalkyl, C₃-C₇-cycloalkyl-C₁-C₂-alkyl and C₁-C₆-haloalkyl, in particular from the group consisting of C₁-C₄-alkyl, C₃-C₇-cycloalkyl and C₁-C₄-haloalkyl and especially from the group consisting of methyl, ethyl, isopropyl (iPr), cyclopropyl (cPr), CHF₂CH₂—, and CF₃CH₂—;
R³ is selected from the group consisting of halogen and C₁-C₂-haloalkyl, especially from the group consisting of Br and CF₃.

In this particular group 9a of embodiments R⁶ is preferably selected from the group consisting of C₁-C₆-alkyl, C₁-C₄-alkoxy-C₁-C₄-alkyl, and phenyl, in particular from CH₃, CH₃CH₂, CH₃OCH₂CH₂ and CH₃OCH₂ with most preference given to CH₃.

Examples of preferred compounds are the individual compounds compiled in the Tables below. Moreover, the meanings mentioned below for the individual variables in the Tables are per se, independently of the combination in which they are mentioned, a particularly preferred embodiment of the substituents in question.

Compounds I.A.I, wherein Q is Q¹, R⁶ is methyl, Z² in R² is a covalent bond and R²ⁿ in R² is H:

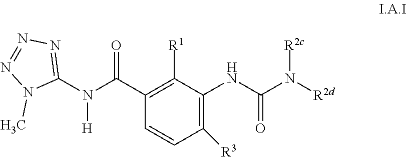

Table 1 Compounds of formula I (I.A.I.1-I.A.I.588) in which the combination of R¹, R^{2c}, R^{2d}, and R³ for a compound corresponds in each case to one row of Table A;

Compounds I.A.II, wherein Q is Q¹, R⁶ is ethyl, Z² in R² is a covalent bond and R²ⁿ in R² is H:

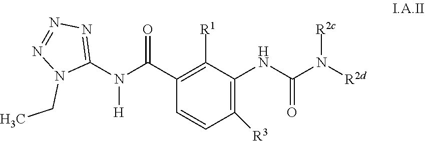

Table 2 Compounds of formula I (I.A.II.1-I.A.II.588) in which the combination of R¹, R^{2c}, R^{2d} and R³ for a compound corresponds in each case to one row of Table A;

Compounds I.A.III, wherein Q is Q¹, R⁶ is methyl, Z² in R² is a covalent bond and R²ⁿ in R² is methyl:

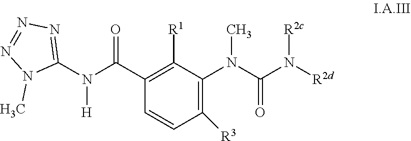

Table 3 Compounds of formula I (I.A.III.1-I.A.III.588) in which the combination of R¹, R^{2c}, R^{2d} and R³ for a compound corresponds in each case to one row of Table A;

Compounds I.A.IV, wherein Q is Q¹, R⁶ is methyl, Z² in R² is a covalent bond and R²ⁿ in R² is ethyl:

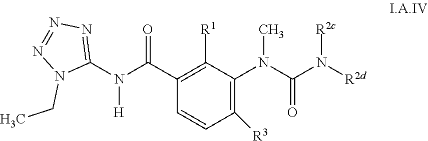

Table 4 Compounds of formula I (I.A.IV.1-I.A.IV.588) in which the combination of R¹, R^{2c}, R^{2d} and R³ for a compound corresponds in each case to one row of Table A;

Compounds I.A.V, wherein Q is Q¹, R⁶ is methyl, Z² in R² is a covalent bond and R²ⁿ in R² is ethyl:

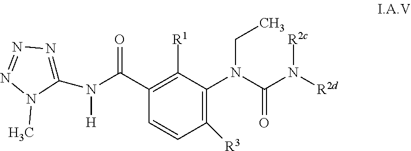

Table 5 Compounds of formula I (I.A.V.1-I.A.V.588) in which the combination of R¹, R^{2c}, R^{2d} and R³ for a compound corresponds in each case to one row of Table A;

Compounds I.A.VI, wherein Q is Q¹, R⁶ is ethyl, Z² in R² is a covalent bond and R²ⁿ in R² is ethyl:

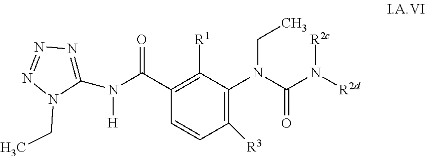

Table 6 Compounds of formula I (I.A.VI.1-I.A.VI.588) in which the combination of R¹, R^{2c}, R^{2d} and R³ for a compound corresponds in each case to one row of Table A;

Compounds I.A.VII, wherein Q is Q¹, R⁶ is ethyl, Z² in R² is a covalent bond and R²ⁿ in R² is H:

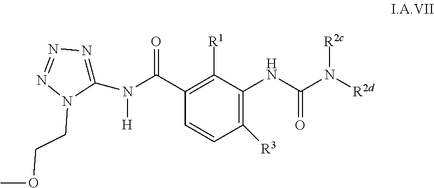

Table 7 Compounds of formula I (I.A.II.1-I.A.II.588) in which the combination of R¹, R^{2c}, R^{2d} and R³ for a compound corresponds in each case to one row of Table A;

Compounds I.B.1, wherein Q is Q², R⁶ is methyl, Z² in R² is a covalent bond and R²ⁿ in R² is H:

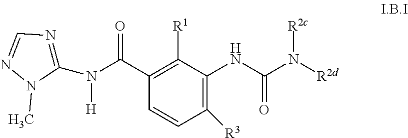

Table 8 Compounds of formula I (I.B.I.1-I.B.I.588) in which the combination of R¹, R^{2c}, R^{2d} and R³ for a compound corresponds in each case to one row of Table A;

Compounds I.B.II, wherein Q is Q², R⁶ is ethyl, Z² in R² is a covalent bond and R²ⁿ in R² is H:

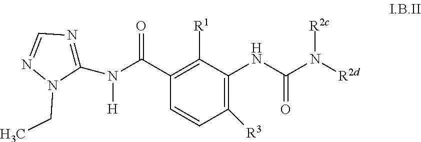

Table 9 Compounds of formula I (I.B.II.1-I.B.II.588) in which the combination of R¹, R^{2c}, R^{2d} and R³ for a compound corresponds in each case to one row of Table A;

Compounds I.B.III, wherein Q is Q², R⁶ is methyl, Z² in R² is a covalent bond and R²ⁿ in R² is methyl:

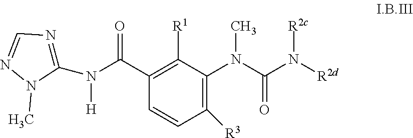

Table 10 Compounds of formula I (I.B.III.1-I.B.III.588) in which the combination of R¹, R^{2c}, R^{2d} and R³ for a compound corresponds in each case to one row of Table A;

Compounds I.B.IV, wherein Q is Q², R⁶ is ethyl, Z² in R² is a covalent bond and R²ⁿ in R² is methyl:

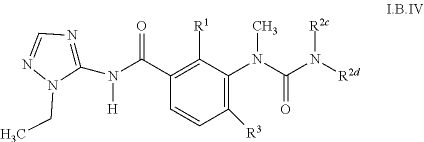

Table 11 Compounds of formula I (I.B.IV.1-I.B.IV.588) in which the combination of R¹, R^{2c}, R^{2d} and R³ for a compound corresponds in each case to one row of Table A;

Compounds I.B.V, wherein Q is Q², R⁶ is methyl, Z² in R² is a covalent bond and R²ⁿ in R² is ethyl:

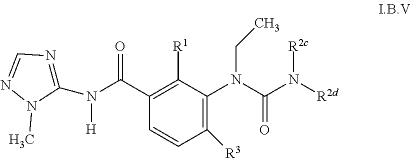

Table 12 Compounds of formula I (I.B.V.1-I.B.V.588) in which the combination of R¹, R^{2c}, R^{2d} and R³ for a compound corresponds in each case to one row of Table A;

Compounds I.B.VI, wherein Q is Q², R⁶ is ethyl, Z² in R² is a covalent bond and R²ⁿ in R² is ethyl:

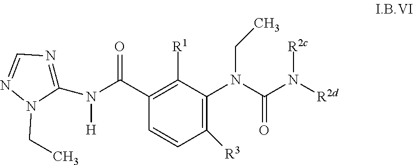

Table 13 Compounds of formula I (l.B.VI.1-I.B.VI.588) in which the combination of R¹, R^{2c}, R^{2d} and R³ for a compound corresponds in each case to one row of Table A;

Compounds I.C.I, wherein Q is Q³, R⁶ is methyl, Z² in R² is a covalent bond and R²ⁿ in R² is H:

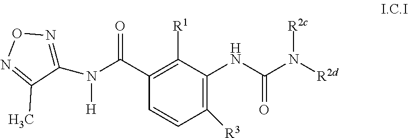

Table 14 Compounds of formula I (I.C.I.1-I.C.I.588) in which the combination of R¹, R^{2c}, R^{2d} and R³ for a compound corresponds in each case to one row of Table A;

Compounds I.C.II, wherein Q is Q³, R⁶ is ethyl, Z² in R² is a covalent bond and R²ⁿ in R² is H:

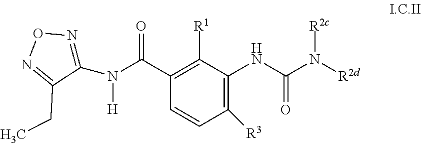

Table 15 Compounds of formula I (I.C.II.1-I.C.II.588) in which the combination of R¹, R^{2c}, R^{2d} and R³ for a compound corresponds in each case to one row of Table A;

Compounds I.C.III, wherein Q is Q³, R⁶ is methyl, Z² in R² is a covalent bond and R²ⁿ in R² is methyl:

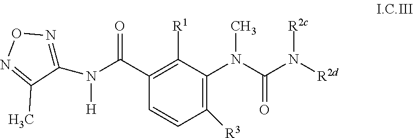

Table 16 Compounds of formula I (I.C.III.1-I.C.III.588) in which the combination of R¹, R^{2c}, R^{2d} and R³ for a compound corresponds in each case to one row of Table A;

Compounds I.C.IV, wherein Q is Q³, R⁶ is methyl, Z² in R² is a covalent bond and R²ⁿ in R² is ethyl:

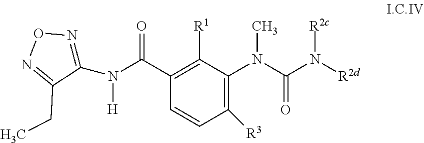

Table 17 Compounds of formula I (I.C.IV.1-I.C.IV.588) in which the combination of R¹, R^{2c}, R^{2d} and R³ for a compound corresponds in each case to one row of Table A;

Compounds I.C.V, wherein Q is Q³, R⁶ is methyl, Z² in R² is a covalent bond and R²ⁿ in R² is ethyl:

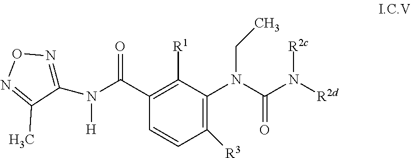

Table 18 Compounds of formula I (I.C.V.1-I.C.V.588) in which the combination of R¹, R^{2c}, R^{2d} and R³ for a compound corresponds in each case to one row of Table A;

Compounds I.C.VI, wherein Q is Q³, R⁶ is ethyl, Z² in R² is a covalent bond and R²ⁿ in R² is ethyl:

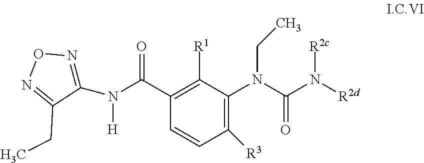

Table 19 Compounds of formula I (l.C.VI.1-I.C.VI.588) in which the combination of R¹, R^{2c}, R^{2d} and R³ for a compound corresponds in each case to one row of Table A;

Compounds I.D.I, wherein Q is Q⁴, R⁶ is methyl, Z² in R² is a covalent bond and R²ⁿ in R² is H:

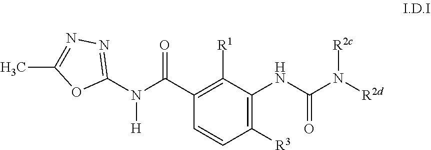

Table 20 Compounds of formula I (I.D.I.1-I.D.1.588) in which the combination of R¹, R^{2c}, R^{2d} and R³ for a compound corresponds in each case to one row of Table A;

Compounds I.D.II, wherein Q is Q⁴, R⁶ is ethyl, Z² in R² is a covalent bond and R²ⁿ in R² is H:

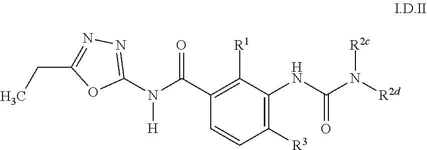

Table 21 Compounds of formula I (I.D.II.1-I.D.11.588) in which the combination of R¹, R^{2c}, R^{2d} and R³ for a compound corresponds in each case to one row of Table A;

Compounds I.D.III, wherein Q is Q⁴, R⁶ is methyl, Z² in R² is a covalent bond and R²ⁿ in R² is methyl:

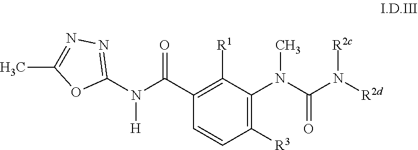

Table 22 Compounds of formula I (I.D.III.1-I.D.111.588) in which the combination of R¹, R^{2c}, R^{2d} and R³ for a compound corresponds in each case to one row of Table A;

Compounds I.D.IV, wherein Q is Q⁴, R⁶ is ethyl, Z² in R² is a covalent bond and R²ⁿ in R² is methyl:

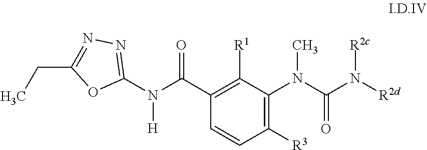

Table 23 Compounds of formula I (I.D.IV.1-I.D.IV.588) in which the combination of R¹, R^{2c}, R^{2d} and R³ for a compound corresponds in each case to one row of Table A;

Compounds I.D.V, wherein Q is Q⁴, R⁶ is methyl, Z² in R² is a covalent bond and R²ⁿ in R² is ethyl:

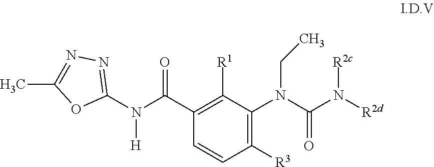

Table 24 Compounds of formula I (I.D.V.1-I.D.V.588) in which the combination of R¹, R^{2c}, R^{2d} and R³ for a compound corresponds in each case to one row of Table A;

Compounds I.D.VI, wherein Q is Q⁴, R⁶ is ethyl, Z² in R² is a covalent bond and R²ⁿ in R²

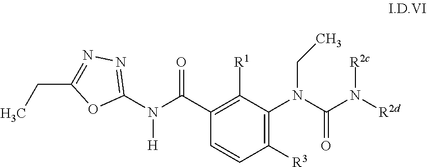

Table 25 Compounds of formula I (I.D.VI.1-I.D.VI.588) in which the combination of R¹, R^{2c}, R^{2d} and R³ for a compound corresponds in each case to one row of Table A;

TABLE A

| | R¹ | R^{2c} | R^{2d} | R³ |
|---|---|---|---|---|
| 1. | Cl | CH₃ | CH₃ | Cl |
| 2. | Cl | CH₃ | CH₃ | Br |
| 3. | Cl | CH₃ | CH₃ | I |
| 4. | Cl | CH₃ | CH₃ | CF₃ |
| 5. | Cl | CH₃ | CH₃ | CH₃S(O) |
| 6. | Cl | CH₃ | CH₃ | CH₃S(O)₂ |
| 7. | Cl | CH₃ | CH₃CH₂ | Cl |
| 8. | Cl | CH₃ | CH₃CH₂ | Br |
| 9. | Cl | CH₃ | CH₃CH₂ | I |
| 10. | Cl | CH₃ | CH₃CH₂ | CF₃ |
| 11. | Cl | CH₃ | CH₃CH₂ | CH₃S(O) |
| 12. | Cl | CH₃ | CH₃CH₂ | CH₃S(O)₂ |
| 13. | Cl | CH₃ | iPr | Cl |
| 14. | Cl | CH₃ | iPr | Br |
| 15. | Cl | CH₃ | iPr | I |
| 16. | Cl | CH₃ | iPr | CF₃ |
| 17. | Cl | CH₃ | iPr | CH₃S(O) |
| 18. | Cl | CH₃ | iPr | CH₃S(O)₂ |
| 19. | Cl | CH₃ | cPr | Cl |
| 20. | Cl | CH₃ | cPr | Br |
| 21. | Cl | CH₃ | cPr | I |
| 22. | Cl | CH₃ | cPr | CF₃ |
| 23. | Cl | CH₃ | cPr | CH₃S(O) |
| 24. | Cl | CH₃ | cPr | CH₃S(O)₂ |
| 25. | Cl | CH₃ | CHF₂CH₂ | Cl |
| 26. | Cl | CH₃ | CHF₂CH₂ | Br |
| 27. | Cl | CH₃ | CHF₂CH₂ | I |
| 28. | Cl | CH₃ | CHF₂CH₂ | CF₃ |
| 29. | Cl | CH₃ | CHF₂CH₂ | CH₃S(O) |
| 30. | Cl | CH₃ | CHF₂CH₂ | CH₃S(O)₂ |
| 31. | Cl | CH₃ | CF₃CH₂ | Cl |
| 32. | Cl | CH₃ | CF₃CH₂ | Br |
| 33. | Cl | CH₃ | CF₃CH₂ | I |
| 34. | Cl | CH₃ | CF₃CH₂ | CF₃ |
| 35. | Cl | CH₃ | CF₃CH₂ | CH₃S(O) |
| 36. | Cl | CH₃ | CF₃CH₂ | CH₃S(O)₂ |
| 37. | Cl | CH₃CH₂ | CH₃CH₂ | Cl |
| 38. | Cl | CH₃CH₂ | CH₃CH₂ | Br |
| 39. | Cl | CH₃CH₂ | CH₃CH₂ | I |
| 40. | Cl | CH₃CH₂ | CH₃CH₂ | CF₃ |
| 41. | Cl | CH₃CH₂ | CH₃CH₂ | CH₃S(O) |
| 42. | Cl | CH₃CH₂ | CH₃CH₂ | CH₃S(O)₂ |
| 43. | Cl | CH₃CH₂ | iPr | Cl |
| 44. | Cl | CH₃CH₂ | iPr | Br |
| 45. | Cl | CH₃CH₂ | iPr | I |
| 46. | Cl | CH₃CH₂ | iPr | CF₃ |
| 47. | Cl | CH₃CH₂ | iPr | CH₃S(O) |
| 48. | Cl | CH₃CH₂ | iPr | CH₃S(O)₂ |
| 49. | Cl | CH₃CH₂ | cPr | Cl |
| 50. | Cl | CH₃CH₂ | cPr | Br |
| 51. | Cl | CH₃CH₂ | cPr | I |
| 52. | Cl | CH₃CH₂ | cPr | CF₃ |
| 53. | Cl | CH₃CH₂ | cPr | CH₃S(O) |
| 54. | Cl | CH₃CH₂ | cPr | CH₃S(O)₂ |
| 55. | Cl | CH₃CH₂ | CHF₂CH₂ | Cl |
| 56. | Cl | CH₃CH₂ | CHF₂CH₂ | Br |
| 57. | Cl | CH₃CH₂ | CHF₂CH₂ | I |
| 58. | Cl | CH₃CH₂ | CHF₂CH₂ | CF₃ |
| 59. | Cl | CH₃CH₂ | CHF₂CH₂ | CH₃S(O) |
| 60. | Cl | CH₃CH₂ | CHF₂CH₂ | CH₃S(O)₂ |
| 61. | Cl | CH₃CH₂ | CF₃CH₂ | Cl |
| 62. | Cl | CH₃CH₂ | CF₃CH₂ | Br |
| 63. | Cl | CH₃CH₂ | CF₃CH₂ | I |
| 64. | Cl | CH₃CH₂ | CF₃CH₂ | CF₃ |
| 65. | Cl | CH₃CH₂ | CF₃CH₂ | CH₃S(O) |
| 66. | Cl | CH₃CH₂ | CF₃CH₂ | CH₃S(O)₂ |
| 67. | Cl | iPr | iPr | Cl |
| 68. | Cl | iPr | iPr | Br |
| 69. | Cl | iPr | iPr | I |
| 70. | Cl | iPr | iPr | CF₃ |
| 71. | Cl | iPr | iPr | CH₃S(O) |
| 72. | Cl | iPr | iPr | CH₃S(O)₂ |
| 73. | Cl | iPr | cPr | Cl |
| 74. | Cl | iPr | cPr | Br |
| 75. | Cl | iPr | cPr | I |
| 76. | Cl | iPr | cPr | CF₃ |
| 77. | Cl | iPr | cPr | CH₃S(O) |
| 78. | Cl | iPr | cPr | CH₃S(O)₂ |
| 79. | Cl | iPr | CHF₂CH₂ | Cl |
| 80. | Cl | iPr | CHF₂CH₂ | Br |
| 81. | Cl | iPr | CHF₂CH₂ | I |
| 82. | Cl | iPr | CHF₂CH₂ | CF₃ |
| 83. | Cl | iPr | CHF₂CH₂ | CH₃S(O) |
| 84. | Cl | iPr | CHF₂CH₂ | CH₃S(O)₂ |
| 85. | Cl | iPr | CF₃CH₂ | Cl |
| 86. | Cl | iPr | CF₃CH₂ | Br |
| 87. | Cl | iPr | CF₃CH₂ | I |
| 88. | Cl | iPr | CF₃CH₂ | CF₃ |
| 89. | Cl | iPr | CF₃CH₂ | CH₃S(O) |
| 90. | Cl | iPr | CF₃CH₂ | CH₃S(O)₂ |
| 91. | Cl | cPr | cPr | Cl |
| 92. | Cl | cPr | cPr | Br |
| 93. | Cl | cPr | cPr | I |
| 94. | Cl | cPr | cPr | CF₃ |
| 95. | Cl | cPr | cPr | CH₃S(O) |
| 96. | Cl | cPr | cPr | CH₃S(O)₂ |
| 97. | Cl | cPr | CHF₂CH₂ | Cl |
| 98. | Cl | cPr | CHF₂CH₂ | Br |
| 99. | Cl | cPr | CHF₂CH₂ | I |
| 100. | Cl | cPr | CHF₂CH₂ | CF₃ |
| 101. | Cl | cPr | CHF₂CH₂ | CH₃S(O) |
| 102. | Cl | cPr | CHF₂CH₂ | CH₃S(O)₂ |
| 103. | Cl | cPr | CF₃CH₂ | Cl |
| 104. | Cl | cPr | CF₃CH₂ | Br |
| 105. | Cl | cPr | CF₃CH₂ | I |
| 106. | Cl | cPr | CF₃CH₂ | CF₃ |
| 107. | Cl | cPr | CF₃CH₂ | CH₃S(O) |
| 108. | Cl | cPr | CF₃CH₂ | CH₃S(O)₂ |
| 109. | Cl | CHF₂CH₂ | CHF₂CH₂ | Cl |
| 110. | Cl | CHF₂CH₂ | CHF₂CH₂ | Br |
| 111. | Cl | CHF₂CH₂ | CHF₂CH₂ | I |
| 112. | Cl | CHF₂CH₂ | CHF₂CH₂ | CF₃ |
| 113. | Cl | CHF₂CH₂ | CHF₂CH₂ | CH₃S(O) |
| 114. | Cl | CHF₂CH₂ | CHF₂CH₂ | CH₃S(O)₂ |
| 115. | Cl | CHF₂CH₂ | CF₃CH₂ | Cl |
| 116. | Cl | CHF₂CH₂ | CF₃CH₂ | Br |
| 117. | Cl | CHF₂CH₂ | CF₃CH₂ | I |
| 118. | Cl | CHF₂CH₂ | CF₃CH₂ | CF₃ |
| 119. | Cl | CHF₂CH₂ | CF₃CH₂ | CH₃S(O) |
| 120. | Cl | CHF₂CH₂ | CF₃CH₂ | CH₃S(O)₂ |
| 121. | Cl | CF₃CH₂ | CF₃CH₂ | Cl |
| 122. | Cl | CF₃CH₂ | CF₃CH₂ | Br |
| 123. | Cl | CF₃CH₂ | CF₃CH₂ | I |
| 124. | Cl | CF₃CH₂ | CF₃CH₂ | CF₃ |
| 125. | Cl | CF₃CH₂ | CF₃CH₂ | CH₃S(O) |
| 126. | Cl | CF₃CH₂ | CF₃CH₂ | CH₃S(O)₂ |
| 127. | Cl | CH₃O | CH₃ | Cl |
| 128. | Cl | CH₃O | CH₃ | Br |
| 129. | Cl | CH₃O | CH₃ | I |
| 130. | Cl | CH₃O | CH₃ | CF₃ |
| 131. | Cl | CH₃O | CH₃ | CH₃S(O) |
| 132. | Cl | CH₃O | CH₃ | CH₃S(O)₂ |
| 133. | Cl | CH₃O | CH₃CH₂ | Cl |
| 134. | Cl | CH₃O | CH₃CH₂ | Br |
| 135. | Cl | CH₃O | CH₃CH₂ | I |
| 136. | Cl | CH₃O | CH₃CH₂ | CF₃ |
| 137. | Cl | CH₃O | CH₃CH₂ | CH₃S(O) |
| 138. | Cl | CH₃O | CH₃CH₂ | CH₃S(O)₂ |
| 139. | Cl | CH₃O | iPr | Cl |
| 140. | Cl | CH₃O | iPr | Br |
| 141. | Cl | CH₃O | iPr | I |
| 142. | Cl | CH₃O | iPr | CF₃ |
| 143. | Cl | CH₃O | iPr | CH₃S(O) |
| 144. | Cl | CH₃O | iPr | CH₃S(O)₂ |
| 145. | Cl | CH₃O | cPr | Cl |
| 146. | Cl | CH₃O | cPr | Br |
| 147. | Cl | CH₃O | cPr | I |
| 148. | Cl | CH₃O | cPr | CF₃ |
| 149. | Cl | CH₃O | cPr | CH₃S(O) |
| 150. | Cl | CH₃O | cPr | CH₃S(O)₂ |
| 151. | Cl | CH₃O | CHF₂CH₂ | Cl |
| 152. | Cl | CH₃O | CHF₂CH₂ | Br |
| 153. | Cl | CH₃O | CHF₂CH₂ | I |
| 154. | Cl | CH₃O | CHF₂CH₂ | CF₃ |
| 155. | Cl | CH₃O | CHF₂CH₂ | CH₃S(O) |
| 156. | Cl | CH₃O | CHF₂CH₂ | CH₃S(O)₂ |
| 157. | Cl | CH₃O | CF₃CH₂ | Cl |
| 158. | Cl | CH₃O | CF₃CH₂ | Br |
| 159. | Cl | CH₃O | CF₃CH₂ | I |
| 160. | Cl | CH₃O | CF₃CH₂ | CF₃ |
| 161. | Cl | CH₃O | CF₃CH₂ | CH₃S(O) |
| 162. | Cl | CH₃O | CF₃CH₂ | CH₃S(O)₂ |
| 163. | Cl | Ph | CH₃ | Cl |
| 164. | Cl | Ph | CH₃ | Br |
| 165. | Cl | Ph | CH₃ | I |
| 166. | Cl | Ph | CH₃ | CF₃ |
| 167. | Cl | Ph | CH₃ | CH₃S(O) |
| 168. | Cl | Ph | CH₃ | CH₃S(O)₂ |
| 169. | Cl | Ph | CH₃CH₂ | Cl |
| 170. | Cl | Ph | CH₃CH₂ | Br |
| 171. | Cl | Ph | CH₃CH₂ | I |
| 172. | Cl | Ph | CH₃CH₂ | CF₃ |
| 173. | Cl | Ph | CH₃CH₂ | CH₃S(O) |
| 174. | Cl | Ph | CH₃CH₂ | CH₃S(O)₂ |
| 175. | Cl | Ph | iPr | Cl |
| 176. | Cl | Ph | iPr | Br |
| 177. | Cl | Ph | iPr | I |
| 178. | Cl | Ph | iPr | CF₃ |
| 179. | Cl | Ph | iPr | CH₃S(O) |
| 180. | Cl | Ph | iPr | CH₃S(O)₂ |
| 181. | Cl | Ph | cPr | Cl |
| 182. | Cl | Ph | cPr | Br |
| 183. | Cl | Ph | cPr | I |
| 184. | Cl | Ph | cPr | CF₃ |
| 185. | Cl | Ph | cPr | CH₃S(O) |
| 186. | Cl | Ph | cPr | CH₃S(O)₂ |
| 187. | Cl | Ph | CHF₂CH₂ | Cl |
| 188. | Cl | Ph | CHF₂CH₂ | Br |
| 189. | Cl | Ph | CHF₂CH₂ | I |
| 190. | Cl | Ph | CHF₂CH₂ | CF₃ |
| 191. | Cl | Ph | CHF₂CH₂ | CH₃S(O) |
| 192. | Cl | Ph | CHF₂CH₂ | CH₃S(O)₂ |
| 193. | Cl | Ph | CF₃CH₂ | Cl |
| 194. | Cl | Ph | CF₃CH₂ | Br |
| 195. | Cl | Ph | CF₃CH₂ | I |
| 196. | Cl | Ph | CF₃CH₂ | CF₃ |
| 197. | Cl | Ph | CF₃CH₂ | CH₃S(O) |
| 198. | Cl | Ph | CF₃CH₂ | CH₃S(O)₂ |
| 199. | Cl | 4-Cl—Ph | CH₃ | Cl |
| 200. | Cl | 4-Cl—Ph | CH₃ | Br |
| 201. | Cl | 4-Cl—Ph | CH₃ | I |
| 202. | Cl | 4-Cl—Ph | CH₃ | CF₃ |
| 203. | Cl | 4-Cl—Ph | CH₃ | CH₃S(O) |
| 204. | Cl | 4-Cl—Ph | CH₃ | CH₃S(O)₂ |
| 205. | Cl | 4-Cl—Ph | CH₃CH₂ | Cl |
| 206. | Cl | 4-Cl—Ph | CH₃CH₂ | Br |
| 207. | Cl | 4-Cl—Ph | CH₃CH₂ | I |
| 208. | Cl | 4-Cl—Ph | CH₃CH₂ | CF₃ |
| 209. | Cl | 4-Cl—Ph | CH₃CH₂ | CH₃S(O) |
| 210. | Cl | 4-Cl—Ph | CH₃CH₂ | CH₃S(O)₂ |
| 211. | Cl | 4-Cl—Ph | iPr | Cl |
| 212. | Cl | 4-Cl—Ph | iPr | Br |
| 213. | Cl | 4-Cl—Ph | iPr | I |
| 214. | Cl | 4-Cl—Ph | iPr | CF₃ |
| 215. | Cl | 4-Cl—Ph | iPr | CH₃S(O) |
| 216. | Cl | 4-Cl—Ph | iPr | CH₃S(O)₂ |
| 217. | Cl | 4-Cl—Ph | cPr | Cl |
| 218. | Cl | 4-Cl—Ph | cPr | Br |
| 219. | Cl | 4-Cl—Ph | cPr | I |
| 220. | Cl | 4-Cl—Ph | cPr | CF₃ |
| 221. | Cl | 4-Cl—Ph | cPr | CH₃S(O) |
| 222. | Cl | 4-Cl—Ph | cPr | CH₃S(O)₂ |
| 223. | Cl | 4-Cl—Ph | CHF₂CH₂ | Cl |
| 224. | Cl | 4-Cl—Ph | CHF₂CH₂ | Br |
| 225. | Cl | 4-Cl—Ph | CHF₂CH₂ | I |
| 226. | Cl | 4-Cl—Ph | CHF₂CH₂ | CF₃ |
| 227. | Cl | 4-Cl—Ph | CHF₂CH₂ | CH₃S(O) |
| 228. | Cl | 4-Cl—Ph | CHF₂CH₂ | CH₃S(O)₂ |
| 229. | Cl | 4-Cl—Ph | CF₃CH₂ | Cl |
| 230. | Cl | 4-Cl—Ph | CF₃CH₂ | Br |
| 231. | Cl | 4-Cl—Ph | CF₃CH₂ | I |
| 232. | Cl | 4-Cl—Ph | CF₃CH₂ | CF₃ |
| 233. | Cl | 4-Cl—Ph | CF₃CH₂ | CH₃S(O) |
| 234. | Cl | 4-Cl—Ph | CF₃CH₂ | CH₃S(O)₂ |
| 235. | Cl | 4-CH₃O—Ph | CH₃ | Cl |
| 236. | Cl | 4-CH₃O—Ph | CH₃ | Br |
| 237. | Cl | 4-CH₃O—Ph | CH₃ | I |
| 238. | Cl | 4-CH₃O—Ph | CH₃ | CF₃ |
| 239. | Cl | 4-CH₃O—Ph | CH₃ | CH₃S(O) |
| 240. | Cl | 4-CH₃O—Ph | CH₃ | CH₃S(O)₂ |
| 241. | Cl | 4-CH₃O—Ph | CH₃CH₂ | Cl |
| 242. | Cl | 4-CH₃O—Ph | CH₃CH₂ | Br |
| 243. | Cl | 4-CH₃O—Ph | CH₃CH₂ | I |
| 244. | Cl | 4-CH₃O—Ph | CH₃CH₂ | CF₃ |
| 245. | Cl | 4-CH₃O—Ph | CH₃CH₂ | CH₃S(O) |
| 246. | Cl | 4-CH₃O—Ph | CH₃CH₂ | CH₃S(O)₂ |
| 247. | Cl | 4-CH₃O—Ph | iPr | Cl |
| 248. | Cl | 4-CH₃O—Ph | iPr | Br |
| 249. | Cl | 4-CH₃O—Ph | iPr | I |
| 250. | Cl | 4-CH₃O—Ph | iPr | CF₃ |
| 251. | Cl | 4-CH₃O—Ph | iPr | CH₃S(O) |
| 252. | Cl | 4-CH₃O—Ph | iPr | CH₃S(O)₂ |
| 253. | Cl | 4-CH₃O—Ph | cPr | Cl |
| 254. | Cl | 4-CH₃O—Ph | cPr | Br |
| 255. | Cl | 4-CH₃O—Ph | cPr | I |
| 256. | Cl | 4-CH₃O—Ph | cPr | CF₃ |
| 257. | Cl | 4-CH₃O—Ph | cPr | CH₃S(O) |
| 258. | Cl | 4-CH₃O—Ph | cPr | CH₃S(O)₂ |
| 259. | Cl | 4-CH₃O—Ph | CHF₂CH₂ | Cl |
| 260. | Cl | 4-CH₃O—Ph | CHF₂CH₂ | Br |
| 261. | Cl | 4-CH₃O—Ph | CHF₂CH₂ | I |
| 262. | Cl | 4-CH₃O—Ph | CHF₂CH₂ | CF₃ |
| 263. | Cl | 4-CH₃O—Ph | CHF₂CH₂ | CH₃S(O) |
| 264. | Cl | 4-CH₃O—Ph | CHF₂CH₂ | CH₃S(O)₂ |
| 265. | Cl | 4-CH₃O—Ph | CF₃CH₂ | Cl |
| 266. | Cl | 4-CH₃O—Ph | CF₃CH₂ | Br |
| 267. | Cl | 4-CH₃O—Ph | CF₃CH₂ | I |
| 268. | Cl | 4-CH₃O—Ph | CF₃CH₂ | CF₃ |
| 269. | Cl | 4-CH₃O—Ph | CF₃CH₂ | CH₃S(O) |
| 270. | Cl | 4-CH₃O—Ph | CF₃CH₂ | CH₃S(O)₂ |
| 271. | Cl | 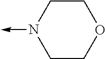 | | Cl |
| 272. | Cl | 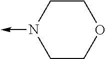 | | Br |
| 273. | Cl | 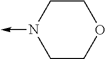 | | I |
| 274. | Cl | 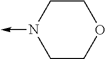 | | CF₃ |
| 275. | Cl | 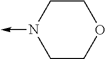 | | CH₃S(O) |
| 276. | Cl | 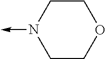 | | CH₃S(O)₂ |
| 277. | CH₃ | CH₃ | CH₃ | Cl |
| 278. | CH₃ | CH₃ | CH₃ | Br |
| 279. | CH₃ | CH₃ | CH₃ | I |
| 280. | CH₃ | CH₃ | CH₃ | CF₃ |
| 281. | CH₃ | CH₃ | CH₃ | CH₃S(O) |
| 282. | CH₃ | CH₃ | CH₃ | CH₃S(O)₂ |
| 283. | CH₃ | CH₃ | CH₃CH₂ | Cl |
| 284. | CH₃ | CH₃ | CH₃CH₂ | Br |
| 285. | CH₃ | CH₃ | CH₃CH₂ | I |
| 286. | CH₃ | CH₃ | CH₃CH₂ | CF₃ |
| 287. | CH₃ | CH₃ | CH₃CH₂ | CH₃S(O) |
| 288. | CH₃ | CH₃ | CH₃CH₂ | CH₃S(O)₂ |
| 289. | CH₃ | CH₃ | iPr | Cl |
| 290. | CH₃ | CH₃ | iPr | Br |
| 291. | CH₃ | CH₃ | iPr | I |
| 292. | CH₃ | CH₃ | iPr | CF₃ |
| 293. | CH₃ | CH₃ | iPr | CH₃S(O) |
| 294. | CH₃ | CH₃ | iPr | CH₃S(O)₂ |
| 295. | CH₃ | CH₃ | cPr | Cl |
| 296. | CH₃ | CH₃ | cPr | Br |
| 297. | CH₃ | CH₃ | cPr | I |
| 298. | CH₃ | CH₃ | cPr | CF₃ |
| 299. | CH₃ | CH₃ | cPr | CH₃S(O) |
| 300. | CH₃ | CH₃ | cPr | CH₃S(O)₂ |
| 301. | CH₃ | CH₃ | CHF₂CH₂ | Cl |
| 302. | CH₃ | CH₃ | CHF₂CH₂ | Br |
| 303. | CH₃ | CH₃ | CHF₂CH₂ | I |
| 304. | CH₃ | CH₃ | CHF₂CH₂ | CF₃ |
| 305. | CH₃ | CH₃ | CHF₂CH₂ | CH₃S(O) |
| 306. | CH₃ | CH₃ | CHF₂CH₂ | CH₃S(O)₂ |
| 307. | CH₃ | CH₃ | CF₃CH₂ | Cl |
| 308. | CH₃ | CH₃ | CF₃CH₂ | Br |
| 309. | CH₃ | CH₃ | CF₃CH₂ | I |
| 310. | CH₃ | CH₃ | CF₃CH₂ | CF₃ |
| 311. | CH₃ | CH₃ | CF₃CH₂ | CH₃S(O) |
| 312. | CH₃ | CH₃ | CF₃CH₂ | CH₃S(O)₂ |
| 313. | CH₃ | CH₃CH₂ | CH₃CH₂ | Cl |
| 314. | CH₃ | CH₃CH₂ | CH₃CH₂ | Br |
| 315. | CH₃ | CH₃CH₂ | CH₃CH₂ | I |
| 316. | CH₃ | CH₃CH₂ | CH₃CH₂ | CF₃ |
| 317. | CH₃ | CH₃CH₂ | CH₃CH₂ | CH₃S(O) |
| 318. | CH₃ | CH₃CH₂ | CH₃CH₂ | CH₃S(O)₂ |
| 319. | CH₃ | CH₃CH₂ | iPr | Cl |
| 320. | CH₃ | CH₃CH₂ | iPr | Br |
| 321. | CH₃ | CH₃CH₂ | iPr | I |
| 322. | CH₃ | CH₃CH₂ | iPr | CF₃ |
| 323. | CH₃ | CH₃CH₂ | iPr | CH₃S(O) |
| 324. | CH₃ | CH₃CH₂ | iPr | CH₃S(O)₂ |
| 325. | CH₃ | CH₃CH₂ | cPr | Cl |
| 326. | CH₃ | CH₃CH₂ | cPr | Br |
| 327. | CH₃ | CH₃CH₂ | cPr | I |
| 328. | CH₃ | CH₃CH₂ | cPr | CF₃ |
| 329. | CH₃ | CH₃CH₂ | cPr | CH₃S(O) |
| 330. | CH₃ | CH₃CH₂ | cPr | CH₃S(O)₂ |
| 331. | CH₃ | CH₃CH₂ | CHF₂CH₂ | Cl |
| 332. | CH₃ | CH₃CH₂ | CHF₂CH₂ | Br |
| 333. | CH₃ | CH₃CH₂ | CHF₂CH₂ | I |
| 334. | CH₃ | CH₃CH₂ | CHF₂CH₂ | CF₃ |
| 335. | CH₃ | CH₃CH₂ | CHF₂CH₂ | CH₃S(O) |
| 336. | CH₃ | CH₃CH₂ | CHF₂CH₂ | CH₃S(O)₂ |
| 337. | CH₃ | CH₃CH₂ | CF₃CH₂ | Cl |
| 338. | CH₃ | CH₃CH₂ | CF₃CH₂ | Br |
| 339. | CH₃ | CH₃CH₂ | CF₃CH₂ | I |
| 340. | CH₃ | CH₃CH₂ | CF₃CH₂ | CF₃ |
| 341. | CH₃ | CH₃CH₂ | CF₃CH₂ | CH₃S(O) |
| 342. | CH₃ | CH₃CH₂ | CF₃CH₂ | CH₃S(O)₂ |
| 343. | CH₃ | iPr | iPr | Cl |
| 344. | CH₃ | iPr | iPr | Br |
| 345. | CH₃ | iPr | iPr | I |
| 346. | CH₃ | iPr | iPr | CF₃ |
| 347. | CH₃ | iPr | iPr | CH₃S(O) |
| 348. | CH₃ | iPr | iPr | CH₃S(O)₂ |
| 349. | CH₃ | iPr | cPr | Cl |
| 350. | CH₃ | iPr | cPr | Br |
| 351. | CH₃ | iPr | cPr | I |
| 352. | CH₃ | iPr | cPr | CF₃ |
| 353. | CH₃ | iPr | cPr | CH₃S(O) |
| 354. | CH₃ | iPr | cPr | CH₃S(O)₂ |
| 355. | CH₃ | iPr | CHF₂CH₂ | Cl |
| 356. | CH₃ | iPr | CHF₂CH₂ | Br |
| 357. | CH₃ | iPr | CHF₂CH₂ | I |
| 358. | CH₃ | iPr | CHF₂CH₂ | CF₃ |
| 359. | CH₃ | iPr | CHF₂CH₂ | CH₃S(O) |
| 360. | CH₃ | iPr | CHF₂CH₂ | CH₃S(O)₂ |
| 361. | CH₃ | iPr | CF₃CH₂ | Cl |
| 362. | CH₃ | iPr | CF₃CH₂ | Br |
| 363. | CH₃ | iPr | CF₃CH₂ | I |
| 364. | CH₃ | iPr | CF₃CH₂ | CF₃ |
| 365. | CH₃ | iPr | CF₃CH₂ | CH₃S(O) |
| 366. | CH₃ | iPr | CF₃CH₂ | CH₃S(O)₂ |
| 367. | CH₃ | cPr | cPr | Cl |
| 368. | CH₃ | cPr | cPr | Br |
| 369. | CH₃ | cPr | cPr | I |
| 370. | CH₃ | cPr | cPr | CF₃ |
| 371. | CH₃ | cPr | cPr | CH₃S(O) |
| 372. | CH₃ | cPr | cPr | CH₃S(O)₂ |
| 373. | CH₃ | cPr | CHF₂CH₂ | Cl |
| 374. | CH₃ | cPr | CHF₂CH₂ | Br |
| 375. | CH₃ | cPr | CHF₂CH₂ | I |
| 376. | CH₃ | cPr | CHF₂CH₂ | CF₃ |
| 377. | CH₃ | cPr | CHF₂CH₂ | CH₃S(O) |
| 378. | CH₃ | cPr | CHF₂CH₂ | CH₃S(O)₂ |
| 379. | CH₃ | cPr | CF₃CH₂ | Cl |
| 380. | CH₃ | cPr | CF₃CH₂ | Br |
| 381. | CH₃ | cPr | CF₃CH₂ | I |
| 382. | CH₃ | cPr | CF₃CH₂ | CF₃ |
| 383. | CH₃ | cPr | CF₃CH₂ | CH₃S(O) |
| 384. | CH₃ | cPr | CF₃CH₂ | CH₃S(O)₂ |
| 385. | CH₃ | CHF₂CH₂ | CHF₂CH₂ | Cl |
| 386. | CH₃ | CHF₂CH₂ | CHF₂CH₂ | Br |
| 387. | CH₃ | CHF₂CH₂ | CHF₂CH₂ | I |
| 388. | CH₃ | CHF₂CH₂ | CHF₂CH₂ | CF₃ |
| 389. | CH₃ | CHF₂CH₂ | CHF₂CH₂ | CH₃S(O) |
| 390. | CH₃ | CHF₂CH₂ | CHF₂CH₂ | CH₃S(O)₂ |
| 391. | CH₃ | CHF₂CH₂ | CF₃CH₂ | Cl |
| 392. | CH₃ | CHF₂CH₂ | CF₃CH₂ | Br |
| 393. | CH₃ | CHF₂CH₂ | CF₃CH₂ | I |
| 394. | CH₃ | CHF₂CH₂ | CF₃CH₂ | CF₃ |
| 395. | CH₃ | CHF₂CH₂ | CF₃CH₂ | CH₃S(O) |
| 396. | CH₃ | CHF₂CH₂ | CF₃CH₂ | CH₃S(O)₂ |
| 397. | CH₃ | CF₃CH₂ | CF₃CH₂ | Cl |
| 398. | CH₃ | CF₃CH₂ | CF₃CH₂ | Br |
| 399. | CH₃ | CF₃CH₂ | CF₃CH₂ | I |
| 400. | CH₃ | CF₃CH₂ | CF₃CH₂ | CF₃ |
| 401. | CH₃ | CF₃CH₂ | CF₃CH₂ | CH₃S(O) |
| 402. | CH₃ | CF₃CH₂ | CF₃CH₂ | CH₃S(O)₂ |
| 403. | CH₃ | CH₃O | CH₃ | Cl |
| 404. | CH₃ | CH₃O | CH₃ | Br |
| 405. | CH₃ | CH₃O | CH₃ | I |
| 406. | CH₃ | CH₃O | CH₃ | CF₃ |
| 407. | CH₃ | CH₃O | CH₃ | CH₃S(O) |
| 408. | CH₃ | CH₃O | CH₃ | CH₃S(O)₂ |
| 409. | CH₃ | CH₃O | CH₃CH₂ | Cl |
| 410. | CH₃ | CH₃O | CH₃CH₂ | Br |
| 411. | CH₃ | CH₃O | CH₃CH₂ | I |
| 412. | CH₃ | CH₃O | CH₃CH₂ | CF₃ |
| 413. | CH₃ | CH₃O | CH₃CH₂ | CH₃S(O) |
| 414. | CH₃ | CH₃O | CH₃CH₂ | CH₃S(O)₂ |
| 415. | CH₃ | CH₃O | iPr | Cl |
| 416. | CH₃ | CH₃O | iPr | Br |
| 417. | CH₃ | CH₃O | iPr | I |
| 418. | CH₃ | CH₃O | iPr | CF₃ |
| 419. | CH₃ | CH₃O | iPr | CH₃S(O) |
| 420. | CH₃ | CH₃O | iPr | CH₃S(O)₂ |
| 421. | CH₃ | CH₃O | cPr | Cl |
| 422. | CH₃ | CH₃O | cPr | Br |
| 423. | CH₃ | CH₃O | cPr | I |
| 424. | CH₃ | CH₃O | cPr | CF₃ |
| 425. | CH₃ | CH₃O | cPr | CH₃S(O) |
| 426. | CH₃ | CH₃O | cPr | CH₃S(O)₂ |
| 427. | CH₃ | CH₃O | CHF₂CH₂ | Cl |
| 428. | CH₃ | CH₃O | CHF₂CH₂ | Br |
| 429. | CH₃ | CH₃O | CHF₂CH₂ | I |
| 430. | CH₃ | CH₃O | CHF₂CH₂ | CF₃ |
| 431. | CH₃ | CH₃O | CHF₂CH₂ | CH₃S(O) |
| 432. | CH₃ | CH₃O | CHF₂CH₂ | CH₃S(O)₂ |
| 433. | CH₃ | CH₃O | CF₃CH₂ | Cl |
| 434. | CH₃ | CH₃O | CF₃CH₂ | Br |
| 435. | CH₃ | CH₃O | CF₃CH₂ | I |
| 436. | CH₃ | CH₃O | CF₃CH₂ | CF₃ |
| 437. | CH₃ | CH₃O | CF₃CH₂ | CH₃S(O) |
| 438. | CH₃ | CH₃O | CF₃CH₂ | CH₃S(O)₂ |
| 439. | CH₃ | Ph | CH₃ | Cl |
| 440. | CH₃ | Ph | CH₃ | Br |
| 441. | CH₃ | Ph | CH₃ | I |
| 442. | CH₃ | Ph | CH₃ | CF₃ |
| 443. | CH₃ | Ph | CH₃ | CH₃S(O) |
| 444. | CH₃ | Ph | CH₃ | CH₃S(O)₂ |
| 445. | CH₃ | Ph | CH₃CH₂ | Cl |
| 446. | CH₃ | Ph | CH₃CH₂ | Br |
| 447. | CH₃ | Ph | CH₃CH₂ | I |
| 448. | CH₃ | Ph | CH₃CH₂ | CF₃ |
| 449. | CH₃ | Ph | CH₃CH₂ | CH₃S(O) |
| 450. | CH₃ | Ph | CH₃CH₂ | CH₃S(O)₂ |
| 451. | CH₃ | Ph | iPr | Cl |
| 452. | CH₃ | Ph | iPr | Br |
| 453. | CH₃ | Ph | iPr | I |
| 454. | CH₃ | Ph | iPr | CF₃ |
| 455. | CH₃ | Ph | iPr | CH₃S(O) |
| 456. | CH₃ | Ph | iPr | CH₃S(O)₂ |
| 457. | CH₃ | Ph | cPr | Cl |
| 458. | CH₃ | Ph | cPr | Br |
| 459. | CH₃ | Ph | cPr | I |
| 460. | CH₃ | Ph | cPr | CF₃ |
| 461. | CH₃ | Ph | cPr | CH₃S(O) |
| 462. | CH₃ | Ph | cPr | CH₃S(O)₂ |
| 463. | CH₃ | Ph | CHF₂CH₂ | Cl |
| 464. | CH₃ | Ph | CHF₂CH₂ | Br |
| 465. | CH₃ | Ph | CHF₂CH₂ | I |
| 466. | CH₃ | Ph | CHF₂CH₂ | CF₃ |
| 467. | CH₃ | Ph | CHF₂CH₂ | CH₃S(O) |
| 468. | CH₃ | Ph | CHF₂CH₂ | CH₃S(O)₂ |
| 469. | CH₃ | Ph | CF₃CH₂ | Cl |
| 470. | CH₃ | Ph | CF₃CH₂ | Br |
| 471. | CH₃ | Ph | CF₃CH₂ | I |
| 472. | CH₃ | Ph | CF₃CH₂ | CF₃ |
| 473. | CH₃ | Ph | CF₃CH₂ | CH₃S(O) |
| 474. | CH₃ | Ph | CF₃CH₂ | CH₃S(O)₂ |
| 475. | CH₃ | 4-Cl—Ph | CH₃ | Cl |
| 476. | CH₃ | 4-Cl—Ph | CH₃ | Br |
| 477. | CH₃ | 4-Cl—Ph | CH₃ | I |
| 478. | CH₃ | 4-Cl—Ph | CH₃ | CF₃ |
| 479. | CH₃ | 4-Cl—Ph | CH₃ | CH₃S(O) |
| 480. | CH₃ | 4-Cl—Ph | CH₃ | CH₃S(O)₂ |
| 481. | CH₃ | 4-Cl—Ph | CH₃CH₂ | Cl |
| 482. | CH₃ | 4-Cl—Ph | CH₃CH₂ | Br |
| 483. | CH₃ | 4-Cl—Ph | CH₃CH₂ | I |
| 484. | CH₃ | 4-Cl—Ph | CH₃CH₂ | CF₃ |
| 485. | CH₃ | 4-Cl—Ph | CH₃CH₂ | CH₃S(O) |
| 486. | CH₃ | 4-Cl—Ph | CH₃CH₂ | CH₃S(O)₂ |
| 487. | CH₃ | 4-Cl—Ph | iPr | Cl |
| 488. | CH₃ | 4-Cl—Ph | iPr | Br |
| 489. | CH₃ | 4-Cl—Ph | iPr | I |
| 490. | CH₃ | 4-Cl—Ph | iPr | CF₃ |
| 491. | CH₃ | 4-Cl—Ph | iPr | CH₃S(O) |
| 492. | CH₃ | 4-Cl—Ph | iPr | CH₃S(O)₂ |
| 493. | CH₃ | 4-Cl—Ph | cPr | Cl |
| 494. | CH₃ | 4-Cl—Ph | cPr | Br |
| 495. | CH₃ | 4-Cl—Ph | cPr | I |
| 496. | CH₃ | 4-Cl—Ph | cPr | CF₃ |
| 497. | CH₃ | 4-Cl—Ph | cPr | CH₃S(O) |
| 498. | CH₃ | 4-Cl—Ph | cPr | CH₃S(O)₂ |
| 499. | CH₃ | 4-Cl—Ph | CHF₂CH₂ | Cl |
| 500. | CH₃ | 4-Cl—Ph | CHF₂CH₂ | Br |
| 501. | CH₃ | 4-Cl—Ph | CHF₂CH₂ | I |
| 502. | CH₃ | 4-Cl—Ph | CHF₂CH₂ | CF₃ |
| 503. | CH₃ | 4-Cl—Ph | CHF₂CH₂ | CH₃S(O) |
| 504. | CH₃ | 4-Cl—Ph | CHF₂CH₂ | CH₃S(O)₂ |
| 505. | CH₃ | 4-Cl—Ph | CF₃CH₂ | Cl |
| 506. | CH₃ | 4-Cl—Ph | CF₃CH₂ | Br |
| 507. | CH₃ | 4-Cl—Ph | CF₃CH₂ | I |
| 508. | CH₃ | 4-Cl—Ph | CF₃CH₂ | CF₃ |
| 509. | CH₃ | 4-Cl—Ph | CF₃CH₂ | CH₃S(O) |
| 510. | CH₃ | 4-Cl—Ph | CF₃CH₂ | CH₃S(O)₂ |
| 511. | CH₃ | 4-CH₃O—Ph | CH₃ | Cl |
| 512. | CH₃ | 4-CH₃O—Ph | CH₃ | Br |
| 513. | CH₃ | 4-CH₃O—Ph | CH₃ | I |
| 514. | CH₃ | 4-CH₃O—Ph | CH₃ | CF₃ |
| 515. | CH₃ | 4-CH₃O—Ph | CH₃ | CH₃S(O) |
| 516. | CH₃ | 4-CH₃O—Ph | CH₃ | CH₃S(O)₂ |
| 517. | CH₃ | 4-CH₃O—Ph | CH₃CH₂ | Cl |
| 518. | CH₃ | 4-CH₃O—Ph | CH₃CH₂ | Br |
| 519. | CH₃ | 4-CH₃O—Ph | CH₃CH₂ | I |
| 520. | CH₃ | 4-CH₃O—Ph | CH₃CH₂ | CF₃ |
| 521. | CH₃ | 4-CH₃O—Ph | CH₃CH₂ | CH₃S(O) |
| 522. | CH₃ | 4-CH₃O—Ph | CH₃CH₂ | CH₃S(O)₂ |
| 523. | CH₃ | 4-CH₃O—Ph | iPr | Cl |
| 524. | CH₃ | 4-CH₃O—Ph | iPr | Br |
| 525. | CH₃ | 4-CH₃O—Ph | iPr | I |
| 526. | CH₃ | 4-CH₃O—Ph | iPr | CF₃ |
| 527. | CH₃ | 4-CH₃O—Ph | iPr | CH₃S(O) |
| 528. | CH₃ | 4-CH₃O—Ph | iPr | CH₃S(O)₂ |
| 529. | CH₃ | 4-CH₃O—Ph | cPr | Cl |
| 530. | CH₃ | 4-CH₃O—Ph | cPr | Br |
| 531. | CH₃ | 4-CH₃O—Ph | cPr | I |
| 532. | CH₃ | 4-CH₃O—Ph | cPr | CF₃ |
| 533. | CH₃ | 4-CH₃O—Ph | cPr | CH₃S(O) |
| 534. | CH₃ | 4-CH₃O—Ph | cPr | CH₃S(O)₂ |
| 535. | CH₃ | 4-CH₃O—Ph | CHF₂CH₂ | Cl |
| 536. | CH₃ | 4-CH₃O—Ph | CHF₂CH₂ | Br |
| 537. | CH₃ | 4-CH₃O—Ph | CHF₂CH₂ | I |
| 538. | CH₃ | 4-CH₃O—Ph | CHF₂CH₂ | CF₃ |
| 539. | CH₃ | 4-CH₃O—Ph | CHF₂CH₂ | CH₃S(O) |
| 540. | CH₃ | 4-CH₃O—Ph | CHF₂CH₂ | CH₃S(O)₂ |
| 541. | CH₃ | 4-CH₃O—Ph | CF₃CH₂ | Cl |
| 542. | CH₃ | 4-CH₃O—Ph | CF₃CH₂ | Br |
| 543. | CH₃ | 4-CH₃O—Ph | CF₃CH₂ | I |
| 544. | CH₃ | 4-CH₃O—Ph | CF₃CH₂ | CF₃ |
| 545. | CH₃ | 4-CH₃O—Ph | CF₃CH₂ | CH₃S(O) |
| 546. | CH₃ | 4-CH₃O—Ph | CF₃CH₂ | CH₃S(O)₂ |

| | | | |
|---|---|---|---|
| 547. | CH₃ | 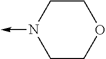 | Cl |
| 548. | CH₃ | 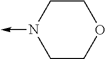 | Br |
| 549. | CH₃ | 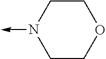 | I |
| 550. | CH₃ | 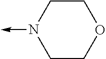 | CF₃ |
| 551. | CH₃ | 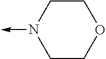 | CH₃S(O) |
| 552. | CH₃ | 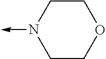 | CH₃S(O)₂ |
| 553. | Cl | 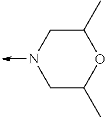 | Cl |
| 554. | Cl | 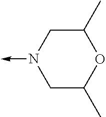 | Br |
| 555. | Cl | 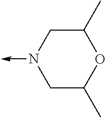 | I |
| 556. | Cl | 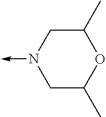 | CF₃ |
| 557. | Cl | 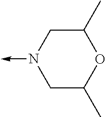 | CH₃S(O) |
| 558. | Cl | 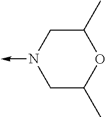 | CH₃S(O)₂ |
| 559. | Cl | 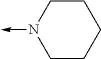 | Cl |
| 560. | Cl | 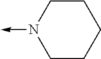 | Br |
| 561. | Cl | 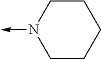 | I |
| 562. | Cl | 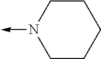 | CF₃ |
| 563. | Cl | 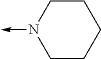 | CH₃S(O) |
| 564. | Cl | 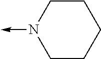 | CH₃S(O)₂ |
| 565. | Cl | 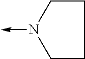 | Cl |
| 566. | Cl | 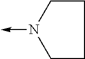 | Br |
| 567. | Cl | 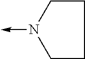 | I |
| 568. | Cl | 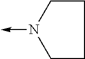 | CF₃ |
| 569. | Cl | 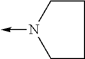 | CH₃S(O) |
| 570. | Cl | 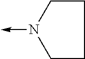 | CH₃S(O)₂ |
| 571. | CH₃ | 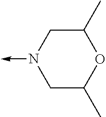 | Cl |
| 572. | CH₃ | 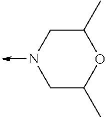 | Br |
| 573. | CH₃ | 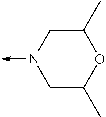 | I |
| 574. | CH₃ | 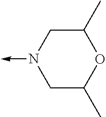 | CF₃ |
| 575. | CH₃ | 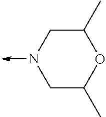 | CH₃S(O) |
| 576. | CH₃ | 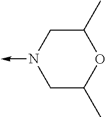 | CH₃S(O)₂ |
| 577. | CH₃ | 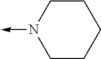 | Cl |
| 578. | CH₃ | 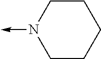 | Br |
| 579. | CH₃ | 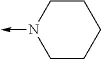 | I |
| 580. | CH₃ | 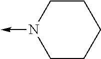 | CF₃ |
| 581. | CH₃ | 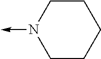 | CH₃S(O) |
| 582. | CH₃ | 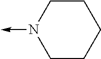 | CH₃S(O)₂ |
| 583. | CH₃ | 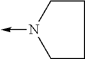 | Cl |
| 584. | CH₃ | 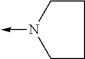 | Br |
| 585. | CH₃ | 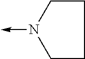 | I |
| 586. | CH₃ | 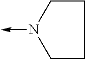 | CF₃ |
| 587. | CH₃ | 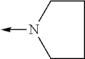 | CH₃S(O) |
| 588. | CH₃ | 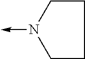 | CH₃S(O)₂ |

In Table A and likewise in tables A′ and A″ below, cPr means cyclopropyl and iPr means isopropyl (=1-methylethyl).

It is apparent to a skilled person that in lines 271 to 276 and in lines 553 to 588 of table A the meanings given for the combination R^{2c}/R^{2d} relate to the moiety NR^{2C}R^{2d} in formulae I.A.I to I.D.VI.

Amongst the combinations of variables R¹, R^{2c}, R^{2d} and R³, those are preferred, where R¹ is CH₃ and where R³ is Cl, Br or CF₃.

Amongst the combinations of variables R¹, R^{2c}, R^{2d} and R³, those are preferred, where R¹ is Cl and where R³ is Cl, Br or CF₃.

Amongst the combinations of variables R¹, R^{2c}, R^{2d} and R³, those are preferred, where R¹ is Cl or CH₃, R^{2c} is CH₃ or C₂H₅, R^{2c} is CH₂CF₃ and where R³ is Cl, Br or CF₃.

Particular preference is given to compounds of the formula I.A.I, where R¹, R^{2c}, R^{2d} and R³ are as defined in Table A and where R¹, R^{2c}, R^{2d} and R³ are in particular as defined in the following tables A′ and A″.

Particular preference is also given to compounds of the formula I.D.I, where R¹, R^{2c}, R^{2d} and R³ are as defined in Table A and where R¹, R^{2c}, R^{2d} and R³ are in particular as defined in the following tables A′ and A″.

TABLE A′

| R¹ | R^{2c} | R^{2d} | R³ |
|---|---|---|---|
| CH₃ | CH₃ | CH₂CH₃ | Cl |
| CH₃ | CH₂CH₃ | CH₂CH₃ | Cl |
| CH₃ | CH₃ | CH₂CF₃ | Cl |
| CH₃ | CH₂CH₃ | CH₂CF₃ | Cl |
| CH₃ | CH₃ | CH₂CHF₂ | Cl |
| CH₃ | CH₂CH₃ | CH₂CHF₂ | Cl |
| CH₃ | CH₃ | CH(CH₃)₂ | Cl |
| CH₃ | cPr | cPr | Cl |
| Cl | CH₃ | CH₂CH₃ | Cl |
| Cl | CH₂CH₃ | CH₂CH₃ | Cl |
| Cl | CH₃ | CH₂CF₃ | Cl |
| Cl | CH₂CH₃ | CH₂CF₃ | Cl |
| Cl | CH₃ | CH₂CHF₂ | Cl |
| Cl | CH₂CH₃ | CH₂CHF₂ | Cl |
| Cl | CH₃ | CH(CH₃)₂ | Cl |
| Cl | cPr | cPr | Cl |
| CH₃ | CH₃ | CH₂CH₃ | Br |
| CH₃ | CH₂CH₃ | CH₂CH₃ | Br |
| CH₃ | CH₃ | CH₂CF3 | Br |
| CH₃ | CH₂CH₃ | CH₃CF₃ | Br |
| CH₃ | CH₃ | CH₃CHF₃ | Br |
| CH₃ | CH₂CH₃ | CH₃CHF₃ | Br |
| CH₃ | CH₃ | CH(CH₃)₂ | Br |
| CH₃ | cPr | cPr | Br |
| Cl | CH₃ | CH₃CH₃ | Br |
| Cl | CH₂CH₃ | CH₃CH₃ | Br |
| Cl | CH₃ | CH₃CF₃ | Br |
| Cl | CH₂CH₃ | CH₃CF₃ | Br |
| Cl | CH₃ | CH₃CHF₃ | Br |
| Cl | CH₂CH₃ | CH₂CHF₂ | Br |
| Cl | CH₃ | CH(CH₃)₂ | Br |
| Cl | cPr | cPr | Br |
| CH₃ | CH₃ | CH₂CH₃ | CF₃ |
| CH₃ | CH₂CH₃ | CH₂CH₃ | CF₃ |
| CH₃ | CH₃ | CH₂CF₃ | CF₃ |
| CH₃ | CH₂CH₃ | CH₂CF₃ | CF₃ |
| CH₃ | CH₃ | CH₂CHF₂ | CF₃ |
| CH₃ | CH₂CH₃ | CH₂CHF₂ | CF₃ |
| CH₃ | CH₃ | CH(CH₃)₂ | CF₃ |
| CH₃ | cPr | cPr | CF₃ |
| Cl | CH₃ | CH₂CH₃ | CF₃ |
| Cl | CH₂CH₃ | CH₂CH₃ | CF₃ |
| Cl | CH₃ | CH₂CF₃ | CF₃ |
| Cl | CH₂CH₃ | CH₂CF₃ | CF₃ |
| Cl | CH₃ | CH₂CHF₂ | CF₃ |
| Cl | CH₂CH₃ | CH₂CHF₂ | CF₃ |
| Cl | CH₃ | CH(CH₃)₂ | CF₃ |
| Cl | cPr | cPr | CF₃ |

TABLE A″

| R¹ | NR^{2c}R^{2d} | R³ |
|---|---|---|
| CH₃ | pyrrolidin-1-yl | Cl |
| CH₃ | piperidin-1-yl | Cl |
| CH₃ | morpholin-4-yl | Cl |
| CH₃ | 2,6-dimethylmorpholin-4-yl | Cl |
| Cl | pyrrolidin-1-yl | Cl |
| Cl | piperidin-1-yl | Cl |
| Cl | morpholin-4-yl | Cl |
| Cl | 2,6-dimethylmorpholin-4-yl | Cl |
| CH₃ | pyrrolidin-1-yl | Br |
| CH₃ | piperidin-1-yl | Br |
| CH₃ | morpholin-4-yl | Br |
| CH₃ | 2,6-dimethylmorpholin-4-yl | Br |
| Cl | pyrrolidin-1-yl | Br |
| Cl | piperidin-1-yl | Br |
| Cl | morpholin-4-yl | Br |
| Cl | 2,6-dimethylmorpholin-4-yl | Br |
| CH₃ | pyrrolidin-1-yl | CF₃ |
| CH₃ | piperidin-1-yl | CF₃ |
| CH₃ | morpholin-4-yl | CF₃ |
| CH₃ | 2,6-dimethylmorpholin-4-yl | CF₃ |
| Cl | pyrrolidin-1-yl | CF₃ |
| Cl | piperidin-1-yl | CF₃ |
| Cl | morpholin-4-yl | CF₃ |
| Cl | 2,6-dimethylmorpholin-4-yl | CF₃ |

The compounds of formula I can be prepared by standard methods of organic chemistry, e.g. by the methods described in the schemes below. The substituents, variables and indices used in the schemes are as defined above for the compounds of formula I, if not specified otherwise.

The compounds of formula I.A can be prepared analogous to Scheme 1 below.

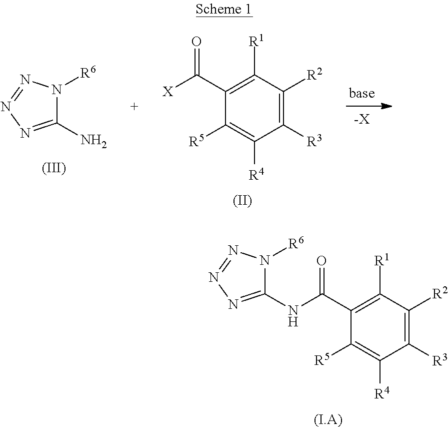

Likewise, the compounds of formula I.B can be prepared analogous to Scheme 2 below:

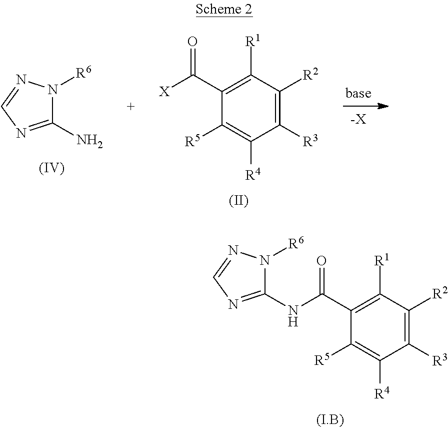

Likewise, the compounds of formula L.C can be prepared analogous to Scheme 3 below:

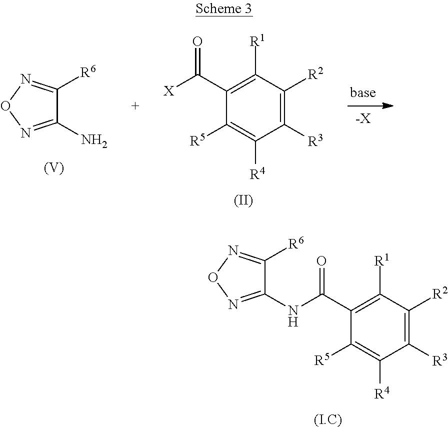

Likewise, the compounds of formula I.D can be prepared analogous to Scheme 4 below:

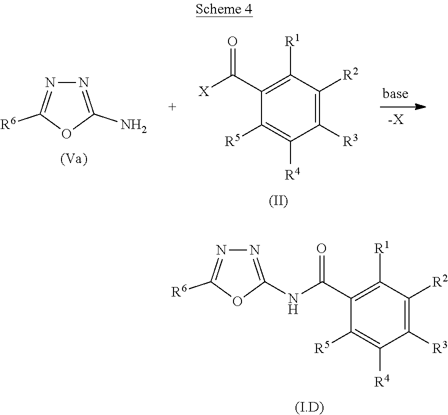

5-Amino-1-R-tetrazole compounds of formula III can be reacted with benzoyl derivatives of formula II to afford compounds of formula I.A. Likewise, 5-amino-1-R-1,2,4-triazole of formula IV can be reacted with benzoyl derivatives of formula II to afford compounds of formula I.B. Likewise, 4-amino-1,2,5-oxadiazole compounds of formula V can be reacted with benzoyl derivatives of formula II to afford compounds of the formula I.C. Likewise, 4-amino-1,3,4-oxadiazole compounds of formula Va can be reacted with benzoyl derivatives of formula II to afford compounds of the formula I.D. Herein, X is a leaving group, such as halogen, in particular Cl, an anhydride residue or an active ester residue. Especially in case of X being halogen the reaction is suitably carried out in the presence of a base. Suitable bases are for example carbonates, such as lithium, sodium or potassium carbonates, amines, such as trimethylamine or triethylamine, and basic N-heterocycles, such as pyridine, 2,6-dimethylpyridine or 2,4,6-trimethylpyridine. Suitable solvents are in particular aprotic solvents such as pentane, hexane, heptane, octane, cyclohexane, dichloromethane, chloroform, 1,2-dichlorethane, benzene, chlorobenzene, toluene, the xylenes, dichlorobenzene, trimethylbenzene, pyridine, 2,6-dimethylpyridine, 2,4,6-trimethylpyridine, acetonitrile, diethyl ether, tetrahydrofuran, 2-methyl tetrahydrofuran, methyl tert-butylether, 1,4-dioxane, N,N-dimethyl formamide, N-methyl pyrrolidinone or mixtures thereof. The starting materials are generally reacted with one another in equimolar or nearly equimolar amounts at a reaction temperature usually in the range of −20° C. to 100° C. and preferably in the range of −5° C. to 50° C. Alternatively, compounds of formula I can also be prepared as shown in Schemes 5, 6, 7 and 8. Reaction of 5-amino-1-R-tetrazole of formula III with a benzoic acid derivative of formula VI yields compound I.A. Likewise, reaction of 5-amino-1-R-1,2,4-triazole of formula IV with a benzoic acid derivative of formula VI yields compound I.B. Likewise, reaction of a 4-amino-1,2,5-oxadiazole compound V with a benzoic acid derivative of formula VI yields compound I.C.

Likewise, reaction of a 4-amino-1,3,4-oxadiazole compound Va with a benzoic acid derivative of formula VI yields compound I.D. The reaction is preferably carried out in the presence of a suitable activating agent, which converts the acid group of compound VI into an activated ester or amide. For this purpose activating agents known in the art, such as 1,1′,carbonyldiimidazole (CDI), dicyclohexyl carbodiimide (DCC), 1-ethyl-3-(3-dimethylaminopropyl)carbodiimide (EDC) or 2,4,6-tripropyl-1,3,5,2,4,6-trioxatriphosphorinane-2,4,6-trioxide (T3P) can be employed. The activated ester or amide can be formed, depending in particular on the specific activating agent used, either in situ by contacting compound V with the activating agent in the presence of compound III or IV, or in a separate step prior to the reaction with compound III or IV. It may be advantageous, especially in cases where DCC or EDC are used as activating agent, to include further additives in the activating reaction, such as hydroxybenzotriazole (HOBt), nitrophenol, pentafluorophenol, 2,4,5-trichlorophenol or N-hydroxysuccinimide. It may further be advantageous to prepare the activated ester or amide in the presence of a base, for example a tertiary amine. The activated ester or amide is either in situ or subsequently reacted with the amine of formula III or IV to afford the amide of formula I. The reaction normally takes place in anhydrous inert solvents, such as chlorinated hydrocarbons, e.g. dichloromethane or dichloroethane, ethers, e.g. tetrahydrofuran or 1,4-dioxane or carboxamides, e.g. N,N-dimethylformamide, N,N-dimethylacetamide or N-methylpyrrolidone. The reaction is ordinarily carried out at temperatures in the range from −20° C. to +25° C.

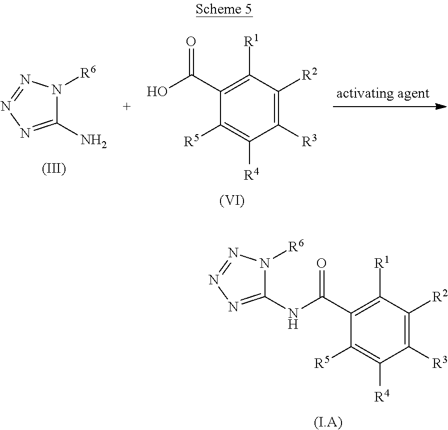

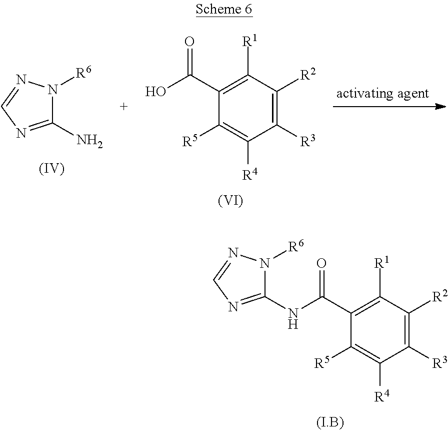

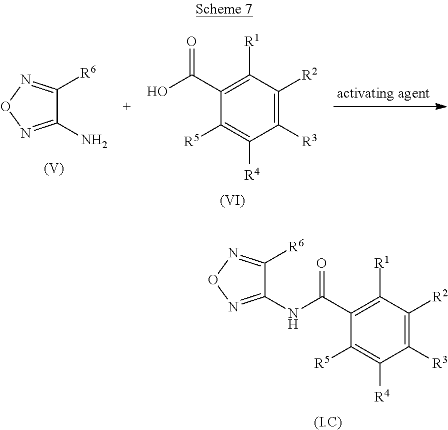

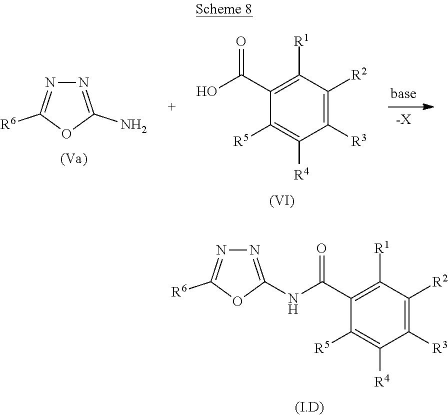

The 5-amino-1-R-tetrazoles of formula III, where R⁶ is, for example, hydrogen or an alkyl, are either commercially available or are obtainable according to methods known from the literature. For example, 5-amino-1-R-tetrazole can be prepared from 5-aminotetrazole according to the method described in the Journal of the American Chemical Society, 1954, 76, 923-924 (Scheme 9).

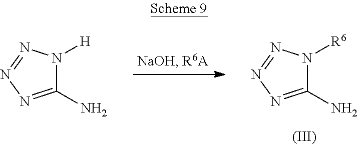

Alternatively, 5-amino-1-R-tetrazole compounds of formula III can be prepared according to the method described in the Journal of the American Chemical Society, 1954, 76, 88-89 Scheme 10).

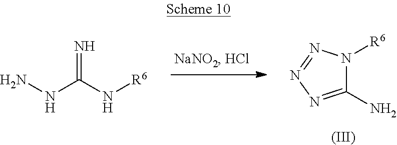

As shown in Scheme 11, 5-amino-1-R-triazoles of formula IV are either commercially available or are obtainable according to methods described in the literature. For example, 5-amino-1-R-triazole can be prepared from 5-aminotriazole according to the method described in Zeitschrift für Chemie, 1990, 30, 12, 436-437.

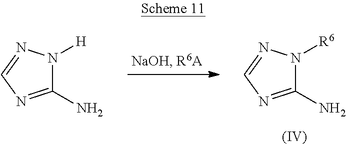

Alternatively, 5-amino-1-R-triazole compounds of formula IV, can also be prepared analogous to the synthesis described in Chemische Berichte, 1964, 97, 2, 396-404, as shown in Scheme 12.

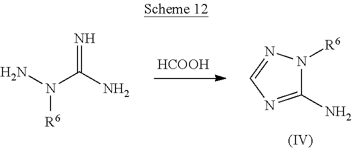

The compounds of formulae III, IV and V and the benzoic acid precursors of formulae II and V can be obtained by purchase or can be prepared by processes known in the art or disclosed in the literature, e.g. in WO 9746530, WO 9831676, WO 9831681, WO 2002/018352, WO 2000/003988, US 2007/0191335, U.S. Pat. No. 6,277,847.

The 4-amino-1,2,5-oxadiazole compounds of the formula V are either commercially available or are obtainable according to methods known from the literature. For example, 3-alkyl-4-amino-1,2,5-oxadiazoles can be prepared from β-ketoesters pursuant to a procedure described in Russian Chemical Bulletin, Int. Ed., 54(4), 1032-1037 (2005), as depicted in Scheme 13.

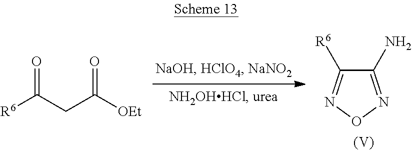

As shown in Scheme 14, the compounds of the formula V, where R⁶ is halogen, can be prepared from commercially available 3,4-diamino-1,2,5-oxadiazole according to procedures described in the literature, e.g. by the Sandmeyer-type reaction disclosed in Heteroatom Chemistry, 15(3), 199-207 (2004).

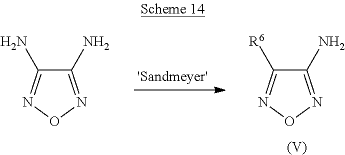

As shown in Scheme 15, the compounds of the formula V, where R⁶ is a nucleophilic residue, can be prepared by introducing the nucleophilic residue via the substitution of a leaving group L, e.g. halogene, in the 4-position of the 1,2,5-oxadiazoles compounds of formula IX in accordance to precedures disclosed, for example in Journal of Chemical Research, Synopses (6), 190 (1985), in Izvestiya Akademii Nauk SSSR, Seriya Khimicheskaya (9), 2086-8 (1986) or in Russian Chemical Bulletin (Translation of Izvestiya Akademii Nauk, Seriya Khimicheskaya), 53(3), 596-614 (2004).

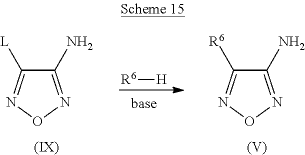

The compounds of the formula (VI), where R² is Z²—NH—C(O)—NR^{2c}R^{2d} can be prepared from the corresponding substituted 3-aminobenzoates of the formula (X), which comprises reacting the compound of formula (X) with phosgene or a phosgene equivalent (XI), such as diphosgene, i.e. trichloromethyl chloroformiate, or triphosgene, i.e. bis-trichloromethylcarbonate, and a secondary amine of the formula (XII), followed by subsequent hydrolysis as depicted in the following scheme 1. Instead of phosgene or the phosgene equivalent carbonyldiimidazole may be used.

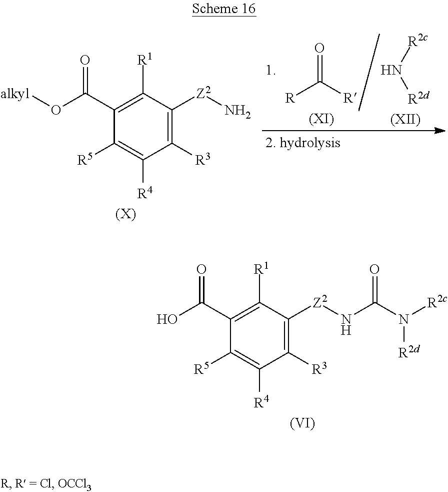

In scheme 16, alkyl means lower alkyl having 1 to 4 carbon atoms, in particular methyl or ethyl. The reaction of the compound of the formula (X) with phosgene or phosgene equivalent (XI) and the secondary amine of formula (XII) can be performed by analogy to the preparation of mixed ureas by reaction of two different amine with phosgene or phosgene equivalent. Preferably, the compound of the formula (X) is firstly reacted with phosgene or phosgene equivalent (XI) to obtain an intermediate compound or compound mixture, which is subsequently reacted with the secondary amine of the formulal (XII). The intermediate compound or compound mixture may be isolated from the reaction mixture. For economical reasons, the intermediate compound or compound mixture is usually not isolated but the reaction mixture obtained from the reaction of the compound (X) with the phosgene or phosgene equivalent (XI) is subjected to the reaction with the secondary amine of formula (XII). Compounds of formula (II) can be easily prepared from the compounds of formula (VI) by standard procedures.

As a rule, the compounds of formula I including their stereoisomers, salts, and tautomers, and their precursors in the synthesis process, can be prepared by the methods described above. If individual compounds can not be prepared via the above-described routes, they can be prepared by derivatization of other compounds I or the respective precursor or by customary modifications of the synthesis routes described. For example, in individual cases, certain compounds of formula I can advantageously be prepared from other compounds of formula I by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

The compounds of formula I and their agriculturally suitable salts are useful as herbicides. They are useful as such or as an appropriately formulated composition. The herbicidal compositions comprising the compound I, in particular the preferred aspects thereof, control vegetation on non-crop areas very efficiently, especially at high rates of application. They act against broad-leaved weeds and weed grasses in crops such as wheat, rice, corn, soybeans and cotton without causing any significant damage to the crop plants. This effect is mainly observed at low rates of application.

Depending on the application method in question, the compounds of formula I, in particular the preferred aspects thereof, or compositions comprising them can additionally be employed in a further number of crop plants for eliminating unwanted plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera, Zea mays.

The compounds of the present invention are particularly suitable for use in crops from the family poaceae, in particular crops of the tribum triticeae, e.g. crops of the generae hordeum, sorghum, triticium and secale, and crops of the generae zea, e.g. Zea mays and oryza, e.g. Oryza sativa.

The term “crop plants” also includes plants which have been modified by breeding, mutagenesis or genetic engineering. Genetically modified plants are plants whose genetic material has been modified in a manner which does not occur under natural conditions by crossing, mutations or natural recombination (i.e. reassembly of the genetic information). Here, in general, one or more genes are integrated into the genetic material of the plant to improve the properties of the plant.

Accordingly, the term “crop plants” also includes plants which, by breeding and genetic engineering, have acquired tolerance to certain classes of herbicides, such as hydroxyphenylpyruvate dioxygenase (HPPD) inhibitors, acetolactate synthase (ALS) inhibitors, such as, for example, sulfonylureas (EP-A-0257993, U.S. Pat. No. 5,013,659) or imidazolinones (see, for example, U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors, such as, for example, glyphosate (see, for example, WO 92/00377), glutamine synthetase (GS) inhibitors, such as, for example, glufosinate (see, for example, EP-A-0242236, EP-A-242246), or oxynil herbicides (see, for example, U.S. Pat. No. 5,559,024).

In a preferred embodiment, the term “crop plants” refers to plants that comprise in their genomes a gene encoding a herbicide-tolerant wild-type or mutated HPPD protein. Such a gene may be an endogenous gene or a transgene, as described hereinafter.

By a “herbicide-tolerant” or “herbicide-resistant” plant, it is intended that a plant that is tolerant or resistant to at least one herbicide at a level that would normally kill, or inhibit the growth of, a normal or wild-type plant. By “herbicide-tolerant wild-type or mutated HPPD protein” or “herbicide-resistant wild-type or mutated HPPD protein”, it is intended that such a HPPD protein displays higher HPPD activity, relative to the HPPD activity of a wild-type or reference HPPD protein, when in the presence of at least one herbicide that is known to interfere with HPPD activity and at a concentration or level of the herbicide that is known to inhibit the HPPD activity of the reference wild-type HPPD protein. Furthermore, the HPPD activity of such a herbicide-tolerant or herbicide-resistant HPPD protein may be referred to herein as “herbicidetolerant” or “herbicide-resistant” HPPD activity.

The term “mutated HPPD nucleic acid” refers to an HPPD nucleic acid having a sequence that is mutated from a wild-type HPPD nucleic acid and that confers increased “HPPDinhibiting herbicide” tolerance to a plant in which it is expressed. Furthermore, the term “mutated hydroxyphenyl pyruvate dioxygenase (mutated HPPD)” refers to the replacement of an amino acid of the wild-type primary sequences SEQ ID NO: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67, a variant, a derivative, a homologue, an orthologue, or paralogue thereof, with another amino acid. The expression “mutated amino acid” will be used below to designate the amino acid which is replaced by another amino acid, thereby designating the site of the mutation in the primary sequence of the protein.

Several HPPDs and their primary sequences have been described in the state of the art, in particular the HPPDs of bacteria such as Pseudomonas (Ruetschi et al., Eur. J. Biochem., 205, 459-466, 1992, WO96/38567), of plants such as Arabidopsis (WO96/38567, Genebank AF047834) or of carrot (WO96/38567, Genebank 87257), of Coccicoides (Genebank COITRP), HPPDs of Brassica, cotton, Synechocystis, and tomato (U.S. Pat. No. 7,297,541), of mammals such as the mouse or the pig. Furthermore, artificial HPPD sequences have been described, for exampie in U.S. Pat. Nos. 6,768,044; 6,268,549; In a preferred embodiment, the nucleotide sequence of (i) comprises the sequence of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 47, 49, 52, 54, 56, 68, 69 or a variant or derivative thereof.

In a particularly preferred embodiment, the mutated HPPD nucleic acid useful for the present invention comprises a mutated nucleic acid sequence of SEQ ID NO: 1 or SEQ ID NO: 52, or a variant or derivative thereof.

Furthermore, it will be understood by the person skilled in the art that the nucleotide sequences of (i) or (ii) encompass homologues, paralogues and orthologues of SEQ ID NO: 1, 51, 3, 4, 6, 7, 9, 10, 12, 13, 15, 16, 18, 19, 21, 23, 25, 27, 29, 31, 33, 35, 37, 39, 41, 43, 45, 47, 49, 52, 54, 56, 68, 69, as defined hereinafter.

The term “variant” with respect to a sequence (e.g., a polypeptide or nucleic acid sequence such as—for example—a transcription regulating nucleotide sequence of the invention) is intended to mean substantially similar sequences. For nucleotide sequences comprising an open reading frame, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis and for open reading frames, encode the native protein, as well as those that encode a polypeptide having amino acid substitutions relative to the native protein. Generally, nucleotide sequence variants of the invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO:1, 51, 3, 4, 6, 7, 9, 10, 12, 13, 15, 16, 18, 19, 21, 23, 25, 27, 29, 31, 33, 35, 37, 39, 41, 43, 45, 47, 49, 52, 54, 56, 68, 69, 47, or 49. By “variant” polypeptide is intended a polypeptide derived from the protein of SEQ ID NO: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

In a preferred embodiment, variants of the polynucleotides useful for the present invention will have at least 30, 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98% and 99% nucleotide “sequence identity” to the nucleotide sequence of SEQ ID NO:1, 47, 49, or SEQ ID NO: 52.

It is recognized that the polynucleotide molecules and polypeptides of the invention encompass polynucleotide molecules and polypeptides comprising a nucleotide or an amino acid sequence that is sufficiently identical to nucleotide sequences set forth in SEQ ID NOs: 1, 51, 3, 4, 6, 7, 9, 10, 12, 13, 15, 16, 18, 19, 21, 23, 25, 27, 29, 31, 33, 35, 37, 39, 41, 43, 45, 47, 49, 52, 54, 56, 68, 69, 47, or 49, or to the amino acid sequences set forth in SEQ ID NOs: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67, 48, or 50. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity.

“Sequence identity” refers to the extent to which two optimally aligned DNA or amino acid sequences are invariant throughout a window of alignment of components, e.g., nucleotides or amino acids. An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence. “Percent identity” is the identity fraction times 100. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG. Wisconsin Package. (Accelrys Inc. Burlington, Mass.)

The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

“Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived.

A deletion refers to removal of one or more amino acids from a protein.

An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag. 100 epitope, c-myc epitope, FLAG®-epitope, lacZ, CMP (calmodulin-binding peptide), HA epitope, protein C epitope and VSV epitope.

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues, but may be clustered depending upon functional constraints placed upon the polypeptide and may range from 1 to 10 amino acids; insertions will usually be of the order of about 1 to 10 amino acid residues. The amino acid substitutions are preferably conservative amino acid substitutions. Conservative substitution tables are well known in the art (see for example Creighton (1984) Proteins. W.H. Freeman and Company (Eds).

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuikChange Site Directed mutagenesis (Stratagene, San Diego, Calif.), PCRmediated site-directed mutagenesis or other site-directed mutagenesis protocols.

“Derivatives” further include peptides, oligopeptides, polypeptides which may, compared to the amino acid sequence of the naturally-occurring form of the protein, such as the protein of interest, comprise substitutions of amino acids with non-naturally occurring amino acid residues, or additions of non-naturally occurring amino acid residues. “Derivatives” of a protein also encompass peptides, oligopeptides, polypeptides which comprise naturally occurring altered (glycosylated, acylated, prenylated, phosphorylated, myristoylated, sulphated etc.) or non-naturally altered amino acid residues compared to the amino acid sequence of a naturally-occurring form of the polypeptide. A derivative may also comprise one or more non-amino acid substituents or additions compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein. Furthermore, “derivatives” also include fusions of the naturally-occurring form of the protein with tagging peptides such as FLAG, HIS6 or thioredoxin (for a review of tagging peptides, see Terpe, Appl. Microbiol. Biotechnol. 60, 523-533, 2003).

“Orthologues” and “paralogues” encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

It is well-known in the art that paralogues and orthologues may share distinct domains harboring suitable amino acid residues at given sites, such as binding pockets for particular substrates or binding motifs for interaction with other proteins.

The term “domain” refers to a set of amino acids conserved at specific positions along an alignment of sequences of evolutionarily related proteins. While amino acids at other positions can vary between homologues, amino acids that are highly conserved at specific positions indicate amino acids that are likely essential in the structure, stability or function of a protein. Identified by their high degree of conservation in aligned sequences of a family of protein homologues, they can be used as identifiers to determine if any polypeptide in question belongs to a previously identified polypeptide family.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

Specialist databases exist for the identification of domains, for example, SMART (Schultz et al. (1998) Proc. Natl. Acad. Sci. USA 95, 5857-5864; Letunic et al. (2002) Nucleic Acids Res 30, 242-244), InterPro (Mulder et al., (2003) Nucl. Acids. Res. 31, 315-318), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAI Press, Menlo Park; Hulo et al., Nucl. Acids. Res. 32:D134-D137, (2004)), or Pfam (Bateman et al., Nucleic Acids Research 30(1): 276-280 (2002)). A set of tools for in silico analysis of protein sequences is available on the ExPASy proteomics server (Swiss Institute of Bioinformatics (Gasteiger et al., ExPASy: the proteomics server for in-depth protein knowledge and analysis, Nucleic Acids Res. 31:3784-3788(2003)). Domains or motifs may also be identified using routine techniques, such as by sequence alignment.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (i.e. spanning the complete sequences) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147(1); 195-7).

By substituting one or more of the key amino acid residues, the herbicide tolerance or resistance of a plant to the herbicide as described herein could be remarkably increased as compared to the activity of the wild type HPPD enzymes with SEQ ID NO: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67. Preferred substitutions of mutated HPPD are those that increase the herbicide tolerance of the plant, but leave the biological activitiy of the dioxygenase activity substantially unaffected.

It will be understood by the person skilled in the art that amino acids located in a close proximity to the positions of amino acids mentioned below may also be substituted. Thus, in another embodiment the mutated HPPD useful for the present invention comprises a sequence of SEQ ID NO: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67, or a variant, derivative, orthologue, paralogue or homologue thereof, wherein an amino acid ±3, ±2 or ±1 amino acid positions from a key amino acid is substituted by any other amino acid.

Based on techniques well-known in the art, a highly characteristic sequence pattern can be developed, by means of which further of mutated HPPD candidates with the desired activity may be searched.

Searching for further mutated HPPD candidates by applying a suitable sequence pattern would also be encompassed by the present invention. It will be understood by a skilled reader that the present sequence pattern is not limited by the exact distances between two adjacent amino acid residues of said pattern. Each of the distances between two neighbours in the above patterns may, for example, vary independently of each other by up to ±10, ±5, ±3, ±2 or ±1 amino acid positions without substantially affecting the desired activity.

In line with said above functional and spatial analysis of individual amino acid residues based on the crystallographic data as obtained according to the present invention, unique partial amino acid sequences characteristic of potentially useful mutated HPPD candidates of the invention may be identified.

In a particularly preferred embodiment, the mutated HPPD refers to a variant or derivative of SEQ ID NO: 2 wherein the substitutions are selected from the following Table B.a.

TABLE B.a
(Sequence ID No: 2): single amino acid substitutions

| Key amino acid position | Substituents |
|---|---|
| Val212 | Ile, Leu |
| Val213 | Thr, Ala |
| Asn215 | Ala, His |
| Ala236 | Leu, Ser, Arg |
| Phe238 | Val, Ala |
| Leu250 | Val, Met |
| Ser252 | Thr |
| Pro265 | Ala |
| Asn267 | Tyr, Gln |
| Gln278 | His, Asn, Ser |
| Ile279 | Thr |
| Arg309 | Lys, Ala |
| Leu320 | Asn, Gln, His, Tyr, |
| Pro321 | Ala, Arg, Gly, Asn |
| Leu334 | Glu, Cys |
| Leu353 | Met, Tyr, Ala, Ser |
| Phe366 | Ile, Leu, Tyr |
| Gly371 | Ile, Phe |
| Thr375 | Pro |
| Phe377 | Ala, Leu, Ser |
| Gly403 | Arg |
| Phe404 | Leu, Pro |
| Lys406 | Thr |
| Gly407 | Cys, His |
| Phe409 | Ile, His |
| Glu411 | Thr |
| Leu412 | Met, Phe, Trp, Ala, Ser |
| Ile416 | Val, Phe |
| Ser410 | Gly |
| Val254 | Ala |

Furthermore, by substituting at least two of the key amino acid residues of SEQ ID NO: 2 with specific residues, the herbicide tolerance or resistance could be remarkably increased as compared to the activity of the wild type HPPD enzymes or HPPD enzymes in which only one amino acid residue had been substituted. Therefore, in another preferred embodiment, the variant or derivative of the mutated HPPD refers to a polypeptide of SEQ ID NO: 2, wherein two, three, four or five key amino acids are substituted by another amino acid residue. Particularly preferred double, triple, quadruple, or quintuple mutations are described in Table B.b.

TABLE B.b
(with reference to Sequence ID No: 2):
combined amino acid substitutions

| Combination No | Key amino acid position and and its substitutents |
|---|---|
| 1 | A236L, E411T |
| 2 | L320H, P321A |
| 3 | L320H, P321R |
| 4 | L320N, P321A |
| 5 | L320N, P321R |
| 6 | L320Q, P321A |
| 7 | L320Q, P321R |
| 8 | L320Y, P321A |
| 9 | L320Y, P321R |
| 10 | L353M, P321R |
| 11 | L353M, P321R, A236L |
| 12 | L353M, P321R, A236L, E411T |
| 13 | L353M, P321R, E411T |
| 14 | L353M, P321R, L320H |
| 15 | L353M, P321R, L320N |
| 16 | L353M, P321R, L320Q |
| 17 | L353M, P321R, L320Y |
| 18 | L353M, P321R, V212I |
| 19 | L353M, P321R, V212I, L334E |
| 20 | L353M, P321R, V212L, L334E |
| 21 | L353M, P321R, V212L, L334E, A236L |
| 22 | L353M, P321R, V212L, L334E, A236L, E411T |
| 23 | L353M, P321R, V212L, L334E, E411T |
| 24 | L353M, P321R, V212L, L334E, L320H |
| 25 | L353M, P321R, V212L, L334E, L320N |
| 26 | L353M, P321R, V212L, L334E, L320Q |
| 27 | L353M, P321R, V212L, L334E, L320Y |
| 28 | L353M, V212I |

In a particularly preferred embodiment, the mutated HPPD enzyme comprising a polypeptide of SEQ ID NO: 2, a variant, derivative, homologue, paralogue or orthologue thereof, useful for the present invention comprises one or more of the following: the amino acid corresponding to or at position 320 is histidine, asparagine or glutamine; the amino acid position 334 is glutamic acid; the amino acid position 353 is methionine; the amino acid corresponding to or at position 321 alanine or arginine; the amino acid corresponding to or at position 212 is isoleucine.

In an especially particularly preferred embodiment, the mutated HPPD refers to a polypeptide comprising SEQ ID NO: 2, wherein the leucine corresponding to or at position 320 is substituted by a histidine, and the proline corresponding to or at position 321 is substituted by an alanine.

In another especially particularly preferred embodiment, the mutated HPPD refers to a polypeptide comprising SEQ ID NO: 2, wherein Leucine corresponding to or at position 353 is substituted by a Methionine, the Proline corresponding to or at position 321 is substituted by an Arginine, and the Leucine corresponding to or at position 320 is substituted by an Asparagine.

In another especially particularly preferred embodiment, the mutated HPPD refers to a polypeptide comprising SEQ ID NO: 2, wherein the Leucine corresponding to or at position 353 is substituted by a Methionine, the Proline corresponding to or at position 321 is substituted by an Arginine, and the Leucine corresponding to or at position 320 is substituted by a glutamine.

In another preferred embodiment, the mutated HPPD refers to a variant or derivative of SEQ ID NO: 53 wherein the substitutions are selected from the following Table B.c.

TABLE B.c
(Sequence ID No: 53): single amino acid substitutions

| Key amino acid position | Substituents | Preferred substituents |
|---|---|---|
| Val228 | Thr, Ala | Thr, Ala |
| Asn230 | Ala, His | Ala, His |
| Ala251 | Ser, Arg | Ser, Arg |
| Phe253 | Val, Ala | Val, Ala |
| Leu265 | Val, Met | Val, Met |
| Ser267 | Thr | Thr |
| Pro280 | Ala | Ala |
| Asn282 | Tyr, Gln | Tyr, Gln |
| Lys291 | Arg, Ala | Arg |
| Gln293 | Ala, Leu, Ile, Val, His, Asn, Ser | His, Asn, Ser |
| Ile294 | Thr | Thr |
| Arg324 | Lys, Ala | Lys, Ala |
| Met335 | Ala, Trp, Phe, Leu, Ile, Val, Asn, Gln, | Gln, Asn, His, Tyr |
| | His, Tyr, Ser, Thr, Cys | |
| Pro336 | Ala, Arg, Gly, Asn | Ala, Gly |
| Ser337 | Ala, Pro, Thr | Pro, Thr |
| Pro339 | Deletion | Deletion |
| Pro340 | Gly | Gly |
| Glu363 | Gln | Gln |
| Leu368 | Met, Tyr, | Met |
| Phe381 | Ile, Leu, Tyr | Ile, Leu |
| Leu385 | Ala, Val, Gln, Asp | Val, Asp |
| Gly386 | Ile, Phe | Ile, Phe |
| Thr390 | Pro | Pro |
| Phe392 | Ala, Leu, Ser | Ala |
| Ile393 | Ala, Leu, Phe, Val | Leu |
| Phe419 | Leu, Pro | Leu, Pro |
| Lys421 | Thr | Thr |
| Gly422 | His, Met, Phe, Cys | His, Cys |
| Phe424 | Ile, His | Ile, His |
| Leu427 | Phe, Trp, Ala, Ser, Met | Phe |
| Ile431 | Val, Phe | Val, Phe |
| Ser425 | Gly | Gly |
| Val269 | Ala | Ala |

In another preferred embodiment, the variant or derivative of the mutated HPPD useful for the present invention refers to a polypeptide of SEQ ID NO: 53, a homologue, orthologue, or paralogue thereof, wherein two, three, four or five key amino acids are substituted by another amino acid residue. Particularly preferred double, triple, quadruple, or quintuple mutations are described in Table B.d.

TABLE B.d
(reference to Sequence ID No: 53): combined amino acid substitutions

| Combination | Key amino acid | | Preferred |
|---|---|---|---|
| No | position | Substituents | substituents |
| 1 | Pro336 | Ala, Arg | Ala |
| | Glu363 | Gln | Gln |
| 2 | Pro336 | Ala, Arg | Ala |
| | Glu363 | Gln | Gln |
| | Leu385 | Ala, Val | Val |
| 3 | Pro336 | Ala, Arg | Ala |
| | Glu363 | Gln | Gln |
| | Leu385 | Ala, Val | Val |
| | Ile393 | Ala, Leu | Leu |
| 4 | Leu385 | Ala, Val | Val |
| | Ile393 | Ala, Leu | Leu |
| 5 | Met335 | Ala, Trp, Phe, Leu, Ile, Val, Asn, | Gln, Asn, |
| | | Gln, His, Tyr, Ser, Thr, Cys | His, Tyr |
| | Pro336 | Ala, Arg, Gly | Ala, Gly |
| 6 | Met335 | Ala, Trp, Phe, Leu, Ile, Val, Asn, | Gln, Asn, |
| | | Gln, His, Tyr, Ser, Thr, Cys | His, Tyr |
| | Pro336 | Ala, Arg, Gly | Ala, Gly |
| | Glu363 | Gln | Gln |
| 7 | Met335 | Ala, Trp, Phe, Leu, Ile, Val, Asn, | Gln, Asn, His, |
| | | Gln, His, Tyr, Ser, Thr, Cys | Tyr, Leu |
| | Pro336 | Ala, Arg, Gly | Ala, Arg, |
| | | | Gly |
| | Ser337 | Ala, Pro, Thr | Pro, Thr |
| | Pro339 | Deletion | Deletion |
| | Pro340 | Gly | Gly |

Furthermore, by substituting the amino acids at some positions in the HPPD polypeptide sequences of Scenedesmus obliquus, the tolerance of crop plants as described herein towards the herbicides as described herein could be remarkably increased.

Thus, in a preferred embodiment, the mutated HPPD of the present invention comprises a variant of the sequence of SEQ ID NO: 50, or a homologue or functional equivalent thereof, which comprises one or more of the following:

the amino acid corresponding to or at position 30 is other than proline, the amino acid corresponding to or at position 39 is other than Phe, the amino acid corresponding to or at position 54 is other than Gly, the amino acid corresponding to or at position 57 is other than Met, the amino acid corresponding to or at position 84 is other than Phe, the amino acid corresponding to or at position 210 is other than Val, the amino acid corresponding to or at position 212 is other than Asn, the amino acid corresponding to or at position 223 is other than Val, the amino acid corresponding to or at position 243 is other than Val, the amino acid corresponding to or at position 247 is other than Leu, the amino acid corresponding to or at position 249 is other than Ser, the amino acid corresponding to or at position 251 is other than Val, the amino acid corresponding to or at position 264 is other than Asn, the amino acid corresponding to or at position 291 is other than Leu, the amino acid corresponding to or at position 306 is other than His, the amino acid corresponding to or at position 317 is other than Gln, the amino acid corresponding to or at position 318 is other than Ala, the amino acid corresponding to or at position 319 is other than Ala, the amino acid corresponding to or at position 321 is other than Gly, the amino acid corresponding to or at position 326 is other than Lys, the amino acid corresponding to or at position 327 is other than Arg, the amino acid corresponding to or at position 331 is other than Lys, the amino acid corresponding to or at position 341 is other than Trp, the amino acid corresponding to or at position 342 is other than Ala, the amino acid corresponding to or at position 345 is other than Glu, the amino acid corresponding to or at position 350 is other than Leu, the amino acid corresponding to or at position 363 is other than Phe, the amino acid corresponding to or at position 367 is other than Leu, the amino acid corresponding to or at position 373 is other than Ile, the amino acid corresponding to or at position 374 is other than Phe, the amino acid corresponding to or at position 375 is other than Ile, the amino acid corresponding to or at position 379 is other than Glu, the amino acid corresponding to or at position 405 is other than Gly, the amino acid corresponding to or at position 407 is other than Phe, the amino acid corresponding to or at position 410 is other than Gly, the amino acid corresponding to or at position 412 is other than Phe, the amino acid corresponding to or at position 414 is other than Glu, the amino acid corresponding to or at position 419 is other than Ile, the amino acid corresponding to or at position 421 is other than Glu, the amino acid corresponding to or at position 422 is other than Tyr.

In another preferred embodiment, the mutated HPPD comprises a variant of the sequence of SEQ ID NO: 50, or a homologue or functional equivalent thereof, in which:

the amino acid corresponding to or at position 367 is Val, and the amino acid corresponding to or at position 375 is Leu.

In another preferred embodiment, the mutated HPPD comprises a variant of the sequence of SEQ ID NO: 50, or a homologue or functional equivalent thereof, in which:

the amino acid corresponding to or at position 367 is Val, and the amino acid corresponding to or at position 375 is Leu, and the amino acid corresponding to or at position 39 is Leu.

In another preferred embodiment, the mutated HPPD comprises a variant of the sequence of SEQ ID NO: 50, or a homologue or functional equivalent thereof, in which:

the amino acid corresponding to or at position 367 is Val, and the amino acid corresponding to or at position 375 is Leu, and the amino acid corresponding to or at position 39 is Trp.

In another preferred embodiment, the mutated HPPD comprises a variant of the sequence of SEQ ID NO: 50, or a homologue or functional equivalent thereof, in which:

the amino acid corresponding to or at position 345 is Ala, Arg, Asn, Asp, Cys, Gln, Gly, His, Ile, Leu, Lys, Met, Phe, Pro, Ser, Thr, Trp, Tyr, or Val, particularly preferred Gln

In another preferred embodiment, the mutated HPPD comprises a variant of the sequence of SEQ ID NO: 50, or a homologue or functional equivalent thereof, in which:

the amino acid corresponding to or at position 345 is Gln, and the amino acid corresponding to or at position 341 is Ile.

In another preferred embodiment, the mutated HPPD comprises a variant of the sequence of SEQ ID NO: 50, or a homologue or functional equivalent thereof, in which:

the amino acid corresponding to or at position 345 is Gln, and the amino acid corresponding to or at position 326 is Glu.

In another preferred embodiment, the mutated HPPD comprises a variant of the sequence of SEQ ID NO: 50, or a homologue or functional equivalent thereof, in which:

the amino acid corresponding to or at position 345 is Gln, and the amino acid corresponding to or at position 326 is Asp.

In another preferred embodiment, the mutated HPPD comprises a variant of the sequence of SEQ ID NO: 50, or a homologue or functional equivalent thereof, in which:

the amino acid corresponding to or at position 345 is Gln, and the amino acid corresponding to or at position 326 is Gln.

In another preferred embodiment, the mutated HPPD comprises a variant of the sequence of SEQ ID NO: 50, or a homologue or functional equivalent thereof, in which:

the amino acid corresponding to or at position 318 is Arg, Asn, Asp, Cys, Gln, Glu, Gly, His, Ile, Leu, Lys, Met, Phe, Pro, Ser, Thr, Trp, Tyr, or Val, particularly preferred Pro.

In another preferred embodiment, the mutated HPPD comprises a variant of the sequence of SEQ ID NO: 50, or a homologue or functional equivalent thereof, in which:

the amino acid corresponding to or at position 319 is Arg, Asn, Asp, Cys, Gln, Glu, Gly, His, Ile, Leu, Lys, Met, Phe, Pro, Ser, Thr, Trp, Tyr, Val, particularly preferred Pro.

In another preferred embodiment, the mutated HPPD comprises a variant of the sequence of SEQ ID NO: 50, or a homologue or functional equivalent thereof, in which:

the amino acid corresponding to or at position 318 is Pro, and the amino acid corresponding to or at position 319 is Pro.

In another preferred embodiment, the mutated HPPD comprises a variant of the sequence of SEQ ID NO: 50, or a homologue or functional equivalent thereof, in which:

the amino acid corresponding to or at position 321 is Ala, Arg, Asn, Asp, Cys, Gln, Glu, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 350 is Ala, Arg, Asn, Asp, Cys, Gln, Glu, Gly, His, Ile, Lys, Met, Phe, Pro, Ser, Thr, Trp, Tyr, or Val, particularly preferred Met.

In another preferred embodiment, the mutated HPPD comprises a variant of the sequence of SEQ ID NO: 50, or a homologue or functional equivalent thereof, in which:

the amino acid corresponding to or at position 405 is Ala, Arg, Asn, Asp, Cys, Gln, Glu, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 251 is Ala, Arg, Asn, Asp, Cys, Gln, Glu, Gly, His, Ile, Leu, Lys, Met, Phe, Pro, Ser, Thr, Trp, or Tyr, particularly preferred Ala.

In another preferred embodiment, the mutated HPPD comprises a variant of the sequence of SEQ ID NO: 50, or a homologue or functional equivalent thereof, in which:

the amino acid corresponding to or at position 317 is Ala, Arg, Asn, Asp, Cys, Glu, Gly, His, Ile, Leu, Lys, Met, Phe, Pro, Ser, Thr, Trp, Tyr, or Val, particularly preferred His or Met.

In another preferred embodiment, the mutated HPPD comprises a variant of the sequence of SEQ ID NO: 50, or a homologue or functional equivalent thereof, in which:

the amino acid corresponding to or at position 379 is Ala, Arg, Asn, Asp, Cys, Gln, Gly, His, Ile, Leu, Lys, Met, Phe, Pro, Ser, Thr, Trp, Tyr, or Val, particularly preferred Gln.

In another preferred embodiment, the mutated HPPD comprises a variant of the sequence of SEQ ID NO: 50, or a homologue or functional equivalent thereof, in which:

the amino acid corresponding to or at position 350 is Met, and the amino acid corresponding to or at position 318 is Arg.

In another preferred embodiment, the mutated HPPD comprises a variant of the sequence of SEQ ID NO: 50, or a homologue or functional equivalent thereof, in which:

the amino acid corresponding to or at position 350 is Met, and the amino acid corresponding to or at position 318 is Gly.

In another preferred embodiment, the mutated HPPD comprises a variant of the sequence of SEQ ID NO: 50, or a homologue or functional equivalent thereof, in which:

the amino acid corresponding to or at position 350 is Met, and the amino acid corresponding to or at position 318 is Arg, and the amino acid corresponding to or at position 317 is Asn.

In another preferred embodiment, the mutated HPPD comprises a variant of the sequence of SEQ ID NO: 50, or a homologue or functional equivalent thereof, in which:

the amino acid corresponding to or at position 210 is Ala, Arg, Asn, Asp, Cys, Gln, Glu, Gly, His, Ile, Leu, Lys, Met, Phe, Pro, Ser, Thr, Trp, or Tyr.

In another preferred embodiment, the mutated HPPD comprises a variant of the sequence of SEQ ID NO: 50, or a homologue or functional equivalent thereof, in which:

the amino acid corresponding to or at position 317 is His, and the amino acid corresponding to or at position 318 is Gly, and the amino acid corresponding to or at position 345 is Gln.

In another preferred embodiment, the mutated HPPD comprises a variant of the sequence of SEQ ID NO: 50, or a homologue or functional equivalent thereof, in which:

the amino acid corresponding to or at position 317 is Met, and the amino acid corresponding to or at position 318 is Gly, and the amino acid corresponding to or at position 345 is Gln.

In another preferred embodiment, the mutated HPPD comprises a variant of the sequence of SEQ ID NO: 50, or a homologue or functional equivalent thereof, in which:

the amino acid corresponding to or at position 363 is Ala, Arg, Asn, Asp, Cys, Gln, Glu, Gly, His, Ile, Leu, Lys, Met, Pro, Ser, Thr, Trp, Tyr, or Val, particularly preferred Ile.

In another preferred embodiment, the mutated HPPD comprises a variant of the sequence of SEQ ID NO: 50, or a homologue or functional equivalent thereof, in which:

the amino acid corresponding to or at position 419 is Ala, Arg, Asn, Asp, Cys, Gln, Glu, Gly, His, Leu, Lys, Met, Phe, Pro, Ser, Thr, Trp, Tyr, or Val, particularly preferred Val.

In another preferred embodiment, the mutated HPPD comprises a variant of the sequence of SEQ ID NO: 50, or a homologue or functional equivalent thereof, in which:

the amino acid corresponding to or at position 249 is Ala, Arg, Asn, Asp, Cys, Gln, Glu, Gly, His, Ile, Leu, Lys, Met, Phe, Pro, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 247 is Ala, Arg, Asn, Asp, Cys, Gln, Glu, Gly, His, Ile, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 407 is Ala, Arg, Asn, Asp, Cys, Gln, Glu, Gly, His, Ile, Leu, Lys, Met, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 306 is Ala, Arg, Asn, Asp, Cys, Gln, Glu, Gly, Ile, Leu, Lys, Met, Phe, Pro, Ser, Thr, Trp, Tyr, or Val, particularly preferred Lys.

In another preferred embodiment, the mutated HPPD comprises a variant of the sequence of SEQ ID NO: 50, or a homologue or functional equivalent thereof, in which:

the amino acid corresponding to or at position 30 is Ala, Arg, Asn, Asp, Cys, Gln, Glu, Gly, His, Ile, Leu, Lys, Met, Phe, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 54 is Ala, Arg, Asn, Asp, Cys, Gln, Glu, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 57 is Ala, Arg, Asn, Asp, Cys, Gln, Glu, Gly, His, Ile, Leu, Lys, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 84 is Ala, Arg, Asn, Asp, Cys, Gln, Glu, Gly, His, Ile, Leu, Lys, Met, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 212 is Ala, Arg, Asp, Cys, Gln, Glu, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 223 is Ala, Arg, Asn, Asp, Cys, Gln, Glu, Gly, His, Ile, Leu, Lys, Met, Phe, Pro, Ser, Thr, Trp, or Tyr.

In another preferred embodiment, the mutated HPPD comprises a variant of the sequence of SEQ ID NO: 50, or a homologue or functional equivalent thereof, in which:

the amino acid corresponding to or at position 243 is Ala, Arg, Asn, Asp, Cys, Gln, Glu, Gly, His, Ile, Leu, Lys, Met, Phe, Pro, Ser, Thr, Trp, or Tyr.

In another preferred embodiment, the mutated HPPD comprises a variant of the sequence of SEQ ID NO: 50, or a homologue or functional equivalent thereof, in which:

the amino acid corresponding to or at position 264 is Ala, Arg, Asp, Cys, Gln, Glu, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 291 is Ala, Arg, Asn, Asp, Cys, Gln, Glu, Gly, His, Ile, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 327 is Ala, Asn, Asp, Cys, Gln, Glu, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 331 is Ala, Arg, Asn, Asp, Cys, Gln, Glu, Gly, His, Ile, Leu, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 342 is Arg, Asn, Asp, Cys, Gln, Glu, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 373 is Ala, Arg, Asn, Asp, Cys, Gln, Glu, Gly, His, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 374 is Ala, Arg, Asn, Asp, Cys, Gln, Glu, Gly, His, Ile, Leu, Lys, Met, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 410 is Ala, Arg, Asn, Asp, Cys, Gln, Glu, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 412 is Ala, Arg, Asn, Asp, Cys, Gln, Glu, Gly, His, Ile, Leu, Lys, Met, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 414 is Ala, Arg, Asn, Asp, Cys, Gln, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 421 is Ala, Arg, Asn, Asp, Cys, Gln, Gly, His, Ile, Leu, Lys, Met, Phe, Pro, Ser, Thr, Trp, Tyr, or Val.

In another preferred embodiment, the mutated HPPD comprises a variant of the sequence of SEQ ID NO: 50, or a homologue or functional equivalent thereof, in which:

the amino acid corresponding to or at position 422 is Ala, Arg, Asn, Asp, Cys, Gln, Glu, Gly, His, Ile, Leu, Lys, Met, Phe, Pro, Ser, Thr, Trp, or Val.

In another preferred embodiment, the mutated HPPD comprises a variant of the sequence of SEQ ID NO: 50, or a homologue or functional equivalent thereof, in which:

the amino acid corresponding to or at position 251 is Ala, and the amino acid corresponding to or at position 405 is Asp.

In another preferred embodiment, the mutated HPPD comprises a variant of the sequence of SEQ ID NO: 50, or a homologue or functional equivalent thereof, in which:

the amino acid corresponding to or at position 327 is Gly, and the amino acid corresponding to or at position 421 is Asp.

In another preferred embodiment, the mutated HPPD comprises a variant of the sequence of SEQ ID NO: 50, or a homologue or functional equivalent thereof, in which:

the amino acid corresponding to or at position 251 is Ala, and the amino acid corresponding to or at position 306 is Arg, and the amino acid corresponding to or at position 317 is Leu, and the amino acid corresponding to or at position 318 is Pro, and the amino acid corresponding to or at position 321 is Pro, and the amino acid corresponding to or at position 331 is Glu, and the amino acid corresponding to or at position 350 is Met.

In another preferred embodiment, the mutated HPPD comprises a variant of the sequence of SEQ ID NO: 50, or a homologue or functional equivalent thereof, in which:

the amino acid corresponding to or at position 407 is Ala, Arg, Asn, Asp, Cys, Gln, Glu, Gly, His, Ile, Leu, Lys, Met, Pro, Ser, Thr, Trp, Tyr, or Val.

Following mutagenesis of one of the sequences as shown herein, the encoded protein can be expressed recombinantly and the activity of the protein can be determined using, for example, assays described herein.

It will be within the knowledge of the skilled artisan to identify conserved regions and motifs shared between the homologues, orthologues and paralogues of of SEQ ID NO: 2, 5, 8, 11, 14, 17, 20, 22, 24, 26, 28, 30, 32, 34, 36, 38, 40, 42, 44, 46, 48, 50, 53, 55, 57, 58, 59, 60, 61, 62, 63, 64, 65, 66, 67, and respectively SEQ ID NO: 48 or 50. Having identified such conserved regions that may represent suitable binding motifs, amino acids corresponding to the amino acids listed in Table B.a and B.b, B.c, and B.d can be chosen to be substituted by any other amino acid by conserved amino acids, and more preferably by the amino acids of tables B.a and B.b, B.c, and B.d.

Numerous crop plants, for example Clearfield® oilseed rape, tolerant to imidazolinones, for example imazamox, have been generated with the aid of classic breeding methods (mutagenesis). Crop plants such as soybeans, cotton, corn, beet and oilseed rape, resistant to glyphosate or glufosinate, which are available under the tradenames RoundupReady® (glyphosate) and Liberty Link® (glufosinate) have been generated with the aid of genetic engineering methods.

Accordingly, the term “crop plants” also includes plants which, with the aid of genetic engineering, produce one or more toxins, for example those of the bacterial strain Bacillus ssp. Toxins which are produced by such genetically modified plants include, for example, insecticidal proteins of Bacillus spp., in particular B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins of nematode-colonizing bacteria, for example Photorhabdus spp. or Xenorhabdus spp.; toxins of animal organisms, for example wasp, spider or scorpion toxins; fungal toxins, for example from Streptomycetes; plant lectins, for example from peas or barley; agglutinins; proteinase inhibitors, for example trypsin inhibitors, serine protease inhibitors, patatin, cystatin or papain inhibitors, ribosome-inactivating proteins (RIPs), for example ricin, corn-RIP, abrin, luffin, saporin or bryodin; steroid-metabolizing enzymes, for example 3-hydroxysteroid oxidase, ecdysteroid-IDP glycosyl transferase, cholesterol oxidase, ecdysone inhibitors, or HMG-CoA reductase; ion channel blockers, for example inhibitors of sodium channels or calcium channels; juvenile hormone esterase; receptors of the diuretic hormone (helicokinin receptors); stilbene synthase, bibenzyl synthase, chitinases and glucanases. In the plants, these toxins may also be produced as pretoxins, hybrid proteins or truncated or otherwise modified proteins. Hybrid proteins are characterized by a novel combination of different protein domains (see, for example, WO 2002/015701). Further examples of such toxins or genetically modified plants which produce these toxins are disclosed in EP-A 374 753, WO 93/007278, WO 95/34656, EP-A 427 529, EPA 451 878, WO 03/018810 and WO 03/052073. The methods for producing these genetically modified plants are known to the person skilled in the art and disclosed, for example, in the publications mentioned above. Numerous of the toxins mentioned above bestow, upon the plants by which they are produced, tolerance to pests from all taxonomic classes of arthropods, in particular to beetles (Coeleropta), dipterans (Diptera) and butterflies (Lepidoptera) and to nematodes (Nematoda).

Genetically modified plants which produce one or more genes coding for insecticidal toxins are described, for example, in the publications mentioned above, and some of them are commercially available, such as, for example, YieldGard® (corn varieties producing the toxin Cry1Ab), YieldGard® Plus (corn varieties which produce the toxins Cry1Ab and Cry3Bb1), Starlink®(corn varieties which produce the toxin Cry9c), Herculex® RW (corn varieties which produce the toxins Cry34Ab1, Cry35Ab1 and the enzyme phosphinothricin-N-acetyltransferase [PAT]); NuCOTN® 33B (cotton varieties which produce the toxin Cry1Ac), Bollgard® I (cotton varieties which produce the toxin Cry1Ac), Bollgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Btl 1 (for example Agrisure® CB) and Bt176 from Syngenta Seeds SAS, France (corn varieties which produce the toxin Cry1Ab and the PAT enyzme), MIR604 from Syngenta Seeds SAS, France (corn varieties which produce a modified version of the toxin Cry3A, see WO 03/018810), MON 863 from Monsanto Europe S.A., Belgium (corn varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (corn varieties which produce the toxin Cry1F and the PAT enzyme).

Accordingly, the term “crop plants” also includes plants which, with the aid of genetic engineering, produce one or more proteins which are more robust or have increased resistance to bacterial, viral or fungal pathogens, such as, for example, pathogenesis-related proteins (PR proteins, see EP-A 0 392 225), resistance proteins (for example potato varieties producing two resistance genes against Phytophthora infestans from the wild Mexican potato Solanum bulbocastanum) or T4 lysozyme (for example potato cultivars which, by producing this protein, are resistant to bacteria such as Erwinia amylvora).

Accordingly, the term “crop plants” also includes plants whose productivity has been improved with the aid of genetic engineering methods, for example by enhancing the potential yield (for example biomass, grain yield, starch, oil or protein content), tolerance to drought, salt or other limiting environmental factors or resistance to pests and fungal, bacterial and viral pathogens.

The term “crop plants” also includes plants whose ingredients have been modified with the aid of genetic engineering methods in particular for improving human or animal diet, for example by oil plants producing health-promoting long-chain omega 3 fatty acids or monounsaturated omega 9 fatty acids (for example Nexera® oilseed rape).

The term “crop plants” also includes plants which have been modified with the aid of genetic engineering methods for improving the production of raw materials, for example by increasing the amylopectin content of potatoes (Amflora® potato).

Furthermore, it has been found that the compounds of formula I are also suitable for the defoliation and/or desiccation of plant parts, for which crop plants such as cotton, potato, oilseed rape, sunflower, soybean or field beans, in particular cotton, are suitable. In this regard, there have been found compositions for the desiccation and/or defoliation of plants, processes for preparing these compositions and methods for desiccating and/or defoliating plants using the compounds of formula I.

As desiccants, the compounds of formula I are particularly suitable for desiccating the above-ground parts of crop plants such as potato, oilseed rape, sunflower and soybean, but also cereals. This makes possible the fully mechanical harvesting of these important crop plants.

Also of economic interest is to facilitate harvesting, which is made possible by concentrating within a certain period of time the dehiscence, or reduction of adhesion to the tree, in citrus fruit, olives and other species and varieties of pomaceous fruit, stone fruit and nuts. The same mechanism, i.e. the promotion of the development of abscission tissue between fruit part or leaf part and shoot part of the plants is also essential for the readily controllable defoliation of useful plants, in particular cotton.

Moreover, a shortening of the time interval in which the individual cotton plants mature leads to an increased fiber quality after harvesting.

The compounds of formula I, or the herbicidal compositions comprising the compounds of formula I, can be used, for example, in the form of ready-to-spray aqueous solutions, powders, suspensions, also highly concentrated aqueous, oily or other suspensions or dispersions, emulsions, oil dispersions, pastes, dusts, materials for broadcasting, or granules, by means of spraying, atomizing, dusting, spreading, watering or treatment of the seed or mixing with the seed. The use forms depend on the intended purpose; in each case, they should ensure the finest possible distribution of the active ingredients according to the invention.

The herbicidal compositions comprise a herbicidally effective amount of at least one compound of the formula I or an agriculturally useful salt of I, and auxiliaries which are customary for the formulation of crop protection agents.

Examples of auxiliaries customary for the formulation of crop protection agents are inert auxiliaries, solid carriers, surfactants (such as dispersants, protective colloids, emulsifiers, wetting agents and tackifiers), organic and inorganic thickeners, bactericides, antifreeze agents, antifoams, if appropriate colorants and, for seed formulations, adhesives.

Examples of thickeners (i.e. compounds which impart to the formulation modified flow properties, i.e. high viscosity in the state of rest and low viscosity in motion) are polysaccharides, such as xanthan gum (Kelzan® from Kelco), Rhodopol® 23 (Rhone Poulenc) or Veegum® (from R.T. Vanderbilt), and also organic and inorganic sheet minerals, such as Attaclay® (from Engelhardt).

Examples of antifoams are silicone emulsions (such as, for example, Silikon® SRE, Wacker or Rhodorsil® from Rhodia), long-chain alcohols, fatty acids, salts of fatty acids, organofluorine compounds and mixtures thereof.

Bactericides can be added for stabilizing the aqueous herbicidal formulation. Examples of bactericides are bactericides based on diclorophen and benzyl alcohol hemiformal (Proxel® from ICI or Acticide® RS from Thor Chemie and Kathon® MK from Rohm & Haas), and also isothiazolinone derivates, such as alkylisothiazolinones and benzisothiazolinones (Acticide MBS from Thor Chemie).

Examples of antifreeze agents are ethylene glycol, propylene glycol, urea or glycerol.

Examples of colorants are both sparingly water-soluble pigments and water-soluble dyes. Examples which may be mentioned are the dyes known under the names Rhodamin B, C.I. Pigment Red 112 and C.I. Solvent Red 1, and also pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of adhesives are polyvinylpyrrolidone, polyvinyl acetate, polyvinyl alcohol and tylose.

Suitable inert auxiliaries are, for example, the following:

mineral oil fractions of medium to high boiling point, such as kerosene and diesel oil, furthermore coal tar oils and oils of vegetable or animal origin, aliphatic, cyclic and aromatic hydrocarbons, for example paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone or strongly polar solvents, for example amines such as N-methylpyrrolidone, and water.

Solid carriers are mineral earths such as silicas, silica gels, silicates, talc, kaolin, limestone, lime, chalk, bole, loess, clay, dolomite, diatomaceous earth, calcium sulfate, magnesium sulfate and magnesium oxide, ground synthetic materials, fertilizers such as ammonium sulfate, ammonium phosphate, ammonium nitrate and ureas, and products of vegetable origin, such as cereal meal, tree bark meal, wood meal and nutshell meal, cellulose powders, or other solid carriers.

Suitable surfactants (adjuvants, wetting agents, tackifiers, dispersants and also emulsifiers) are the alkali metal salts, alkaline earth metal salts and ammonium salts of aromatic sulfonic acids, for example lignosulfonic acids (e.g. Borrespers-types, Borregaard), phenolsulfonic acids, naphthalenesulfonic acids (Morwet types, Akzo Nobel) and dibutylnaphthalenesulfonic acid (Nekal types, BASF SE), and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors and proteins, denatured proteins, polysaccharides (e.g. methylcellulose), hydrophobically modified starches, polyvinyl alcohol (Mowiol types Clariant), polycarboxylates (BASF SE, Sokalan types), polyalkoxylates, polyvinylamine (BASF SE, Lupamine types), polyethyleneimine (BASF SE, Lupasol types), polyvinylpyrrolidone and copolymers thereof.

Powders, materials for broadcasting and dusts can be prepared by mixing or grinding the active ingredients together with a solid carrier.

Granules, for example coated granules, impregnated granules and homogeneous granules, can be prepared by binding the active ingredients to solid carriers.

Aqueous use forms can be prepared from emulsion concentrates, suspensions, pastes, wettable powders or water-dispersible granules by adding water. To prepare emulsions, pastes or oil dispersions, the compounds of formula I or Ia, either as such or dissolved in an oil or solvent, can be homogenized in water by means of a wetting agent, tackifier, dispersant or emulsifier. Alternatively, it is also possible to prepare concentrates comprising active substance, wetting agent, tackifier, dispersant or emulsifier and, if desired, solvent or oil, which are suitable for dilution with water.

The concentrations of the compounds of formula I in the ready-to-use preparations can be varied within wide ranges. In general, the formulations comprise from 0.001 to 98% by weight, preferably 0.01 to 95% by weight of at least one active compound. The active compounds are employed in a purity of from 90% to 100%, preferably 95% to 100% (according to NMR spectrum).

The formulations or ready-to-use preparations may also comprise acids, bases or buffer systems, suitable examples being phosphoric acid or sulfuric acid, or urea or ammonia.

The compounds of formula I of the invention can for example be formulated as follows:

1. Products for Dilution with Water

A. Water-Soluble Concentrates

10 parts by weight of active compound are dissolved in 90 parts by weight of water or a water-soluble solvent. As an alternative, wetters or other adjuvants are added. The active compound dissolves upon dilution with water. This gives a formulation with an active compound content of 10% by weight.

B. Dispersible Concentrates

20 parts by weight of active compound are dissolved in 70 parts by weight of cyclohexanone with addition of 10 parts by weight of a dispersant, for example polyvinylpyrrolidone. Dilution with water gives a dispersion. The active compound content is 20% by weight.

C. Emulsifiable Concentrates

15 parts by weight of active compound are dissolved in 75 parts by weight of an organic solvent (e.g. alkylaromatics) with addition of calcium dodecylbenzenesulfonate and castor oil ethoxylate (in each case 5 parts by weight). Dilution with water gives an emulsion. The formulation has an active compound content of 15% by weight.

D. Emulsions

25 parts by weight of active compound are dissolved in 35 parts by weight of an organic solvent (e.g. alkylaromatics) with addition of calcium dodecylbenzenesulfonate and castor oil ethoxylate (in each case 5 parts by weight). This mixture is introduced into 30 parts by weight of water by means of an emulsifier (e.g. Ultraturrax) and made into a homogeneous emulsion. Dilution with water gives an emulsion. The formulation has an active compound content of 25% by weight.

E. Suspensions

In an agitated ball mill, 20 parts by weight of active compound are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F. Water-Dispersible Granules and Water-Soluble Granules

50 parts by weight of active compound are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G. Water-Dispersible Powders and Water-Soluble Powders

75 parts by weight of active compound are ground in a rotor-stator mill with addition of 25 parts by weight of dispersants, wetters and silica gel. Dilution with water gives a stable dispersion or solution of the active compound. The active compound content of the formulation is 75% by weight.

H. Gel Formulations

In a ball mill, 20 parts by weight of active compound, 10 parts by weight of dispersant, 1 part by weight of gelling agent and 70 parts by weight of water or of an organic solvent are ground to give a fine suspension. Dilution with water gives a stable suspension with active compound content of 20% by weight.

2. Products to be Applied Undiluted

I. Dusts

5 parts by weight of active compound are ground finely and mixed intimately with 95 parts by weight of finely divided kaolin. This gives a dusting powder with an active compound content of 5% by weight.

J. Granules (GR, FG, GG, MG)

0.5 parts by weight of active compound are ground finely and associated with 99.5 parts by weight of carriers. Current methods here are extrusion, spray-drying or the fluidized bed. This gives granules to be applied undiluted with an active compound content of 0.5% by weight.

K. ULV Solutions (UL)

10 parts by weight of active compound are dissolved in 90 parts by weight of an organic solvent, for example xylene. This gives a product to be applied undiluted with an active compound content of 10% by weight.

The compounds of formula I or the herbicidal compositions comprising them can be applied pre- or post-emergence, or together with the seed of a crop plant. It is also possible to apply the herbicidal compositions or active compounds by applying seed, pretreated with the herbicidal compositions or active compounds, of a crop plant. If the active compounds are less well tolerated by certain crop plants, application techniques may be used in which the herbicidal compositions are sprayed, with the aid of the spraying equipment, in such a way that as far as possible they do not come into contact with the leaves of the sensitive crop plants, while the active compounds reach the leaves of undesirable plants growing underneath, or the bare soil surface (post-directed, lay-by).

In a further embodiment, the compounds of formula I or the herbicidal compositions can be applied by treating seed.

The treatment of seed comprises essentially all procedures familiar to the person skilled in the art (seed dressing, seed coating, seed dusting, seed soaking, seed film coating, seed multilayer coating, seed encrusting, seed dripping and seed pelleting) based on the compounds of formula I according to the invention or the compositions prepared therefrom. Here, the herbicidal compositions can be applied diluted or undiluted.

The term seed comprises seed of all types, such as, for example, corns, seeds, fruits, tubers, cuttings and similar forms. Here, preferably, the term seed describes corns and seeds.

The seed used can be seed of the useful plants mentioned above, but also the seed of transgenic plants or plants obtained by customary breeding methods.

The rates of application of active compound are from 0.001 to 3.0, preferably 0.01 to 1.0, kg/ha of active substance (a.s.), depending on the control target, the season, the target plants and the growth stage. To treat the seed, the compounds of formula I are generally employed in amounts of from 0.001 to 10 kg per 100 kg of seed.

It may also be advantageous to use the compounds of formula I in combination with safeners, also termed herbicide safener. Safeners are chemical compounds which prevent or reduce damage to useful plants without substantially affecting the herbicidal action of the compounds of formula I on unwanted plants. They can be used both before sowing (for example in the treatment of seed, or on cuttings or seedlings) and before or after the emergence of the useful plant. The safeners and the compounds of formula I can be used simultaneously or in succession.

Suitable safeners are, for example, (quinolin-8-oxy)acetic acids, 1-phenyl-5-haloalkyl-1H-1,2,4-triazole-3-carboxylic acids, 1-phenyl-4,5-dihydro-5-alkyl-1 Hpyrazole-3,5-dicarboxylic acids, 4,5-dihydro-5,5-diaryl-3-isoxazolecarboxylic acids, dichloroacetamides, alphaoximinophenylacetonitriles, acetophenone oximes, 4,6-dihalo-2-phenylpyrimidines, N-[[4-(aminocarbonyl)phenyl]sulfonyl]-2-benzamides, 1,8-naphthalic anhydride, 2-halo-4-(haloalkyl)-5-thiazolecarboxylic acids, phosphorothiolates and O-phenyl N-alkylcarbamates and their agriculturally useful salts and, provided that they have an acid function, their agriculturally useful derivatives, such as amides, esters and thioesters.

To broaden the activity spectrum and to obtain synergistic effects, the compounds of the formula I can be mixed and jointly applied with numerous representatives of other compounds having herbicidal activity (herbicides B) or growth-regulating activitiy, optionally in combination with safeners. Suitable mixing partners are, for example, 1,2,4-thiadiazoles, 1,3,4-thiadiazoles, amides, aminophosphoric acid and its derivatives, aminotriazoles, anilides, aryloxy/heteroaryloxyalkanoic acids and their derivatives, benzoic acid and its derivatives, benzothiadiazinones, 2-(hetaroyl/aroyl)-1,3-cyclohexanediones, heteroaryl aryl ketones, benzylisoxazolidinones, meta-CF₃-phenyl derivatives, carbamates, quinoline carboxylic acid and its derivatives, chloroacetanilides, cyclohexenone oxime ether derivates, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and heteroaryloxyphenoxypropionic esters, phenylacetic acid and its derivatives, 2-phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils and also phenylpyrazolines and isoxazolines and their derivatives.

Moreover, it may be useful to apply the compounds of formula I alone or in combination with other herbicides B or else also mixed with further crop protection agents, jointly, for example with compositions for controlling pests or phytopathogenic fungi or bacteria. Also of interest is the miscibility with mineral salt solutions which are employed for alleviating nutritional and trace element deficiencies. Other additives such as nonphytotoxic oils and oil concentrates may also be added.

Examples of herbicides B which can be used in combination with the benzamide compounds of formula I according to the present invention are:

b1) from the group of the lipid biosynthesis inhibitors:

alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[, 1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[, 1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[, 1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[, 1′-biphenyl]-3-yl)-5,6-dihydro-2,2,6,6-tetramethyl-5-oxo-2H-pyran-3-yl carbonic acid methyl ester (CAS 1033760-58-5), benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate; b2) from the group of the ALS inhibitors:

amidosulfuron, azimsulfuron, bensulfuron, bensulfuron-methyl, bispyribac, bispyribacsodium, chlorimuron, chlorimuron-ethyl, chlorsulfuron, cinosulfuron, cloransulam, cloransulammethyl, cyclosulfamuron, diclosulam, ethametsulfuron, ethametsulfuron-methyl, ethoxysulfuron, flazasulfuron, florasulam, flucarbazone, flucarbazone-sodium, flucetosulfuron, flumetsulam, flupyrsulfuron, flupyrsulfuron-methyl-sodium, foramsulfuron, halosulfuron, halosulfuron-methyl, imazamethabenz, imazamethabenz-methyl, imazamox, imazapic, imazapyr, imazaquin, imazethapyr, imazosulfuron, iodosulfuron, iodosulfuron-methyl-sodium, mesosulfuron, metosulam, metsulfuron, metsulfuron-methyl, nicosulfuron, orthosulfamuron, oxasulfuron, penoxsulam, primisulfuron, primisulfuron-methyl, propoxycarbazone, propoxycarbazone-sodium, prosulfuron, pyrazosulfuron, pyrazosulfuron-ethyl, pyribenzoxim, pyrimisulfan, pyriftalid, pyriminobac, pyriminobac-methyl, pyrithiobac, pyrithiobac-sodium, pyroxsulam, rimsulfuron, sulfometuron, sulfometuron-methyl, sulfosulfuron, thiencarbazone, thiencarbazone-methyl, thifensulfuron, thifensulfuron-methyl, triasulfuron, tribenuron, tribenuron-methyl, trifloxysulfuron, triflusulfuron, triflusulfuron-methyl and tritosulfuron;

b3) from the group of the photosynthesis inhibitors:

ametryn, amicarbazone, atrazine, bentazone, bentazone-sodium, bromacil, bromofenoxim, bromoxynil and its salts and esters, chlorobromuron, chloridazone, chlorotoluron, chloroxuron, cyanazine, desmedipham, desmetryn, dimefuron, dimethametryn, diquat, diquatdibromide, diuron, fluometuron, hexazinone, ioxynil and its salts and esters, isoproturon, isouron, karbutilate, lenacil, linuron, metamitron, methabenzthiazuron, metobenzuron, metoxuron, metribuzin, monolinuron, neburon, paraquat, paraquat-dichloride, paraquatdimetilsulfate, pentanochlor, phenmedipham, phenmedipham-ethyl, prometon, prometryn, propanil, propazine, pyridafol, pyridate, siduron, simazine, simetryn, tebuthiuron, terbacil, terbumeton, terbuthylazine, terbutryn, thidiazuron, trietazine, 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)₂-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methylpyrazol-3-yl)-4-chloro-2-hydroxy-3-methyl-2H-pyrrol-5-one (CAS 1654057-29-0), 1-(5-tert-butyl-1-methyl-pyrazol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8) and 1-(5-tert-butylisoxazol-3-yl)-4-ethoxy-5-hydroxy-3-methylimidazolidin-2-one (CAS 1844836-64-1);

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, 2-chloro-5-[3,6-dihydro-3-methyl-2,6-dioxo-4-(trifluoromethyl)-1 (2)pyrimidinyl]-4-fluoro-N-[(isopropyl)methylsulfamoyl]benzamide (H-1; CAS 372137-35-4), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (H-2; CAS 353292-31-6), N-ethyl-3-(2,6-dichloro-4-trifluoromethylphenoxy)-5-methyl-1 Hpyrazole-1-carboxamide (H-3; CAS 452098-92-9), N-tetrahydrofurfuryl-3-(2,6-d ichloro-4-trifluoromethylphenoxy)-5-methyl-1 Hpyrazole-1-carboxamide (H-4; CAS 915396-43-9), N-ethyl-3-(2-chloro-6-fluoro-4-trifluoromethylphenoxy)-5-methyl-1H-pyrazole-1-carboxamide (H-5; CAS 452099-05-7), N-tetrahydrofurfuryl-3-(2-chloro-6-fluoro-4-trifluoromethylphenoxy)-5-methyl-1H-pyrazole-1-carboxamide (H-6; CAS 45100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione, 1,5-dimethyl-6-thioxo-3-(2,2,7-trifluoro-3-oxo-4-(prop-2-ynyl)-3,4-dihydro-2H-benzo[b][1,4]oxazin-6-yl)-1,3,5-triazinane-2,4-dione (trifludimoxazin), 2-(2,2,7-Trifluoro-3-oxo-4-prop-2-ynyl-3,4-dihydro-2H-benzo[1,4]oxazin-6-yl)-4,5,6,7-tetrahydroisoindole-1,3-dione (CAS 1300118-96-0), 1-Methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluorophenoxy]-3-methoxy-but-2-enoate (CAS 948893-00-3), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4);

b5) from the group of the bleacher herbicides:

aclonifen, amitrol, beflubutamid, benzobicyclon, benzofenap, clomazone, diflufenican, fenquinotrione, flumeturon, fluridone, flurochloridone, flurtamone, isoxaflutole, mesotrione, norflurazon, oxotrione (CAS 1486617-21-3), picolinafen, pyrasulfotole, pyrazolynate, pyrazoxyfen, sulcotrione, tefuryltrione, tembotrione, tolpyralate, topramezone, 4-hydroxy-3-[[2-[(2-methoxyethoxy)methyl]-6-(trifluoromethyl)-3-pyridyl]carbonyl]bicyclo[3.2.1]oct-3-en-2-one (H-7; CAS 352010-68-5, bicyclopyrone) 4-(3-trifluoromethylphenoxy)-2-(4-trifluoromethylphenyl)pyrimidine (H-8; CAS 180608-33-7), 2-chloro-3-methylsulfanyl-N-(1-methyltetrazol-5-yl)-4-(trifluoromethyl)benzamide (CAS 1361139-71-0), 2-(2,4-dichlorophenyl)methyl-4,4-dimethyl-3-isoxazolidone (CAS 81777-95-9) and 2-(2,5-dichlorophenyl)methyl-4,4-dimethyl-3-isoxazolidinone (CAS 81778-66-7);

b6) from the group of the EPSP synthase inhibitors:

glyphosate, glyphosate-isopropylammonium and glyphosate-trimesium (sulfosate);

b7) from the group of the glutamine synthase inhibitors:

bilanaphos (bialaphos), bilanaphos-sodium, glufosinate and glufosinate-ammonium;

b8) from the group of the DHP synthase inhibitors:

asulam;

b9) from the group of the mitose inhibitors:

amiprophos, amiprophos-methyl, benfluralin, butamiphos, butralin, carbetamide, chlorpropham, chlorthal, chlorthal-dimethyl, dinitramine, dithiopyr, ethalfluralin, fluchloralin, oryzalin, pendimethalin, prodiamine, propham, propyzamide, tebutam, thiazopyr and trifluralin;

b10) from the group of the VLCFA inhibitors:

acetochlor, alachlor, anilofos, butachlor, cafenstrole, dimethachlor, dimethanamid, dimethenamid-P, diphenamid, fentrazamide, flufenacet, mefenacet, metazachlor, metolachlor, metolachlor-S, naproanilide, napropamide, pethoxamid, piperophos, pretilachlor, propachlor, propisochlor, pyroxasulfone (KIH-485) and thenylchlor;

Compounds of the formula 2:

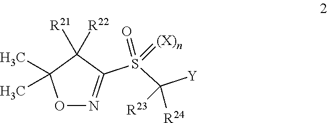

in which the variables have the following meanings:
Y is phenyl or 5- or 6-membered heteroaryl as defined at the outset, which radicals may be substituted by one to three groups R^{aa}; R²¹, R²², R²³, R²⁴ are H, halogen or C₁-C₄-alkyl; X is O or NH; N is 0 or 1.

Compounds of the formula 2 have in particular the following meanings:

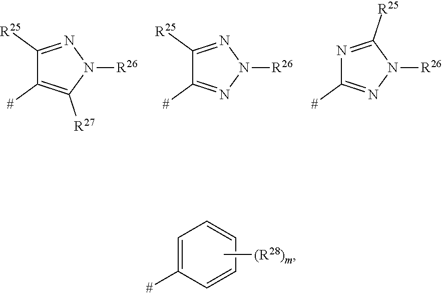

where # denotes the bond to the skeleton of the molecule; and

R²¹, R²², R²³, R²⁴ are H, Cl, F or CH₃; R²⁵ is halogen, C₁-C₄-alkyl or C₁-C₄-haloalkyl; R²⁶ is C₁-C₄-alkyl; R²⁷ is halogen, C₁-C₄-alkoxy or C₁-C₄-haloalkoxy; R²⁸ is H, halogen, C₁-C₄-alkyl, C₁-C₄-haloalkyl or C₁-C₄-haloalkoxy; M is 0, 1, 2 or 3; X is oxygen; N is 0 or 1.

Preferred compounds of the formula 2 have the following meanings:
Y is

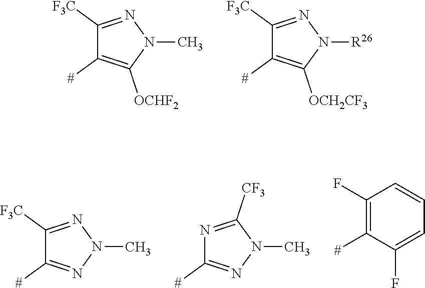

R²¹ is H; R²², R²³ are F; R²⁴ is H or F; X is oxygen; N is 0 or 1.

Particularly preferred compounds of the formula 2 are:

3-[5-(2,2-difluoroethoxy)-1-methyl-3-trifluoromethyl-1H-pyrazol-4-ylmethanesulfonyl]-4-fluoro-5,5-dimethyl-4,5-dihydroisoxazole (2-1); 3-{[5-(2,2-difluoroethoxy)-1-methyl-3-trifluoromethyl-1H-pyrazol-4-yl]fluoromethanesulfonyl}-5,5-dimethyl-4,5-dihydroisoxazole (2-2); 4-(4-fluoro-5,5-dimethyl-4,5-dihydroisoxazole-3-sulfonylmethyl)-2-methyl-5-trifluoromethyl-2H[1,2,3]triazole (2-3); 4-[(5,5-dimethyl-4,5-dihydroisoxazole-3-sulfonyl)fluoromethyl]-2-methyl-5-trifluoromethyl-2H-[1,2,3]triazole (2-4); 4-(5,5-dimethyl-4,5-dihydroisoxazole-3-sulfonylmethyl)-2-methyl-5-trifluoromethyl-2H-[1,2,3]triazole (2-5); 3-{[5-(2,2-difluoroethoxy)-1-methyl-3-trifluoromethyl-1H-pyrazol-4-yl]difluoromethanesulfonyl}-5,5-dimethyl-4,5-dihydroisoxazole (2-6); 4-[(5,5-dimethyl-4,5-dihydroisoxazole-3-sulfonyl)difluoromethyl]-2-methyl-5-trifluoromethyl-2H[1,2,3]triazole (2-7); 3-{[5-(2,2-difluoroethoxy)-1-methyl-3-trifluoromethyl-1H-pyrazol-4-yl]difluoromethanesulfonyl}-4-fluoro-5,5-dimethyl-4,5-dihydroisoxazole (2-8); 4-[difluoro-(4-fluoro-5,5-dimethyl-4,5-dihydroisoxazole-3-sulfonyl)methyl]-2-methyl-5-trifluoromethyl-2H[1,2,3]triazole (2-9);

b11) from the group of the cellulose biosynthesis inhibitors:

chlorthiamid, dichlobenil, flupoxam and isoxaben;

b12) from the group of the decoupler herbicides:

dinoseb, dinoterb and DNOC and its salts;

b13) from the group of the auxin herbicides:

2,4-D and its salts and esters, 2,4-DB and its salts and esters, aminopyralid and its salts such as aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters, MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 5,6-dichloro-2-cyclopropyl-4-pyrimidinecarboxylic acid (H-9; CAS 858956-08-8) and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenolmethyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, maleic hydrazide, mefluidide, metam, methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (H-10; CAS 499223-49-3) and its salts and esters.

Examples of preferred safeners C are benoxacor, cloquintocet, cyometrinil, cyprosulfamide, dichlormid, dicyclonone, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (H-11; MON4660, CAS 71526-07-3) and 2,2,5-trimethyl-3-(dichloroacetyl)-1,3-oxazolidine (H-12; R-29148, CAS 52836-31-4).

The active compounds of groups b1) to b15) and the safeners C are known herbicides and safeners, see, for example, The Compendium of Pesticide Common Names (http://www.alanwood.net/pesticides/); B. Hock, C. Fedtke, R. R. Schmidt, Herbizide [Herbicides], Georg Thieme Verlag, Stuttgart, 1995. Further herbicidally active compounds are known from WO 96/26202, WO 97/41116, WO 97/41117, WO 97/41118, WO 01/83459 and WO 2008/074991 and from W. Kramer et al. (ed.) “Modern Crop Protection Compounds”, Vol. 1, Wiley VCH, 2007 and the literature quoted therein.

The invention also relates to combinations comprising at least one benzamide compound of the formula I and at least one further active compound, in particular a compound having herbicide activity (herbicide B) preferably selected from the active compounds of groups b1 to b15, and/or a safener C.

The invention also relates to compositions in the form of a crop protection composition formulated as a 1-component composition comprising an active compound combination comprising at least one benzamide compound of the formula I and at least one further active compound, in particular a compound having herbicide activity (herbicide B) preferably selected from the active compounds of groups b1 to b15, and at least one solid or liquid carrier and/or one or more surfactants and, if desired, one or more further auxiliaries customary for crop protection compositions.

The invention also relates to compositions in the form of a crop protection composition formulated as a 1-component composition comprising an active compound combination comprising at least one benzamide compound of the formula I and at least one safener C and at least one solid or liquid carrier and/or one or more surfactants and, if desired, one or more further auxiliaries customary for crop protection compositions.

The invention also relates to compositions in the form of a crop protection composition formulated as a 1-component composition comprising an active compound combination comprising at least one benzamide compound of the formula I and at least one further active compound, in particular a compound having herbicide activity (herbicide B) which is preferably selected from the active compounds of groups b1 to b15, a safener C and at least one solid or liquid carrier and/or one or more surfactants and, if desired, one or more further auxiliaries customary for crop protection compositions.

The invention also relates to compositions in the form of a crop protection composition formulated as a 2-component composition comprising a first component comprising at least one compound of the formula I, a solid or liquid carrier and/or one or more surfactants and a second component comprising at least one further active compound, in particular a compound having herbicide activity (herbicide B) which is preferably selected from the active compounds of groups b1 to b15, a solid or liquid carrier and/or one or more surfactants, where additionally both components may also comprise further auxiliaries customary for crop protection compositions.

The invention also relates to compositions in the form of a crop protection composition formulated as a 2-component composition comprising a first component comprising at least one compound of the formula I, a solid or liquid carrier and/or one or more surfactants and a second component comprising at least one further active compound, in particular a compound having herbicide activity (herbicide B) which is preferably selected from the active compounds of groups b1 to b15, a solid or liquid carrier and/or one or more surfactants, where additionally both components may also comprise further auxiliaries customary for crop protection compositions, where the first component or the second component further comprises a safener C.

In binary compositions comprising at least one compound of the formula I as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one compound of the formula I as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising both at least one compound of the formula I as component A, at least one herbicide B and at least one safener C, the relative parts by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1; the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1; and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. Preferably, the weight ratio of the components A+B to the component C is in the range of from 1:500 to 500:1, in particular in the range of from 1:250 to 250:1 and particularly preferably in the range of from 1:75 to 75:1.

Examples of particularly preferred compositions according to the invention comprising in each case one individualized compound of the formula I and one mixing partner or a mixing partner combination are given in Table B below.

A further aspect of the invention relates to the combinations B-1 to B-1406 listed in Table B below, where in each case one row of Table B corresponds to a herbicidal composition comprising one of the compounds of formula I individualized in the above description (component 1) and the further active compound from groups b1) to b15) and/or safener C stated in each case in the row in question (component 2). The active compounds in the compositions described are in each case preferably present in synergistically effective amounts.

TABLE B

| | Herbicide(s) B | Safener C |
|---|---|---|
| B-1 | clodinafop-propargyl | — |
| B-2 | cycloxydim | — |
| B-3 | cyhalofop-butyl | — |
| B-4 | fenoxaprop-P-ethyl | — |
| B-5 | pinoxaden | — |
| B-6 | profoxydim | — |
| B-7 | tepraloxydim | — |
| B-8 | tralkoxydim | — |
| B-9 | esprocarb | — |
| B-10 | prosulfocarb | — |
| B-11 | thiobencarb | — |
| B-12 | triallate | — |
| B-13 | bensulfuron-methyl | — |
| B-14 | bispyribac-sodium | — |
| B-15 | cyclosulfamuron | — |
| B-16 | flumetsulam | — |
| B-17 | flupyrsulfuron-methyl-sodium | — |
| B-18 | foramsulfuron | — |
| B-19 | imazamox | — |
| B-20 | imazapic | — |
| B-21 | imazapyr | — |
| B-22 | imazaquin | — |
| B-23 | imazethapyr | — |
| B-24 | imazosulfuron | — |
| B-25 | iodosulfuron-methyl-sodium | — |
| B-26 | mesosulfuron | — |
| B-27 | nicosulfuron | — |
| B-28 | penoxsulam | — |
| B-29 | propoxycarbazone-sodium | — |
| B-30 | pyrazosulfuron-ethyl | — |
| B-31 | pyroxsulam | — |
| B-32 | rimsulfuron | — |
| B-33 | sulfosulfuron | — |
| B-34 | thiencarbazone-methyl | — |
| B-35 | tritosulfuron | — |
| B-36 | 2,4-D and its salts and esters | — |
| B-37 | aminopyralid and its salts and esters | — |
| B-38 | clopyralid and its salts and esters | — |
| B-39 | dicamba and its salts and esters | — |
| B-40 | fluroxypyr-meptyl | — |
| B-41 | quinclorac | — |
| B-42 | quinmerac | — |
| B-43 | H-9 | — |
| B-44 | diflufenzopyr | — |
| B-45 | diflufenzopyr-sodium | — |
| B-46 | clomazone | — |
| B-47 | diflufenican | — |
| B-48 | fluorochloridone | — |
| B-49 | isoxaflutol | — |
| B-50 | mesotrione | — |
| B-51 | picolinafen | — |
| B-52 | sulcotrione | — |
| B-53 | tefuryltrione | — |
| B-54 | tembotrione | — |
| B-55 | topramezone | — |
| B-56 | H-7 | — |
| B-57 | atrazine | — |
| B-58 | diuron | — |
| B-59 | fluometuron | — |
| B-60 | hexazinone | — |
| B-61 | isoproturon | — |
| B-62 | metribuzin | — |
| B-63 | propanil | — |
| B-64 | terbuthylazine | — |
| B-65 | paraquat dichloride | — |
| B-66 | flumioxazin | — |
| B-67 | oxyfluorfen | — |
| B-68 | saflufenacil | — |
| B-69 | sulfentrazone | — |
| B-70 | H-1 | — |
| B-71 | H-2 | — |
| B-72 | glyphosate | — |
| B-73 | glyphosate-isopropylammonium | — |
| B-74 | glyphosate-trimesium (sulfosate) | — |
| B-75 | glufosinate | — |
| B-76 | glufosinate-ammonium | — |
| B-77 | pendimethalin | — |
| B-78 | trifluralin | — |
| B-79 | acetochlor | — |
| B-80 | cafenstrole | — |
| B-81 | dimethenamid-P | — |
| B-82 | fentrazamide | — |
| B-83 | flufenacet | — |
| B-84 | mefenacet | — |
| B-85 | metazachlor | — |
| B-86 | metolachlor-S | — |
| B-87 | pyroxasulfone | — |
| B-88 | isoxaben | — |
| B-89 | dymron | — |
| B-90 | indanofan | — |
| B-91 | oxaziclomefone | — |
| B-92 | triaziflam | — |
| B-93 | chlorotoluron | — |
| B-94 | pinoxaden | — |
| B-95 | sethoxydim | — |
| B-96 | clethodim | — |
| B-97 | diclofop | — |
| B-98 | quizalofop | — |
| B-99 | thifensulfuron | — |
| B-100 | tribenuron | — |
| B-101 | metsulfuron | — |
| B-102 | foramsulfuron | — |
| B-103 | chlorimuron | — |
| B-104 | chlorsulfuron | — |
| B-105 | flucarbazone-sodium | — |
| B-106 | propoxycarbazone-sodium | — |
| B-107 | ethalfluralin | — |
| B-108 | halauxifen | — |
| B-109 | MCPA | — |
| B-110 | bromoxynil | — |
| B-111 | bentazone | — |
| B-112 | carfentrazone | — |
| B-113 | trifludimoxazin | — |
| B-114 | bicyclopyrone | — |
| B-115 | benzobicyclon | — |
| B-116 | pyrasulfotole | — |
| B-117 | diquat | — |
| B-118 | cinmethylin | — |
| B-119 | acetochlor | — |
| B-120 | naptalam | — |
| B-121 | atrazine + H-1 | — |
| B-122 | atrazine + glyphosate | — |
| B-123 | atrazine + mesotrione | — |
| B-124 | atrazine + nicosulfuron | — |
| B-125 | atrazine + tembotrione | — |
| B-126 | atrazine + topramezone | — |
| B-127 | clomazone + glyphosate | — |
| B-128 | diflufenican + clodinafop-propargyl | — |
| B-129 | diflufenican + fenoxaprop-P-ethyl | — |
| B-130 | diflufenican + flupyrsulfuron-methyl-sodium | — |
| B-131 | diflufenican + glyphosate | — |
| B-132 | diflufenican + mesosulfuron-methyl | — |
| B-133 | diflufenican + pinoxaden | — |
| B-134 | diflufenican + pyroxsulam | — |
| B-135 | flumetsulam + glyphosate | — |
| B-136 | flumioxazin + glyphosate | — |
| B-137 | imazapic + glyphosate | — |
| B-138 | imazethapyr + glyphosate | — |
| B-139 | isoxaflutol + H-1 | — |
| B-140 | isoxaflutol + glyphosate | — |
| B-141 | metazachlor + H-1 | — |
| B-142 | metazachlor + glyphosate | — |
| B-143 | metazachlor + mesotrione | — |
| B-144 | metazachlor + nicosulfuron | — |
| B-145 | metazachlor + terbuthylazine | — |
| B-146 | metazachlor + topramezone | — |
| B-147 | metribuzin + glyphosate | — |
| B-148 | pendimethalin + H-1 | — |
| B-149 | pendimethalin + clodinafop-propargyl | — |
| B-150 | pendimethalin + fenoxaprop-P-ethyl | — |
| B-151 | pendimethalin + flupyrsulfuron-methyl-sodium | — |
| B-152 | pendimethalin + glyphosate | — |
| B-153 | pendimethalin + mesosulfuron-methyl | — |
| B-154 | pendimethalin + mesotrione | — |
| B-155 | pendimethalin + nicosulfuron | — |
| B-156 | pendimethalin + pinoxaden | — |
| B-157 | pendimethalin + pyroxsulam | — |
| B-158 | pendimethalin + tembotrione | — |
| B-159 | pendimethalin + topramezone | — |
| B-160 | pyroxasulfone + tembotrione | — |
| B-161 | pyroxasulfone + topramezone | — |
| B-162 | sulfentrazone + glyphosate | — |
| B-163 | terbuthylazine + H-1 | — |
| B-164 | terbuthylazine + foramsulfuron | — |
| B-165 | terbuthylazine + glyphosate | — |
| B-166 | terbuthylazine + mesotrione | — |
| B-167 | terbuthylazine + nicosulfuron | — |
| B-168 | terbuthylazine + tembotrione | — |
| B-169 | terbuthylazine + topramezone | — |
| B-170 | trifluralin + glyphosate | — |
| B-171 | — | benoxacor |
| B-172 | — | cloquintocet |
| B-173 | — | cyprosulfamide |
| B-174 | — | dichlormid |
| B-175 | — | fenchlorazole |
| B-176 | — | fenclorim |
| B-177 | — | isoxadifen |
| B-178 | — | mefenpyr |
| B-179 | — | H-11 |
| B-180 | — | H-12 |
| B-181 | clodinafop-propargyl | benoxacor |
| B-182 | cycloxydim | benoxacor |
| B-183 | cyhalofop-butyl | benoxacor |
| B-184 | fenoxaprop-P-ethyl | benoxacor |
| B-185 | pinoxaden | benoxacor |
| B-186 | profoxydim | benoxacor |
| B-187 | tepraloxydim | benoxacor |
| B-188 | tralkoxydim | benoxacor |
| B-189 | esprocarb | benoxacor |
| B-190 | prosulfocarb | benoxacor |
| B-191 | thiobencarb | benoxacor |
| B-192 | triallate | benoxacor |
| B-193 | bensulfuron-methyl | benoxacor |
| B-194 | bispyribac-sodium | benoxacor |
| B-195 | cyclosulfamuron | benoxacor |
| B-196 | flumetsulam | benoxacor |
| B-197 | flupyrsulfuron-methyl-sodium | benoxacor |
| B-198 | foramsulfuron | benoxacor |
| B-199 | imazamox | benoxacor |
| B-200 | imazapic | benoxacor |
| B-201 | imazapyr | benoxacor |
| B-202 | imazaquin | benoxacor |
| B-203 | imazethapyr | benoxacor |
| B-204 | imazosulfuron | benoxacor |
| B-205 | iodosulfuron-methyl-sodium | benoxacor |
| B-206 | mesosulfuron | benoxacor |
| B-207 | nicosulfuron | benoxacor |
| B-208 | penoxsulam | benoxacor |
| B-209 | propoxycarbazone-sodium | benoxacor |
| B-210 | pyrazosulfuron-ethyl | benoxacor |
| B-211 | pyroxsulam | benoxacor |
| B-212 | rimsulfuron | benoxacor |
| B-213 | sulfosulfuron | benoxacor |
| B-214 | thiencarbazone-methyl | benoxacor |
| B-215 | tritosulfuron | benoxacor |
| B-216 | 2,4-D and its salts and esters | benoxacor |
| B-217 | aminopyralid and its salts and esters | benoxacor |
| B-218 | clopyralid and its salts and esters | benoxacor |
| B-219 | dicamba and its salts and esters | benoxacor |
| B-220 | fluroxypyr-meptyl | benoxacor |
| B-221 | quinclorac | benoxacor |
| B-222 | quinmerac | benoxacor |
| B-223 | H-9 | benoxacor |
| B-224 | diflufenzopyr | benoxacor |
| B-225 | diflufenzopyr-sodium | benoxacor |
| B-226 | clomazone | benoxacor |
| B-227 | diflufenican | benoxacor |
| B-228 | fluorochloridone | benoxacor |
| B-229 | isoxaflutol | benoxacor |
| B-230 | mesotrione | benoxacor |
| B-231 | picolinafen | benoxacor |
| B-232 | sulcotrione | benoxacor |
| B-233 | tefuryltrione | benoxacor |
| B-234 | tembotrione | benoxacor |
| B-235 | topramezone | benoxacor |
| B-236 | H-7 | benoxacor |
| B-237 | atrazine | benoxacor |
| B-238 | diuron | benoxacor |
| B-239 | fluometuron | benoxacor |
| B-240 | hexazinone | benoxacor |
| B-241 | isoproturon | benoxacor |
| B-242 | metribuzin | benoxacor |
| B-243 | propanil | benoxacor |
| B-244 | terbuthylazine | benoxacor |
| B-245 | paraquat dichloride | benoxacor |
| B-246 | flumioxazin | benoxacor |
| B-247 | oxyfluorfen | benoxacor |
| B-248 | saflufenacil | benoxacor |
| B-249 | sulfentrazone | benoxacor |
| B-250 | H-1 | benoxacor |
| B-251 | H-2 | benoxacor |
| B-252 | glyphosate | benoxacor |
| B-253 | glyphosate-isopropylammonium | benoxacor |
| B-254 | glyphosate-trimesium (sulfosate) | benoxacor |
| B-255 | glufosinate | benoxacor |
| B-256 | glufosinate-ammonium | benoxacor |
| B-257 | pendimethalin | benoxacor |
| B-258 | trifluralin | benoxacor |
| B-259 | acetochlor | benoxacor |
| B-260 | cafenstrole | benoxacor |
| B-261 | dimethenamid-P | benoxacor |
| B-262 | fentrazamide | benoxacor |
| B-263 | flufenacet | benoxacor |
| B-264 | mefenacet | benoxacor |
| B-265 | metazachlor | benoxacor |
| B-266 | metolachlor-S | benoxacor |
| B-267 | pyroxasulfone | benoxacor |
| B-268 | isoxaben | benoxacor |
| B-269 | dymron | benoxacor |
| B-270 | indanofan | benoxacor |
| B-271 | oxaziclomefone | benoxacor |
| B-272 | triaziflam | benoxacor |
| B-273 | atrazine + H-1 | benoxacor |
| B-274 | atrazine + glyphosate | benoxacor |
| B-275 | atrazine + mesotrione | benoxacor |
| B-276 | atrazine + nicosulfuron | benoxacor |
| B-277 | atrazine + tembotrione | benoxacor |
| B-278 | atrazine + topramezone | benoxacor |
| B-279 | clomazone + glyphosate | benoxacor |
| B-280 | diflufenican + clodinafop-propargyl | benoxacor |
| B-281 | diflufenican + fenoxaprop-P-ethyl | benoxacor |
| B-282 | diflufenican + flupyrsulfuron-methyl-sodium | benoxacor |
| B-283 | diflufenican + glyphosate | benoxacor |
| B-284 | diflufenican + mesosulfuron-methyl | benoxacor |
| B-285 | diflufenican + pinoxaden | benoxacor |
| B-286 | diflufenican + pyroxsulam | benoxacor |
| B-287 | flumetsulam + glyphosate | benoxacor |
| B-288 | flumioxazin + glyphosate | benoxacor |
| B-289 | imazapic + glyphosate | benoxacor |
| B-290 | imazethapyr + glyphosate | benoxacor |
| B-291 | isoxaflutol + H-1 | benoxacor |
| B-292 | isoxaflutol + glyphosate | benoxacor |
| B-293 | metazachlor + H-1 | benoxacor |
| B-294 | metazachlor + glyphosate | benoxacor |
| B-295 | metazachlor + mesotrione | benoxacor |
| B-296 | metazachlor + nicosulfuron | benoxacor |
| B-297 | metazachlor + terbuthylazine | benoxacor |
| B-298 | metazachlor + topramezone | benoxacor |
| B-299 | metribuzin + glyphosate | benoxacor |
| B-300 | pendimethalin + H-1 | benoxacor |
| B-301 | pendimethalin + clodinafop-propargyl | benoxacor |
| B-302 | pendimethalin + fenoxaprop-P-ethyl | benoxacor |
| B-303 | pendimethalin + flupyrsulfuron-methyl-sodium | benoxacor |
| B-304 | pendimethalin + glyphosate | benoxacor |
| B-305 | pendimethalin + mesosulfuron-methyl | benoxacor |
| B-306 | pendimethalin + mesotrione | benoxacor |
| B-307 | pendimethalin + nicosulfuron | benoxacor |
| B-308 | pendimethalin + pinoxaden | benoxacor |
| B-309 | pendimethalin + pyroxsulam | benoxacor |
| B-310 | pendimethalin + tembotrione | benoxacor |
| B-311 | pendimethalin + topramezone | benoxacor |
| B-312 | pyroxasulfone + tembotrione | benoxacor |
| B-313 | pyroxasulfone + topramezone | benoxacor |
| B-314 | sulfentrazone + glyphosate | benoxacor |
| B-315 | terbuthylazine + H-1 | benoxacor |
| B-316 | terbuthylazine + foramsulfuron | benoxacor |
| B-317 | terbuthylazine + glyphosate | benoxacor |
| B-318 | terbuthylazine + mesotrione | benoxacor |
| B-319 | terbuthylazine + nicosulfuron | benoxacor |
| B-320 | terbuthylazine + tembotrione | benoxacor |
| B-321 | terbuthylazine + topramezone | benoxacor |
| B-322 | trifluralin + glyphosate | benoxacor |
| B-323 | clodinafop-propargyl | cloquintocet |
| B-324 | cycloxydim | cloquintocet |
| B-325 | cyhalofop-butyl | cloquintocet |
| B-326 | fenoxaprop-P-ethyl | cloquintocet |
| B-327 | pinoxaden | cloquintocet |
| B-328 | profoxydim | cloquintocet |
| B-329 | tepraloxydim | cloquintocet |
| B-330 | tralkoxydim | cloquintocet |
| B-331 | esprocarb | cloquintocet |
| B-332 | prosulfocarb | cloquintocet |
| B-333 | thiobencarb | cloquintocet |
| B-334 | triallate | cloquintocet |
| B-335 | bensulfuron-methyl | cloquintocet |
| B-336 | bispyribac-sodium | cloquintocet |
| B-337 | cyclosulfamuron | cloquintocet |
| B-338 | flumetsulam | cloquintocet |
| B-339 | flupyrsulfuron-methyl-sodium | cloquintocet |
| B-340 | foramsulfuron | cloquintocet |
| B-341 | imazamox | cloquintocet |
| B-342 | imazapic | cloquintocet |
| B-343 | imazapyr | cloquintocet |
| B-344 | imazaquin | cloquintocet |
| B-345 | imazethapyr | cloquintocet |
| B-346 | imazosulfuron | cloquintocet |
| B-347 | iodosulfuron-methyl-sodium | cloquintocet |
| B-348 | mesosulfuron | cloquintocet |
| B-349 | nicosulfuron | cloquintocet |
| B-350 | penoxsulam | cloquintocet |
| B-351 | propoxycarbazone-sodium | cloquintocet |
| B-352 | pyrazosulfuron-ethyl | cloquintocet |
| B-353 | pyroxsulam | cloquintocet |
| B-354 | rimsulfuron | cloquintocet |
| B-355 | sulfosulfuron | cloquintocet |
| B-356 | thiencarbazone-methyl | cloquintocet |
| B-357 | tritosulfuron | cloquintocet |
| B-358 | 2,4-D and its salts and esters | cloquintocet |
| B-359 | aminopyralid and its salts and esters | cloquintocet |
| B-360 | clopyralid and its salts and esters | cloquintocet |
| B-361 | dicamba and its salts and esters | cloquintocet |
| B-362 | fluroxypyr-meptyl | cloquintocet |
| B-363 | quinclorac | cloquintocet |
| B-364 | quinmerac | cloquintocet |
| B-365 | H-9 | cloquintocet |
| B-366 | diflufenzopyr | cloquintocet |
| B-367 | diflufenzopyr-sodium | cloquintocet |
| B-368 | clomazone | cloquintocet |
| B-369 | diflufenican | cloquintocet |
| B-370 | fluorochloridone | cloquintocet |
| B-371 | isoxaflutol | cloquintocet |
| B-372 | mesotrione | cloquintocet |
| B-373 | picolinafen | cloquintocet |
| B-374 | sulcotrione | cloquintocet |
| B-375 | tefuryltrione | cloquintocet |
| B-376 | tembotrione | cloquintocet |
| B-377 | topramezone | cloquintocet |
| B-378 | H-7 | cloquintocet |
| B-379 | atrazine | cloquintocet |
| B-380 | diuron | cloquintocet |
| B-381 | fluometuron | cloquintocet |
| B-382 | hexazinone | cloquintocet |
| B-383 | isoproturon | cloquintocet |
| B-384 | metribuzin | cloquintocet |
| B-385 | propanil | cloquintocet |
| B-386 | terbuthylazine | cloquintocet |
| B-387 | paraquat dichloride | cloquintocet |
| B-388 | flumioxazin | cloquintocet |
| B-389 | oxyfluorfen | cloquintocet |
| B-390 | saflufenacil | cloquintocet |
| B-391 | sulfentrazone | cloquintocet |
| B-392 | H-1 | cloquintocet |
| B-393 | H-2 | cloquintocet |
| B-394 | glyphosate | cloquintocet |
| B-395 | glyphosate-isopropylammonium | cloquintocet |
| B-396 | glyphosate-trimesium (sulfosate) | cloquintocet |
| B-397 | glufosinate | cloquintocet |
| B-398 | glufosinate-ammonium | cloquintocet |
| B-399 | pendimethalin | cloquintocet |
| B-400 | trifluralin | cloquintocet |
| B-401 | acetochlor | cloquintocet |
| B-402 | cafenstrole | cloquintocet |
| B-403 | dimethenamid-P | cloquintocet |
| B-404 | fentrazamide | cloquintocet |
| B-405 | flufenacet | cloquintocet |
| B-406 | mefenacet | cloquintocet |
| B-407 | metazachlor | cloquintocet |
| B-408 | metolachlor-S | cloquintocet |
| B-409 | pyroxasulfone | cloquintocet |
| B-410 | isoxaben | cloquintocet |
| B-411 | dymron | cloquintocet |
| B-412 | indanofan | cloquintocet |
| B-413 | oxaziclomefone | cloquintocet |
| B-414 | triaziflam | cloquintocet |
| B-415 | atrazine + H-1 | cloquintocet |
| B-416 | atrazine + glyphosate | cloquintocet |
| B-417 | atrazine + mesotrione | cloquintocet |
| B-418 | atrazine + nicosulfuron | cloquintocet |
| B-419 | atrazine + tembotrione | cloquintocet |
| B-420 | atrazine + topramezone | cloquintocet |
| B-421 | clomazone + glyphosate | cloquintocet |
| B-422 | diflufenican + clodinafop-propargyl | cloquintocet |
| B-423 | diflufenican + fenoxaprop-p-ethyl | cloquintocet |
| B-424 | diflufenican + flupyrsulfuron-methyl-sodium | cloquintocet |
| B-425 | diflufenican + glyphosate | cloquintocet |
| B-426 | diflufenican + mesosulfuron-methyl | cloquintocet |
| B-427 | diflufenican + pinoxaden | cloquintocet |
| B-428 | diflufenican + pyroxsulam | cloquintocet |
| B-429 | flumetsulam + glyphosate | cloquintocet |
| B-430 | flumioxazin + glyphosate | cloquintocet |
| B-431 | imazapic + glyphosate | cloquintocet |
| B-432 | imazethapyr + glyphosate | cloquintocet |
| B-433 | isoxaflutol + H-1 | cloquintocet |
| B-434 | isoxaflutol + glyphosate | cloquintocet |
| B-435 | metazachlor + H-1 | cloquintocet |
| B-436 | metazachlor + glyphosate | cloquintocet |
| B-437 | metazachlor + mesotrione | cloquintocet |
| B-438 | metazachlor + nicosulfuron | cloquintocet |
| B-439 | metazachlor + terbuthylazine | cloquintocet |
| B-440 | metazachlor + topramezone | cloquintocet |
| B-441 | metribuzin + glyphosate | cloquintocet |
| B-442 | pendimethalin + H-1 | cloquintocet |
| B-443 | pendimethalin + clodinafop-propargyl | cloquintocet |
| B-444 | pendimethalin + fenoxaprop-P-ethyl | cloquintocet |
| B-445 | pendimethalin + flupyrsulfuron-methyl-sodium | cloquintocet |
| B-446 | pendimethalin + glyphosate | cloquintocet |
| B-447 | pendimethalin + mesosulfuron-methyl | cloquintocet |
| B-448 | pendimethalin + mesotrione | cloquintocet |
| B-449 | pendimethalin + nicosulfuron | cloquintocet |
| B-450 | pendimethalin + pinoxaden | cloquintocet |
| B-451 | pendimethalin + pyroxsulam | cloquintocet |
| B-452 | pendimethalin + tembotrione | cloquintocet |
| B-453 | pendimethalin + topramezone | cloquintocet |
| B-454 | pyroxasulfone + tembotrione | cloquintocet |
| B-455 | pyroxasulfone + topramezone | cloquintocet |
| B-456 | sulfentrazone + glyphosate | cloquintocet |
| B-457 | terbuthylazine + H-1 | cloquintocet |
| B-458 | terbuthylazine + foramsulfuron | cloquintocet |
| B-459 | terbuthylazine + glyphosate | cloquintocet |
| B-460 | terbuthylazine + mesotrione | cloquintocet |
| B-461 | terbuthylazine + nicosulfuron | cloquintocet |
| B-462 | terbuthylazine + tembotrione | cloquintocet |
| B-463 | terbuthylazine + topramezone | cloquintocet |
| B-464 | trifluralin + glyphosate | cloquintocet |
| B-465 | clodinafop-propargyl | dichlormid |
| B-466 | cycloxydim | dichlormid |
| B-467 | cyhalofop-butyl | dichlormid |
| B-468 | fenoxaprop-P-ethyl | dichlormid |
| B-469 | pinoxaden | dichlormid |
| B-470 | profoxydim | dichlormid |
| B-471 | tepraloxydim | dichlormid |
| B-472 | tralkoxydim | dichlormid |
| B-473 | esprocarb | dichlormid |
| B-474 | prosulfocarb | dichlormid |
| B-475 | thiobencarb | dichlormid |
| B-476 | triallate | dichlormid |
| B-477 | bensulfuron-methyl | dichlormid |
| B-478 | bispyribac-sodium | dichlormid |
| B-479 | cyclosulfamuron | dichlormid |
| B-480 | flumetsulam | dichlormid |
| B-481 | flupyrsulfuron-methyl-sodium | dichlormid |
| B-482 | foramsulfuron | dichlormid |
| B-483 | imazamox | dichlormid |
| B-484 | imazapic | dichlormid |
| B-485 | imazapyr | dichlormid |
| B-486 | imazaquin | dichlormid |
| B-487 | imazethapyr | dichlormid |
| B-488 | imazosulfuron | dichlormid |
| B-489 | iodosulfuron-methyl-sodium | dichlormid |
| B-490 | mesosulfuron | dichlormid |
| B-491 | nicosulfuron | dichlormid |
| B-492 | penoxsulam | dichlormid |
| B-493 | propoxycarbazone-sodium | dichlormid |
| B-494 | pyrazosulfuron-ethyl | dichlormid |
| B-495 | pyroxsulam | dichlormid |
| B-496 | rimsulfuron | dichlormid |
| B-497 | sulfosulfuron | dichlormid |
| B-498 | thiencarbazone-methyl | dichlormid |
| B-499 | tritosulfuron | dichlormid |
| B-500 | 2,4-D and its salts and esters | dichlormid |
| B-501 | aminopyralid and its salts and esters | dichlormid |
| B-502 | clopyralid and its salts and esters | dichlormid |
| B-503 | dicamba and its salts and esters | dichlormid |
| B-504 | fluroxypyr-meptyl | dichlormid |
| B-505 | quinclorac | dichlormid |
| B-506 | quinmerac | dichlormid |
| B-507 | H-9 | dichlormid |
| B-508 | diflufenzopyr | dichlormid |
| B-509 | diflufenzopyr-sodium | dichlormid |
| B-510 | clomazone | dichlormid |
| B-511 | diflufenican | dichlormid |
| B-512 | fluorochloridone | dichlormid |
| B-513 | isoxaflutol | dichlormid |
| B-514 | mesotrione | dichlormid |
| B-515 | picolinafen | dichlormid |
| B-516 | sulcotrione | dichlormid |
| B-517 | tefuryltrione | dichlormid |
| B-518 | tembotrione | dichlormid |
| B-519 | topramezone | dichlormid |
| B-520 | H-7 | dichlormid |
| B-521 | atrazine | dichlormid |
| B-522 | diuron | dichlormid |
| B-523 | fluometuron | dichlormid |
| B-524 | hexazinone | dichlormid |
| B-525 | isoproturon | dichlormid |
| B-526 | metribuzin | dichlormid |
| B-527 | propanil | dichlormid |
| B-528 | terbuthylazine | dichlormid |
| B-529 | paraquat dichloride | dichlormid |
| B-530 | flumioxazin | dichlormid |
| B-531 | oxyfluorfen | dichlormid |
| B-532 | saflufenacil | dichlormid |
| B-533 | sulfentrazone | dichlormid |
| B-534 | H-1 | dichlormid |
| B-535 | H-2 | dichlormid |
| B-536 | glyphosate | dichlormid |
| B-537 | glyphosate-isopropylammonium | dichlormid |
| B-538 | glyphosate-trimesium (sulfosate) | dichlormid |
| B-539 | glufosinate | dichlormid |
| B-540 | glufosinate-ammonium | dichlormid |
| B-541 | pendimethalin | dichlormid |
| B-542 | trifluralin | dichlormid |
| B-543 | acetochlor | dichlormid |
| B-544 | cafenstrole | dichlormid |
| B-545 | dimethenamid-P | dichlormid |
| B-546 | fentrazamide | dichlormid |
| B-547 | flufenacet | dichlormid |
| B-548 | mefenacet | dichlormid |
| B-549 | metazachlor | dichlormid |
| B-550 | metolachlor-S | dichlormid |
| B-551 | pyroxasulfone | dichlormid |
| B-552 | isoxaben | dichlormid |
| B-553 | dymron | dichlormid |
| B-554 | indanofan | dichlormid |
| B-555 | oxaziclomefone | dichlormid |
| B-556 | triaziflam | dichlormid |
| B-557 | atrazine + H-1 | dichlormid |
| B-558 | atrazine + glyphosate | dichlormid |
| B-559 | atrazine + mesotrione | dichlormid |
| B-560 | atrazine + nicosulfuron | dichlormid |
| B-561 | atrazine + tembotrione | dichlormid |
| B-562 | atrazine + topramezone | dichlormid |
| B-563 | clomazone + glyphosate | dichlormid |
| B-564 | diflufenican + clodinafop-propargyl | dichlormid |
| B-565 | diflufenican + fenoxaprop-p-ethyl | dichlormid |
| B-566 | diflufenican + flupyrsulfuron-methyl-sodium | dichlormid |
| B-567 | diflufenican + glyphosate | dichlormid |
| B-568 | diflufenican + mesosulfuron-methyl | dichlormid |
| B-569 | diflufenican + pinoxaden | dichlormid |
| B-570 | diflufenican + pyroxsulam | dichlormid |
| B-571 | flumetsulam + glyphosate | dichlormid |
| B-572 | flumioxazin + glyphosate | dichlormid |
| B-573 | imazapic + glyphosate | dichlormid |
| B-574 | imazethapyr + glyphosate | dichlormid |
| B-575 | isoxaflutol + H-1 | dichlormid |
| B-576 | isoxaflutol + glyphosate | dichlormid |
| B-577 | metazachlor + H-1 | dichlormid |
| B-578 | metazachlor + glyphosate | dichlormid |
| B-579 | metazachlor + mesotrione | dichlormid |
| B-580 | metazachlor + nicosulfuron | dichlormid |
| B-581 | metazachlor + terbuthylazine | dichlormid |
| B-582 | metazachlor + topramezone | dichlormid |
| B-583 | metribuzin + glyphosate | dichlormid |
| B-584 | pendimethalin + H-1 | dichlormid |
| B-585 | pendimethalin + clodinafop-propargyl | dichlormid |
| B-586 | pendimethalin + fenoxaprop-P-ethyl | dichlormid |
| B-587 | pendimethalin + flupyrsulfuron-methyl-sodium | dichlormid |
| B-588 | pendimethalin + glyphosate | dichlormid |
| B-589 | pendimethalin + mesosulfuron-methyl | dichlormid |
| B-590 | pendimethalin + mesotrione | dichlormid |
| B-591 | pendimethalin + nicosulfuron | dichlormid |
| B-592 | pendimethalin + pinoxaden | dichlormid |
| B-593 | pendimethalin + pyroxsulam | dichlormid |
| B-594 | pendimethalin + tembotrione | dichlormid |
| B-595 | pendimethalin + topramezone | dichlormid |
| B-596 | pyroxasulfone + tembotrione | dichlormid |
| B-597 | pyroxasulfone + topramezone | dichlormid |
| B-598 | sulfentrazone + glyphosate | dichlormid |
| B-599 | terbuthylazine + H-1 | dichlormid |
| B-600 | terbuthylazine + foramsulfuron | dichlormid |
| B-601 | terbuthylazine + glyphosate | dichlormid |
| B-602 | terbuthylazine + mesotrione | dichlormid |
| B-603 | terbuthylazine + nicosulfuron | dichlormid |
| B-604 | terbuthylazine + tembotrione | dichlormid |
| B-605 | terbuthylazine + topramezone | dichlormid |
| B-606 | trifluralin + glyphosate | dichlormid |
| B-607 | clodinafop-propargyl | fenchlorazole |
| B-608 | cycloxydim | fenchlorazole |
| B-609 | cyhalofop-butyl | fenchlorazole |
| B-610 | fenoxaprop-P-ethyl | fenchlorazole |
| B-611 | pinoxaden | fenchlorazole |
| B-612 | profoxydim | fenchlorazole |
| B-613 | tepraloxydim | fenchlorazole |
| B-614 | tralkoxydim | fenchlorazole |
| B-615 | esprocarb | fenchlorazole |
| B-616 | prosulfocarb | fenchlorazole |
| B-617 | thiobencarb | fenchlorazole |
| B-618 | triallate | fenchlorazole |
| B-619 | bensulfuron-methyl | fenchlorazole |
| B-620 | bispyribac-sodium | fenchlorazole |
| B-621 | cyclosulfamuron | fenchlorazole |
| B-622 | flumetsulam | fenchlorazole |
| B-623 | flupyrsulfuron-methyl-sodium | fenchlorazole |
| B-624 | foramsulfuron | fenchlorazole |
| B-625 | imazamox | fenchlorazole |
| B-626 | imazapic | fenchlorazole |
| B-627 | imazapyr | fenchlorazole |
| B-628 | imazaquin | fenchlorazole |
| B-629 | imazethapyr | fenchlorazole |
| B-630 | imazosulfuron | fenchlorazole |
| B-631 | iodosulfuron-methyl-sodium | fenchlorazole |
| B-632 | mesosulfuron | fenchlorazole |
| B-633 | nicosulfuron | fenchlorazole |
| B-634 | penoxsulam | fenchlorazole |
| B-635 | propoxycarbazone-sodium | fenchlorazole |
| B-636 | pyrazosulfuron-ethyl | fenchlorazole |
| B-637 | pyroxsulam | fenchlorazole |
| B-638 | rimsulfuron | fenchlorazole |
| B-639 | sulfosulfuron | fenchlorazole |
| B-640 | thiencarbazone-methyl | fenchlorazole |
| B-641 | tritosulfuron | fenchlorazole |
| B-642 | 2,4-D and its salts and esters | fenchlorazole |
| B-643 | aminopyralid and its salts and esters | fenchlorazole |
| B-644 | clopyralid and its salts and esters | fenchlorazole |
| B-645 | dicamba and its salts and esters | fenchlorazole |
| B-646 | fluroxypyr-meptyl | fenchlorazole |
| B-647 | quinclorac | fenchlorazole |
| B-648 | quinmerac | fenchlorazole |
| B-649 | H-9 | fenchlorazole |
| B-650 | diflufenzopyr | fenchlorazole |
| B-651 | diflufenzopyr-sodium | fenchlorazole |
| B-652 | clomazone | fenchlorazole |
| B-653 | diflufenican | fenchlorazole |
| B-654 | fluorochloridone | fenchlorazole |
| B-655 | isoxaflutol | fenchlorazole |
| B-656 | mesotrione | fenchlorazole |
| B-657 | picolinafen | fenchlorazole |
| B-658 | sulcotrione | fenchlorazole |
| B-659 | tefuryltrione | fenchlorazole |
| B-660 | tembotrione | fenchlorazole |
| B-661 | topramezone | fenchlorazole |
| B-662 | H-7 | fenchlorazole |
| B-663 | atrazine | fenchlorazole |
| B-664 | diuron | fenchlorazole |
| B-665 | fluometuron | fenchlorazole |
| B-666 | hexazinone | fenchlorazole |
| B-667 | isoproturon | fenchlorazole |
| B-668 | metribuzin | fenchlorazole |
| B-669 | propanil | fenchlorazole |
| B-670 | terbuthylazine | fenchlorazole |
| B-671 | paraquat dichloride | fenchlorazole |
| B-672 | flumioxazin | fenchlorazole |
| B-673 | oxyfluorfen | fenchlorazole |
| B-674 | saflufenacil | fenchlorazole |
| B-675 | sulfentrazone | fenchlorazole |
| B-676 | H-1 | fenchlorazole |
| B-677 | H-2 | fenchlorazole |
| B-678 | glyphosate | fenchlorazole |
| B-679 | glyphosate-isopropylammonium | fenchlorazole |
| B-680 | glyphosate-trimesium (sulfosate) | fenchlorazole |
| B-681 | glufosinate | fenchlorazole |
| B-682 | glufosinate-ammonium | fenchlorazole |
| B-683 | pendimethalin | fenchlorazole |
| B-684 | trifluralin | fenchlorazole |
| B-685 | acetochlor | fenchlorazole |
| B-686 | cafenstrole | fenchlorazole |
| B-687 | dimethenamid-P | fenchlorazole |
| B-688 | fentrazamide | fenchlorazole |
| B-689 | flufenacet | fenchlorazole |
| B-690 | mefenacet | fenchlorazole |
| B-691 | metazachlor | fenchlorazole |
| B-692 | metolachlor-S | fenchlorazole |
| B-693 | pyroxasulfone | fenchlorazole |
| B-694 | isoxaben | fenchlorazole |
| B-695 | dymron | fenchlorazole |
| B-696 | indanofan | fenchlorazole |
| B-697 | oxaziclomefone | fenchlorazole |
| B-698 | triaziflam | fenchlorazole |
| B-699 | atrazine + H-1 | fenchlorazole |
| B-700 | atrazine + glyphosate | fenchlorazole |
| B-701 | atrazine + mesotrione | fenchlorazole |
| B-702 | atrazine + nicosulfuron | fenchlorazole |
| B-703 | atrazine + tembotrione | fenchlorazole |
| B-704 | atrazine + topramezone | fenchlorazole |
| B-705 | clomazone + glyphosate | fenchlorazole |
| B-706 | diflufenican + clodinafop-propargyl | fenchlorazole |
| B-707 | diflufenican + fenoxaprop-P-ethyl | fenchlorazole |
| B-708 | diflufenican + flupyrsulfuron-methyl-sodium | fenchlorazole |
| B-709 | diflufenican + glyphosate | fenchlorazole |
| B-710 | diflufenican + mesosulfuron-methyl | fenchlorazole |
| B-711 | diflufenican + pinoxaden | fenchlorazole |
| B-712 | diflufenican + pyroxsulam | fenchlorazole |
| B-713 | flumetsulam + glyphosate | fenchlorazole |
| B-714 | flumioxazin + glyphosate | fenchlorazole |
| B-715 | imazapic + glyphosate | fenchlorazole |
| B-716 | imazethapyr + glyphosate | fenchlorazole |
| B-717 | isoxaflutol + H-1 | fenchlorazole |
| B-718 | isoxaflutol + glyphosate | fenchlorazole |
| B-719 | metazachlor + H-1 | fenchlorazole |
| B-720 | metazachlor + glyphosate | fenchlorazole |
| B-721 | metazachlor + mesotrione | fenchlorazole |
| B-722 | metazachlor + nicosulfuron | fenchlorazole |
| B-723 | metazachlor + terbuthylazine | fenchlorazole |
| B-724 | metazachlor + topramezone | fenchlorazole |
| B-725 | metribuzin + glyphosate | fenchlorazole |
| B-726 | pendimethalin + H-1 | fenchlorazole |
| B-727 | pendimethalin + clodinafop-propargyl | fenchlorazole |
| B-728 | pendimethalin + fenoxaprop-P-ethyl | fenchlorazole |
| B-729 | pendimethalin + flupyrsulfuron-methyl-sodium | fenchlorazole |
| B-730 | pendimethalin + glyphosate | fenchlorazole |
| B-731 | pendimethalin + mesosulfuron-methyl | fenchlorazole |
| B-732 | pendimethalin + mesotrione | fenchlorazole |
| B-733 | pendimethalin + nicosulfuron | fenchlorazole |
| B-734 | pendimethalin + pinoxaden | fenchlorazole |
| B-735 | pendimethalin + pyroxsulam | fenchlorazole |
| B-736 | pendimethalin + tembotrione | fenchlorazole |
| B-737 | pendimethalin + topramezone | fenchlorazole |
| B-738 | pyroxasulfone + tembotrione | fenchlorazole |
| B-739 | pyroxasulfone + topramezone | fenchlorazole |
| B-740 | sulfentrazone + glyphosate | fenchlorazole |
| B-741 | terbuthylazine + H-1 | fenchlorazole |
| B-742 | terbuthylazine + foramsulfuron | fenchlorazole |
| B-743 | terbuthylazine + glyphosate | fenchlorazole |
| B-744 | terbuthylazine + mesotrione | fenchlorazole |
| B-745 | terbuthylazine + nicosulfuron | fenchlorazole |
| B-746 | terbuthylazine + tembotrione | fenchlorazole |
| B-747 | terbuthylazine + topramezone | fenchlorazole |
| B-748 | trifluralin + glyphosate | fenchlorazole |
| B-749 | clodinafop-propargyl | fenclorim |
| B-750 | cycloxydim | fenclorim |
| B-751 | cyhalofop-butyl | fenclorim |
| B-752 | fenoxaprop-P-ethyl | fenclorim |
| B-753 | pinoxaden | fenclorim |
| B-754 | profoxydim | fenclorim |
| B-755 | tepraloxydim | fenclorim |
| B-756 | tralkoxydim | fenclorim |
| B-757 | esprocarb | fenclorim |
| B-758 | prosulfocarb | fenclorim |
| B-759 | thiobencarb | fenclorim |
| B-760 | triallate | fenclorim |
| B-761 | bensulfuron-methyl | fenclorim |
| B-762 | bispyribac-sodium | fenclorim |
| B-763 | cyclosulfamuron | fenclorim |
| B-764 | flumetsulam | fenclorim |
| B-765 | flupyrsulfuron-methyl-sodium | fenclorim |
| B-766 | foramsulfuron | fenclorim |
| B-767 | imazamox | fenclorim |
| B-768 | imazapic | fenclorim |
| B-769 | imazapyr | fenclorim |
| B-770 | imazaquin | fenclorim |
| B-771 | imazethapyr | fenclorim |
| B-772 | imazosulfuron | fenclorim |
| B-773 | iodosulfuron-methyl-sodium | fenclorim |
| B-774 | mesosulfuron | fenclorim |
| B-775 | nicosulfuron | fenclorim |
| B-776 | penoxsulam | fenclorim |
| B-777 | propoxycarbazone-sodium | fenclorim |
| B-778 | pyrazosulfuron-ethyl | fenclorim |
| B-779 | pyroxsulam | fenclorim |
| B-780 | rimsulfuron | fenclorim |
| B-781 | sulfosulfuron | fenclorim |
| B-782 | thiencarbazone-methyl | fenclorim |
| B-783 | tritosulfuron | fenclorim |
| B-784 | 2,4-D and its salts and esters | fenclorim |
| B-785 | aminopyralid and its salts and esters | fenclorim |
| B-786 | clopyralid and its salts and esters | fenclorim |
| B-787 | dicamba and its salts and esters | fenclorim |
| B-788 | fluroxypyr-meptyl | fenclorim |
| B-789 | quinclorac | fenclorim |
| B-790 | quinmerac | fenclorim |
| B-791 | H-9 | fenclorim |
| B-792 | diflufenzopyr | fenclorim |
| B-793 | diflufenzopyr-sodium | fenclorim |
| B-794 | clomazone | fenclorim |
| B-795 | diflufenican | fenclorim |
| B-796 | fluorochloridone | fenclorim |
| B-797 | isoxaflutol | fenclorim |
| B-798 | mesotrione | fenclorim |
| B-799 | picolinafen | fenclorim |
| B-800 | sulcotrione | fenclorim |
| B-801 | tefuryltrione | fenclorim |
| B-802 | tembotrione | fenclorim |
| B-803 | topramezone | fenclorim |
| B-804 | H-7 | fenclorim |
| B-805 | atrazine | fenclorim |
| B-806 | diuron | fenclorim |
| B-807 | fluometuron | fenclorim |
| B-808 | hexazinone | fenclorim |
| B-809 | isoproturon | fenclorim |
| B-810 | metribuzin | fenclorim |
| B-811 | propanil | fenclorim |
| B-812 | terbuthylazine | fenclorim |
| B-813 | paraquat dichloride | fenclorim |
| B-814 | flumioxazin | fenclorim |
| B-815 | oxyfluorfen | fenclorim |
| B-816 | saflufenacil | fenclorim |
| B-817 | sulfentrazone | fenclorim |
| B-818 | H-1 | fenclorim |
| B-819 | H-2 | fenclorim |
| B-820 | glyphosate | fenclorim |
| B-821 | glyphosate-isopropylammonium | fenclorim |
| B-822 | glyphosate-trimesium (sulfosate) | fenclorim |
| B-823 | glufosinate | fenclorim |
| B-824 | glufosinate-ammonium | fenclorim |
| B-825 | pendimethalin | fenclorim |
| B-826 | trifluralin | fenclorim |
| B-827 | acetochlor | fenclorim |
| B-828 | cafenstrole | fenclorim |
| B-829 | dimethenamid-P | fenclorim |
| B-830 | fentrazamide | fenclorim |
| B-831 | flufenacet | fenclorim |
| B-832 | mefenacet | fenclorim |
| B-833 | metazachlor | fenclorim |
| B-834 | metolachlor-S | fenclorim |
| B-835 | pyroxasulfone | fenclorim |
| B-836 | isoxaben | fenclorim |
| B-837 | dymron | fenclorim |
| B-838 | indanofan | fenclorim |
| B-839 | oxaziclomefone | fenclorim |
| B-840 | triaziflam | fenclorim |
| B-841 | atrazine + H-1 | fenclorim |
| B-842 | atrazine + glyphosate | fenclorim |
| B-843 | atrazine + mesotrione | fenclorim |
| B-844 | atrazine + nicosulfuron | fenclorim |
| B-845 | atrazine + tembotrione | fenclorim |
| B-846 | atrazine + topramezone | fenclorim |
| B-847 | clomazone + glyphosate | fenclorim |
| B-848 | diflufenican + clodinafop-propargyl | fenclorim |
| B-849 | diflufenican + fenoxaprop-P-ethyl | fenclorim |
| B-850 | diflufenican + flupyrsulfuron-methyl-sodium | fenclorim |
| B-851 | diflufenican + glyphosate | fenclorim |
| B-852 | diflufenican + mesosulfuron-methyl | fenclorim |
| B-853 | diflufenican + pinoxaden | fenclorim |
| B-854 | diflufenican + pyroxsulam | fenclorim |
| B-855 | flumetsulam + glyphosate | fenclorim |
| B-856 | flumioxazin + glyphosate | fenclorim |
| B-857 | imazapic + glyphosate | fenclorim |
| B-858 | imazethapyr + glyphosate | fenclorim |
| B-859 | isoxaflutol + H-1 | fenclorim |
| B-860 | isoxaflutol + glyphosate | fenclorim |
| B-861 | metazachlor + H-1 | fenclorim |
| B-862 | metazachlor + glyphosate | fenclorim |
| B-863 | metazachlor + mesotrione | fenclorim |
| B-864 | metazachlor + nicosulfuron | fenclorim |
| B-865 | metazachlor + terbuthylazine | fenclorim |
| B-866 | metazachlor + topramezone | fenclorim |
| B-867 | metribuzin + glyphosate | fenclorim |
| B-868 | pendimethalin + H-1 | fenclorim |
| B-869 | pendimethalin + clodinafop-propargyl | fenclorim |
| B-870 | pendimethalin + fenoxaprop-P-ethyl | fenclorim |
| B-871 | pendimethalin + flupyrsulfuron-methyl-sodium | fenclorim |
| B-872 | pendimethalin + glyphosate | fenclorim |
| B-873 | pendimethalin + mesosulfuron-methyl | fenclorim |
| B-874 | pendimethalin + mesotrione | fenclorim |
| B-875 | pendimethalin + nicosulfuron | fenclorim |
| B-876 | pendimethalin + pinoxaden | fenclorim |
| B-877 | pendimethalin + pyroxsulam | fenclorim |
| B-878 | pendimethalin + tembotrione | fenclorim |
| B-879 | pendimethalin + topramezone | fenclorim |
| B-880 | pyroxasulfone + tembotrione | fenclorim |
| B-881 | pyroxasulfone + topramezone | fenclorim |
| B-882 | sulfentrazone + glyphosate | fenclorim |
| B-883 | terbuthylazine + H-1 | fenclorim |
| B-884 | terbuthylazine + foramsulfuron | fenclorim |
| B-885 | terbuthylazine + glyphosate | fenclorim |
| B-886 | terbuthylazine + mesotrione | fenclorim |
| B-887 | terbuthylazine + nicosulfuron | fenclorim |
| B-888 | terbuthylazine + tembotrione | fenclorim |
| B-889 | terbuthylazine + topramezone | fenclorim |
| B-890 | trifluralin + glyphosate | fenclorim |
| B-891 | clodinafop-propargyl | isoxadifen |
| B-892 | cycloxydim | isoxadifen |
| B-893 | cyhalofop-butyl | isoxadifen |
| B-894 | fenoxaprop-P-ethyl | isoxadifen |
| B-895 | pinoxaden | isoxadifen |
| B-896 | profoxydim | isoxadifen |
| B-897 | tepraloxydim | isoxadifen |
| B-898 | tralkoxydim | isoxadifen |
| B-899 | esprocarb | isoxadifen |
| B-900 | prosulfocarb | isoxadifen |
| B-901 | thiobencarb | isoxadifen |
| B-902 | triallate | isoxadifen |
| B-903 | bensulfuron-methyl | isoxadifen |
| B-904 | bispyribac-sodium | isoxadifen |
| B-905 | cyclosulfamuron | isoxadifen |
| B-906 | flumetsulam | isoxadifen |
| B-907 | flupyrsulfuron-methyl-sodium | isoxadifen |
| B-908 | foramsulfuron | isoxadifen |
| B-909 | imazamox | isoxadifen |
| B-910 | imazapic | isoxadifen |
| B-911 | imazapyr | isoxadifen |
| B-912 | imazaquin | isoxadifen |
| B-913 | imazethapyr | isoxadifen |
| B-914 | imazosulfuron | isoxadifen |
| B-915 | iodosulfuron-methyl-sodium | isoxadifen |
| B-916 | mesosulfuron | isoxadifen |
| B-917 | nicosulfuron | isoxadifen |
| B-918 | penoxsulam | isoxadifen |
| B-919 | propoxycarbazone-sodium | isoxadifen |
| B-920 | pyrazosulfuron-ethyl | isoxadifen |
| B-921 | pyroxsulam | isoxadifen |
| B-922 | rimsulfuron | isoxadifen |
| B-923 | sulfosulfuron | isoxadifen |
| B-924 | thiencarbazone-methyl | isoxadifen |
| B-925 | tritosulfuron | isoxadifen |
| B-926 | 2,4-D and its salts and esters | isoxadifen |
| B-927 | aminopyralid and its salts and esters | isoxadifen |
| B-928 | clopyralid and its salts and esters | isoxadifen |
| B-929 | dicamba and its salts and esters | isoxadifen |
| B-930 | fluroxypyr-meptyl | isoxadifen |
| B-931 | quinclorac | isoxadifen |
| B-932 | quinmerac | isoxadifen |
| B-933 | H-9 | isoxadifen |
| B-934 | diflufenzopyr | isoxadifen |
| B-935 | diflufenzopyr-sodium | isoxadifen |
| B-936 | clomazone | isoxadifen |
| B-937 | diflufenican | isoxadifen |
| B-938 | fluorochloridone | isoxadifen |
| B-939 | isoxaflutol | isoxadifen |
| B-940 | mesotrione | isoxadifen |
| B-941 | picolinafen | isoxadifen |
| B-942 | sulcotrione | isoxadifen |
| B-943 | tefuryltrione | isoxadifen |
| B-944 | tembotrione | isoxadifen |
| B-945 | topramezone | isoxadifen |
| B-946 | H-7 | isoxadifen |
| B-947 | atrazine | isoxadifen |
| B-948 | diuron | isoxadifen |
| B-949 | fluometuron | isoxadifen |
| B-950 | hexazinone | isoxadifen |
| B-951 | isoproturon | isoxadifen |
| B-952 | metribuzin | isoxadifen |
| B-953 | propanil | isoxadifen |
| B-954 | terbuthylazine | isoxadifen |
| B-955 | paraquat dichloride | isoxadifen |
| B-956 | flumioxazin | isoxadifen |
| B-957 | oxyfluorfen | isoxadifen |
| B-958 | saflufenacil | isoxadifen |
| B-959 | sulfentrazone | isoxadifen |
| B-960 | H-1 | isoxadifen |
| B-961 | H-2 | isoxadifen |
| B-962 | glyphosate | isoxadifen |
| B-963 | glyphosate-isopropylammonium | isoxadifen |
| B-964 | glyphosate-trimesium (sulfosate) | isoxadifen |
| B-965 | glufosinate | isoxadifen |
| B-966 | glufosinate-ammonium | isoxadifen |
| B-967 | pendimethalin | isoxadifen |
| B-968 | trifluralin | isoxadifen |
| B-969 | acetochlor | isoxadifen |
| B-970 | cafenstrole | isoxadifen |
| B-971 | dimethenamid-P | isoxadifen |
| B-972 | fentrazamide | isoxadifen |
| B-973 | flufenacet | isoxadifen |
| B-974 | mefenacet | isoxadifen |
| B-975 | metazachlor | isoxadifen |
| B-976 | metolachlor-S | isoxadifen |
| B-977 | pyroxasulfone | isoxadifen |
| B-978 | isoxaben | isoxadifen |
| B-979 | dymron | isoxadifen |
| B-980 | indanofan | isoxadifen |
| B-981 | oxaziclomefone | isoxadifen |
| B-982 | triaziflam | isoxadifen |
| B-983 | atrazine + H-1 | isoxadifen |
| B-984 | atrazine + glyphosate | isoxadifen |
| B-985 | atrazine + mesotrione | isoxadifen |
| B-986 | atrazine + nicosulfuron | isoxadifen |
| B-987 | atrazine + tembotrione | isoxadifen |
| B-988 | atrazine + topramezone | isoxadifen |
| B-989 | clomazone + glyphosate | isoxadifen |
| B-990 | diflufenican + clodinafop-propargyl | isoxadifen |
| B-991 | diflufenican + fenoxaprop-P-ethyl | isoxadifen |
| B-992 | diflufenican + flupyrsulfuron-methyl-sodium | isoxadifen |
| B-993 | diflufenican + glyphosate | isoxadifen |
| B-994 | diflufenican + mesosulfuron-methyl | isoxadifen |
| B-995 | diflufenican + pinoxaden | isoxadifen |
| B-996 | diflufenican + pyroxsulam | isoxadifen |
| B-997 | flumetsulam + glyphosate | isoxadifen |
| B-998 | flumioxazin + glyphosate | isoxadifen |
| B-999 | imazapic + glyphosate | isoxadifen |
| B-1000 | imazethapyr + glyphosate | isoxadifen |
| B-1001 | isoxaflutol + H-1 | isoxadifen |
| B-1002 | isoxaflutol + glyphosate | isoxadifen |
| B-1003 | metazachlor + H-1 | isoxadifen |
| B-1004 | metazachlor + glyphosate | isoxadifen |
| B-1005 | metazachlor + mesotrione | isoxadifen |
| B-1006 | metazachlor + nicosulfuron | isoxadifen |
| B-1007 | metazachlor + terbuthylazine | isoxadifen |
| B-1008 | metazachlor + topramezone | isoxadifen |
| B-1009 | metribuzin + glyphosate | isoxadifen |
| B-1010 | pendimethalin + H-1 | isoxadifen |
| B-1011 | pendimethalin + clodinafop-propargyl | isoxadifen |
| B-1012 | pendimethalin + fenoxaprop-P-ethyl | isoxadifen |
| B-1013 | pendimethalin + flupyrsulfuron-methyl-sodium | isoxadifen |
| B-1014 | pendimethalin + glyphosate | isoxadifen |
| B-1015 | pendimethalin + mesosulfuron-methyl | isoxadifen |
| B-1016 | pendimethalin + mesotrione | isoxadifen |
| B-1017 | pendimethalin + nicosulfuron | isoxadifen |
| B-1018 | pendimethalin + pinoxaden | isoxadifen |
| B-1019 | pendimethalin + pyroxsulam | isoxadifen |
| B-1020 | pendimethalin + tembotrione | isoxadifen |
| B-1021 | pendimethalin + topramezone | isoxadifen |
| B-1022 | pyroxasulfone + tembotrione | isoxadifen |
| B-1023 | pyroxasulfone + topramezone | isoxadifen |
| B-1024 | sulfentrazone + glyphosate | isoxadifen |
| B-1025 | terbuthylazine + H-1 | isoxadifen |
| B-1026 | terbuthylazine + foramsulfuron | isoxadifen |
| B-1027 | terbuthylazine + glyphosate | isoxadifen |
| B-1028 | terbuthylazine + mesotrione | isoxadifen |
| B-1029 | terbuthylazine + nicosulfuron | isoxadifen |
| B-1030 | terbuthylazine + tembotrione | isoxadifen |
| B-1031 | terbuthylazine + topramezone | isoxadifen |
| B-1032 | trifluralin + glyphosate | isoxadifen |
| B-1033 | clodinafop-propargyl | mefenpyr |
| B-1034 | cycloxydim | mefenpyr |
| B-1035 | cyhalofop-butyl | mefenpyr |
| B-1036 | fenoxaprop-P-ethyl | mefenpyr |
| B-1037 | pinoxaden | mefenpyr |
| B-1038 | profoxydim | mefenpyr |
| B-1039 | tepraloxydim | mefenpyr |
| B-1040 | tralkoxydim | mefenpyr |
| B-1041 | esprocarb | mefenpyr |
| B-1042 | prosulfocarb | mefenpyr |
| B-1043 | thiobencarb | mefenpyr |
| B-1044 | triallate | mefenpyr |
| B-1045 | bensulfuron-methyl | mefenpyr |
| B-1046 | bispyribac-sodium | mefenpyr |
| B-1047 | cyclosulfamuron | mefenpyr |
| B-1048 | flumetsulam | mefenpyr |
| B-1049 | flupyrsulfuron-methyl-sodium | mefenpyr |
| B-1050 | foramsulfuron | mefenpyr |
| B-1051 | imazamox | mefenpyr |
| B-1052 | imazapic | mefenpyr |
| B-1053 | imazapyr | mefenpyr |
| B-1054 | imazaquin | mefenpyr |
| B-1055 | imazethapyr | mefenpyr |
| B-1056 | imazosulfuron | mefenpyr |
| B-1057 | iodosulfuron-methyl-sodium | mefenpyr |
| B-1058 | mesosulfuron | mefenpyr |
| B-1059 | nicosulfuron | mefenpyr |
| B-1060 | penoxsulam | mefenpyr |
| B-1061 | propoxycarbazone-sodium | mefenpyr |
| B-1062 | pyrazosulfuron-ethyl | mefenpyr |
| B-1063 | pyroxsulam | mefenpyr |
| B-1064 | rimsulfuron | mefenpyr |
| B-1065 | sulfosulfuron | mefenpyr |
| B-1066 | thiencarbazone-methyl | mefenpyr |
| B-1067 | tritosulfuron | mefenpyr |
| B-1068 | 2,4-D and its salts and esters | mefenpyr |
| B-1069 | aminopyralid and its salts and esters | mefenpyr |
| B-1070 | clopyralid and its salts and esters | mefenpyr |
| B-1071 | dicamba and its salts and esters | mefenpyr |
| B-1072 | fluroxypyr-meptyl | mefenpyr |
| B-1073 | quinclorac | mefenpyr |
| B-1074 | quinmerac | mefenpyr |
| B-1075 | H-9 | mefenpyr |
| B-1076 | diflufenzopyr | mefenpyr |
| B-1077 | diflufenzopyr-sodium | mefenpyr |
| B-1078 | clomazone | mefenpyr |
| B-1079 | diflufenican | mefenpyr |
| B-1080 | fluorochloridone | mefenpyr |
| B-1081 | isoxaflutol | mefenpyr |
| B-1082 | mesotrione | mefenpyr |
| B-1083 | picolinafen | mefenpyr |
| B-1084 | sulcotrione | mefenpyr |
| B-1085 | tefuryltrione | mefenpyr |
| B-1086 | tembotrione | mefenpyr |
| B-1087 | topramezone | mefenpyr |
| B-1088 | H-7 | mefenpyr |
| B-1089 | atrazine | mefenpyr |
| B-1090 | diuron | mefenpyr |
| B-1091 | fluometuron | mefenpyr |
| B-1092 | hexazinone | mefenpyr |
| B-1093 | isoproturon | mefenpyr |
| B-1094 | metribuzin | mefenpyr |
| B-1095 | propanil | mefenpyr |
| B-1096 | terbuthylazine | mefenpyr |
| B-1097 | paraquat dichloride | mefenpyr |
| B-1098 | flumioxazin | mefenpyr |
| B-1099 | oxyfluorfen | mefenpyr |
| B-1100 | saflufenacil | mefenpyr |
| B-1101 | sulfentrazone | mefenpyr |
| B-1102 | H-1 | mefenpyr |
| B-1103 | H-2 | mefenpyr |
| B-1104 | glyphosate | mefenpyr |
| B-1105 | glyphosate-isopropylammonium | mefenpyr |
| B-1106 | glyphosate-trimesium (sulfosate) | mefenpyr |
| B-1107 | glufosinate | mefenpyr |
| B-1108 | glufosinate-ammonium | mefenpyr |
| B-1109 | pendimethalin | mefenpyr |
| B-1110 | trifluralin | mefenpyr |
| B-1111 | acetochlor | mefenpyr |
| B-1112 | cafenstrole | mefenpyr |
| B-1113 | dimethenamid-P | mefenpyr |
| B-1114 | fentrazamide | mefenpyr |
| B-1115 | flufenacet | mefenpyr |
| B-1116 | mefenacet | mefenpyr |
| B-1117 | metazachlor | mefenpyr |
| B-1118 | metolachlor-S | mefenpyr |
| B-1119 | pyroxasulfone | mefenpyr |
| B-1120 | isoxaben | mefenpyr |
| B-1121 | dymron | mefenpyr |
| B-1122 | indanofan | mefenpyr |
| B-1123 | oxaziclomefone | mefenpyr |
| B-1124 | triaziflam | mefenpyr |
| B-1125 | atrazine + H-1 | mefenpyr |
| B-1126 | atrazine + glyphosate | mefenpyr |
| B-1127 | atrazine + mesotrione | mefenpyr |
| B-1128 | atrazine + nicosulfuron | mefenpyr |
| B-1129 | atrazine + tembotrione | mefenpyr |
| B-1130 | atrazine + topramezone | mefenpyr |
| B-1131 | clomazone + glyphosate | mefenpyr |
| B-1132 | diflufenican + clodinafop-propargyl | mefenpyr |
| B-1133 | diflufenican + fenoxaprop-P-ethyl | mefenpyr |
| B-1134 | diflufenican + flupyrsulfuron-methyl-sodium | mefenpyr |
| B-1135 | diflufenican + glyphosate | mefenpyr |
| B-1136 | diflufenican + mesosulfuron-methyl | mefenpyr |
| B-1137 | diflufenican + pinoxaden | mefenpyr |
| B-1138 | diflufenican + pyroxsulam | mefenpyr |
| B-1139 | flumetsulam + glyphosate | mefenpyr |
| B-1140 | flumioxazin + glyphosate | mefenpyr |
| B-1141 | imazapic + glyphosate | mefenpyr |
| B-1142 | imazethapyr + glyphosate | mefenpyr |
| B-1143 | isoxaflutol + H-1 | mefenpyr |
| B-1144 | isoxaflutol + glyphosate | mefenpyr |
| B-1145 | metazachlor + H-1 | mefenpyr |
| B-1146 | metazachlor + glyphosate | mefenpyr |
| B-1147 | metazachlor + mesotrione | mefenpyr |
| B-1148 | metazachlor + nicosulfuron | mefenpyr |
| B-1149 | metazachlor + terbuthylazine | mefenpyr |
| B-1150 | metazachlor + topramezone | mefenpyr |
| B-1151 | metribuzin + glyphosate | mefenpyr |
| B-1152 | pendimethalin + H-1 | mefenpyr |
| B-1153 | pendimethalin + clodinafop-propargyl | mefenpyr |
| B-1154 | pendimethalin + fenoxaprop-P-ethyl | mefenpyr |
| B-1155 | pendimethalin + flupyrsulfuron-methyl-sodium | mefenpyr |
| B-1156 | pendimethalin + glyphosate | mefenpyr |
| B-1157 | pendimethalin + mesosulfuron-methyl | mefenpyr |
| B-1158 | pendimethalin + mesotrione | mefenpyr |
| B-1159 | pendimethalin + nicosulfuron | mefenpyr |
| B-1160 | pendimethalin + pinoxaden | mefenpyr |
| B-1161 | pendimethalin + pyroxsulam | mefenpyr |
| B-1162 | pendimethalin + tembotrione | mefenpyr |
| B-1163 | pendimethalin + topramezone | mefenpyr |
| B-1164 | pyroxasulfone + tembotrione | mefenpyr |
| B-1165 | pyroxasulfone + topramezone | mefenpyr |
| B-1166 | sulfentrazone + glyphosate | mefenpyr |
| B-1167 | terbuthylazine + H-1 | mefenpyr |
| B-1168 | terbuthylazine + foramsulfuron | mefenpyr |
| B-1169 | terbuthylazine + glyphosate | mefenpyr |
| B-1170 | terbuthylazine + mesotrione | mefenpyr |
| B-1171 | terbuthylazine + nicosulfuron | mefenpyr |
| B-1172 | terbuthylazine + tembotrione | mefenpyr |
| B-1173 | terbuthylazine + topramezone | mefenpyr |
| B-1174 | trifluralin + glyphosate | mefenpyr |
| B-1175 | clodinafop-propargyl | H-12 |
| B-1176 | cycloxydim | H-12 |
| B-1177 | cyhalofop-butyl | H-12 |
| B-1178 | fenoxaprop-P-ethyl | H-12 |
| B-1179 | pinoxaden | H-12 |
| B-1180 | profoxydim | H-12 |
| B-1181 | tepraloxydim | H-12 |
| B-1182 | tralkoxydim | H-12 |
| B-1183 | esprocarb | H-12 |
| B-1184 | prosulfocarb | H-12 |
| B-1185 | thiobencarb | H-12 |
| B-1186 | triallate | H-12 |
| B-1187 | bensulfuron-methyl | H-12 |
| B-1188 | bispyribac-sodium | H-12 |
| B-1189 | cyclosulfamuron | H-12 |
| B-1190 | flumetsulam | H-12 |
| B-1191 | flupyrsulfuron-methyl-sodium | H-12 |
| B-1192 | foramsulfuron | H-12 |
| B-1193 | imazamox | H-12 |
| B-1194 | imazapic | H-12 |
| B-1195 | imazapyr | H-12 |
| B-1196 | imazaquin | H-12 |
| B-1197 | imazethapyr | H-12 |
| B-1198 | imazosulfuron | H-12 |
| B-1199 | iodosulfuron-methyl-sodium | H-12 |
| B-1200 | mesosulfuron | H-12 |
| B-1201 | nicosulfuron | H-12 |
| B-1202 | penoxsulam | H-12 |
| B-1203 | propoxycarbazone-sodium | H-12 |
| B-1204 | pyrazosulfuron-ethyl | H-12 |
| B-1205 | pyroxsulam | H-12 |
| B-1206 | rimsulfuron | H-12 |
| B-1207 | sulfosulfuron | H-12 |
| B-1208 | thiencarbazone-methyl | H-12 |
| B-1209 | tritosulfuron | H-12 |
| B-1210 | 2,4-D and its salts and esters | H-12 |
| B-1211 | aminopyralid and its salts and esters | H-12 |
| B-1212 | clopyralid and its salts and esters | H-12 |
| B-1213 | dicamba and its salts and esters | H-12 |
| B-1214 | fluroxypyr-meptyl | H-12 |
| B-1215 | quinclorac | H-12 |
| B-1216 | quinmerac | H-12 |
| B-1217 | B-9 | H-12 |
| B-1218 | diflufenzopyr | H-12 |
| B-1219 | diflufenzopyr-sodium | H-12 |
| B-1220 | clomazone | H-12 |
| B-1221 | diflufenican | H-12 |
| B-1222 | fluorochloridone | H-12 |
| B-1223 | isoxaflutol | H-12 |
| B-1224 | mesotrione | H-12 |
| B-1225 | picolinafen | H-12 |
| B-1226 | sulcotrione | H-12 |
| B-1227 | tefuryltrione | H-12 |
| B-1228 | tembotrione | H-12 |
| B-1229 | topramezone | H-12 |
| B-1230 | H-7 | H-12 |
| B-1231 | atrazine | H-12 |
| B-1232 | diuron | H-12 |
| B-1233 | fluometuron | H-12 |
| B-1234 | hexazinone | H-12 |
| B-1235 | isoproturon | H-12 |
| B-1236 | metribuzin | H-12 |
| B-1237 | propanil | H-12 |
| B-1238 | terbuthylazine | H-12 |
| B-1239 | paraquat dichloride | H-12 |
| B-1240 | flumioxazin | H-12 |
| B-1241 | oxyfluorfen | H-12 |
| B-1242 | saflufenacil | H-12 |
| B-1243 | sulfentrazone | H-12 |
| B-1244 | H-1 | H-12 |
| B-1245 | H-2 | H-12 |
| B-1246 | glyphosate | H-12 |
| B-1247 | glyphosate-isopropylammonium | H-12 |
| B-1248 | glyphosate-trimesium (sulfosate) | H-12 |
| B-1249 | glufosinate | H-12 |
| B-1250 | glufosinate-ammonium | H-12 |
| B-1251 | pendimethalin | H-12 |
| B-1252 | trifluralin | H-12 |
| B-1253 | acetochlor | H-12 |
| B-1254 | cafenstrole | H-12 |
| B-1255 | dimethenamid-P | H-12 |
| B-1256 | fentrazamide | H-12 |
| B-1257 | flufenacet | H-12 |
| B-1258 | mefenacet | H-12 |
| B-1259 | metazachlor | H-12 |
| B-1260 | metolachlor-S | H-12 |
| B-1261 | pyroxasulfone | H-12 |
| B-1262 | isoxaben | H-12 |
| B-1263 | dymron | H-12 |
| B-1264 | indanofan | H-12 |
| B-1265 | oxaziclomefone | H-12 |
| B-1266 | triaziflam | H-12 |
| B-1267 | atrazine + H-1 | H-12 |
| B-1268 | atrazine + glyphosate | H-12 |
| B-1269 | atrazine + mesotrione | H-12 |
| B-1270 | atrazine + nicosulfuron | H-12 |
| B-1271 | atrazine + tembotrione | H-12 |
| B-1272 | atrazine + topramezone | H-12 |
| B-1273 | clomazone + glyphosate | H-12 |
| B-1274 | diflufenican + clodinafop-propargyl | H-12 |
| B-1275 | diflufenican + fenoxaprop-P-ethyl | H-12 |
| B-1276 | diflufenican + flupyrsulfuron-methyl-sodium | H-12 |
| B-1277 | diflufenican + glyphosate | H-12 |
| B-1278 | diflufenican + mesosulfuron-methyl | H-12 |
| B-1279 | diflufenican + pinoxaden | H-12 |
| B-1280 | diflufenican + pyroxsulam | H-12 |
| B-1281 | flumetsulam + glyphosate | H-12 |
| B-1282 | flumioxazin + glyphosate | H-12 |
| B-1283 | imazapic + glyphosate | H-12 |
| B-1284 | imazethapyr + glyphosate | H-12 |
| B-1285 | isoxaflutol + H-1 | H-12 |
| B-1286 | isoxaflutol + glyphosate | H-12 |
| B-1287 | metazachlor + H-1 | H-12 |
| B-1288 | metazachlor + glyphosate | H-12 |
| B-1289 | metazachlor + mesotrione | H-12 |
| B-1290 | metazachlor + nicosulfuron | H-12 |
| B-1291 | metazachlor + terbuthylazine | H-12 |
| B-1292 | metazachlor + topramezone | H-12 |
| B-1293 | metribuzin + glyphosate | H-12 |
| B-1294 | pendimethalin + H-1 | H-12 |
| B-1295 | pendimethalin + clodinafop-propargyl | H-12 |
| B-1296 | pendimethalin + fenoxaprop-P-ethyl | H-12 |
| B-1297 | pendimethalin + flupyrsulfuron-methyl-sodium | H-12 |
| B-1298 | pendimethalin + glyphosate | H-12 |
| B-1299 | pendimethalin + mesosulfuron-methyl | H-12 |
| B-1300 | pendimethalin + mesotrione | H-12 |
| B-1301 | pendimethalin + nicosulfuron | H-12 |
| B-1302 | pendimethalin + pinoxaden | H-12 |
| B-1303 | pendimethalin + pyroxsulam | H-12 |
| B-1304 | pendimethalin + tembotrione | H-12 |
| B-1305 | pendimethalin + topramezone | H-12 |
| B-1306 | pyroxasulfone + tembotrione | H-12 |
| B-1307 | pyroxasulfone + topramezone | H-12 |
| B-1308 | sulfentrazone + glyphosate | H-12 |
| B-1309 | terbuthylazine + H-1 | H-12 |
| B-1310 | terbuthylazine + foramsulfuron | H-12 |
| B-1311 | terbuthylazine + glyphosate | H-12 |
| B-1312 | terbuthylazine + mesotrione | H-12 |
| B-1313 | terbuthylazine + nicosulfuron | H-12 |
| B-1314 | terbuthylazine + tembotrione | H-12 |
| B-1315 | terbuthylazine + topramezone | H-12 |
| B-1316 | trifluralin + glyphosate | H-12 |
| B-1317 | 2-1 | — |
| B-1318 | 2-2 | — |
| B-1319 | 2-3 | — |
| B-1320 | 2-4 | — |
| B-1321 | 2-5 | — |
| B-1322 | 2-6 | — |
| B-1323 | 2-7 | — |
| B-1324 | 2-8 | — |
| B-1325 | 2-9 | — |
| B-1326 | 2-1 | benoxacor |
| B-1327 | 2-2 | benoxacor |
| B-1328 | 2-3 | benoxacor |
| B-1329 | 2-4 | benoxacor |
| B-1330 | 2-5 | benoxacor |
| B-1331 | 2-6 | benoxacor |
| B-1332 | 2-7 | benoxacor |
| B-1333 | 2-8 | benoxacor |
| B-1334 | 2-9 | benoxacor |
| B-1335 | 2-1 | cloquintocet |
| B-1336 | 2-2 | cloquintocet |
| B-1337 | 2-3 | cloquintocet |
| B-1338 | 2-4 | cloquintocet |
| B-1339 | 2-5 | cloquintocet |
| B-1340 | 2-6 | cloquintocet |
| B-1341 | 2-7 | cloquintocet |
| B-1342 | 2-8 | cloquintocet |
| B-1343 | 2-9 | cloquintocet |
| B-1344 | 2-1 | cyprosulfamide |
| B-1345 | 2-2 | cyprosulfamide |
| B-1346 | 2-3 | cyprosulfamide |
| B-1347 | 2-4 | cyprosulfamide |
| B-1348 | 2-5 | cyprosulfamide |
| B-1349 | 2-6 | cyprosulfamide |
| B-1350 | 2-7 | cyprosulfamide |
| B-1351 | 2-8 | cyprosulfamide |
| B-1352 | 2-9 | cyprosulfamide |
| B-1353 | 2-1 | dichlormid |
| B-1354 | 2-2 | dichlormid |
| B-1355 | 2-3 | dichlormid |
| B-1356 | 2-4 | dichlormid |
| B-1357 | 2-5 | dichlormid |
| B-1358 | 2-6 | dichlormid |
| B-1359 | 2-7 | dichlormid |
| B-1360 | 2-8 | dichlormid |
| B-1361 | 2-9 | dichlormid |
| B-1362 | 2-1 | fenchlorazole |
| B-1363 | 2-2 | fenchlorazole |
| B-1364 | 2-3 | fenchlorazole |
| B-1365 | 2-4 | fenchlorazole |
| B-1366 | 2-5 | fenchlorazole |
| B-1367 | 2-6 | fenchlorazole |
| B-1368 | 2-7 | fenchlorazole |
| B-1369 | 2-8 | fenchlorazole |
| B-1370 | 2-9 | fenchlorazole |
| B-1371 | 2-1 | isoxadifen |
| B-1372 | 2-2 | isoxadifen |
| B-1373 | 2-3 | isoxadifen |
| B-1374 | 2-4 | isoxadifen |
| B-1375 | 2-5 | isoxadifen |
| B-1376 | 2-6 | isoxadifen |
| B-1377 | 2-7 | isoxadifen |
| B-1378 | 2-8 | isoxadifen |
| B-1379 | 2-9 | isoxadifen |
| B-1380 | 2-1 | mefenpyr |
| B-1381 | 2-2 | mefenpyr |
| B-1382 | 2-3 | mefenpyr |
| B-1383 | 2-4 | mefenpyr |
| B-1384 | 2-5 | mefenpyr |
| B-1385 | 2-6 | mefenpyr |
| B-1386 | 2-7 | mefenpyr |
| B-1387 | 2-8 | mefenpyr |
| B-1388 | 2-9 | mefenpyr |
| B-1389 | 2-1 | H-11 |
| B-1390 | 2-2 | H-11 |
| B-1391 | 2-3 | H-11 |
| B-1392 | 2-4 | H-11 |
| B-1393 | 2-5 | H-11 |
| B-1394 | 2-6 | H-11 |
| B-1395 | 2-7 | H-11 |
| B-1396 | 2-8 | H-11 |
| B-1397 | 2-9 | H-11 |
| B-1398 | 2-1 | H-12 |
| B-1399 | 2-2 | H-12 |
| B-1400 | 2-3 | H-12 |
| B-1401 | 2-4 | H-12 |
| B-1402 | 2-5 | H-12 |
| B-1403 | 2-6 | H-12 |
| B-1404 | 2-7 | H-12 |
| B-1405 | 2-8 | H-12 |
| B-1406 | 2-9 | H-12 |

The compounds of formula I and the compositions according to the invention may also have a plant-strengthening action. Accordingly, they are suitable for mobilizing the defense system of the plants against attack by unwanted microorganisms, such as harmful fungi, but also viruses and bacteria. Plant-strengthening (resistance-inducing) substances are to be understood as meaning, in the present context, those substances which are capable of stimulating the defense system of treated plants in such a way that, when subsequently inoculated by unwanted microorganisms, the treated plants display a substantial degree of resistance to these microorganisms.

The compounds of formula I can be employed for protecting plants against attack by unwanted microorganisms within a certain period of time after the treatment. The period of time within which their protection is effected generally extends from 1 to 28 days, preferably from 1 to 14 days, after the treatment of the plants with the compounds of formula I, or, after treatment of the seed, for up to 9 months after sowing.

The compounds of formula I and the compositions according to the invention are also suitable for increasing the harvest yield.

Moreover, they have reduced toxicity and are tolerated well by the crop plants.

The following examples will further illustrate the invention:

With appropriate modification of the starting materials, the procedures given in the synthesis examples below were used to obtain further compounds I. The compounds obtained in this manner are listed in the tables that follow, together with physical data. The products shown below were characterized by determination of the melting point, NMR spectroscopy or the masses ([m/z]) determined by HPLC-MS spectrometry.

HPLC-MS=high performance liquid chromatography coupled with mass spectrometry; HPLC column: RP-18 column (Chromolith Speed ROD from Merck KgaA, Germany), 50*4.6 mm; mobile phase: acetonitrile+0.1% trifluoroacetic acid (TFA)/water+0.1% TFA, using a gradient from 5:95 to 100:0 over 5 minutes at 40° C., flow rate 1.8 mL/min.

MS: quadrupole electrospray ionization, 80 V (positive mode).

HPLC column: Luna-C18(2) 5 μm column (Phenomenex), 2.0*50 mm; mobile phase: acetonitrile+0.0625% trifluoroacetic acid (TFA)/water+0.0675% TFA, using a gradient from 10:90 to 80:20 over 4.0 minutes at 40° C., flow rate 0.8 mL/min.

MS: quadrupole electrospray ionization, 70 V (positive mode).

EtOAc: acetic acid ethyl ester

THF: tetrahydrofuran

LiHMDS: lithium bis(trimethylsilyl)amide

m-CPBA: 3-chloroperoxybenzoic acid

TFA: trifluoroacetic acid

n-BuLi: n-butyllithium

DMF: N,N-dimethylformamide

Et₂O: diethyl ether

### EXAMPLE 1: PREPARATION OF: 4-CHLORO-3-[[ETHYL(METHYL)CARBAMOYL]AMINO]-2-METHYL-N-(1-METHYLTETRAZOL-5-yl)benzamide (entry 3 in Table 26)

### Step 1: Methyl 4-chloro-3-isocyanato-2-methyl-benzoate

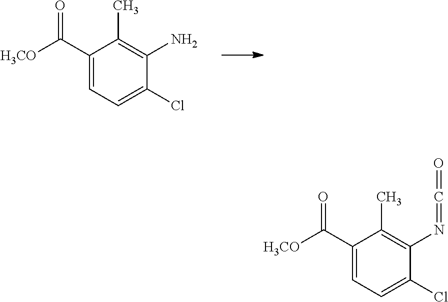

Triphosgene (bis(trichloromethyl) carbonate, 44.6 g, 0.15 mol) was dissolved in toluene (550 ml) and treated with a solution of methyl 3-amino-4-chloro-2-methyl-benzoate (15 g, 75 mmol) in 50 ml of toluene at a temperature of 70° C. Temperature was increased stepwise to reflux. Reflux was kept for 6 h and the reaction mixture stirred overnight at room temperature. Afterwards the reaction mixture was evaporated in vacuoto leave the product (16.8 g, 99%) as an orange solid which was used without further purification for the next step.

¹H NMR (400 MHz, CDCl₃), δ 7.65 (d, 1H) 7.3 (d, 1H), 3.9 (s, 3H), 2.6 (s, 3H).

### Step 2: 4-Chloro-3-[[ethyl(methyl)carbamoyl]amino]-2-methyl-benzoic acid

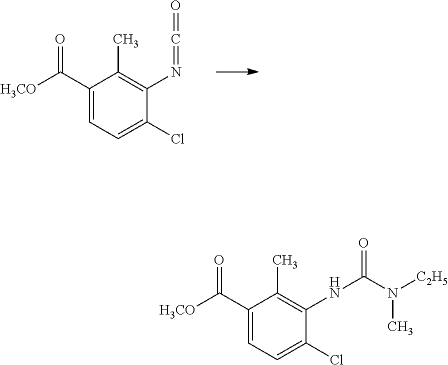 [SMILES: COC(=O)c1ccc(Cl)c(N=C=O)c1C ; CCN(C)C(=O)Nc1c(Cl)ccc(C(=O)OC)c1C]

Methyl 4-chloro-3-isocyanato-2-methyl-benzoate (2.50 g, 11.1 mmol) was dissolved in THF (60 ml) and diethylamine (720 mg, 12.2 mmol) was added dropwise at room temperature. Stirring was continued for 2.5 h. Water (10 ml) and lithium hydroxide (796 mg, ca. 33 mmol) was added and the mixture stirred overnight. The reaction mixture was concentrated in vacuo and the remaining aqueous phase was adjusted to a pH value of ca. 1-2 by addition of hydrochloric acid (1 molar). The product precipitated upon this treatment was filtered off. Drying in vacuo yielded 2.20 g of a solid (73%) of the title compound which was used without further purification in the next step.

### Step 3: 4-chloro-3-[[ethyl(methyl)carbamoyl]amino]-2-methyl-N-(1-methyltetrazol-5-yl)benzamide

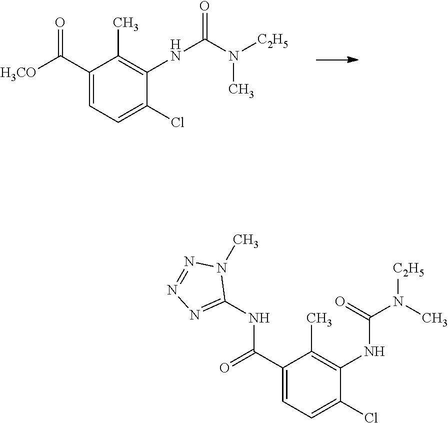 [SMILES: CCN(C)C(=O)Nc1c(Cl)ccc(C(=O)OC)c1C ; CCN(C)C(=O)Nc1c(Cl)ccc(C(=O)Nc2nnnn2C)c1C]

1-Methyltetrazol-5-amine (1.37 g, 13.9 mmol) was dissolved in anhydrous THF (100 ml) and cooled to −70° C. under argon atmosphere. A solution of methyl lithium in THF (3.1 molar, 13.9 mmol) was added dropwise at this temperature and the reaction mixture warmed up to 0° C. At this temperature the solution prepared in the following manner was added dropwise: 4-chloro-3-[[ethyl(methyl)carbamoyl]amino]-2-methyl-benzoic acid (1.50 g, 5.54 mmol) was suspended in dichloromethane (DCM, 60 ml) and cooled to −78° C. under argon atmosphere and DAST (diethlyamino sulfur trifluoride, 982 mg, 6.10 mmol) was added via syringe. Cooling was continued for 3 h and the reaction mixture afterwards allowed to warm up to RT overnight. The solvent was evaporated in vacuo and re-dissolved in anhydrous THF (20 ml).

After addition if this solution stirring was continued at RT for 17 h and the reaction mixture carefully quenched by addition of water (40 ml). The major amounts of THF were evaporated in vacuo and the remainder adjusted to a pH value of 1-2 by addition of aqueous hydrochloric acid (2 molar). Extraction with ethyl acetate (3×30 ml), drying of the combined organic phases with magnesium sulfate and evaporation of the solvent yielded crude product which was purified by column chromatography to yield the title compound (390 mg, 20%).

¹H NMR (400 MHz, d6-DMSO), δ 13 (brs, 1H) 7.95 (s, 1H), 7.6 (d, 1H), 7.4 (d, 1H), 3.3 (m, 2H, hidden by solvent), 2.9 (s, 3H), 2.35 (s, 3H), 1.1 (t, 3H).

By analogy to the methods described in the Examples above, the following compounds of formulae I.A, I.B and I.D according to Table 26 were prepared:

TABLE 26

| | | | | | | | I.A |
|---|---|---|---|---|---|---|---|
| 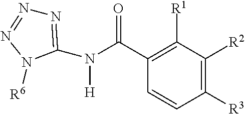 | | | | | | | |
| | | | | | | | I.B |
| 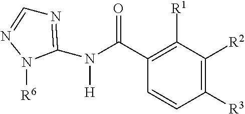 | | | | | | | |
| | | | | | | | I.D |
| 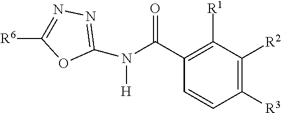 | | | | | | | |
| | | in tables | | | | | MS |
| cpd. no | formula | 1 to 25 | R¹ | R² | R³ | R⁶ | (m/z) |
| 1 | I.A | I.A.I.547 | CH₃ | 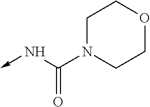 | Cl | CH₃ | 380.0 |
| 2 | I.A | I.A.I.313 | CH₃ | 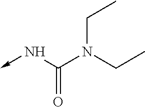 | Cl | CH₃ | 366.1 |
| 3 | I.A | I.A.I.283 | CH₃ | 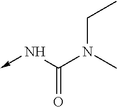 | Cl | CH₃ | 352.1 |
| 4 | I.A | I.A.I.289 | CH₃ | 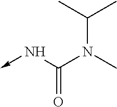 | Cl | CH₃ | 366.0 |
| 5 | I.A | I.A.I.367 | CH₃ | 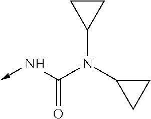 | Cl | CH₃ | 390.0 |
| 6 | I.A | I.A.I.571 | CH₃ | 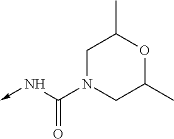 | Cl | CH₃ | 408.0 |
| 7 | I.A | I.A.I.577 | CH₃ | 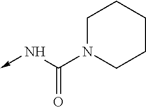 | Cl | CH₃ | 378.0 |
| 8 | I.A | I.A.I.583 | CH₃ | 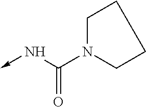 | Cl | CH₃ | 364.0 |
| 9 | I.A | I.A.I.307 | CH₃ | 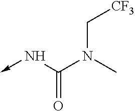 | Cl | CH₃ | 405.9 |
| 10 | I.A | 1.A.I.31 | Cl | 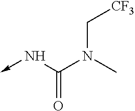 | Cl | CH₃ | 425.8 |
| 11 | I.A | I.A.VII.31 | Cl | 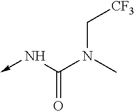 | Cl | CH₂CH₂OCH₃ | 469.9 |
| 12 | I.A | I.A.I.337 | CH₃ | 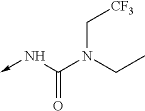 | Cl | CH₃ | 419.9 |
| 13 | I.A | I.A.I.61 | Cl | 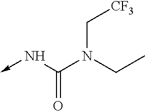 | Cl | CH₃ | 439.9 |
| 14 | I.A | I.A.I.55 | Cl | 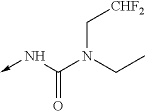 | Cl | CH₃ | 421.9 |
| 15 | I.A | I.A.I.25 | Cl | 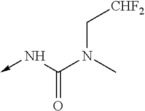 | Cl | CH₃ | 407.8 |
| 16 | I.A | I.A.I.301 | CH₃ | 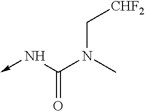 | Cl | CH₃ | 378.0 |
| 17 | I.B | I.B.I.31 | Cl | 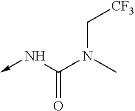 | Cl | CH₃ | 424.8 |
| 18 | I.D | I.D.I.313 | CH₃ | 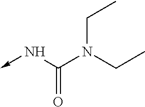 | Cl | CH₃ | 365.9 |
| 19 | I.D | I.D.I.283 | CH₃ | 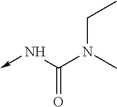 | Cl | CH₃ | 351.8 |
| 20 | I.D | I.D.I.307 | CH₃ | 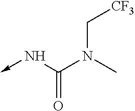 | Cl | CH₃ | 405.9 |
| 21 | I.D | I.D.I.337 | CH₃ | 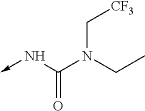 | Cl | CH₃ | 419.9 |

### Use Examples

The herbicidal activity of the compounds of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted.

This cover caused uniform germination of the test plants, unless this had been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-25° C., respectively.

The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

At an application rate of 250 g/ha the following compounds were tested in post-emergence tests against:

| | in tables | | | | |
|---|---|---|---|---|---|
| cpd. no | 1 to 25 | ALOMY | ECHCG | AMARE | CHEAL |
| 1 | I.A.I.547 | 98 | 100 | 100 | 100 |
| 2 | I.A.I.313 | 98 | 100 | 100 | 100 |
| 3 | I.A.I.283 | 98 | 100 | 100 | 100 |
| 4 | I.A.I.289 | 98 | 100 | 100 | 100 |
| 5 | I.A.I.367 | 98 | 100 | 100 | 100 |
| 9 | I.A.I.307 | >80 | >80 | >80 | >80 |
| 10 | I.A.I.31 | >80 | >80 | >80 | >80 |
| 11 | I.A.VII.31 | >80 | >80 | >80 | >80 |
| 12 | I.A.I.337 | >80 | >80 | >80 | >80 |
| 13 | I.A.I.61 | >80 | >80 | >80 | >80 |
| 14 | I.A.1.55 | >80 | >80 | >80 | >80 |
| 17 | I.B.I.31 | >80 | >80 | >80 | >80 |
| 18 | I.D.I.313 | >80 | >80 | >80 | >80 |
| 20 | I.D.I.307 | >80 | >80 | >80 | >80 |
| 21 | I.D.I.337 | >80 | >80 | >80 | >80 |

ALOMY (Alopecurus myosuroiedes)
ECHCG (Echinocloa crus-galli)
AMARE (Amaranthus retroflexus)
CHEAL (Chenopodium album)

1. A compound of formula I, 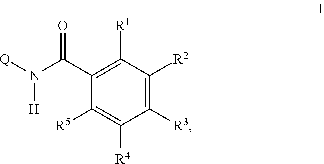
wherein
Q is Q¹ or Q² Or Q³ or Q⁴, 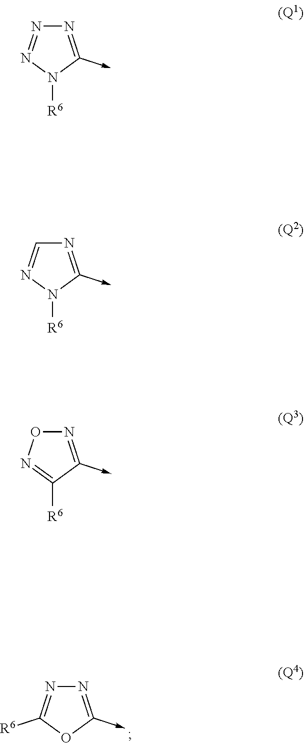
R¹ is selected from the group consisting of cyano, halogen, nitro, C₁-C₈-alkyl, C₁-C₈-haloalkyl, C₁-C₄-alkoxy-C₁-C₄-alkyl, C₁-C₄-haloalkoxy-C₁-C₄-alkyl, C₁-C₈-alkoxy, C₁-C₄-alkoxy-C₁-C₄-alkoxy-Z¹, C₁-C₆-haloalkoxy, R^{1b}—S(O)ₖ—Z¹;
R² is R^{2c}R^{2d}NC(O)NR²ⁿ—Z²;
R³ is selected from the group consisting of hydrogen, cyano, thiocyanato, halogen, hydroxy-Z², nitro, C₁-C₆-alkyl, C₂-C₈-alkenyl, C₂-C₈-alkynyl, C₃-C₁₀-cycloalkyl-Z², C₃-C₆-cycloalkenyl-Z², C₃-C₁₀-cycloalkoxy-Z², C₃-C₁₀-cycloalkyl-C₁-C₂-alkoxy, where the cyclic groups of the four aforementioned radicals are unsubstituted or partially or completely halogenated, C₁-C₄-cyanoalkyl, C₁-C₈-haloalkyl, C₂-C₈-haloalkenyl, C₃-C₈-haloalkynyl, C₁-C₈-alkoxy-Z², C₁-C₈-haloalkoxy-Z², C₁-C₄-alkoxy-C₁-C₄-alkoxy-Z², C₁-C₄-haloalkoxy-C₁-C₄-alkoxy-Z², C₂-C₈-alkenyloxy-Z², C₂-C₈-alkynyloxy-Z², C₂-C₈-haloalkenyloxy-Z², C₃-C₈haloalkynyloxy-Z², R^{2b}—S(O)ₖ—Z², R^{2c}—C(O)—Z², R^{2d}O—C(O)—Z², R^{2d}O—N═CH—Z², R^{2e}R^{2f}N—C(O)—Z², R^{2g}R^{2h}N—Z², R²²C(O)O—Z², R²⁵OC(O)O—Z², (R²²)₂NC(O)O—Z², R²⁵S(O)₂O—Z², R²²OS(O)₂—Z², (R²²)₂NS(O)₂—Z², R²⁵OC(O)N(R²²)—Z², (R²²)₂NC(O)N(R²²)—Z², (R²²)₂NS(O)₂N(R²²)—Z², (OH)₂P(O)—Z², (C₁-C₄-alkoxy)₂P(O)—Z², phenyl-Z^{2a}, heterocyclyl-Z^{2a}, where heterocyclyl is a 3-, 4-, 5- or 6-membered monocyclic or 8-, 9- or 10-membered bicyclic saturated, partially unsaturated or aromatic heterocycle, which contains 1, 2, 3 or 4 heteroatoms as ring members, which are selected from the group consisting of O, N and S, where cyclic groups in phenyl-Z^{2a} and heterocyclyl-Z^{2a} are unsubstituted or substituted by 1, 2, 3 or 4 groups R²¹, which are identical or different;
R⁴ is hydrogen;
R⁵ is hydrogen;
R⁶ is selected from the group consisting of cyano, C₁-C₆-alkyl, C₃-C₇-cycloalkyl, C₃-C₇-cycloalkyl-C₁-C₄-alkyl, where the C₃-C₇-cycloalkyl groups of the two aforementioned radicals are unsubstituted or partially or completely halogenated, C₁-C₆-haloalkyl, C₂-C₆-alkenyl, C₂-C₆-alkynyl, C₂-C₆-haloalkenyl, C₃-C₆-haloalkynyl, C₁-C₄-alkoxy-C₁-C₄-alkyl, C₁-C₄-haloalkoxy-C₁-C₄-alkyl, R^{b}—S(O)ₙ—C₁-C₃-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R²¹ is selected from the group consisting of cyano, halogen, nitro, C₁-C₆-alkyl, C₃-C₇-cycloalkyl, C₃-C₇-halocycloalkyl, C₁-C₆-haloalkyl, C₂-C₆-alkenyl, C₂-C₆-haloalkenyl, C₂-C₆-alkynyl, C₃-C₆-haloalkynyl, C₁-C₆-alkoxy, C₁-C₄-alkoxy-C₁-C₄-alkyl, C₁-C₄-alkylthio-C₁-C₄-alkyl, C₁-C₄-haloalkoxy-C₁-C₄-alkyl, C₁-C₄-alkoxy-C₁-C₄-alkoxy, C₃-C₇-cycloalkoxy and C₁-C₆-haloalkoxy, or two radicals R²¹ bound to the same carbon atom together may form a group ═O;
Z¹, Z² independently of each other are selected from the group consisting of a covalent bond and C₁-C₄-alkanediyl;
Z^{2a} is selected from the group consisting of a covalent bond, C₁-C₄-alkanediyl, O—C₁-C₄-alkanediyl, C₁-C₄-alkanediyl-O and C₁-C₄-alkanediyl-O—C₁-C₄-alkanediyl;
R^{b}, R^{1b}, R^{2b} independently of each other are selected from the group consisting of C₁-C₆-alkyl, C₃-C₇-cycloalkyl, C₁-C₆-haloalkyl, C₂-C₆-alkenyl, C₂-C₆-haloalkenyl, C₂-C₆-alkynyl, C₃-C₆-haloalkynyl, phenyl and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2c} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₁-C₆-alkoxy, C₂-C₆-alkenyl, C₁-C₄-alkyl-C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkyl-S(O)ₙ—C₁-C₄-alkyl, C₁-C₄-alkylamino-C₁-C₄-alkyl, C₁-C₄-dialkylamino-C₁-C₄-alkyl, C₁-C₆-cyanoalkyl, phenyl, benzyl and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, benzyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2d} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₁-C₆-alkoxy, C₂-C₆-alkenyl, C₁-C₄-alkyl-C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkyl-S(O)ₙ—C₁-C₄-alkyl, C₁-C₄-alkylamino-C₁-C₄-alkyl, C₁-C₄-dialkylamino-C₁-C₄-alkyl, C₁-C₆-cyanoalkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2c}, R^{2d} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R²ⁿ is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-cyanoalkyl, C₁-C₆-haloalkyl, C₂-C₆-alkenyl, C₂-C₆-haloalkenyl, C₂-C₆-alkynyl, C₁-C₄-alkoxy-C₁-C₄-alkyl;
R^{2e}, R^{2f} independently of each other are selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2e}, R^{2f} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R^{2g} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylsulfonyl, C₁-C₄-alkylcarbonyl, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy;
R^{2h} is selected from the group consisting of hydrogen, C₁-C₆-alkyl, C₃-C₇-cycloalkyl, C₃-C₇-cycloalkyl-C₁-C₄-alkyl, where the C₃-C₇-cycloalkyl groups in the two aforementioned radicals are unsubstituted or partially or completely halogenated, C₁-C₆-haloalkyl, C₂-C₆-alkenyl, C₂-C₆-haloalkenyl, C₂-C₆-alkynyl, C₃-C₆-haloalkynyl, C₁-C₄-alkoxy-C₁-C₄-alkyl, C₁-C₄-alkylsulfonyl, C₁-C₄-alkylcarbonyl, a radical C(O)R^{k}, phenyl and benzyl, where phenyl and benzyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy; or
R^{2g}, R^{2h} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy;
R²² is selected from the group consisting of hydrogen, C₁-C₆-alkyl, C₁-C₆-haloalkyl, C₂-C₆-alkenyl, C₂-C₆-haloalkenyl, C₂-C₆-alkynyl, C₃-C₆-haloalkynyl, C₃-C₆-cycloalkyl, C₃-C₆-cycloalkenyl, C₃-C₆-halocycloalkyl, C₃-C₆-cycloalkyl-C₁-C₆-alkyl, C₁-C₆-alkoxy-C₁-C₆-alkyl, C₃-C₆-cycloalkyl-C₁-C₆-alkoxy-C₁-C₆-alkyl, phenyl-Z¹, phenyl-O—C₁-C₆-alkyl, phenyl-N(R²³)—C₁-C₆-alkyl, phenyl-S(O)ₙ—C₁-C₆-alkyl, heterocyclyl-Z¹, heterocyclyl-N(R²³)—C₁-C₆-alkyl, heterocyclyl-O—C₁-C₆-alkyl, heterocyclyl-S(O)ₙ—C₁-C₆-alkyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, heterocyclyl are unsubstituted or substituted by 1, 2, 3 or 4 groups, which are identical or different and selected from the group consisting of cyano, halogen, nitro, thiocyanato, C₁-C₆-alkyl, C₁-C₆-haloalkyl, C₃-C₆-cycloalkyl, C(O)OR²³, C(O)N(R²³)₂, OR²³, N(R²³)₂, S(O)ₙR²⁴, S(O)₂OR²³, S(O)₂N(R²³)₂, and R²³O—C₁-C₆-alkyl, and where heterocyclyl bears 0, 1 or 2 oxo groups;
R²³ is selected from the group consisting of hydrogen, C₁-C₆-alkyl, C₁-C₆-haloalkyl, C₂-C₆-alkenyl, C₂-C₆-alkynyl, C₃-C₆-cycloalkyl, C₃-C₆-cycloalkyl-C₁-C₆-alkyl, and phenyl;
R²⁴ is selected from the group consisting of C₁-C₆-alkyl, C₁-C₆-haloalkyl, C₂-C₆-alkenyl, C₂-C₆-alkynyl, C₃-C₆-cycloalkyl, C₃-C₆-cycloalkyl-C₁-C₆-alkyl, and phenyl;
R²⁵ is selected from the group consisting of C₁-C₆-alkyl, C₁-C₆-haloalkyl, C₂-C₆-alkenyl, C₂-C₆-haloalkenyl, C₂-C₆-alkynyl, C₃-C₆-haloalkynyl, C₃-C₆-cycloalkyl, C₃-C₆-cycloalkenyl, C₃-C₆-halocycloalkyl, C₃-C₆-cycloalkyl-C₁-C₆-alkyl, C₁-C₆-alkoxy-C₁-C₆-alkyl, C₃-C₆-cycloalkyl-C₁-C₆-alkoxy-C₁-C₆-alkyl, phenyl-Z¹, phenyl-O—C₁-C₆-alkyl, phenyl-N(R²³)—C₁-C₆-alkyl, phenyl-S(O)ₙ—C₁-C₆-alkyl, heterocyclyl-Z¹, heterocyclyl-N(R²³)—C₁-C₆-alkyl, heterocyclyl-O—C₁-C₆-alkyl, heterocyclyl-S(O)ₙ—C₁-C₆-alkyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl, heterocyclyl are unsubstituted or substituted by 1, 2, 3 or 4 groups, which are identical or different and selected from the group consisting of cyano, halogen, nitro, thiocyanato, C₁-C₆-alkyl, C₁-C₆-haloalkyl, C₃-C₆-cycloalkyl, C(O)OR²³, C(O)N(R²³)₂, OR²³, N(R²³)₂, S(O)ₙR²⁴, S(O)₂OR²³, S(O)₂N(R²³)₂, and R²³O—C₁-C₆-alkyl, and where heterocyclyl bears 0, 1 or 2 oxo groups;
k is 0, 1 or 2;
n is 0, 1 or 2;
R^{k} has the meanings of R^{2c};
or an agriculturally suitable salt thereof;
with the exception of
Benzamide, 4-bromo-2-chloro-N-[1-(2-methoxyethyl)-1H-tetrazol-5-yl]-3-[[(methoxymethylamino)carbonyl]amino]-, CAS Registry Number: 1421777-21 0;
Benzamide, 2,4-dichloro-N-[1-(2-methoxyethyl)-1H-tetrazol-5-yl]-3-[[(methoxymethylamino)carbonyl]amino]-, CAS Registry Number: 1421776-96-6;
Benzamide, 2,4-dichloro-3-[[(methoxymethylamino)carbonyl]amino]-N-(1-phenyl-1H-tetrazol-5-yl)-, CAS Registry Number: 1361245-23-9;
Benzamide, N-(1-ethyl-1H-tetrazol-5-yl)-2-methyl-3-[[[methyl(2-methylpropyl)amino]carbonyl]amino]-4-(methylsulfonyl)-, CAS Registry Number: 1361209-74-6;
Benzamide, 2,4-dichloro-3-[[(dimethylamino)carbonyl]amino]-N-(1-methyl-1H-tetrazol-5-yl)-, CAS Registry Number: 1361139-93-6;
Benzamide, 2,4-dichloro-3-[[(methoxymethylamino)carbonyl]amino]-N-(1-phenyl-1H-1,2,4-triazol-5-yl)-, CAS Registry Number: 1361103-06-1;
Benzamide, 2,4-dichloro-N-(1-ethyl-1H-1,2,4-triazol-5-yl)-3-[[(methoxymethylamino)carbonyl]amino]-, CAS Registry Number: 1361040-89-2;
Benzamide, N-(4-cyano-1,2,5-oxadiazol-3-yl)-2-methyl-3-[[[methyl(2-methylpropyl)amino]carbonyl]amino]-4-(methylsulfonyl)-, CAS Registry Number: 1279687-63-6;
Benzamide, 2-methyl-3-[[[methyl(2-methylpropyl)amino]carbonyl]amino]-4-(methylsulfonyl)-N-[4-(trifluoromethyl)-1,2,5-oxadiazol-3-yl]-, CAS Registry Number: 1279679-31-0;
Benzamide, N-[4-(1,1-dimethylethyl)-1,2,5-oxadiazol-3-yl]-2-methyl-3-[[[methyl(2-methylpropyl)amino] carbonyl]amino]-4-(methyl sulfonyl)-, CAS Registry Number: 1279665-87-0;
Benzamide, N-[4-(chloromethyl)-1,2,5-oxadiazol-3-yl]-2-methyl-3-[[[methyl(2-methylpropyl)amino] carbonyl]amino]-4-(methyl sulfonyl)-, CAS Registry Number: 1279659-60-7;
Benzamide, 2-methyl-3-[[[methyl(2-methylpropyl)amino]carbonyl]amino]-4-(methylsulfonyl)-N-(4-propyl-1,2,5-oxadiazol-3-yl)-, CAS Registry Number: 1279644-16-4;
Benzamide, 2-methyl-N-[4-(1-methylethyl)-1,2,5-oxadiazol-3-yl]-3-[[[methyl(2-methylpropyl)amino] carbonyl]amino]-4-(methyl sulfonyl)-, CAS Registry Number: 1279635-24-3;
Benzamide, N-(4-ethyl-1,2,5-oxadiazol-3-yl)-2-methyl-3-[[[methyl(2-methylpropyl)amino] carbonyl]amino]-4-(methyl sulfonyl)-, CAS Registry Number: 1279127-99-9;
Benzamide, 2-methyl-3-[[[methyl(2-methylpropyl)amino]carbonyl]amino]-N-(4-methyl-1,2,5-oxadiazol-3-yl)-4-(methylsulfonyl)-, CAS Registry Number: 1279114-20-3.

2. The compound as claimed in claim 1, or an agriculturally suitable salt thereof, where Q is Q¹.

3. The compound as claimed in claim 1, or an agriculturally suitable salt thereof, where Q is Q².

4. The compound as claimed in claim 1, or an agriculturally suitable salt thereof, where Q is Q³.

5. The compound as claimed in claim 1, or an agriculturally suitable salt thereof, where Q is Q⁴.

6. The compound as claimed in claim 1, or an agriculturally suitable salt thereof, where R¹ is selected from the group consisting of halogen, nitro, C₁-C₄-alkyl, C₁-C₄-alkoxy-C₁-C₄-alkoxy-Z¹, R^{1b}—S(O)ₖ—Z¹.

7. The compound as claimed in claim 1, or an agriculturally suitable salt thereof, where R² is R^{2c}R^{2d}NC(O)NR²ⁿ—Z², where Z² is a covalent bond and R²ⁿ is selected from the group consisting of hydrogen and C₁-C₆-alkyl.

8. The compound as claimed in claim 1, or an agriculturally suitable salt thereof, where R² is R^{2c}R^{2d}NC(O)NR²ⁿ—Z², where Z² is C₁-C₄-alkanediyl and R²ⁿ is selected from the group consisting of hydrogen and C₁-C₆-alkyl.

9. The compound as claimed in claim 1, or an agriculturally suitable salt thereof, where Z² in R² is a covalent bond and R^{2c} and R^{2d} independently of each other are selected from the group consisting of C₁-C₆-alkyl, C₃-C₇-cycloalkyl, C₃-C₇-cycloalkyl-C₁-C₄-alkyl, C₁-C₆-haloalkyl, phenyl, and heterocyclyl, where heterocyclyl is a 5- or 6-membered monocyclic saturated, partially unsaturated or aromatic heterocycle, which contains 1, 2, 3 or 4 heteroatoms as ring members, which are selected from the group consisting of O, N and S, where phenyl and heterocyclyl are unsubstituted or substituted by 1, 2, 3 or 4 groups, which are identical or different and selected from the group consisting of halogen, C₁-C₄-alkyl, C₁-C₄-haloalkyl, C₁-C₄-alkoxy and C₁-C₄-haloalkoxy.

10. The compound as claimed in claim 9, or an agriculturally suitable salt thereof, where Z² in R² is a covalent bond R²C is selected from the group consisting of C₁-C₄-alkyl, C₃-C₇-cycloalkyl and C₁-C₄-haloalkyl and R^{2d} is selected from the group consisting of C₂-C₆-alkyl, C₃-C₇-cycloalkyl, C₃-C₇-cycloalkyl-C₁-C₂-alkyl and C₁-C₆-haloalkyl.

11. The compound as claimed in claim 1, or an agriculturally suitable salt thereof, where Z² in R² is a covalent bond and R^{2c} and R^{2d} together with the nitrogen atom, to which they are bound may form a 4-, 5-, 6- or 7-membered, saturated or unsaturated heterocyclic radical, which may carry as a ring member a further heteroatom selected from O, S and N and which is unsubstituted or may carry 1, 2, 3 or 4 groups, which are identical or different and selected from the group consisting of ═O, halogen, C₁-C₄-alkyl, C₁-C₄-haloalkyl, C₁-C₄-alkoxy and C₁-C₄-haloalkoxy.

12. The compound as claimed in claim 1, or an agriculturally suitable salt thereof, where R³ is selected from the group consisting of halogen, nitro, C₁-C₄-alkyl, C₁-C₄-haloalkyl, C₁-C₄-haloalkoxy, and R^{2b-}S(O)ₖ.

13. The compound as claimed in claim 1, or an agriculturally suitable salt thereof, where R³ is selected from the group consisting of halogen and C₁-C₂-haloalkyl.

14. The compound as claimed in claim 1, or an agriculturally suitable salt thereof, where R¹ is methyl and R³ is selected from the group consisting of Cl, Br and CF₃.

15. The compound as claimed in claim 1, or an agriculturally suitable salt thereof, where R¹ is Cl and R³ is selected from the group consisting of Br and CF₃.

16. The compound as claimed in claim 1, or an agriculturally suitable salt thereof, where
R¹ is C₁-C₂-alkyl,
R³ is selected from the group consisting of halogen and C₁-C₂-haloalkyl and where Z² in R² is a covalent bond,
R^{2c} is selected from the group consisting C₁-C₄-alkyl, C₃-C₇-cycloalkyl and C₁-C₄-haloalkyl, and
R^{2d} is selected from the group consisting of C₂-C₆-alkyl, C₃-C₇-cycloalkyl, C₃-C₇-cycloalkyl-C₁-C₂-alkyl and C₁-C₄-haloalkyl.

17. The compound as claimed in claim 1, or an agriculturally suitable salt thereof, where
R¹ is methyl and R³ is selected from the group consisting of Cl, Br and CF₃ and where Z² in R² is a covalent bond,
R^{2c} is selected from the group consisting C₁-C₄-alkyl, C₃-C₇-cycloalkyl and C₁-C₄-haloalkyl, and
R^{2d} is selected from the group consisting of C₁-C₆-alkyl, C₃-C₇-cycloalkyl, C₃-C₇-cycloalkyl-C₁-C₂-alkyl and C₁-C₆-haloalkyl.

18. The compound as claimed in claim 1, or an agriculturally suitable salt thereof, where
R¹ is halogen,
R³ is selected from the group consisting of halogen and C₁-C₂-haloalkyl and where Z² in R² is a covalent bond,
R^{2c} is selected from the group consisting C₁-C₄-alkyl C₃-C₇-cycloalkyl, and C₁-C₄-haloalkyl, and
R^{2d} is selected from the group consisting of C₂-C₆-alkyl, C₃-C₇-cycloalkyl, C₃-C₇-cycloalkyl-C₁-C₂-alkyl and C₁-C₆-haloalkyl.

19. The compound as claimed in claim 1, or an agriculturally suitable salt thereof, where
R¹ is Cl and R³ is selected from the group consisting of Br and CF₃. and where Z² in R² is a covalent bond,
R^{2c} is selected from the group consisting C₁-C₄-alkyl, C₃-C₇-cycloalkyl and C₁-C₄-haloalkyl, and
R^{2d} is selected from the group consisting of C₁-C₆-alkyl, C₃-C₇-cycloalkyl, C₃-C₇-cycloalkyl-C₁-C₂-alkyl and C₁-C₆-haloalkyl.

20. The compound as claimed in claim 1, or an agriculturally suitable salt thereof, where R¹ is Cl and R³ is Cl and where Z² in R² is a covalent bond,
R^{2c} is selected from the group consisting C₁-C₄-alkyl, C₃-C₇-cycloalkyl and C₁-C₄-haloalkyl, and
R^{2d} is selected from the group consisting of C₂-C₆-alkyl, C₃-C₇-cycloalkyl, C₃-C₇-cycloalkyl-C₁-C₂-alkyl and C₁-C₆-haloalkyl.

21. The compound as claimed in claim 1, or an agriculturally suitable salt thereof, where R⁶ is selected from the group consisting of C₁-C₆-alkyl, C₁-C₄-alkoxy-C₁-C₄-alkyl, and phenyl.

22. The compound as claimed in claim 21, or an agriculturally suitable salt thereof, where R⁶ is methyl.

23. The compound as claimed in claim 1, or an agriculturally suitable salt thereof, which is selected from the compounds of the following formulae I.A.I and I.D.I, 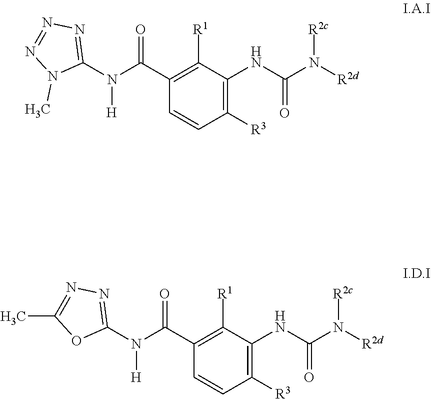
where, R¹, R^{2c}, R^{2d} and R³ are as defined in the following tables:

| | Formula | R¹ | R^{2c} | R^{2d} | R³ |
|---|---|---|---|---|---|
| 1. | I.A.I | CH₃ | CH₃ | CH₂CH₃ | Cl |
| 2. | I.A.I | CH₃ | CH₂CH₃ | CH₂CH₃ | Cl |
| 3. | I.A.I | CH₃ | CH₃ | CH₂CF₃ | Cl |
| 4. | I.A.I | CH₃ | CH₂CH₃ | CH₂CF₃ | Cl |
| 5. | I.A.I | CH₃ | CH₃ | CH₂CHF₂ | Cl |
| 6. | I.A.I | CH₃ | CH₂CH₃ | CH₂CHF₂ | Cl |
| 7. | I.A.I | CH₃ | CH₃ | CH(CH₃)₂ | Cl |
| 8. | I.A.I | CH₃ | c-C₃H₅ | c-C₃H₅ | Cl |
| 9. | I.A.I | Cl | CH₃ | CH₂CH₃ | Cl |
| 10. | I.A.I | Cl | CH₂CH₃ | CH₂CH₃ | Cl |
| 11. | I.A.I | Cl | CH₃ | CH₂CF₃ | Cl |
| 12. | I.A.I | Cl | CH₂CH₃ | CH₂CF₃ | Cl |
| 13. | I.A.I | Cl | CH₃ | CH₂CHF₂ | Cl |
| 14. | I.A.I | Cl | CH₂CH₃ | CH₂CHF₂ | Cl |
| 15. | I.A.I | Cl | CH₃ | CH(CH₃)₂ | Cl |
| 16. | I.A.I | Cl | c-C₃H₅ | c-C₃H₅ | Cl |
| 17. | I.A.I | CH₃ | CH₃ | CH₂CH₃ | Br |
| 18. | I.A.I | CH₃ | CH₂CH₃ | CH₂CH₃ | Br |
| 19. | I.A.I | CH₃ | CH₃ | CH₂CF₃ | Br |
| 20. | I.A.I | CH₃ | CH₂CH₃ | CH₂CF₃ | Br |
| 21. | I.A.I | CH₃ | CH₃ | CH₂CHF₂ | Br |
| 22. | I.A.I | CH₃ | CH₂CH₃ | CH₂CHF₂ | Br |
| 23. | I.A.I | CH₃ | CH₃ | CH(CH₃)₂ | Br |
| 24. | I.A.I | CH₃ | c-C₃H₅ | c-C₃H₅ | Br |
| 25. | I.A.I | Cl | CH₃ | CH₂CH₃ | Br |
| 26. | I.A.I | Cl | CH₂CH₃ | CH₂CH₃ | Br |
| 27. | I.A.I | Cl | CH₃ | CH₂CF₃ | Br |
| 28. | I.A.I | Cl | CH₂CH₃ | CH₂CF₃ | Br |
| 29. | I.A.I | Cl | CH₃ | CH₂CHF₂ | Br |
| 30. | I.A.I | Cl | CH₂CH₃ | CH₂CHF₂ | Br |
| 31. | I.A.I | Cl | CH₃ | CH(CH₃)₂ | Br |
| 32. | I.A.I | Cl | c-C₃H₅ | c-C₃H₅ | Br |
| 33. | I.A.I | CH₃ | CH₃ | CH₂CH₃ | CF₃ |
| 34. | I.A.I | CH₃ | CH₂CH₃ | CH₂CH₃ | CF₃ |
| 35. | I.A.I | CH₃ | CH₃ | CH₂CF₃ | CF₃ |
| 36. | I.A.I | CH₃ | CH₂CH₃ | CH₂CF₃ | CF₃ |
| 37. | I.A.I | CH₃ | CH₃ | CH₂CHF₂ | CF₃ |
| 38. | I.A.I | CH₃ | CH₂CH₃ | CH₂CHF₂ | CF₃ |
| 39. | I.A.I | CH₃ | CH₃ | CH(CH₃)₂ | CF₃ |
| 40. | I.A.I | CH₃ | c-C₃H₅ | c-C₃H₅ | CF₃ |
| 41. | I.A.I | Cl | CH₃ | CH₂CH₃ | CF₃ |
| 42. | I.A.I | Cl | CH₂CH₃ | CH₂CH₃ | CF₃ |
| 43. | I.A.I | Cl | CH₃ | CH₂CF₃ | CF₃ |
| 44. | I.A.I | Cl | CH₂CH₃ | CH₂CF₃ | CF₃ |
| 45. | I.A.I | Cl | CH₃ | CH₂CHF₂ | CF₃ |
| 46. | I.A.I | Cl | CH₂CH₃ | CH₂CHF₂ | CF₃ |
| 47. | I.A.I | Cl | CH₃ | CH(CH₃)₂ | CF₃ |
| 48. | I.A.I | Cl | c-C₃H₅ | c-C₃H₅ | CF₃ |
| 49. | I.D.I | CH₃ | CH₃ | CH₂CH₃ | Cl |
| 50. | I.D.I | CH₃ | CH₂CH₃ | CH₂CH₃ | Cl |
| 51. | I.D.I | CH₃ | CH₃ | CH₂CF₃ | Cl |
| 52. | I.D.I | CH₃ | CH₂CH₃ | CH₂CF₃ | Cl |
| 53. | I.D.I | CH₃ | CH₃ | CH₂CHF₂ | Cl |
| 54. | I.D.I | CH₃ | CH₂CH₃ | CH₂CHF₂ | Cl |
| 55. | I.D.I | CH₃ | CH₃ | CH(CH₃)₂ | Cl |
| 56. | I.D.I | CH₃ | c-C₃H₅ | c-C₃H₅ | Cl |
| 57. | I.D.I | Cl | CH₃ | CH₂CH₃ | Cl |
| 58. | I.D.I | Cl | CH₂CH₃ | CH₂CH₃ | Cl |
| 59. | I.D.I | Cl | CH₃ | CH₂CF₃ | Cl |
| 60. | I.D.I | Cl | CH₂CH₃ | CH₂CF₃ | Cl |
| 61. | I.D.I | Cl | CH₃ | CH₂CHF₂ | Cl |
| 62. | I.D.I | Cl | CH₂CH₃ | CH₂CHF₂ | Cl |
| 63. | I.D.I | Cl | CH₃ | CH(CH₃)₂ | Cl |
| 64. | I.D.I | Cl | c-C₃H₅ | c-C₃H₅ | Cl |
| 65. | I.D.I | CH₃ | CH₃ | CH₂CH₃ | Br |
| 66. | I.D.I | CH₃ | CH₂CH₃ | CH₂CH₃ | Br |
| 67. | I.D.I | CH₃ | CH₃ | CH₂CF₃ | Br |
| 68. | I.D.I | CH₃ | CH₂CH₃ | CH₂CF₃ | Br |
| 69. | I.D.I | CH₃ | CH₃ | CH₂CHF₂ | Br |
| 70. | I.D.I | CH₃ | CH₂CH₃ | CH₂CHF₂ | Br |
| 71. | I.D.I | CH₃ | CH₃ | CH(CH₃)₂ | Br |
| 72. | I.D.I | CH₃ | c-C₃H₅ | c-C₃H₅ | Br |
| 73. | I.D.I | Cl | CH₃ | CH₂CH₃ | Br |
| 74. | I.D.I | Cl | CH₂CH₃ | CH₂CH₃ | Br |
| 75. | I.D.I | Cl | CH₃ | CH₂CF₃ | Br |
| 76. | I.D.I | Cl | CH₂CH₃ | CH₂CF₃ | Br |
| 77. | I.D.I | Cl | CH₃ | CH₂CHF₂ | Br |
| 78. | I.D.I | Cl | CH₂CH₃ | CH₂CHF₂ | Br |
| 79. | I.D.I | Cl | CH₃ | CH(CH₃)₂ | Br |
| 80. | I.D.I | Cl | c-C₃H₅ | c-C₃H₅ | Br |
| 81. | I.D.I | CH₃ | CH₃ | CH₂CH₃ | CF₃ |
| 82. | I.D.I | CH₃ | CH₂CH₃ | CH₂CH₃ | CF₃ |
| 83. | I.D.I | CH₃ | CH₃ | CH₂CF₃ | CF₃ |
| 84. | I.D.I | CH₃ | CH₂CH₃ | CH₂CF₃ | CF₃ |
| 85. | I.D.I | CH₃ | CH₃ | CH₂CHF₂ | CF₃ |
| 86. | I.D.I | CH₃ | CH₂CH₃ | CH₂CHF₂ | CF₃ |
| 87. | I.D.I | CH₃ | CH₃ | CH(CH₃)₂ | CF₃ |
| 88. | I.D.I | CH₃ | c-C₃H₅ | c-C₃H₅ | CF₃ |
| 89. | I.D.I | Cl | CH₃ | CH₂CH₃ | CF₃ |
| 90. | I.D.I | Cl | CH₂CH₃ | CH₂CH₃ | CF₃ |
| 91. | I.D.I | Cl | CH₃ | CH₂CF₃ | CF₃ |
| 92. | I.D.I | Cl | CH₂CH₃ | CH₂CF₃ | CF₃ |
| 93. | I.D.I | Cl | CH₃ | CH₂CHF₂ | CF₃ |
| 94. | I.D.I | Cl | CH₂CH₃ | CH₂CHF₂ | CF₃ |
| 95. | I.D.I | Cl | CH₃ | CH(CH₃)₂ | CF₃ |
| 96. | I.D.I | Cl | c-C₃H₅ | c-C₃H₅ | CF₃ |

where c-C₃H₅ is cyclopropyl

| | Formula | R¹ | NR^{2c}R^{2d} | R³ |
|---|---|---|---|---|
| 97. | I.A.I | CH₃ | pyrrolidin-1-yl | Cl |
| 98. | I.A.I | CH₃ | piperidin-1-yl | Cl |
| 99. | I.A.I | CH₃ | morpholin-4-yl | Cl |
| 100. | I.A.I | CH₃ | 2,6-dimethylmorpholin-4- | Cl |
| | | | yl | |
| 101. | I.A.I | Cl | pyrrolidin-1-yl | Cl |
| 102. | I.A.I | Cl | piperidin-1-yl | Cl |
| 103. | I.A.I | Cl | morpholin-4-yl | Cl |
| 104. | I.A.I | Cl | 2,6-dimethylmorpholin-4- | Cl |
| | | | yl | |
| 105. | I.A.I | CH₃ | pyrrolidin-1-yl | Br |
| 106. | I.A.I | CH₃ | piperidin-1-yl | Br |
| 107. | I.A.I | CH₃ | morpholin-4-yl | Br |
| 108. | I.A.I | CH₃ | 2,6-dimethylmorpholin-4- | Br |
| | | | yl | |
| 109. | I.A.I | Cl | pyrrolidin-1-yl | Br |
| 110. | I.A.I | Cl | piperidin-1-yl | Br |
| 111. | I.A.I | Cl | morpholin-4-yl | Br |
| 112. | I.A.I | Cl | 2,6-dimethylmorpholin-4- | Br |
| | | | yl | |
| 113. | I.A.I | CH₃ | pyrrolidin-1-yl | CF₃ |
| 114. | I.A.I | CH₃ | piperidin-1-yl | CF₃ |
| 115. | I.A.I | CH₃ | morpholin-4-yl | CF₃ |
| 116. | I.A.I | CH₃ | 2,6-dimethylmorpholin-4- | CF₃ |
| | | | yl | |
| 117. | I.A.I | Cl | pyrrolidin-1-yl | CF₃ |
| 118. | I.A.I | Cl | piperidin-1-yl | CF₃ |
| 119. | I.A.I | Cl | morpholin-4-yl | CF₃ |
| 120. | I.A.I | Cl | 2,6-dimethylmorpholin-4- | CF₃ |
| | | | yl | |
| 121. | I.D.I | CH₃ | pyrrolidin-1-yl | Cl |
| 122. | I.D.I | CH₃ | piperidin-1-yl | Cl |
| 123. | I.D.I | CH₃ | morpholin-4-yl | Cl |
| 124. | I.D.I | CH₃ | 2,6-dimethylmorpholin-4- | Cl |
| | | | yl | |
| 125. | I.D.I | Cl | pyrrolidin-1-yl | Cl |
| 126. | I.D.I | Cl | piperidin-1-yl | Cl |
| 127. | I.D.I | Cl | morpholin-4-yl | Cl |
| 128. | I.D.I | Cl | 2,6-dimethylmorpholin-4- | Cl |
| | | | yl | |
| 129. | I.D.I | CH₃ | pyrrolidin-1-yl | Br |
| 130. | I.D.I | CH₃ | piperidin-1-yl | Br |
| 131. | I.D.I | CH₃ | morpholin-4-yl | Br |
| 132. | I.D.I | CH₃ | 2,6-dimethylmorpholin-4- | Br |
| | | | yl | |
| 133. | I.D.I | Cl | pyrrolidin-1-yl | Br |
| 134. | I.D.I | Cl | piperidin-1-yl | Br |
| 135. | I.D.I | Cl | morpholin-4-yl | Br |
| 136. | I.D.I | Cl | 2,6-dimethylmorpholin-4- | Br |
| | | | yl | |
| 137. | I.D.I | CH₃ | pyrrolidin-1-yl | CF₃ |
| 138. | I.D.I | CH₃ | piperidin-1-yl | CF₃ |
| 139. | I.D.I | CH₃ | morpholin-4-yl | CF₃ |
| 140. | I.D.I | CH₃ | 2,6-dimethylmorpholin-4- | CF₃ |
| | | | yl | |
| 141. | I.D.I | Cl | pyrrolidin-1-yl | CF₃ |
| 142. | I.D.I | Cl | piperidin-1-yl | CF₃ |
| 143. | I.D.I | Cl | morpholin-4-yl | CF₃ |
| 144. | I.D.I | Cl | 2,6-dimethylmorpholin-4- | CF₃ |
| | | | yl | |

24. A composition comprising the compound as claimed in claim 1, or an agriculturally suitable salt thereof, and at least one auxiliary for formulating crop protection compounds.

25. A method for controlling unwanted vegetation comprising applying the compound of claim 1, or an agriculturally suitable salt thereof, or of the composition to plants, their seed and/or their habitat.

26. A method for controlling unwanted vegetation comprising allowing a herbicidally effective amount of the compound as claimed in claim 1, or an agriculturally suitable salt thereof, or of the composition thereof to act on plants, their seed and/or their habitat.


SEQUENCE LISTING (69 sequences)

SEQ ID NO: 1 - DNA, 1305 bp, Hordeum vulgare
atgccgcccacccccaccacccccgcggctaccggcgccgccgccgcggtgacgccggagcacgcgcgaccgcaccgaatggtccgcttcaacccgcgcagcgaccgcttccacacgctctccttccaccacgtcgagttctggtgcgcggacgccgcctccgccgccggccgcttcgcgttcgcgctcggcgcgccgctcgccgccaggtccgacctctccacggggaactccgcgcacgcctcccagctgctccgctcgggctccctcgccttcctcttcaccgcgccctacgccaacggctgcgacgccgccaccgcctccctgccctccttctccgccgacgccgcgcgccggttctccgccgaccacgggatcgcggtgcgctccgtagcgctgcgcgtcgcagacgccgccgaggccttccgcgccagcgtcgacgggggcgcgcgcccggccttcgcccccgtggacctcggccgcggcttcggcttcgcggaggtcgagctctacggcgacgtcgtgctccgcttcgtcagccacccggacggcacggacgtgcccttcttgccggggttcgagggcgtgaccaacccggacgccgtggactacggcctgacgcggttcgaccacgtcgtcggcaacgtcccggagcttgcccccgccgcagcctacatcgccgggttcacggggttccacgagttcgccgagttcacggcggaggacgtgggcacgaccgagagcgggctcaactcggtggtgctcgccaacaactcggagggcgtgctgctgccgctcaacgagccggtgcacggcaccaagcgccggagccagatacagacgttcctggaacaccacggcggcccgggcgtgcagcacatcgcggtggccagcagtgacgtgctcaggacgctcaggaagatgcgtgcgcgctccgccatgggcggcttcgacttcctgccacccccgctgccgaagtactacgaaggcgtgcgacgccttgccggggatgtcctctcggaggcgcagatcaaggaatgccaggagctgggtgtgctcgtcgatagggacgaccaaggggtgttgctccaaatcttcaccaagccagtaggggacaggccgaccttgttcctggagatgatccagaggatcgggtgcatggagaaggacgagagaggggaagagtaccagaagggtggctgcggcgggttcggcaaaggcaacttctccgagctgttcaagtccattgaagattacgagaagtcccttgaagccaagcaatctgctgcagttcagggatcatag

SEQ ID NO: 2 - protein, 434 aa, Hordeum vulgare
MPPTPTTPAATGAAAAVTPEHARPHRMVRFNPRSDRFHTLSFHHVEFWCADAASAAGRFAFALGAPLAARSDLSTGNSAHASQLLRSGSLAFLFTAPYANGCDAATASLPSFSADAARRFSADHGIAVRSVALRVADAAEAFRASVDGGARPAFAPVDLGRGFGFAEVELYGDVVLRFVSHPDGTDVPFLPGFEGVTNPDAVDYGLTRFDHVVGNVPELAPAAAYIAGFTGFHEFAEFTAEDVGTTESGLNSVVLANNSEGVLLPLNEPVHGTKRRSQIQTFLEHHGGPGVQHIAVASSDVLRTLRKMRARSAMGGFDFLPPPLPKYYEGVRRLAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 3 - DNA, 1470 bp, Fragilariopsis cylindrus
atgggtttctcttcagtatcgttatttccgctatcgtggtcggcgtcggcagtaacatcaacctatctcgagacgtccactagtacacaatcgaatgaagctgctactaccacttcgacaacggcgtgggaaccaaaattatcatcattagaagaaagaatagaaacggaaacgattgagtcgatcggtttccatcatatagaattttattgtggagatgctcgtagtatggcaaatcaattcgctgtctcattgggtatgtccgtcacgggtatcaccggccaatccacggggaatgatcaatgcatttcctatggattacaaagtggagagcagtttcgactattattaactgctccctattcacgagcgagagccactactcgcgatgacgacgacgacgacgacgacaacagtcctgatttggatgccgacgctccgatgccactccctaattataatgtagaagatgctcatactttcttccaaaatcatggcttagcagctcgagcggttggcatagaagtcatggatgccaaaaaagctttcgaggtatccgtggccaatggcgcaattccagtactggaaccaacctttcttcccaacggatgctacatctcagaagttgaattgtacggtgacgttgtgttgagatacgtgagtttcatcacatcgaatgaaaatcatacttataatgatgatgcatcacaaccatttctacctcatttagcaccaataattgatcaaagtaggaaagaggatgatgataataatgatgatggttttgggttatataaaatcgaccatgccgttgggaatgttcccaatttacaagaggtatactcacatatccaaaaatttacaggatttcatgaatttgctgaatttacatcagaagatgttggaactgtagactctggattaaattctgttgttttagccagtgacagtgaagcgattctgttacctataaatgaaccaactaatggacgacgaaaatcacaaattcaaacgtatctagaacagaacgagggccctggtctacaacatttagcagtcaaaacgaaagatatattttcaaccgtccgaaagatgcgaagaagtcaacaaggtatgtcgggatttgaattgatgaaacgaccgagtgaggaatattacaaagaacttcctgatcgacttggtgatcaattgacacccacgcagtatcaagaattagaggaacttggtatccttgcggattccgatgaggaaggaattttgatgcaaatttttaccaagcccgtcggtgatcgacctacattcttttttgaactaattcaacgaatcggttgcgtcattgagcatgacgatgacgacaggcaggagttatcagttgatcttgaacgaccaggatgtggtggttttggtaagggtaatttccgagaacttttcagatcaattgaagagcacgagaaaactttaaaggtatag

SEQ ID NO: 4 - DNA, 1467 bp, Fragilariopsis cylindrus
atgggttttagcagcgttagcctgtttccgctgagctggtcagcaagcgcagttaccagcacctatctggaaaccagcaccagcacccagagcaatgaagcagcaaccaccaccagtaccaccgcatgggaaccgaaactgagcagcctggaagaacgtattgaaaccgaaaccattgaaagcattggctttcatcacattgaattttattgcggtgatgcacgtagcatggcaaatcagtttgcagttagcctgggtatgagcgttaccggtattaccggtcagagcaccggtaatgatcagtgtattagctatggtctgcagagcggtgaacagtttcgtctgctgctgaccgcaccgtatagccgtgcacgtgcaaccacccgtgatgatgatgatgacgatgacgataatagtccggatctggatgcagatgcaccgatgccgctgccgaattataatgttgaagatgcccataccttttttcagaatcatggtctggcagcacgtgcagttggtattgaagttatggatgccaaaaaagcctttgaagttagcgttgcaaatggtgcaattccggttctggaaccgacctttctgccgaatggttgttatatttctgaagtggaactgtatggtgatgttgttctgcgttatgtgagctttattaccagcaatgaaaaccacacctacaatgatgatgccagccagccgtttctgccgcatctggcaccgattattgatcagagccgtaaagaagatgatgataataatgatgatggctttggcctgtataaaattgatcatgccgttggtaatgtgccgaatctgcaagaagtttatagccatattcagaaatttaccggctttcatgaatttgccgaatttaccagcgaagatgttggcaccgttgatagcggtctgaatagcgttgttctggcaagcgatagcgaagcaattctgctgccgattaatgaaccgaccaatggtcgtcgtaaaagccagattcagacatatctggaacagaatgaaggtccgggtctgcagcatctggccgttaaaaccaaagatatttttagcaccgtgcgtaaaatgcgtcgtagccagcagggtatgagcggttttgaactgatgaaacgtccgagcgaagaatattataaagaactgccggatcgtctgggtgatcagctgaccccgacccagtatcaagaattagaagaactgggtattctggcagatagtgatgaagaaggtattctgatgcagatttttaccaaaccggttggtgatcgtccgacctttttttttgaactgattcagcgtattggctgcgtgattgaacatgatgatgatgatcgtcaagaactgagcgttgatctggaacgtccgggttgtggtggttttggtaaaggtaattttcgtgaactgtttcgcagcattgaagaacatgaaaaaaccctgaaagtg

SEQ ID NO: 5 - protein, 489 aa, Fragilariopsis cylindrus
MGFSSVSLFPLSWSASAVTSTYLETSTSTQSNEAATTTSTTAWEPKLSSLEERIETETIESIGFHHIEFYCGDARSMANQFAVSLGMSVTGITGQSTGNDQCISYGLQSGEQFRLLLTAPYSRARATTRDDDDDDDDNSPDLDADAPMPLPNYNVEDAHTFFQNHGLAARAVGIEVMDAKKAFEVSVANGAIPVLEPTFLPNGCYISEVELYGDVVLRYVSFITSNENHTYNDDASQPFLPHLAPIIDQSRKEDDDNNDDGFGLYKIDHAVGNVPNLQEVYSHIQKFTGFHEFAEFTSEDVGTVDSGLNSVVLASDSEAILLPINEPTNGRRKSQIQTYLEQNEGPGLQHLAVKTKDIFSTVRKMRRSQQGMSGFELMKRPSEEYYKELPDRLGDQLTPTQYQELEELGILADSDEEGILMQIFTKPVGDRPTFFFELIQRIGCVIEHDDDDRQELSVDLERPGCGGFGKGNFRELFRSIEEHEKTLKV

SEQ ID NO: 6 - DNA, 1341 bp, Chlorella sp.
atggtggtcgaggctgcggccgcctccaacggcaatggcgcaggggaggaggtgttcagcaagaagctcgtgggatatgacggtttccagcgccacaacccacgctccgaccgcttccccatgcacaagttccaccacgtcgagttctggtgcggcgatgccaccaccaccagctgcaggcaagcgcgccccccagaagcacccatgtttgggtatgggctgggcctgactctggtggccaagagcgaccagtccacgggcaaccaccactacgcgtcgtacgtcatgcagtcgggcgatcttgtgatggcctttaccgcgccctacagcacccagacagacaagagcggcagcagcccgcccgcagcgtacgaccaggacgccgcctacgccttcctcaagaagcacggcatggcggtgcgcgcctttggaatcctggtggacgatgccgcggaggcgtaccgcatagccactgcccacggcggggtgggtgtggcgccacccaccacccgcacggacgcagccagcggcaccagcctgacgtggagcgaggtgcagctgtacggcgactgcgtgctgcgctttgtcagcggcgactacgagggcgccttcatccccggctaccagcccgtggaagacgcgccccaagtctcctacggcctgcagcgcctggaccatgcggtgggcaacgtgccagagctgatccctcaagtggagtacatggctcgcagcctgggctggcacgagtttgctgagttcactgccgaggatgtgggcactgtggactcgggcctcaactccatggtcatggccaacaacaacgagatgattctgctgccggtcaacgagcccacccacggcaccaagcgcaagagccagatccagaccttcctggagcagaatgaggggcccgggctgcagcacatggccctgaaaacagacgacatcgtagccaccatgcgacagctccgggccaggtctgcgtttggcggcttcgacttcatgcccaggccttcgcctgactactaccgcaagctgcctgcccgcatcggcagcctgctgacggcgcagcagtacaaggacgttgaggagctggggctgcttgtggacaaggatgaccagggcgtgctgctccagatcttcaccaagccgctgggcgaccgacccaccgtgtttttcgaaatcatccagcgcctgtgcgccctggagccgcaggcgcccaagagccagcgcggcgcggtgccttccgaggtcggcggctgcggcggctttggcaagggcaacttcagtgagctgttcaagagcatcgaggtgtacgagacggatctgggcatcaactaa

SEQ ID NO: 7 - DNA, 1341 bp, Chlorella sp
atggttgttgaagcagcagcagcaagcaatggtaatggtgccggtgaagaagtgtttagcaaaaaactggtgggctatgatggttttcagcgtcataatccgcgtagcgatcgttttccgatgcataaatttcatcatgtggaattttggtgtggtgatgcaaccaccaccagttgtcgtcaggcacgtcctccggaagcaccgatgtttggttatggtctgggtctgaccctggttgcaaaaagcgatcagagcaccggtaatcatcattatgcaagctatgttatgcagagcggtgatctggttatggcatttaccgcaccgtatagcacccagaccgataaaagcggtagcagtccgcctgcagcatacgatcaggatgcagcatacgcctttctgaaaaaacatggtatggcagttcgtgcatttggtattctggttgatgatgcagcagaagcatatcgtattgcaaccgcacatggtggtgttggtgttgcacctccgaccacccgtaccgatgcagcaagcggcaccagcctgacctggtctgaagttcagctgtatggtgattgtgttctgcgttttgttagcggtgattatgaaggtgcatttattccgggttatcagccggttgaagatgcaccgcaggttagctatggtctgcagcgtctggatcatgcagttggtaatgttccggaactgattccgcaggttgaatatatggcacgtagcctgggttggcatgaatttgcagaatttaccgcagaagatgttggcaccgttgatagcggtctgaatagcatggttatggccaataacaatgaaatgattctgctgccggtgaatgaaccgacccacggcaccaaacgtaaaagccagattcagacctttctggaacagaatgaaggtccgggtctgcagcacatggcactgaaaaccgatgatattgttgcaaccatgcgtcagctgcgtgcacgtagcgcatttggtggttttgattttatgcctcgtccgagtccggattattatcgtaaactgcctgcacgtattggtagcctgctgaccgcacagcagtataaagatgttgaagaactgggtctgctggttgataaagatgatcagggtgttctgctgcagatttttaccaaaccgctgggtgatcgtccgaccgttttttttgaaattattcagcgtctgtgtgcactggaaccgcaggcaccgaaaagccagcgtggtgcagttccgagcgaagttggtggttgtggtggttttggtaaaggtaattttagcgaactgtttaaaagcattgaagtgtatgaaaccgatctgggcatcaattaa

SEQ ID NO: 8 - protein, 446 aa, Chlorella sp
MVVEAAAASNGNGAGEEVFSKKLVGYDGFQRHNPRSDRFPMHKFHHVEFWCGDATTTSCRQARPPEAPMFGYGLGLTLVAKSDQSTGNHHYASYVMQSGDLVMAFTAPYSTQTDKSGSSPPAAYDQDAAYAFLKKHGMAVRAFGILVDDAAEAYRIATAHGGVGVAPPTTRTDAASGTSLTWSEVQLYGDCVLRFVSGDYEGAFIPGYQPVEDAPQVSYGLQRLDHAVGNVPELIPQVEYMARSLGWHEFAEFTAEDVGTVDSGLNSMVMANNNEMILLPVNEPTHGTKRKSQIQTFLEQNEGPGLQHMALKTDDIVATMRQLRARSAFGGFDFMPRPSPDYYRKLPARIGSLLTAQQYKDVEELGLLVDKDDQGVLLQIFTKPLGDRPTVFFEIIQRLCALEPQAPKSQRGAVPSEVGGCGGFGKGNFSELFKSIEVYETDLGIN

SEQ ID NO: 9 - DNA, 1257 bp, Thalassiosira
tttcatcatattgaattcttcgccagtgatgcgcttacgacagccaagcggtttgagctagcgttgggattgccaattacgtgttggagttcattggctacggggaacgatgtttgtgttacctacggattggaggggatgcaaactcgaaagattgaaaccgacaacgcaaacaagaacggaggggcaggatccaatgtacaaatgactgcacctcttcctcttccggggtatgatattgacaaagctcatgagttttactcgaagcacggtttggcagtacgagctgtgggagtggaagtgaaggatgcaactgtagcttatgcaaatgctgttgagaatggtgctacaggagtattggagcctacgattgttgaaaactttaacagcgatggagattcgcagaagtgtcatatggctgaggtggaattgtatggtgatgtggtgttgagattagtcagttttcatggagattgtagtgccgaacaatctacattccttcctcacttgtcgccgtatccatccaacagcaacaagaacaaaccaacttacggacttgctcgtctagatcacacggtgggcaatgttcccaacctcctcgcgacgcaacgatacattcaaacattcaccaactaccatcccttcgcagagttcactccggaagatgtgggaacagtcgactctggtcttaatagtgtagtacttgcatcagacaacgagaatgttttgctgcctctcaatgaacctaccgaaggtaaacgaaagagtcagattcaaacatatctagagcagaacgagggaccgggactgcagcatattgccatcaagacgaatgatatctttgataccattgcaaagatgagacacgcagaagagaactttggaggtttcgagttgatgaaacgtccatcggatgagtattacaaagagttgccttcgagattgggtgataagttgactgtcgagcaatacaaacagctggaagagttgggaatactggcagatgcagatgacgaaggtatattgcttcaaatattcacaaagccattgggagacaggcccaccttgttccttgagattattcagcgaattgggtgtgtgttaccggatgacgatgaagcaactgatgaaggggaagctaagaatgcacacaatagaatagtcagagaacgtcccggatgtggtggatttggtcagggcaacttccgtgaactcttcaaagcaattgaagaacatgagaagacactcaaggtctaa

SEQ ID NO: 10 - DNA, 1257 bp, Thalassiosira
tttcatcatatcgaattttttgcctcagatgcactgaccaccgcaaaacgctttgaactggcactgggtctgccgattacctgttggagcagcctggcaaccggtaatgatgtttgtgttacctatggtctggaaggtatgcagacccgtaaaattgaaaccgataacgccaataaaaacggtggtgcaggtagcaatgttcagatgaccgcaccgctgccgctgcctggttatgatattgataaagcccatgaattctatagcaaacatggtctggcagttcgtgcagttggtgttgaagttaaagatgcaaccgttgcctatgcaaatgcagttgaaaatggtgcaaccggtgttctggaaccgaccattgttgaaaactttaatagtgatggcgatagccagaaatgtcacatggccgaagttgaactgtatggtgatgttgttctgcgtctggttagctttcatggtgattgtagcgcagaacagagcacctttctgccgcatctgagcccgtatccgagcaatagcaataaaaacaaaccgacctatggcctggcacgtctggatcataccgttggtaatgttccgaatctgctggcaacccagcgttatattcagacctttaccaactatcatccgtttgcagaatttacaccggaagatgttggcaccgttgatagcggtctgaatagcgttgttctggcaagcgataatgaaaatgttctgctgccgctgaatgaaccgaccgaaggtaaacgtaaaagccagattcagacctatctggaacagaatgaaggtccgggtctgcagcatattgcaattaaaaccaatgatatttttgataccattgccaaaatgcgccatgccgaagaaaattttggtggttttgaactgatgaaacgtccgtccgatgaatactataaagaactgccgagccgtctgggtgataaactgaccgttgaacagtataaacagctggaagaactgggtattctggcagatgcagatgatgaaggtattctgctgcagatttttaccaaaccgctgggtgatcgtccgaccctgtttctggaaattattcagcgtattggttgtgttctgccggatgatgatgaagcaaccgatgaaggtgaagcaaaaaatgcccataatcgtattgttcgtgaacgtccgggttgtggtggttttggtcagggtaattttcgcgaactgtttaaagccattgaagaacatgaaaaaaccctgaaagtttaa

SEQ ID NO: 11 - protein, 418 aa, Thalassiosira pseudonana
FHHIEFFASDALTTAKRFELALGLPITCWSSLATGNDVCVTYGLEGMQTRKIETDNANKNGGAGSNVQMTAPLPLPGYDIDKAHEFYSKHGLAVRAVGVEVKDATVAYANAVENGATGVLEPTIVENFNSDGDSQKCHMAEVELYGDVVLRLVSFHGDCSAEQSTFLPHLSPYPSNSNKNKPTYGLARLDHTVGNVPNLLATQRYIQTFTNYHPFAEFTPEDVGTVDSGLNSVVLASDNENVLLPLNEPTEGKRKSQIQTYLEQNEGPGLQHIAIKTNDIFDTIAKMRHAEENFGGFELMKRPSDEYYKELPSRLGDKLTVEQYKQLEELGILADADDEGILLQIFTKPLGDRPTLFLEIIQRIGCVLPDDDEATDEGEAKNAHNRIVRERPGCGGFGQGNFRELFKAIEEHEKTLKV

SEQ ID NO: 12 - DNA, 1092 bp, Cyanothece
atggaaatcgatcatattcatttctacgttgaagatgcagcacatcaacgagattggtttattgataaaatggggtttcaatccatcagcaacagtatccatgatgacacttatagcgaagtagtagggaatcagtctgtttactttatcttatcttctcccctcaacgatgctagtccagtttcttattacttgaaatctcatcctccgggggttgctgatgttgcttttcgtgttgacaatcttaattttttattagacaaagtatcccgttttaaggtcgaaattattaatcaatctagtctaacagcttttcctctaaataaaccagtgaaattcgcgaaacttaaaggatggggttctgtcaatcataccttaattgatcaggcaagtcctaggacttttattagctcaaaaatgattgctaaaagcgatattattgggattgatcatgttgttttaaatgttcctcaaggtgaactccccttagccataaattggtacaaaaatgtatttgattttataagtcatcaacagttcaacatccaaacagaacattcggggttatctagtgaagccttagttgatagttcaggaaaagtacaatttaatattaatcaaccaagttctactaattctcagattcaggaatttttagaccataataacggttcaggcattcaacatattggtttaaaatcaagtaatattttacaaagtgttgcacaaatgcgtcaaaggggattaccctttttatccgttcctaattcctattaccaaaacctaaaagaattgattagaaaatcgacaatttcttgtttaagccaacaggaactagaacaaattgaaactgaacaaattctagtttgttggccagaagataacccgacttcaatcctgatgcaaattttcactcaacccatttttaagcagccgactttcttttttgaattaattcaaagacgcaaccaagcacagggatttggccaaggtaattttcaagcgttatttgaagccatagaatcagaacaaatcaagagaaatagggtatcctcacgagtcactttacaggctgtaacaccccaatcttga

SEQ ID NO: 13 - DNA, 1089 bp, Cyanothece
atggaaattgatcatattcatttttatgtggaagatgcagcccatcagcgtgattggtttattgataaaatgggctttcagagcattagcaatagcattcatgatgatacctatagcgaagttgtgggtaatcagagcgtgtatttcattctgagcagtccgctgaatgatgcaagtccggttagctattatctgaaaagccatcctccgggtgttgcagatgttgcatttcgtgttgataatctgaattttctgctggataaagtgagccgctttaaagtggaaatcattaatcagagcagcctgaccgcatttccgctgaataaaccggttaaatttgccaaactgaaaggttggggtagcgttaatcataccctgattgatcaggcaagtccgcgtacctttattagcagcaaaatgattgccaaaagcgatattattggcattgatcatgtggttctgaatgttccgcagggtgaactgccgctggcaattaattggtacaaaaatgtgtttgattttattagccatcagcagtttaatattcagaccgaacatagcggtctgagcagcgaagcactggttgatagcagcggtaaagttcagtttaatattaatcagccgagcagcaccaatagccagattcaagaatttctggatcataataatggcagcggcattcagcatattggtctgaaaagcagcaatattctgcagagcgttgcacagatgcgtcagcgtggtctgccgtttctgagcgttccgaatagctattatcagaatctgaaagaactgattcgcaaaagcaccattagctgtctgagccagcaagaactggaacaaattgaaaccgaacaaattctggtttgttggcctgaagataatccgaccagcattctgatgcagatttttacccagccgatttttaaacagccgacctttttttttgaactgattcagcgtcgtaatcaggcacagggttttggtcagggtaattttcaggcactgtttgaagcaattgaaagtgaacaaattaaacgtaatcgtgttagcagccgtgttaccctgcaggcagttacaccgcagagc

SEQ ID NO: 14 - protein, 363 aa, Cyanothece
MEIDHIHFYVEDAAHQRDWFIDKMGFQSISNSIHDDTYSEVVGNQSVYFILSSPLNDASPVSYYLKSHPPGVADVAFRVDNLNFLLDKVSRFKVEIINQSSLTAFPLNKPVKFAKLKGWGSVNHTLIDQASPRTFISSKMIAKSDIIGIDHVVLNVPQGELPLAINWYKNVFDFISHQQFNIQTEHSGLSSEALVDSSGKVQFNINQPSSTNSQIQEFLDHNNGSGIQHIGLKSSNILQSVAQMRQRGLPFLSVPNSYYQNLKELIRKSTISCLSQQELEQIETEQILVCWPEDNPTSILMQIFTQPIFKQPTFFFELIQRRNQAQGFGQGNFQALFEAIESEQIKRNRVSSRVTLQAVTPQS

SEQ ID NO: 15 - DNA, 1068 bp, Acaryochloris marina
atggattttgatcatattcatttttatgttcatgattccaagcaatgccagcgttggtttactaacgttttaggatttcaatatcttgggagcaatactacggctgatcggcagattgaagttgtctcttctggggcgattgtctgtatattttccagccctctaaaccgaaccagcccagttgcccagtatctacaacaacaccctcctggtgtcgttgatttggctttcttggtcccggacgttcaggctacgcttacctgcgctgtccagtcaggagcaacccttttacaacctttgaccgaagaaaaaaacgaccaaggaacgttaacttggggaaaagtacgagggtggggagcgttagaacataccttggtagagcgaagagggcaaacctccactctgccatccagcatttttcccatctcaattcatgggcatgatgcccatcagagtctatttacccagattgatcatggggttttgaatgtgggtaagcatcagctgcaagctgctgtgagttggtatcagcgcatatttggatttaaaactcaccgatattttgatattcaaacgcgtcgttcaggtctgcgcagtgaagtgttgacccatccccaaggccaaatcaagtttccgatcaatgaacccacctcagcgaattcccaaatccaagaatttctagaggtcaatcggggggcaggtattcaacatatcgcattgggaacttctaatattgttgaaacggttactcagcttaagcatcgagggctatccatcctagatattccacccagctactatcaacgcttacgacaccagtttgagcaagtctattcccacctcgattggcatgccctggaaacacaacatattctggctgattttgaggaagatgctggagccggaattctattgcaaaccttcacaaagcctatctttccacaacccacttttttctttgaaattattgagcgccaacggcaggcccaagggttcggacaacgaaactttttggccctttttcaagccatggagcgggaacaacagaaacggggagtattgctatag

SEQ ID NO: 16 - DNA, 1065 bp, Acaryochloris marina
atggattttgatcacatccacttttatgtgcatgacagcaaacagtgtcagcgttggtttaccaatgttctgggttttcagtatctgggtagcaataccaccgcagatcgtcagattgaagttgttagcagcggtgcaattgtttgcatttttagcagtccgctgaatcgtaccagtccggttgcacagtatctgcagcagcatccgcctggtgttgttgatctggcatttctggttccggatgttcaggcaaccctgacctgtgcagttcagagcggtgccaccctgctgcagccgctgaccgaagaaaaaaatgatcagggtacactgacctggggtaaagttcgtggttggggtgcactggaacacaccctggttgaacgtcgtggtcagaccagcaccctgccgagcagcatttttccgattagcattcatggtcatgatgcacatcagagcctgtttacccagattgatcatggtgttctgaatgttggtaaacatcagctgcaggcagcagttagctggtatcagcgtatttttggctttaaaacccaccgctattttgatattcagacccgtcgtagcggtctgcgtagcgaagttctgacccatccgcagggtcagatcaaatttccgatcaatgaaccgaccagcgcaaatagccagattcaagaatttctggaagttaatcgtggtgccggtattcagcatattgcactgggcaccagcaatattgttgaaaccgttacccagctgaaacatcgtggtctgagcattctggatattccgcctagctattatcagcgtctgcgtcatcagtttgaacaggtttatagccatctggattggcatgccctggaaacccagcatattctggcagattttgaagaagatgcaggcgcaggtattctgctgcaaacctttaccaaaccgatttttccgcagccgaccttttttttcgaaattattgaacgtcagcgtcaggcacagggttttggtcagcgcaattttctggcactgtttcaggcaatggaacgtgaacagcagaaacgtggtgtgctgctg

SEQ ID NO: 17 - protein, 355 aa, Acaryochloris marina
MDFDHIHFYVHDSKQCQRWFTNVLGFQYLGSNTTADRQIEVVSSGAIVCIFSSPLNRTSPVAQYLQQHPPGVVDLAFLVPDVQATLTCAVQSGATLLQPLTEEKNDQGTLTWGKVRGWGALEHTLVERRGQTSTLPSSIFPISIHGHDAHQSLFTQIDHGVLNVGKHQLQAAVSWYQRIFGFKTHRYFDIQTRRSGLRSEVLTHPQGQIKFPINEPTSANSQIQEFLEVNRGAGIQHIALGTSNIVETVTQLKHRGLSILDIPPSYYQRLRHQFEQVYSHLDWHALETQHILADFEEDAGAGILLQTFTKPIFPQPTFFFEIIERQRQAQGFGQRNFLALFQAMEREQQKRGVLL

SEQ ID NO: 18 - DNA, 1020 bp, Synechocystis sp. PCC 6803
atggaattcgactatcttcatttatacgttgacgattatcagtcagctcatcgttgttatcaacgtcaatggggtttcacttgcgtaaataaaattattactgaccaaggaattactggcatctaccaacaggggcaaatacttctgctaatttcggcatcggaatctagtttgagtagatatgccgactatctccagaaacatccccccggcgtaggtgaagtcgcttggcaggtggccaattggcaaaaaattcagcatcaattatcagaattacagatagaaaccacaccagttattcatcctctgactaaagcagaaggattaacttttttgctctggggagatgtgcaccatagcatttatcctgttcgttctgagctaaatcagaataaaacattgcatggtgttggtttaacgaccatcgaccatgtggtgctaaacattgccgccgatcaatttacccaggcttcccaatggtatcaacaggtgtttggctggtcggtgcagcagagttttactgtcaatacgccccattctggtctgtatagcgaagccctggccagtgccaatgggaaagtccaatttaacctcaattgtcccaccaataacagttcccaaattcaaacttttttagccaataaccatggggctggtattcaacatgtcgctttttccactacgagtattacgcgaactgtggctcatctgcgggaaaggggcgtaaattttttaaaaatccccactggctattatcaacagcaaagaaacagtagctattttaattatgcaagtttggattgggataccttacagtgcctagaaattttgctggatgatcaagataatacgggggagcgattactgctacaaatttttagtcagccttgctatggagtaggcactctattttgggaaattattgaacgccgccaccgggcaaaaggatttggtcaaggaaactttcaagctctctatgaagcggtggagactttagaaaaacagttagaagtgccataa

SEQ ID NO: 19 - DNA, 1020 bp, Synechocystis sp. PCC 6803
atggaattcgattacctgcatctgtatgttgatgactatcagagcgctcaccgttgttaccagcgtcagtggggctttacttgtgtgaacaaaatcatcaccgaccagggtatcactggtatttaccagcagggtcagatcctgctgctgatcagcgcttctgaatcttccctctctcgctatgccgattacctccagaaacatccgccaggtgtaggtgaagtcgcctggcaggtcgcaaactggcagaaaattcagcaccagctgtccgaactgcagattgaaactaccccggtgattcacccactgaccaaagcagaaggcctgactttcctgctgtggggtgacgttcaccactccatctacccagtacgtagcgagctgaaccagaacaaaaccctgcatggcgttggtctgaccactatcgatcacgtggttctgaacatcgcagcggaccagttcacccaggcgagccagtggtatcagcaggtattcggttggtccgttcagcagtctttcacggttaacaccccgcattccggtctgtactctgaagctctggcgtctgcgaacggcaaagttcagttcaacctgaactgcccgaccaacaacagctcccagattcagaccttcctggcgaacaaccacggtgctggtatccagcacgttgcattctctactacctctatcacccgtacggtcgctcacctgcgtgaacgtggcgtgaacttcctgaaaatcccgaccggttactatcagcagcagcgcaacagctcctacttcaactacgcgtctctggattgggataccctgcagtgcctggagattctgctggacgaccaggacaacactggcgaacgcctgctgctgcagatcttttctcagccgtgctatggcgtgggtacgctgttttgggaaattatcgagcgccgtcaccgtgctaaaggctttggccagggcaactttcaggcactgtacgaggcagtagaaaccctggaaaaacagctcgaagtgccataa

SEQ ID NO: 20 - protein, 339 aa, Synechocystis sp. PCC 6803
MEFDYLHLYVDDYQSAHRCYQRQWGFTCVNKIITDQGITGIYQQGQILLLISASESSLSRYADYLQKHPPGVGEVAWQVANWQKIQHQLSELQIETTPVIHPLTKAEGLTFLLWGDVHHSIYPVRSELNQNKTLHGVGLTTIDHVVLNIAADQFTQASQWYQQVFGWSVQQSFTVNTPHSGLYSEALASANGKVQFNLNCPTNNSSQIQTFLANNHGAGIQHVAFSTTSITRTVAHLRERGVNFLKIPTGYYQQQRNSSYFNYASLDWDTLQCLEILLDDQDNTGERLLLQIFSQPCYGVGTLFWEIIERRHRAKGFGQGNFQALYEAVETLEKQLEVP

SEQ ID NO: 21 - DNA, 1326 bp, Alopecurus myosuroides
atgcctccgaccaccgcaaccgcaaccggtgctgcagcagcagccgttacaccggaacatgcagcacgtcgttttccgcgtgttgttcgtgttaatccgcgtagcgatcgttttccggttctggcatttcatcatgttgaattttggtgtgccgatgcagcaagcgcagcaggtcgttttagctttgcactgggtgcaccgctggcagcacgtagcgatctgagcaccggtaatagcagccatgcaagccatctgctgcgtagtggtgcactggcatttctgtttaccgcaccgtatgcaccgcctccgcaggatgcagcagatgcagccgctaccgccagcattccgagctttagcaccgaagcagcacgtacctttagcagcgcacatggtctggcagttcgtagcgttgcaattcgtgttgcagatgccgcagaagcatttcataccagcgttgcgggtggtgcacgtccggcatttgcaccggcagatctgggtagcggttttggtctggccgaagttgaactgtatggtgatgttgttctgcgttttgttagtcatccggatggtgatgatgttccgtttctgccgggttttgaaggtgttagccgtccgggtgcaatggattatggtctgacccgttttgatcatgttgttggtaatgttccggaaatggcaccggttgcagcatatatgaaaggttttaccggctttcatgaatttgccgaatttaccgcagaagatgttggcaccgcagaaagcggtctgaatagcgttgttctggcaaataatagcgaagcagttctgctgccgctgaatgaaccggtgcatggcaccaaacgtcgtagccagattcagacctatctggattatcatggtggtccgggtgttcagcatattgcactggcaagcagtgatgttctgcgtaccctgcgtgaaatgcgtgcacgtagcgcaatgggtggttttgaatttatggcaccgccgcaggcaaaatattatgaaggtgttcgtcgtctggctggtgatgttctgagcgaagcacagattaaagaatgtcaggaactgggcgttctggttgatcgtgatgatcagggtgttctgctgcagatttttaccaaaccggttggtgatcgtcgtccgaccttttttctggaaatgattcagcgtattggctgcatggaaaaagatgaaattggccaggaatatcagaaaggcggctgtggtggttttggtaaaggtaattttagcgaactgtttaaaagcattgaagattatgaaaaaagcctggaagccaaacagagcgcagttgcacagcagagctaa

SEQ ID NO: 22 - protein, 441 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSTEAARTFSSAHGLAVRSVAIRVADAAEAFHTSVAGGARPAFAPADLGSGFGLAEVELYGDVVLRFVSHPDGDDVPFLPGFEGVSRPGAMDYGLTRFDHVVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMAPPQAKYYEGVRRLAGDVLSEAQIKECQELGVLVDRDDQGVLLQIFTKPVGDRRPTFFLEMIQRIGCMEKDEIGQEYQKGGCGGFGKGNFSELFKSIEDYEKSLEAKQSAVAQQS

SEQ ID NO: 23 - DNA, 1323 bp, Alopecurus myosuroides
atgccaccaactactgctactgctacaggtgctgctgctgcagctgttactccagaacatgctgctagaaggttcccaagagttgttagagttaacccaaggtctgataggttcccagttcttgctttccatcatgttgagttttggtgtgctgatgctgcttctgctgctggaagattttcttttgctcttggtgctccacttgctgctagatctgatttgtctactggaaactcttctcacgcttctcaccttttgagatctggtgctcttgctttccttttcactgctccttatgctccaccaccacaagatgctgcagatgcagcagctactgcttctattccatctttttcaactgaggctgctaggactttctcttctgctcatggattggctgttagatctgtggctattagagttgcagatgctgcagaggctttccatacttctgttgctggtggtgctagaccagcttttgctccagctgatcttggatctggatttggacttgctgaggttgagctttacggtgatgttgttcttagattcgtgtctcacccagatggtgatgatgttccatttcttccaggattcgagggtgttagtagaccaggtgctatggattatggactcactaggttcgatcacgttgtgggaaatgttccagaaatggctccagttgctgcttacatgaagggattcactggatttcatgagttcgctgagttcactgctgaggatgttggaactgctgagtctggacttaactctgttgtgcttgctaacaactctgaggctgttcttttgccacttaatgagccagttcacggcactaagagaagatctcagattcagacttacctcgattaccatggtggaccaggtgttcaacatattgctcttgcttcatctgatgtgcttaggactcttagagagatgagagctagatctgctatgggaggatttgagtttatggctccaccacaagctaagtattacgaaggtgttagaaggcttgctggtgatgttctttctgaggctcaaatcaaagagtgccaagagcttggagttcttgtggatagagatgatcagggtgtgcttctccagattttcactaagccagttggagataggccaacattcttcttggagatgattcagaggatcggctgcatggaaaaggatgagattggacaagagtaccaaaagggcggatgtggtggatttggaaagggaaatttctccgagcttttcaagtccatcgaggattacgagaagtctcttgaggctaagcaatctgctgttgctcaacagtcttga

SEQ ID NO: 24 - protein, 440 aa, Alopecurus myosuroides
MPPTTATATGAAAAAVTPEHAARRFPRVVRVNPRSDRFPVLAFHHVEFWCADAASAAGRFSFALGAPLAARSDLSTGNSSHASHLLRSGALAFLFTAPYAPPPQDAADAAATASIPSFSTEAARTFSSAHGLAVRSVAIRVADAAEAFHTSVAGGARPAFAPADLGSGFGLAEVELYGDVVLRFVSHPDGDDVPFLPGFEGVSRPGAMDYGLTRFDHVVGNVPEMAPVAAYMKGFTGFHEFAEFTAEDVGTAESGLNSVVLANNSEAVLLPLNEPVHGTKRRSQIQTYLDYHGGPGVQHIALASSDVLRTLREMRARSAMGGFEFMAPPQAKYYEGVRRLAGDVLSEAQIKECQELGVLVDRDDQGVLLQIFTKPVGDRPTFFLEMIQRIGCMEKDEIGQEYQKGGCGGFGKGNFSELFKSIEDYEKSLEAKQSAVAQQS

SEQ ID NO: 25 - DNA, 1323 bp, Sorghum halapense
atgcctccgaccccgaccaccgcagcagcaacaggtgccgcagttgcagcagcaagcgcagaacaggcagcatttcgtctggttggtcatcgtaattttgttcgtgttaatccgcgtagcgatcgttttcataccctggcatttcatcatgttgaactgtggtgtgccgatgcagccagcgcagcaggtcgttttagctttggtctgggtgcaccgctggcagcacgtagcgatctgagcaccggtaataccgcacatgcaagcctgctgctgcgttcaggtgcactggcatttctgtttaccgcaccgtatgcccatggtgctgatgcagcaaccgcaagcctgccgagctttagcgcagcagaagcacgtcgttttgcagcagatcatggtctggcagttcgtgccgttgcactgcgtgttgcagatgccgaagatgcatttcgtgcaagcgttgcagccggtgcacgtccggcatttgaaccggttgaactgggtctgggttttcgtctggccgaagttgaactgtatggtgatgttgttctgcgttatgttagctatccggatgatgcagatgcaagctttctgccgggttttgttggtgttagcagtccgggtgcggcagattatggcctgcgtcgttttgatcatattgtgggtaatgttccggaactggcaccggcagcggcatattttgcaggttttaccggctttcatgaatttgcagaatttaccgcagaagatgttggcaccaccgaaagcggtctgaatagcatggttctggcaaataatgccgaaaatgttctgctgccgctgaatgaaccggtgcatggcaccaaacgtcgtagccagattcagacctttctggatcatcatggtggtccgggtgttcagcacatggcactggcaagtgatgatgtgctgcgtaccctgcgtgaaatgcaggcatgtagtgcaatgggtggttttgaatttatggcaccgccggcaccggaatattatgatggtgttcgtcgtcgtgccggtgatgttctgaccgaagcacagattaaagaatgtcaggaactgggcgttctggttgatcgtgatgatcagggtgttctgctgcagatttttaccaaaccggttggtgatcgcccgaccttttttctggaaattattcagcgtattggttgcatggaaaaagatgaaaaaggccaggaatatcagaaaggcggttgtggtggttttggtaaaggtaattttagccagctgtttaaaagcattgaagattatgaaaaaagcctggaagcaaaacaggcagctgcagcacagggtccgtaa

SEQ ID NO: 26 - protein, 440 aa, Sorghum halapense
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPDDADASFLPGFVGVSSPGAADYGLRRFDHIVGNVPELAPAAAYFAGFTGFHEFAEFTAEDVGTTESGLNSMVLANNAENVLLPLNEPVHGTKRRSQIQTFLDHHGGPGVQHMALASDDVLRTLREMQACSAMGGFEFMAPPAPEYYDGVRRRAGDVLTEAQIKECQELGVLVDRDDQGVLLQIFTKPVGDRPTFFLEIIQRIGCMEKDEKGQEYQKGGCGGFGKGNFSQLFKSIEDYEKSLEAKQAAAAQGP

SEQ ID NO: 27 - DNA, 1323 bp, Sorghum halapense
atgcctccgaccccgaccaccgcagcagcaacaggtgccgcagttgcagcagcaagcgcagaacaggcagcatttcgtctggttggtcatcgtaattttgttcgtgttaatccgcgtagcgatcgttttcataccctggcatttcatcatgttgaactgtggtgtgccgatgcagccagcgcagcaggtcgttttagctttggtctgggtgcaccgctggcagcacgtagcgatctgagcaccggtaataccgcacatgcaagcctgctgctgcgttcaggtgcactggcatttctgtttaccgcaccgtatgcccatggtgctgatgcagcaaccgcaagcctgccgagctttagcgcagcagaagcacgtcgttttgcagcagatcatggtctggcagttcgtgccgttgcactgcgtgttgcagatgccgaagatgcatttcgtgcaagcgttgcagccggtgcacgtccggcatttgaaccggttgaactgggtctgggttttcgtctggccgaagttgaactgtatggtgatgttgttctgcgttatgttagctatccggatgatgcagatgcaagctttctgccgggttttgttggtgttaccagtccgggtgcggcagattatggcctgaaacgttttgatcatattgtgggtaatgttccggaactggcaccggcagcggcatattttgcaggttttaccggctttcatgaatttgcagaatttaccgcagaagatgttggcaccaccgaaagcggtctgaatagcatggttctggcaaataatgccgaaaatgttctgctgccgctgaatgaaccggtgcatggcaccaaacgtcgtagccagattcagacctttctggatcatcatggtggtccgggtgttcagcacatggcactggcaagtgatgatgtgctgcgtaccctgcgtgaaatgcaggcacgtagtgcaatgggtggttttgaatttatggcaccgccggcaccggaatattatgatggtgttcgtcgtcgtgccggtgatgttctgaccgaagcacagattaaagaatgtcaggaactgggcgttctggttgatcgtgatgatcagggtgttctgctgcagatttttaccaaaccggttggtgatcgcccgaccttttttctggaaattattcagcgtattggttgcatggaaaaagatgaaaaaggccaggaatatcagaaaggcggttgtggtggttttggtaaaggtaattttagccagctgtttaaaagcattgaagattatgaaaaaagcctggaagcaaaacaggcagctgcagcacagggtccgtaa

SEQ ID NO: 28 - protein, 453 aa, Sorghum halapense
MPPTPTTAAATGAAVAAASAEQAAFRLVGHRNFVRVNPRSDRFHTLAFHHVELWCADAASAAGRFSFGLGAPLAARSDLSTGNTAHASLLLRSGALAFLFTAPYAHGADAATASLPSFSAAEARRFAADHGLAVRAVALRVADAEDAFRASVAAGARPAFEPVELGLGFRLAEVELYGDVVLRYVSYPTTRTRPSCRGSWADDADASFLPGFVGVTSPGAADYGLKRFDHIVGNVPELAPAAAYFAGFTGFHEFAEFTAEDVGTTESGLNSMVLANNAENVLLPLNEPVHGTKRRSQIQTFLDHHGGPGVQHMALASDDVLRTLREMQARSAMGGFEFMAPPAPEYYDGVRRRAGDVLTEAQIKECQELGVLVDRDDQGVLLQIFTKPVGDRPTFFLEIIQRIGCMEKDEKGQEYQKGGCGGFGKGNFSQLFKSIEDYEKSLEAKQAAAAQGP

SEQ ID NO: 29 - DNA, 1311 bp, Poa annua
atgcctccgaccaccgcaaccgccaccgcagcagcaaccgttacaccggaacatgcagcacgtcgttttccgcgtgttgttcgtgttaatccgcgtagcgatcgttttccggttctgagctttcatcatgttgaattttggtgtgccgatgcagcaagcgcagcaggtcgttttagctttgcactgggtgcaccgctggcagcacgtagcgatctgagcaccggtaatagcgcacatgcaagcctgctgctgcgttcaggtgcactggcatttctgtttaccgcaccgtatgcaccgcagccgcaggatgcagataccgcaagcattccgagctttagcgcagatgcagcacgtgcatttagcgcagcacatggtctggcagttcgtagcgttgcagttcgtgttgcagatgccgcagatgcatttcgtgcaagcattgcagccggtgcacgtccggcatttgcaccggcagatctgggtcgtggttttggtctggccgaagttgaactgtatggtgatgttgttctgcgttttgttagccatccggatgcagatgatgcaccgccgtttctgccgggttttgaagcagttagccgtcgtccgggtgccgttgattatggtctgacccgttttgatcatgttgttggtaatgttccggaaatgggtccggtgattgattatattaaaggctttatgggctttcatgaatttgccgaatttaccgcagaagatgttggcaccaccgaaagcggtctgaatagcgttgttctggcaaataatagcgaagcagttctgctgccgctgaatgaaccggtgcatggcaccaaacgtcgtagccagattcagacctatctggaatatcatggtggtccgggtgttcagcatattgcactggcaagcagtgatgttctgcgtaccctgcgtgaaatgcaggcacgttcagcaatgggtggttttgaatttatggcaccgccgcagccgaaatattatgaaggtgttcgtcgtattgccggtgatgttctgagcgaagcacagattaaagaatgtcaggaactgggcgttctggttgatcgtgatgatcagggtgttctgctgcagatttttaccaaaccggttggtgatcgtccgaccttttttctggaaatgattcagcgtattggctgcatggaaaaagatgaacgtggtcaggaatatcagaaaggcggttgtggcggttttggtaaaggtaattttagcgaactgtttaaaagcattgaagattatgaaaaaagcctggaagccaaacagagcgcagttgcacagcagagctaa

SEQ ID NO: 30 - protein, 436 aa, Poa annua
MPPTTATATAAATVTPEHAARRFPRVVRVNPRSDRFPVLSFHHVEFWCADAASAAGRFSFALGAPLAARSDLSTGNSAHASLLLRSGALAFLFTAPYAPQPQDADTASIPSFSADAARAFSAAHGLAVRSVAVRVADAADAFRASIAAGARPAFAPADLGRGFGLAEVELYGDVVLRFVSHPDADDAPPFLPGFEAVSRRPGAVDYGLTRFDHVVGNVPEMGPVIDYIKGFMGFHEFAEFTAEDVGTTESGLNSVVLANNSEAVLLPLNEPVHGTKRRSQIQTYLEYHGGPGVQHIALASSDVLRTLREMQARSAMGGFEFMAPPQPKYYEGVRRIAGDVLSEAQIKECQELGVLVDRDDQGVLLQIFTKPVGDRPTFFLEMIQRIGCMEKDERGQEYQKGGCGGFGKGNFSELFKSIEDYEKSLEAKQSAVAQQS

SEQ ID NO: 31 - DNA, 1305 bp, Poa annua
atgccaccaactactgctactgctacagctgctgctactgttactccagaacatgctgctagaaggttcccaagagttgttagagttaacccaaggtctgataggttcccagttctttctttccaccacgttgaattttggtgtgctgatgctgcttctgctgctggaagattttcttttgctcttggtgctccacttgctgctagatctgatttgtctactggaaattctgctcacgcttctttgcttttgaggtctggtgctcttgctttcctttttactgctccttatgctccacaaccacaggatgctgatactgcatcaattccatctttctcagctgatgctgcaagggctttttctgctgctcatggattggctgttagatctgttgctgttagagttgctgatgcagctgatgctttcagagcttctattgctgcaggtgctagaccagcttttgctccagctgatcttggaagaggatttggacttgctgaggttgagctttacggtgatgttgttcttagattcgtgtctcacccagatgctgatgatgctccatttcttccaggatttgaggctgtttctagaccaggtgctgttgattatggactcactaggttcgatcacgttgtgggaaatgttccagaaatgggaccagtgatcgattacatcaagggattcatgggattccatgagttcgctgagtttactgctgaggatgttggaactactgagtctggacttaactctgttgtgcttgctaacaactctgaggctgttcttttgccacttaatgagccagttcacggcactaagagaagatctcagattcagacttaccttgagtaccatggtggaccaggtgttcaacatattgctcttgcttcatctgatgtgcttaggactcttagagagatgcaagctagatctgctatgggaggatttgagtttatggctccaccacaacctaagtattacgagggtgttagaaggattgctggtgatgttctttccgaggctcaaatcaaagagtgtcaagagcttggagtgcttgtggatagagatgatcagggtgtgcttctccagattttcactaagccagttggagataggccaacattcttcttggagatgattcagaggatcggctgcatggaaaaggatgagagaggtcaagagtatcaaaagggcggatgtggtggatttggaaagggaaatttctccgagcttttcaagtccatcgaggattacgagaagtctcttgaggctaagcaatctgctgttgctcaacagtcttga

SEQ ID NO: 32 - protein, 434 aa, Poa annua
MPPTTATATAAATVTPEHAARRFPRVVRVNPRSDRFPVLSFHHVEFWCADAASAAGRFSFALGAPLAARSDLSTGNSAHASLLLRSGALAFLFTAPYAPQPQDADTASIPSFSADAARAFSAAHGLAVRSVAVRVADAADAFRASIAAGARPAFAPADLGRGFGLAEVELYGDVVLRFVSHPDADDAPFLPGFEAVSRPGAVDYGLTRFDHVVGNVPEMGPVIDYIKGFMGFHEFAEFTAEDVGTTESGLNSVVLANNSEAVLLPLNEPVHGTKRRSQIQTYLEYHGGPGVQHIALASSDVLRTLREMQARSAMGGFEFMAPPQPKYYEGVRRIAGDVLSEAQIKECQELGVLVDRDDQGVLLQIFTKPVGDRPTFFLEMIQRIGCMEKDERGQEYQKGGCGGFGKGNFSELFKSIEDYEKSLEAKQSAVAQQS

SEQ ID NO: 33 - DNA, 1323 bp, Lolium multiflorum
atgcctccgacaccggcaaccgcaaccggtgctgcagcagcagcagttacaccggaacatgcagcacgtagctttccgcgtgttgttcgtgttaatccgcgtagcgatcgttttccggttctgagctttcatcatgttgaactgtggtgtgccgatgcagcaagcgcagcaggtcgttttagctttgcactgggtgctccgctggcagcccgtagcgatctgagcaccggtaatagcgcacatgcaagcctgctgctgcgtagcggtgcactggcatttctgtttaccgcaccgtatgcaccgcctccgcaggaagcagcaaccgcagctgcaaccgcaagcattccgagctttagcgcagatgcagcccgtacctttgcagcagcacatggtctggcagttcgtagcgttggtgttcgtgttgccgatgcagcggaagcatttcgtgttagcgttgccggtggtgcacgtccggcatttgcaccggcagatctgggtcatggttttggtctggccgaagttgaactgtatggtgatgttgttctgcgttttgttagctatccggatgaaaccgatctgccgtttctgccgggttttgaacgtgttagcagtccgggtgccgttgattatggtctgacccgttttgatcatgttgttggtaatgttccggaaatggcaccggttattgattatatgaaaggctttctgggctttcatgaatttgcagaatttaccgcagaagatgttggcaccaccgaaagcggtctgaatagcgttgttctggcaaataatagcgaaaatgttctgctgccgctgaatgaaccggtgcatggcaccaaacgtcgtagccagattcagacctatctggattatcatggtggtccgggtgttcagcatattgcactggcaagcaccgatgttctgcgtaccctgcgtgaaatgcgtgcacgtaccccgatgggtggttttgaatttatggcaccgccgcaggcaaaatattatgaaggtgttcgtcgtattgccggtgatgttctgagcgaagaacaaattaaagaatgtcaggaactgggcgttctggttgatcgtgatgatcagggtgttctgctgcagatttttaccaaaccggttggtgatcgtccgaccttttttctggaaatgattcagcgtattggctgcatggaaaaagatgaagttggtcaggaatatcagaaaggcggttgtggtggttttggtaaaggtaattttagcgaactgtttaaaagcattgaagattatgaaaaaaccctggaagccaaacagagcgttgttgcacagaaaagctaa

SEQ ID NO: 34 - protein, 440 aa, Lolium multiflorum
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSENVLLPLNEPVHGTKRRSQIQTYLDYHGGPGVQHIALASTDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKTLEAKQSVVAQKS

SEQ ID NO: 35 - DNA, 1053 bp, Synechococcus sp
atgaacccgtccattcgaattgtccaagggatccaccacctgcacttctacctttgggatctgccccgttggcgggaacacttttgtcgggtttggggcttccgggtggcaagcgacgccggcaacaccctggagctggagcagggatccctgcgcttgcgcctgtctcagccggcacgggcgggggacgaggtggaccgccatttgcagcggcatgggccgggggtggtggatgtggccttggcggtgggagagcaggagctaccggccttggcggagctgttgcggggccgaggcgcccaactggcgtggatcccggcagcagcggcgctctgcctccacaccccctacgggatccggcattctctgatccctggccccttggatgccgcccctgccgaagcgggcctgttttcccactgggatcacgtggtgttgaacgtggagcagggatccctgcaggcggcagccgactggtatgggcgggtgctgggctggcggcggctgtaccgctacagcatcggcaccgccacctccggcctggaaagcgtggtggtgggggatccggaagcggggatccaatgggccatcaacgagcccacctgtgccgcttcccagattcaggagtttttgcatgcccatggcggcccgggcattcagcacgcggcgctgcacagctcagacattgttgccagcctgcgccggttgcggcaggggggagtggactttttgcaagtggcgccgcagtactacaccagcctggaaagggagctggggttggcgctccgttctgcccttgggcaggccatctcctggcaagacctggtggagcagcagatccttctggatgctaccctgcccgcttctgatggccaggatcgcccccttctgctgcagacctttacccagcccctctttggtcggcccacctttttctttgaagtcattcaacggctaggcggggccacgggctttggcgaggccaattttcaggctttgttcgaggccctggaacggcaacagcgacagcgacaccaggcgctgaccccttag

SEQ ID NO: 36 - protein, 350 aa, Synechococcus sp
MNPSIRIVQGIHHLHFYLWDLPRWREHFCRVWGFRVASDAGNTLELEQGSLRLRLSQPARAGDEVDRHLQRHGPGVVDVALAVGEQELPALAELLRGRGAQLAWIPAAAALCLHTPYGIRHSLIPGPLDAAPAEAGLFSHWDHVVLNVEQGSLQAAADWYGRVLGWRRLYRYSIGTATSGLESVVVGDPEAGIQWAINEPTCAASQIQEFLHAHGGPGIQHAALHSSDIVASLRRLRQGGVDFLQVAPQYYTSLERELGLALRSALGQAISWQDLVEQQILLDATLPASDGQDRPLLLQTFTQPLFGRPTFFFEVIQRLGGATGFGEANFQALFEALERQQRQRHQALTP

SEQ ID NO: 37 - DNA, 1149 bp, Blepharisma japonicum
atgacttattacgacaagcaagaaacgcgtccagatcttggcgaattctatggtttccatcacgttcgtttttacgtctccaactcagagcaagccgcttcgttctacacatctcgctttgggttttctccggttgcctatgaaggattggaaacaggaaaccaaaaattctgtaccaatgtcgtccgaagcaaccatgtagtcatcgcttttacctcagctctcactcctgaagacaatgaagtgaaccgtcacgttggcaagcatagtgatggagttcaagacattgcctttagtgtaagtgacgcaagagggatgtatgagaaagcgatagctaaaggctgtaaaagcttccgtgagccacaggttttacaagatcaatttggatctgttataatagcgtctctccagacttatggagacactgttcacacattagtccaaaatgtcgactatacaggaccctttttgcctggcttcagagcaatcacaaaagatgatccattaaactctgcctttcctcaggtaaattatgacattattgatcatgttgtaggaaatcagcctggtggcgatatgactcctacagtagaatggtatgagaaatatctagaatttcatcgatattggtctgctgatgagtctgtaatccataccgattattcagcattaaggtctgttgtggttgctgattgggatgaagtgatcaaaatgcctattaatgagcctgctgatggacttagaaaaagtcaaatccaagaatatgtcgaatattatggtggagcaggcgtacaacatattgccttaaaagtcaatgatattatttcagtaataagcaccttaagggctagaggtgtggaattcttagaagttcctcctaaatattatgatagcttaagaaaaagacttgcgcattctgcggtacaaattgaagaagacttaaaaagaattgaagaccttcatattttggttgactttgacgaccgtgggtatttacttcagattttcacaaaaccagtagaagacagacctactctgttttatgaaattattcaaagacataataacaatggattcggaattggaaattttaaagccctatttgaatcattggaacaagagcaagaaagaagaggtaatttgatctaa

SEQ ID NO: 38 - protein, 350 aa, Blephrisman japonicum; identical to SEQ ID NO: 36

SEQ ID NO: 39 - DNA, 1107 bp, Picrophilus torridus
atgtatggcaaaaatttaatctcagaactaagggaaaaggagatctttaaacgattacatcacgtggaattttacgttagcagtgccaaaacatggtcatatttcatgaacaggggtcttggatttaaaacagtggcatatgccggtccagaaaccgggataagggacaagatatcctatgttatgtcccagggcactgcaaggatatcttttacatcatcaatgaatgatgatagctatatatcgaatcatgttaaaaaacacggggatggcgtaaaggatatagcacttgaggtcgatgatctggacgaggcaaaaagcctgatagaaaagtatggaacaaaggtttcaaaaataaatgaaataaaggatggaaatggaaagataagaactgcagagataaaaacgtacggtgaaaccgttcatacattaatagaaaccggggattacaatggcgtattcatgcccggttatgaggaatctgaaataaattcaaaaaacactgggataaaaaagatcgatcatatagttggaaatgtctatgagggcgagatggatagctgggttaatttttacatagaaaaacttggctttgagcatttaataacctttgatgataaagatataagaactgattacagcgcattaagatcaaaggttgtaaaatacaatgacgatatcgtatttccaataaatgagcctgcaaagggcttaagaaaatcacagatagaggaatatcttgactattacaggtctgagggcgttcagcacatagcactgttaactgatgatataataaaaactgtatccatgatggaggaaaacggcatagaatttttaaaaacaccaggatcatactatgaatccctatcatcaaggataggctcaatagacgaggatttaaatgaaatagagaaacataacatacttgtggatcgtgatgagaacggatacctattacagatcttcacaaagcctgttactgacaggccaacgttcttctttgaggtcatacagagaaagggtgcaaggtcattcggcaacggtaactttaaggcactttttgaggcgatagaaagggagcaggcaaagagaggaaacctatga

SEQ ID NO: 40 - protein, 368 aa, Picrophilus torridus
MYGKNLISELREKEIFKRLHHVEFYVSSAKTWSYFMNRGLGFKTVAYAGPETGIRDKISYVMSQGTARISFTSSMNDDSYISNHVKKHGDGVKDIALEVDDLDEAKSLIEKYGTKVSKINEIKDGNGKIRTAEIKTYGETVHTLIETGDYNGVFMPGYEESEINSKNTGIKKIDHIVGNVYEGEMDSWVNFYIEKLGFEHLITFDDKDIRTDYSALRSKVVKYNDDIVFPINEPAKGLRKSQIEEYLDYYRSEGVQHIALLTDDIIKTVSMMEENGIEFLKTPGSYYESLSSRIGSIDEDLNEIEKHNILVDRDENGYLLQIFTKPVTDRPTFFFEVIQRKGARSFGNGNFKALFEAIEREQAKRGNL

SEQ ID NO: 41 - DNA, 1164 bp, Kordia algicida
atggcagcagaaataaaaaacttaaaagatttacaaaatacagaatacggactcaaaaaattatttgacgaagcagaagactttcttccacttttaggaacagactacgtagaattatacgtcgggaacgccaaacaatcggcacatttctacaaaacggcttttggttttcaatcagaagcttacgcaggattggaaacaggattaaccgacagagtttcatacgtattaaaacaagataaaattcgcttggtcttaacaacaccattaggaaaaggtggcgaaatcaatgagcatatcgatttacacggcgatggcgtaaaagtagtagcactttgggtagaagatgctacaaaagcctttgaagaaacgaccaaaagaggcgcaaaaccgtacatggaaccaacaaaagaagaagatgaaaacggatatgtaattcgctcaggaatctatacgtacggagaaacggttcatgtttttgtagaacgtaaaaactataacggagtctttttaccaggatatcaaagatgggaatctcactacaatccggagccagttggcttaaaattcatcgatcacatggtaggaaatgtaggttggggagaaatgaaagaatggtgtgaattctacgcgaaagtaatgggatttgcgcaaattatctcctttacagatgatgatatttctaccgattttactgcgttgatgagtaaagtaatgagtaatggaaatggtagaatcaaatttccaatcaatgaacccgcagaaggaaaaaagaaatcgcaaattgaagaatatctagacttttacaatggttcaggagtacaacatattgcggttgctacagacaatattattgatacggtttcgcaaatgcgcgaacgtggagtagaattcttatacgttccagatacatattatgatgacttgttagaacgtgttggcgacatcgatgaagatgtagaagaactcaaaaaacacggaatcttaattgatcgtgatgaagaaggatacttattgcagttatttaccaaaaccattgtagacagaccaacaatgttctttgaagtcattcagcgtaaaggcgcacaatcatttggagtaggaaactttaaagctttatttgaagcgatagaaagagaacaagctgctcgcggaacattgtaa

SEQ ID NO: 42 - protein, 368 aa, Kordia algicida; identical to SEQ ID NO: 40

SEQ ID NO: 43 - DNA, 1206 bp, Rhodococcus sp.
atgacgatcgagcagactctcaccgacaaggaacgcctggcaggtctcgacctcggccagctcgagcagttggtcgggctcgtcgagtacgacggcacccgcgacccgttcccggtcagcggctgggatgccgtcgtctgggtggtcggcaacgccacccagaccgcccactacttccagtccgcgttcgggatgaccctcgtcgcctactccggacccaccaccggcaaccgcgaccaccacagcttcgtcctcgaatccggggccgtccgcttcgtcatcaaaggcgccgtgaacccggacagccccctgatcgaccaccaccgcacccacggcgacggcgtcgtcgacatcgccctcgccgtccccgacgtcgacaagtgcatcgcccacgcccgcgcccagggcgccaccgtcctcgacgaaccccacgacgtgaccgacgaccacggcaccgtccgcctcgccgcgatcgccacctacggcgacacccgccacaccctcgtcgaccgcagccactacaccggcccctacctgcccggctacaccgcccgcacctccggccacaccaaacgggacggggcacccaagcgcctgttccaggccctcgaccacgtcgtcggcaacgtcgaactcggcaagatggaccactgggtcgacttctacaaccgggtcatgggctttacgaacatggccgagttcgtcggcgaggacatcgccaccgactactccgcgctgatgagcaaggtcgtctccaacggcaaccaccgggtcaagttccccctcaacgaacccgccctcgccaagaaacgctcgcagatcgacgaatacctcgacttctaccgcggccccggcgcccagcacctggccctggccaccaatgacatcctcaccgccgtcgaccagctgaccgccgagggcgtcgagttcctggccacccccgactcctactacgaggaccccgaactgcgggcccggatcggcaacgtccgcgcccccatcgccgaactgcagaaacgcggcatcctcgtcgaccgcgacgaagacggctacctgctgcagatcttcaccaaacccctcgtcgaccggcccaccgtgttcttcgaactcatcgaacgccacggctccctcggcttcggcatcggcaacttcaaagccctcttcgaggccatcgaacgcgaacaagccgcccgcggaaacttctga

SEQ ID NO: 44 - protein, 401 aa, Rhodococcus sp.
MTIEQTLTDKERLAGLDLGQLEQLVGLVEYDGTRDPFPVSGWDAVVWVVGNATQTAHYFQSAFGMTLVAYSGPTTGNRDHHSFVLESGAVRFVIKGAVNPDSPLIDHHRTHGDGVVDIALAVPDVDKCIAHARAQGATVLDEPHDVTDDHGTVRLAAIATYGDTRHTLVDRSHYTGPYLPGYTARTSGHTKRDGAPKRLFQALDHVVGNVELGKMDHWVDFYNRVMGFTNMAEFVGEDIATDYSALMSKVVSNGNHRVKFPLNEPALAKKRSQIDEYLDFYRGPGAQHLALATNDILTAVDQLTAEGVEFLATPDSYYEDPELRARIGNVRAPIAELQKRGILVDRDEDGYLLQIFTKPLVDRPTVFFELIERHGSLGFGIGNFKALFEAIEREQAARGNF

SEQ ID NO: 45 - DNA, 1209 bp, Rhodococcus sp
atgactaccgccgacattcgcctgacgccccgcgaggtggccgcacatctggagaccgacgagctccggcagttggtcgggctcgtcgaacacgacgacgcgtcggatccgtttcccgtggtcgcgatggatgccgtggtgttcgtgtgcggcaacgcgacgcagagcacgcagtacttcgtctccacgtggggcatgaccctcgtcgcctacgccgggccggagaccggtcagcgctcgcacaagtccttcgtcctcgagtcggggtcggcacggttcgtgctgcacggcgccgtcgatccgaagagcccgctcgcggaccatcaccgggcgcacggcgacggcgtggtggacctggcgatggaagttctcgacgtcgaccgctgcatcgcgcatgcacgctcgcagggggccaccattctcgaggagccgcgcgacgtcacggatcagttcggcaccgtgcggctcgcggcgatcgccacgtacggcagcacccggcacaccatcgtcgaccgaagccgatacgacggcccctacctccccggattcgtcgcgcgctccagcggtttcgcggcgcgaccgggtaaacccccgcgattgttccaggcgctcgaccacgccgtcggcaacgtcgagatgggccggatggatcactgggtccggttctacaaccgcgtcatgggcttcacgaacatggccgaattcgtcggcgacgacatcgccacggagtactcggcgctgatgtcgaaggtcgtggcgaacggcaatcaccgggtgaagttcccgctcaacgaacccgcggtgggaaagaagaagtcgcagatcgacgaatatctcgagttctacggtgagccgggctgccagcatctggccctcgcgaccggagacatcctcgcgacggtggacgcgttgcgggccgagggtgtcgaattcctgaacacacccgacgcgtactacgaggacccacagctgcgcgcccggatcggcagggtgcgggtgccggtggaggaactgcagaagcgcggaatcctcgtcgaccgcgacgaggacggatacctcctgcagatcttcaccaaaccgctcggcgaccggccgaccgtgttcttcgaggtgatcgaacggcacggttcgctcgggttcggggcgggtaacttccaggccctgttcgaatccatcgagcgtgagcaggcggcgcgcggcaatctgtga

SEQ ID NO: 46 - protein, 402 aa, Rhodococcus sp
MTTADIRLTPREVAAHLETDELRQLVGLVEHDDASDPFPVVAMDAVVFVCGNATQSTQYFVSTWGMTLVAYAGPETGQRSHKSFVLESGSARFVLHGAVDPKSPLADHHRAHGDGVVDLAMEVLDVDRCIAHARSQGATILEEPRDVTDQFGTVRLAAIATYGSTRHTIVDRSRYDGPYLPGFVARSSGFAARPGKPPRLFQALDHAVGNVEMGRMDHWVRFYNRVMGFTNMAEFVGDDIATEYSALMSKVVANGNHRVKFPLNEPAVGKKKSQIDEYLEFYGEPGCQHLALATGDILATVDALRAEGVEFLNTPDAYYEDPQLRARIGRVRVPVEELQKRGILVDRDEDGYLLQIFTKPLGDRPTVFFEVIERHGSLGFGAGNFQALFESIEREQAARGNL

SEQ ID NO: 47 - DNA, 1395 bp, Scenedesmus obliquus
atgggcgcaggtggagctcagggcccgaaggttgagctggttggctacgccaatttcgttcgcaacaaccctcgcagcgataagttccctgtgcacaagtttcaccacatcgagttttggtgcgcggacgccaccaacaccttcaagcgcttccagcatggcctgggcatgaccctagtggccaagtccgaccacagcacaggcaacagcaagtactgcagctacgtcttgcaaagcaacgacctcgtgttcaccttcacagcaccctactcgcgcaagtgcgcagcagcagcgccttctagcagcgagccgctgccagattatgaccagcagcaggcctttgaattcatctgcacccacggcctggcggcaagggctgtaggcctgcaggtgggcgatgcagcgcaggcgtacgaggtttctgtggcaaacggcgcgaagggtgtgcgtccacccaccaagctggaggatggcggcggctgtgctgtggtcagcgaggtgctgctgtatggtgacgtggtgctgcgctacatcagcggcaagtgggagggcccctacctgccgggctacacagccacgcctgatgagccgcagatctgctacggcctgcaccgcctggaccacgccgtgggcaatgtgcccaagctcatcgagcacttggagcacgtcattggcttcacaggcttccacgagtttgccgagtttgtggcggaggacgtgggcactgtggacagcgggctgaacagcatggtgctggccagcaacaacgagatggtgctgctgcccatgaacgagcccacctttggcaccaagcgcaagtcgcagatccagacgtacctggagcagaacgagggccccgggctgcagcacctggcgctgaagacgcacgacatcctgtccaccatgcgcgagatgcacgcacgctcgcgctgcggcggcttcgagttccaggcggcacccgggcacgactactacaagcgcgtggcggagaaagtgggtgacgtgctgtccccggaggagtgggcagccgttgagcagctgggcatcctggtggaccaggacgaccagggcgtgctgctgcagatcttcaccaagccgctgggggacaggcccaccatcttcattgagatcattgagcggcgcggctgcctcaaggagagcgcggcacaggcaggcagtgcggcagcagcagcagcagagcctacagcagctggcggtgatgcagatgcagatggagcagcagcagcagtggctgacaagttcaaagacatcgtgcaggtgcgtgaggatggcgtggttgtggagcaagctgctggctgtggaggcttcggcaaaggcaacttcagcgagctgttcaaaagcattgaggagtacgagcgcacgctgcaggtgtaa

SEQ ID NO: 48 - protein, 464 aa, Scenedesmus obliquus
MGAGGAQGPKVELVGYANFVRNNPRSDKFPVHKFHHIEFWCADATNTFKRFQHGLGMTLVAKSDHSTGNSKYCSYVLQSNDLVFTFTAPYSRKCAAAAPSSSEPLPDYDQQQAFEFICTHGLAARAVGLQVGDAAQAYEVSVANGAKGVRPPTKLEDGGGCAVVSEVLLYGDVVLRYISGKWEGPYLPGYTATPDEPQICYGLHRLDHAVGNVPKLIEHLEHVIGFTGFHEFAEFVAEDVGTVDSGLNSMVLASNNEMVLLPMNEPTFGTKRKSQIQTYLEQNEGPGLQHLALKTHDILSTMREMHARSRCGGFEFQAAPGHDYYKRVAEKVGDVLSPEEWAAVEQLGILVDQDDQGVLLQIFTKPLGDRPTIFIEIIERRGCLKESAAQAGSAAAAAAEPTAAGGDADADGAAAAVADKFKDIVQVREDGVVVEQAAGCGGFGKGNFSELFKSIEEYERTLQV

SEQ ID NO: 49 - DNA, 1395 bp, Scenedesmus obliquus; identical to SEQ ID NO: 47

SEQ ID NO: 50 - protein, 430 aa, Scenedesmus obliquus
MGAGGAQGPKVELVGYANFVRNNPRSDKFPVHKFHHIEFWCADATNTFKRFQHGLGMTLVAKSDHSTGNSKYCSYVLQSNDLVFTFTAPYSRKCAAAAPSSSEPLPDYDQQQAFEFICTHGLAARAVGLQVGDAAQAYEVSVANGAKGVRPPTKLEDGGGCAVVSEVLLYGDVVLRYISGKWEGPYLPGYTATPDEPQICYGLHRLDHAVGNVPKLIEHLEHVIGFTGFHEFAEFVAEDVGTVDSGLNSMVLASNNEMVLLPMNEPTFGTKRKSQIQTYLEQNEGPGLQHLALKTHDILSTMREMHARSRCGGFEFQAAPGHDYYKRVAEKVGDVLSPEEWAAVEQLGILVDQDDQGVLLQIFTKPLGDRPTIFIEIIERRGCLKESAAQAGSAAAAVEQAAGCGGFGKGNFSELFKSIEEYERTLQVLE

SEQ ID NO: 51 - DNA, 1305 bp, Hordeum vulgare
atggcacctccaactccaacaacacctgccgctactggtgcagccgcagctgtaactcctgaacatgcgaggccacatcggatggttcgattcaatccgagatctgatagattccatactctgagcttccatcatgtggaattctggtgtgctgatgcagcttctgcagctggacgtttcgcttttgcccttggagctcctttagcagcgagatcagacttgagcacaggaaacagtgcacacgcatctcaacttctgcgttcaggaagccttgcgttcctgtttactgcaccgtatgctaacggatgcgacgcagcaactgcctcacttccttctttcagtgcagatgcagctagacgattctcagccgatcatggaattgcagtcagatctgtggctttgcgagttgctgatgctgccgaagctttcagggcatcagttgatggaggtgctaggcctgcttttgctcctgtggacttgggtagaggatttggctttgccgaggtcgaactctatggtgatgtggttctccggtttgtctctcacccagatggaacagatgttcctttcttgccagggtttgagggagtgacaaacccagatgcggtagattacggtctcacgagattcgaccatgtagtgggcaatgtaccggaattggctcctgcggctgcttacatagctggctttacgggatttcacgaattcgcggaattcaccgctgaggatgtcggaaccacagaatcagggctgaattccgtcgtccttgccaacaattccgaaggggtattgctgcctcttaacgagcctgtgcatggcacgaaaagacgtagccagatacagaccttcctagaacatcacggtggaccaggtgttcaacacattgctgttgccagcagtgatgtactcaggacgcttcgtaagatgagagctaggagtgcgatgggagggtttgactttctaccacctccgctgccaaaatactatgagggtgtgaggagactggctggtgatgttttgtctgaagcgcagatcaaggagtgtcaggaattaggggtgctcgttgacagagatgatcaaggggtgcttctccagatctttactaagccggttggtgataggcctaccctctttctagagatgattcaacgtatcgggtgtatggaaaaggacgagagaggtgaggagtatcaaaagggtggatgcggcggttttgggaaaggtaatttctccgagctgttcaagtcgatcgaagattacgagaaatcccttgaggcgaaacaatctgcagctgttcaaggatcg

SEQ ID NO: 52 - DNA, 1338 bp, Arabidopsis
atgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggccgacgatatttatagagataatccagagagtaggatgcatgatgaaagatgaggaagggaaggcttaccagagtggaggatgtggtggttttggcaaaggcaatttctctgagctcttcaagtccattgaagaatacgaaaagactcttgaagccaaacagttagtgggatga

SEQ ID NO: 53 - protein, 445 aa, Arabidopsis
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRPTIFIEIIQRVGCMMKDEEGKAYQSGGCGGFGKGNFSELFKSIEEYEKTLEAKQLVG

SEQ ID NO: 54 - DNA, 1299 bp, Chlamydomonas
atgggcgctggtggcgcttctaccacggtagcgaatggcgggatcaagttggtagggcacaagaattttgtgcgctataatccacaatccgaccggtttgctattaagaggttccatagcttcgagttctggtgcgcggatgcgaccaacacatacaagcggttctcttatggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagttgcgggatgaggcgagcggcaccagccaggtcatctcggaggtcattgtttacggggacgtcgttttccgctacgtgtcgggctccttcgagggccctttcatggccggctacacgccagtcacagactcgccggtcgcgtcgattgggttacagcgcgtggaccacgcggtgggcaacacacacgacctgatcaaggccgtggagtacatcaccgggttctgtggcttccacgagttctcagagtttgttgcggaggacgtgggcactgtggacagcggcctgaacagcatggtgcttgccaacaacgaggagaccatattgatgcctgtgaacgagcccaccttcggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccgcgggcaaatgcgaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaggagcctgctacgggcgctgtggtggggacggagcaggcggctggctgcggcggcttcgggaaaggcaacttcggcgccctcttcaagtccattgaggactatgagcgcaccctaaatgtgtag

SEQ ID NO: 55 - protein, 432 aa, Chamydomonas
MGAGGASTTVANGGIKLVGHKNFVRYNPQSDRFAIKRFHSFEFWCADATNTYKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVIVYGDVVFRYVSGSFEGPFMAGYTPVTDSPVASIGLQRVDHAVGNTHDLIKAVEYITGFCGFHEFSEFVAEDVGTVDSGLNSMVLANNEETILMPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 56 - DNA, 1299 bp, Chlamydomonas
atgggagcgggtggtgcaggcaccggagatcgggaggggggcattaagctcgtgggctacaagaatttcgtgcgccagaacccgctttcagacaaattcaccgtccacaagtttcatcacatcgatttctggtgcggagatgcaacaaacacatcgaagcggttctcctacggcctgggcatgccgctggtcgccaagtccgaccagtccaccaacaaccagctctttgcctcctacgtgctgcgctccaacgacctggtcttcaccttcaccgcgccctacagccgcaagtgcgcctcggtcagcgagggcgttccgctgcgtcactacaacatcgaccatgcgtatgagttcatcaactcgcacgggctggcggtgcgggcagtaggcctgctggtggatgacgccaagacggcgtacgaggtgtctgtggcgcacggggccaagggcgtgctgccgccggtggagctgcgggatgaggcgagcggcaccagccaggtcatctcggaggtgctgctgtacggcgaggtcgtgctgcgctacgtgtcgggctccttccagggccccttcctggccggctacacgcccgtcacagactcggccgtgacctccttcggcctgcaacgtctggaccacgcggtgggcaacacccatgacctgatcaaggccgtggagtacatcaccggcttcacaggtttccacgagttctcagagtttgttgcggaggacgtgggcactgtggacagcggcctgaacagcatggtgctggcctccaacaacgaggcagtgctgctgcctgtgaacgagcccacctttggcacgccgcgcaagagccaaatccagacctacctggagcagaacgaggggccggggctgcagcacctggcgctgctcagcaacgacatcttcaccaccctgcgggagatgcgcgcgcgcagcgagctgggtggcttcgagttcatgccacgggcaaatgccaagtactacaaagacatgtacgcccgcatcggcgactcgctcacgccgcagcagtacagggaggtggaggagctgggcatcctggtggacaaggacgaccagggcgtgctgctgcagatcttcaccaagccgctgggcgaccggcccacggtgtttattgagatcatccagcgtgtgggctgcatgcgggaggtgaaagagcctgctacgggcgctgtggtggggacggagcaggcggctggctgcggcggcttcgggaaaggcaacttcggtgccctcttcaagtccattgaggactatgagcgcaccttaaatgtttaa

SEQ ID NO: 57 - protein, 432 aa, Chlamydomonas
MGAGGAGTGDREGGIKLVGYKNFVRQNPLSDKFTVHKFHHIDFWCGDATNTSKRFSYGLGMPLVAKSDQSTNNQLFASYVLRSNDLVFTFTAPYSRKCASVSEGVPLRHYNIDHAYEFINSHGLAVRAVGLLVDDAKTAYEVSVAHGAKGVLPPVELRDEASGTSQVISEVLLYGEVVLRYVSGSFQGPFLAGYTPVTDSAVTSFGLQRLDHAVGNTHDLIKAVEYITGFTGFHEFSEFVAEDVGTVDSGLNSMVLASNNEAVLLPVNEPTFGTPRKSQIQTYLEQNEGPGLQHLALLSNDIFTTLREMRARSELGGFEFMPRANAKYYKDMYARIGDSLTPQQYREVEELGILVDKDDQGVLLQIFTKPLGDRPTVFIEIIQRVGCMREVKEPATGAVVGTEQAAGCGGFGKGNFGALFKSIEDYERTLNV

SEQ ID NO: 58 - protein, 433 aa, Physcomitrella
MGLDKSESEGSVVGPLHLVGCERFVRNNPKTDRFGVERFHHVEFWCGDASNTWRRFSWGLGMHLVAKSDQTTGNQTYCSYAIQSNELVFAFTAPYSSTIDQTNTKMPHPGYKSDEARSFTDSHGLAVRAVGILVDDADEAFRISVEHGAVSVLEPHVLSDDAKGGKMVMAEVKLYGDVVLRYVSEQGYKGSMLPNYEEVESLPLSYGLVRLDHAVGNVHNLAEAVNYIAKFTGFHEFAEFTAGDVGTTESGLNSMVVASNNEMVLLPINEPTFGTKRKSQIQTYLEHNEGPGLQHLALICDNIFSTLREMRTRTHIGGFDFMPKPPPTYYKNLANRVGDILTAEQIKECDELGILVDKDDQGVLLQIFTKPVGDRPSIFVEIIQRIGCMDKDESTGATVQKGGCGGFGKGNFSELFKSIEEYEKTLDGTLKVH

SEQ ID NO: 59 - protein, 446 aa, Oryza
MPPTPTPTATTGAVSAAAAAGENAGFRLVGHRRFVRANPRSDRFQALAFHHVELWCADAASAAGRFAFALGAPLAARSDLSTGNSAHASLLLRSASVAFLFTAPYGGDHGVGADAATTASIPSFSPGAARRFAADHGLAVHAVALRVADAADAFRASVAAGARPAFQPADLGGGFGLAEVELYGDVVLRFVSHPDGADAPFLPGFEGVSNPGAVDYGLRRFDHVVGNVPELAPVAAYISGFTGFHEFAEFTAEDVGTAESGLNSVVLANNAETVLLPLNEPVHGTKRRSQIQTYLDHHGGPGVQHIALASDDVLGTLREMRARSAMGGFEFLAPPPPNYYDGVRRRAGDVLSEEQINECQELGVLVDRDDQGVLLQIFTKPVGDRPTFFLEMIQRIGCMEKDESGQEYQKGGCGGFGKGNFSELFKSIEEYEKSLEAKQAPTVQGS

SEQ ID NO: 60 - protein, 436 aa, Triticum
MPPTPTTPAATGAGAAAAVTPEHARPRRMVRFNPRSDRFHTLSFHHVEFWCADAASAAGRFAFALGAPLAARSDLSTGNSVHASQLLRSGNLAFLFTAPYANGCDAATASLPSFSADAARRFSADHGLAVRSIALRVADAAEAFRASVDGGARPAFSPVDLGRGFGFAEVELYGDVVLRFVSHPDDTDVPFLPGFEGVSNPDAVDYGLTRFDHVVGNVPELAPAAAYVAGFAGFHEFAEFTTEDVGTAESGLNSMVLANNSEGVLLPLNEPVHGTKRRSQIQTFLEHHGGSGVQHIAVASSDVLRTLREMRARSAMGGFDFLPPRCRKYYEGVRRIAGDVLSEAQIKECQELGVLVDRDDQGVLLQIFTKPVGDRPTLFLEMIQRIGCMEKDERGEEYQKGGCGGFGKGNFSELFKSIEDYEKSLEAKQSAAVQGS

SEQ ID NO: 61 - protein, 444 aa, Zea
MPPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEEAFRTSVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFPKPVGDRPTLFLEIIQRIGCMERDEKGQEYQKGGCGGFGKGNFSQLFKSIEDYEKSLEAMQAAAAATAQGS

SEQ ID NO: 62 - protein, 443 aa, Glycine
  VARIANT 393..393
MCNEIQAQAQAQAQPGFKLVGFKNFVRTNPKSDRFQVNRFHHIEFWCTDATNASRRFSWGLGMPIVAKSDLSTGNQIHASYLLRSGDLSFLFSAPYSPSLSAGSSAASSASIPSFDAATCLAFAAKHGFGVRAIALEVADAEAAFSASVAKGAEPASPPVLVDDRTGFAEVRLYGDVVLRYVSYKDAAPQAPHADPSRWFLPGFEAAASSSSFPELDYGIRRLDHAVGNVPELAPAVRYLKGFSGFHEFAEFTAEDVGTSESGLNSVVLANNSETVLLPLNEPVYGTKRKSQIETYLEHNEGAGVQHLALVTHDIFTTLREMRKRSFLGGFEFMPSPPPTYYANLHNRAADVLTVDQIKQCEELGILVDRDDQGTLLQIFTKPVGDRPTIFIXIIQRIGCMVEDEEGKVYQKGACGGFGKGNFSELFKSIEEYEKTLEAKRTA

SEQ ID NO: 63 - protein, 445 aa, Vitis
MGKQNTTTNNPAPGFKLVGFSNFLRTNPMSDRFGVKRFHHIEFWSTDATNLARRFSWGLGMPIVAKSDLSTGNVIHASYLTRSGDLNFLFTAPYSPSIAGDLENAAATASIPSFDHSACHAFAASHGLGVRAIAIEVDDAEGAFHTSVAHGARPMSPPVTMGGSVVISEVHLYGDAVLRYVSYKNPNPNATSDPSSWFLPGFEAVDEGSSFPVDFGLRRVDHTVGNVPKLAPVVTYLKQFTGFHEFAEFTAEDVGTSESGLNSVVLASNNEMVLLPLNEPVFGTKRKSQIQTYLEHNEGPGVQHLALMSDDIFRTLREMRRRSGVGGFDFMPSPPPTYYRNVKKRAGDVLTDDQIKECEELGILVDKDDQGTLLQIFTKPLGDRPTIFIEIIQRLGCMVKDDEGKVSQKGGCGGFGKGNFSELFKSIEEYEKTLGAKRIVDPAPV

SEQ ID NO: 64 - protein, 358 aa, Pseudomonas fluorescens
MADQYENPMGLMGFEFIEFASPTPGTLEPIFEIMGFTKVATHRSKNVHLYRQGEINLILNNQPDSLASYFAAEHGPSVCGMAFRVKDSQQAYNRALELGAQPIHIETGPMELNLPAIKGIGGAPLYLIDRFGEGSSIYDIDFVYLEGVDRNPVGAGLKVIDHLTHNVYRGRMAYWANFYEKLFNFREARYFDIKGEYTGLTSKAMSAPDGMIRIPLNEESSKGAGQIEEFLMQFNGEGIQHVAFLTEDLVKTWDALKKIGMRFMTAPPDTYYEMLEGRLPNHGEPVDQLQARGILLDGSSIEGDKRLLLQIFSETLMGPVFFEFIQRKGDDGFGEGNFKALFESIERDQVRRGVLTTD

SEQ ID NO: 65 - protein, 358 aa, Pseudomonas fluorescens
MADLYENPMGLMGFEFIELASPTPNTLEPIFEIMGFTKVATHRSKDVHLYRQGAINLILNNEPHSVASYFAAEHGPSVCGMAFRVKDSQKAYKRALELGAQPIHIETGPMELNLPAIKGIGGAPLYLIDRFGEGSSIYDIDFVFLEGVDRHPVGAGLKIIDHLTHNVYRGRMAYWANFYEKLFNFREIRYFDIKGEYTGLTSKAMTAPDGMIRIPLNEESSKGAGQIEEFLMQFNGEGIQHVAFLSDDLIKTWDHLKSIGMRFMTAPPDTYYEMLEGRLPNHGEPVGELQARGILLDGSSESGDKRLLLQIFSETLMGPVFFEFIQRKGDDGFGEGNFKALFESIERDQVRRGVLSTD

SEQ ID NO: 66 - protein, 440 aa, Avena sativa
MPPTPATATGAAAAAVTPEHAARSFPRVVRVNPRSDRFPVLSFHHVELWCADAASAAGRFSFALGAPLAARSDLSTGNSAHASLLLRSGALAFLFTAPYAPPPQEAATAAATASIPSFSADAARTFAAAHGLAVRSVGVRVADAAEAFRVSVAGGARPAFAPADLGHGFGLAEVELYGDVVLRFVSYPDETDLPFLPGFERVSSPGAVDYGLTRFDHVVGNVPEMAPVIDYMKGFLGFHEFAEFTAEDVGTTESGLNSVVLANNSEAVLLPLNEPVHGTKRRSQIQTYLEYHGGPGVQHIALASNDVLRTLREMRARTPMGGFEFMAPPQAKYYEGVRRIAGDVLSEEQIKECQELGVLVDRDDQGVLLQIFTKPVGDRPTFFLEMIQRIGCMEKDEVGQEYQKGGCGGFGKGNFSELFKSIEDYEKSLEVKQSVVAQKS

SEQ ID NO: 67 - protein, 444 aa, Zea mays
MGPTPTAAAAGAAVAAASAAEQAAFRLVGHRNFVRFNPRSDRFHTLAFHHVELWCADAASAAGRFSFGLGAPLAARSDLSTGNSAHASLLLRSGSLSFLFTAPYAHGADAATAALPSFSAAAARRFAADHGLAVRAVALRVADAEDAFRASVAAGARPAFGPVDLGRGFRLAEVELYGDVVLRYVSYPDGAAGEPFLPGFEGVASPGAADYGLSRFDHIVGNVPELAPAAAYFAGFTGFHEFAEFTTEDVGTAESGLNSMVLANNSENVLLPLNEPVHGTKRRSQIQTFLDHHGGPGVQHMALASDDVLRTLREMQARSAMGGFEFMAPPTSDYYDGVRRRAGDVLTEAQIKECQELGVLVDRDDQGVLLQIFTKPVGDRPTLFLEIIQRIGCMEKDEKGQEYQKGGCGGFGKGNFSQLFKSIEDYEKSLEAKQAAAAAAAQGS

SEQ ID NO: 68 - DNA, 1332 bp, Zea mays
atgggtccgaccccgaccgcaacagcagccggtgcagcagttgcagcagcaagcgcagcagaacaggcagcatttcgtctggttggtcatcgtaattttgttcgttttaatccgcgttccgatcgttttcataccctggcatttcatcatgttgaactgtggtgtgcagatgcagcatcagcagcaggtcgttttagctttgcactgggtgcaccgctggcagcacgtagcgatctgagcaccggtaatagcgcacatgcaagtctgctgctgcgtagcggtagcctgagctttctgtttaccgcaccgtatgcacatggtgccgatgcagccaccgcagcactgccgagctttagcgcagccgcagcccgtcgttttgcagcagatcatggtctggcagttcgtgcagttgcactgcgtgttgcagatgccgaagatgcatttcgtgcaagcgttgcagcgggtgcacgtccggcatttggtccggttgatctgggtcgtggttttcgcctggccgaagtggaactgtatggtgatgttgttctgcgttatgttagctatccggatggtgccgcaggcgaaccgtttctgcctggttttgaaggtgttgcaagtccgggtgcagccgattatggcctgagccgttttgatcatattgttggtaatgtgccggaactggcaccggcagcagcatattttgcaggttttaccggttttcatgaatttgccgaatttaccaccgaagatgttggcaccgcagaaagcggtctgaatagcatggcactggcaaataatagcgaaaatgtactgctgccgctgaatgaaccggtgcatggcaccaaacgtcgtagccagattcagacctttctggatcatcatggtggtccgggtgttcagcatattgcactggcatcagatgatgtgctgcgtaccctgcgtgaaatgcaggcacgtagtgcaatgggtggctttgaatttctgcctccgcctccgagcgattattatgatggtgttcgtcgtgaagccggtgatgttctgaccgaagcacagattaatgaatgtcaagaactgggtgttatggtggatcgtgatgatcagggtgtcctgctgcagatttttaccaaaccggttggtgatcgtccgaccttttttctggaaattattcagcgtattggctgcatggaaaaagatgaaaaaggccaagaatatcagaaaggtggttgtggtggttttggcaaagataactttagccagctgtttaaaagcatcgaggattatgaaaaaagcctggaagcaaaacaagccgcagcagccgcaaccgcacagggtagc

SEQ ID NO: 69 - DNA, 1332 bp, Zea mays
atgggtccgaccccgaccgcaacagcagccggtgcagcagttgcagcagcaagcgcagcagaacaggcagcatttcgtctggttggtcatcgtaattttgttcgttttaatccgcgtagcgatcgttttcagaccctggcatttcatcatgttgaactgtggtgtgcagatgcagcatcagcagcaggtcgttttagctttgcactgggtgttccgctggcagcacgtagtgatctgagcaccggtaatagcgcacatgcaagtctgctgctgcgtagcggtagcctgagcctgctgtttaccgcaccgtatgcacatggtgccgatgcagccaccgcagcactgccgagctttagcgcagccgcagcccgtcgttttgcagcagatcatggtctggcagttcgtgcagttgccctgcgtgttgcagatgccgaagatgcatttcgtgcaagcgttgcagcgggtgcacgtccggcatttggtccggttgatctgggtcgtggttttcgcctggccgaagtggaactgtatggtgatgttgttctgcgttatgttagctatccggatggtgccgcaggcgaaccgtttctgcctggttttgaaggtgttgcaagtccgggtgcagccgattatggcctgagccgttttgatcatattgttggtaatgtgccggaactggcaccggcagcagcatatatggcaggttttaccggttttcatgaatttgcagaatttaccaccgaagatgttggcaccaccgaaagcggtctgaatagcatggcactggcaaataatagcgaaaatgtactgctgccgctgaatgaaccggtgcatggcaccaaacgtcgtagccagattcagacctttctggatcatcatggtggtccgggtgttcagcatattgcactggcatcagatgatgtgctgcgtaccctgcgtgaaatgcgtgcacgtagcgcaatgggtggctttgaatttctgcctccgcctctgagcgattattatgatggtgttcgtcgttgtgccggtgatgttctgaccgaagcacagattaatgaatgtcaagaactgggtgtgatggtggatcgtgatgatcagggtgtcctgctgcagatttttaccaaaccggttggtgatcgtccgaccttttttctggaaattattcagcgtattggctgcatggaaaaagatgaaaaaggccaagaatatcagaaaggtggttgtggtggttttggcaaaggtaattttggtcagctgtttaaaagcatcgaggactatgaaaaaagcctggaagcaaaacaagccgcagcagccgcaaccgcacagggtagc